SEQUENCE LISTING (4 sequences)

SEQ ID NO: 1 - DNA, 19 bp, Artificial Sequence
  source: /note=“Description of Artificial Sequence Synthetic primer”
ctgtgccttgggtggcttt

SEQ ID NO: 2 - DNA, 24 bp, Artificial Sequence
  source: /note=“Description of Artificial Sequence Synthetic primer”
aaggaaagaagtcagaaggcaaaa

SEQ ID NO: 3 - DNA, 25 bp, Artificial Sequence
  source: /note=“Description of Artificial Sequence Synthetic probe”
tcctttataagggtcgatgtccatg

SEQ ID NO: 4 - protein, 36 aa, Artificial Sequence
  source: /note=“Description of Artificial Sequence Synthetic polypeptide”
YTSLIHSLIEESQNQQEKNEQELLELDKWASLWNWF

(19) United States
(12) Patent Application Publication
(10) Pub. No.: US 2022/0048912 A1
(43) Pub. Date: Feb. 17, 2022

(54) CYCLOBUTYL NUCLEOSIDE ANALOGS AS ANTI-VIRALS

(71) Applicant: Janssen BioPharma, Inc., South San Francisco, CA (US)

(72) Inventors: Minghong ZHONG, San Bruno, CA (US); Guangyi WANG, Irvine, CA (US)

(21) Appl. No.: 17/413,185

(22) PCT Filed: Dec. 4, 2019
(86) PCT No.: PCT/IB2019/060430

§ 371 Date: Jun. 11, 2021

Related U.S. Application Data
(60) Provisional application No. 62/778,820, filed on Dec. 12, 2018.

(51) Int. Cl. C07D 473/18; C07D 239/47; C07D 473/34; C07D 239/54; C07D 487/04; C07F 9/6561; C07F 9/6512; A61K 45/06

(52) U.S. Cl. CPC C07D 473/18; C07D 239/47; C07D 473/34; A61K 45/06; C07D 487/04; C07F 9/65616; C07F 9/6512; C07D 239/54

(57) ABSTRACT

Described herein are cyclobutyl nucleoside analogs of Formula (I), pharmaceutical compositions that include one or more cyclobutyl nucleoside analogs and methods of using the same to treat HBV, HDV and/or HIV.

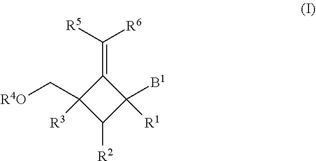

### FIELD

The present application relates to the fields of chemistry, biochemistry and medicine. More particularly, disclosed herein are cyclobutyl nucleoside analogs, pharmaceutical compositions that include one or more cyclobutyl nucleoside analogs and methods of synthesizing the same. Also disclosed herein are methods of treating viral diseases and/or conditions with a cyclobutyl nucleoside analog, alone or in combination therapy with one or more other agents.

### DESCRIPTION

Nucleoside analogs are a class of compounds that have been shown to exert antiviral activity both in vitro and in vivo, and thus, have been the subject of widespread research for the treatment of viral infections. Nucleoside analogs can be converted by host or viral enzymes to their respective active moieties, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments described herein relate to a compound of Formula (I), or a pharmaceutically acceptable salt thereof. Other embodiments described herein related to a pharmaceutical composition that includes a compound of Formula (I), or a pharmaceutically acceptable salt thereof.

Some embodiments described herein relate to a method of treating a HBV and/or HDV infection that can include administering to a subject identified as suffering from the HBV and/or HDV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof) in the manufacture of a medicament for treating a HBV and/or HDV infection. Still other embodiments described herein relate to the use of a compound described herein (for example, a compound of Formula (I), or a pharmaceutically acceptable salt thereof), or a pharmaceutical composition that includes a compound described herein (such as, a compound of Formula (I), or a pharmaceutically acceptable salt thereof) for treating a HBV and/or HDV infection.

Some embodiments disclosed herein relate to a method of treating a HBV and/or HDV infection that can include contacting a cell infected with the HBV and/or HDV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HBV and/or HDV infection that can include contacting a cell infected with the HBV and/or HDV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HBV and/or HDV infection, wherein the use includes contacting a cell infected with the HBV and/or HDV with an effective amount of said compound(s) and/or pharmaceutical composition.

Some embodiments disclosed herein relate to a method of inhibiting replication of HBV and/or HDV that can include contacting a cell infected with the HBV and/or HDV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HBV and/or HDV that can include contacting a cell infected with HBV and/or HDV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HBV and/or HDV, wherein the use includes contacting a cell infected with the HBV and/or HDV with an effective amount of said compound(s) and/or pharmaceutical composition.

Some embodiments described herein relate to a method of treating a HIV infection that can include administering to a subject identified as suffering from the HIV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof), in the manufacture of a medicament for treating a HIV infection. Still other embodiments described herein relate to the use of a compound described herein (such as, a compound of Formula (I), or a pharmaceutically acceptable salt thereof), or a pharmaceutical composition that includes an effective amount of a compound described herein (for example, a compound of Formula (I), or a pharmaceutically acceptable salt thereof) for treating a HIV infection.

Some embodiments disclosed herein relate to a method of treating a HIV infection that can include contacting a cell infected with the HIV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HIV infection that can include contacting a cell infected with the HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HIV infection, wherein the use includes contacting a cell infected with the HIV with an effective amount of said compound(s) and/or pharmaceutical composition.

Some embodiments disclosed herein relate to a method of inhibiting replication of HIV that can include contacting a cell infected with the HIV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HIV that can include contacting a cell infected with HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HIV, wherein the use includes contacting a cell infected with the HIV with an effective amount of said compound(s) and/or pharmaceutical composition.

### BRIEF DESCRIPTION OF THE DRAWINGS

FIG. 1 shows example non-nucleoside reverse transcriptase inhibitors (NNRTI's).

FIG. 2 shows example nucleoside reverse transcriptase inhibitor (NRTI's).

FIG. 3A shows example HIV protease inhibitors. FIG. 3B shows additional HIV, HBV and/or HDV protease inhibitors.

FIG. 4A shows HIV fusion/entry inhibitors. FIG. 4B shows HBV and/or HDV fusion/entry inhibitors.

FIG. 5 shows HIV integrase strand transfer inhibitor (INSTI's).

FIG. 6A shows additional HIV antiviral compounds. FIG. 6B shows additional antiviral compounds.

FIG. 7 shows example HIV, HBV and/or HDV viral maturation inhibitors.

FIG. 8 shows example HIV, HBV and/or HDV capsid assembly modulators.

FIG. 9 shows example anti-HBV and/or anti-HDV farnesoid X receptor (FXR) agonists.

FIG. 10 shows example anti-HBV and/or anti-HDV tumor necrosis factor (TNF)/cyclophilin inhibitors.

FIG. 11 shows example anti-HBV and/or anti-HDV toll-like receptor (TLR) agonists.

FIG. 12 shows example HBV and/or HDV polymerase inhibitors.

FIG. 13 shows example HBV and/or HDV vaccines.

### DETAILED DESCRIPTION

The Hepadnavirus family is a family of enveloped viruses utilizing partially double-stranded, partially single-stranded circular DNA genomes. This family includes a group of viruses that cause liver disease in various organisms, and is divided between two genera: the Avihepadnaviruses, affecting birds, and the Orthohepdnaviruses, affecting mammals. Hepatitis B is a causative agent of acute/chronic hepatitis, and has a partially double-stranded 3.2 kb circular DNA from which four proteins are synthesized: the core, polymerase, surface antigen and X-gene product.

During hepatitis infection, HBV virions enter hepatocytes through a receptor-mediated process. Viral replication occurs through a multi-step mechanism. First, the circular, partially double-stranded DNA genome is transcribed by the host cell machinery, and then the full length RNA transcript is packaged into viral procapsids. The transcript is then reverse-transcribed within the capsid by the P protein, utilizing the P protein's intrinsic protein priming activity. The RNA component is then degraded by an intrinsic RNase H activity of the P protein, to yield a full-length minus-strand circular DNA. Finally, a subsequent partial plus-strand DNA is synthesized to yield the final viral genome assembly.

Viral capsids also may release the circular, partially double stranded genome into the nucleus of host cells, where synthesis of the complementary strand to the single stranded region is completed and the remaining viral ends are ligated to form the covalently closed circular DNA (cccDNA), which persists in host cell nuclei and can be passed on to daughter cells during cell division. The presence of the cccDNA gives rise to the risk of viral reemergence throughout the life of the host organism. Additionally, HBV carriers can transmit the disease for many years. Immunosuppressed individuals are especially at risk for the establishment of persistent (chronic) or latent HBV infection.

HDV is a subviral satellite of HBV, and thus, may only propagate in the presence of HBV. See, e.g., Shieh, et al., Nature, 329(6137), pp. 343-346 (1987). Replication of the single-stranded circular RNA HDV genome produces two forms of a RNA-binding protein known as the long and small delta antigens (Ag). After entering a hepatocyte, the virus is uncoated and the nucleocapsid translocated to the nucleus. The virus then uses the host cell's RNA polymerases, which treat the RNA genome as dsDNA due to its tertiary structure. Three forms of RNA are produced during replication: circular genomic RNA, circular complementary antigenomic RNA and a linear polyadenylated antigenomic RNA.

HBV and HDV are primarily transmitted by blood or mucosal contact, including by sexual activity. Infection with HBV and/or HDV leads to a wide spectrum of liver disease ranging from acute (including fulminant hepatic failure) to chronic hepatitis, cirrhosis and hepatocellular carcinoma. Acute HBV and/or HDV infection can be asymptomatic, or present with symptomatic acute effects, including fever, headaches, joint aches, and diarrhea, leading to the more severe symptoms of liver enlargement and/or jaundice associated with conjugated hyperbilirubinemia and cholestasis. Most adults infected with the virus recover, but 5%-10% are unable to clear the virus and become chronically infected. Many chronically infected individuals have persistent mild liver disease (latent HBV and/or HDV), presenting with lymphoid aggregates and bile duct damage, steatosis and/or increased fibrosis that may lead to cirrhosis. Others with chronic HBV and/or HDV infection develop the active disease, which can lead to life-threatening conditions such as cirrhosis and liver cancer. Some subjects with latent HBV and/or HDV may relapse and develop acute hepatitis.

HIV is a lentivirus that belongs to the Retroviridae family. HIV is an enveloped virus with a core consisting of two copies of a positive single-stranded RNA. HIV relies upon reverse transcriptase for reverse transcription of RNA into DNA, which becomes incorporated into host genome as a provirus. HIV uses viral glycoprotein 120 (gp 120) to bind to and infect CD4+ T lymphocytes. An increase in viral plasma load corresponds to a decrease in CD4+ T lymphocyte counts. Normal CD4+ T lymphocyte levels are from about 500 to 1,200 cells/mL. Two types of HIV have been characterized, HIV-1 and HIV-2. HIV-1 is more virulent and more infective, and has a global prevalence, whereas HIV-2 is less virulent and is geographically confined.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, anwhat is y “R” group(s) such as, without limitation, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocyclyl. For example, without limitation, if R^{a} and R^{b} of an NR^{a}R^{b} group are indicated to be “taken together,” it means that they are covalently bonded to one another to forma ring:

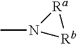

In addition, if two “R” groups are described as being “taken together” with the atom(s) to which they are attached to form a ring as an alternative, the R groups are not limited to the variables or substituents defined previously, when the R group are not taken together.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amine group and a di-substituted amine group. The number and type of atoms present in each of the groups of this paragraph are as defined herein, unless stated otherwise.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heterocyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; for example, “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. An alkenyl may include 2 to 20 carbon atoms, 2 to 10 carbon atoms or 2 to 6 carbon atoms. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. An alkynyl may include 2 to 20 carbon atoms, 2 to 10 carbon atoms or 2 to 6 carbon atoms. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s), 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. Cycloalkenyl groups can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, phenyl, naphthyl and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl).

As used herein, “aryl(alkyl)” refers to an aryl group connected, as a substituent, via an alkylene group. The alkylene and aryl group of an aryl(alkyl) may be substituted or unsubstituted. Examples include but are not limited to benzyl, 2-phenyl(alkyl), 3-phenyl(alkyl) and naphthyl(alkyl).

As used herein, “heteroaryl(alkyl)” refers to a heteroaryl group connected, as a substituent, via an alkylene group. The alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl) and their benzo-fused analogs.

A “(heterocyclyl)alkyl” refers to a heterocyclic group connected, as a substituent, via an alkylene group. The alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Alkylene groups” are straight-chained —CH₂— tethering groups having between one and ten carbon atoms, one to five carbon atoms or one to three carbon atoms that form bonds to connect molecular fragments via their terminal carbon atoms. Examples include, but are not limited to, methylene (—CH₂—), ethylene (—CH₂CH₂—), propylene (—CH₂CH₂CH₂—), butylene (—CH₂CH₂CH₂CH₂—) and pentlyene (—CH₂CH₂CH₂CH₂CH₂—). An alkylene group can be substituted by replacing one or more hydrogen of the alkylene group with a substituent(s) listed under the definition of “optionally substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, aralkyl, heteroaryl(alkyl) or heterocyclyl(alkyl) is defined herein. A non-limiting list of alkoxys are methoxy, ethoxy, n-propoxy, 1-methylethoxy(isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “hydroxyalkyl” refers to an alkyl group in which one or more of the hydrogen or deuterium atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

As used herein, “haloalkyl” refers to an alkyl group in which one or more of the hydrogen atoms are replaced by a halogen (for example, mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an —O-alkyl group in which one or more of the hydrogen atoms are replaced by a halogen (for example, mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “sulfenyl” group refers to an “—SR” group in which R can be hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} is hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-amido may be substituted or unsubstituted.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amine groups include, but are not limited to, —NH(methyl), —NH(ethyl), —NH(isopropyl), —NH(phenyl), —NH(benzyl), and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), —N(ethyl)(methyl), —N(ethyl)₂, —N(isopropyl)₂ and the like.

The term “halogen atom” or “halogen” as used herein, means any one of the radio-stable atoms of column 7 of the Periodic Table of the Elements, such as, fluorine, chlorine, bromine and iodine.

Where the number of substituents is not specified (for example, haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any chemical compounds are, unless indicated otherwise, in accord with their common usage, recognized abbreviations, or the IUPAC-IUB Commission on Biochemical Nomenclature. See, Biochem. 11:942-944 (1972).

As used herein, the term “N-linked heterocyclic base” refers to an optionally substituted nitrogen-containing heterocyclyl or an optionally substituted nitrogen-containing heteroaryl that can be attached via a ring nitrogen. The N-linked heterocyclic base can be monocyclic or multicyclic (such as, bicyclic). When compared of two or more rings, the rings can be connected in a fused-fashion. In some embodiments, the N-linked heterocyclic base can be an optionally substituted N-linked purine-base or an optionally substituted N-linked pyrimidine-base. The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g. 7-methylguanine), theobromine, caffeine, uric acid and isoguanine. Examples of pyrimidine-bases include, but are not limited to, cytosine, thymine, uracil, 5,6-dihydrouracil and 5-alkylcytosine (e.g., 5-methylcytosine). Other non-limiting examples of heterocyclic bases include diaminopurine, 8-oxo-N⁶-alkyladenine (e.g., 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

As used herein, the term “C-linked heterocyclic base” refers to an optionally substituted nitrogen-containing heterocyclyl or an optionally substituted nitrogen-containing heteroaryl that can be attached via a ring carbon. The C-linked heterocyclic base can be monocyclic or multicyclic (for example, bicyclic). When compared of two or more rings, the rings can be connected in a fused-fashion. In some embodiments, the C-linked heterocyclic base can be an optionally substituted imidazo[2,1-f][1,2,4]triazine base or an optionally substituted pyrazolo[1,5-a][1,3,5]triazine base. In some embodiments, a N-linked heterocyclic base and/or a C-linked heterocyclic base can be include an amino or an enol protecting group(s).

The term “—N-linked α-amino acid” refers to an α-amino acid that is attached to the indicated moiety via a main-chain amino or mono-substituted amine group. The —N-linked α-amino acid can be attached via one of the hydrogens that is part of the main-chain amino or mono-substituted amine group such that the —N-linked α-amino acid is attached via the nitrogen of the main-chain amino or mono-substituted amine group. N-linked α-amino acids can be substituted or unsubstituted.

The term “—N-linked α-amino acid ester derivative” refers to an α-amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include substituted and unsubstituted versions of the following: methyl-O—C(═O)—, ethyl-O—C(═O)—, n-propyl-O—C(═O)—, isopropyl-O—C(═O)—, n-butyl-O—C(═O)—, isobutyl-O—C(═O)—, tert-butyl-O—C(═O)—, neopentyl-O—C(═O)—, cyclopropyl-O—C(═O)—, cyclobutyl-O—C(═O)—, cyclopentyl-O—C(═O)—, cyclohexyl-O—C(═O)—, phenyl-O—C(═O)—, benzyl-O—C(═O)— and naphthyl-O—C(═O)—. N-linked α-amino acid ester derivatives can be substituted or unsubstituted.

The term “—O-linked α-amino acid” refers to an α-amino acid that is attached to the indicated moiety via the hydroxy from its main-chain carboxylic acid group. The —O-linked α-amino acid can be attached via the hydrogen that is part of the hydroxy from its main-chain carboxylic acid group such that the —O-linked α-amino acid is attached via the oxygen or the main-chain carboxylic acid group. O-linked amino α-acids can be substituted or unsubstituted.

As used herein, the term “α-amino acid” refers to any amino acid (both standard and non-standard amino acids). Examples of suitable α-amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable α-amino acids include, but are not limited to, ornithine, hypusine, 2-aminoisobutyric acid, dehydroalanine, citrulline and norleucine.

As used herein, the term “phosphate” is used in its ordinary sense as understood by those skilled in the art, and includes its protonated forms (for example,

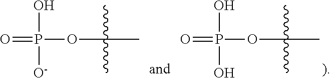 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (for example, hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Where the compounds described herein have at least one chiral center, they may accordingly exist as enantiomers. Where the compounds possess two or more chiral centers, they may additionally exist as diastereomers. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is to be understood that all such isomers and mixtures thereof are encompassed, unless stated otherwise.

Likewise, it is understood that, in any compound described, all tautomeric forms are also intended to be included. For example all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, for example, hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, at any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds, methods and combinations described herein include crystalline forms (also known as polymorphs, which include the different crystal packing arrangements of the same elemental composition of a compound), amorphous phases, solvates and hydrates. In some embodiments, the compounds described herein (including those described in methods and combinations) exist in solvated forms with pharmaceutically acceptable solvents such as water, ethanol, or the like. In other embodiments, the compounds described herein (including those described in methods and combinations) exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

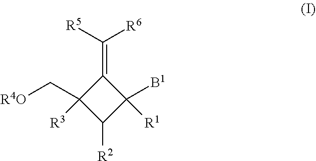

wherein: B¹ can be an optionally substituted N-linked heterocyclic base or an optionally substituted C-linked heterocyclic base; R¹ can be selected from hydrogen, halogen, cyano, an optionally substituted C₁₋₆ alkyl, an unsubstituted C₂₋₆ alkenyl and an unsubstituted C₂₋₆ alkynyl, wherein when the C₁₋₆ alkyl is substituted, the C₁₋₆ alkyl can be substituted with at least one halogen; R² can be selected from hydrogen, halogen, hydroxy, cyano and an optionally substituted C₁₋₄ alkyl, wherein when the C₁₋₄ alkyl is substituted, the C₁₋₄ alkyl can be substituted with a hydroxy or at least one halogen; R³ can be selected from hydrogen, halogen, cyano, an optionally substituted C₁₋₄ alkyl, an optionally substituted C₂₋₄ alkenyl and an unsubstituted C₂₋₄ alkynyl, wherein when the C₁₋₄ alkyl or C₂₋₄ alkenyl are substituted, the C₁₋₄ alkyl and C₂₋₄ alkenyl can be independently substituted with at least one halogen; R⁴ can be selected from hydrogen, an optionally substituted acyl, an optionally substituted O-linked α-amino acid,

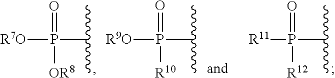

R⁵ and R⁶ can be independently hydrogen or halogen; R⁷ and R⁸ can be independently selected from absent, hydrogen,

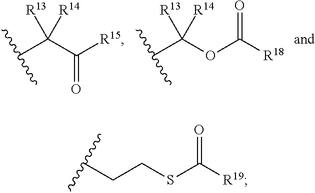

### or R⁷ can be

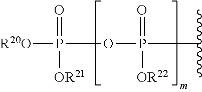

and R⁸ can be absent or hydrogen; R⁹ can be absent, hydrogen, an optionally substituted aryl or an optionally substituted heteroaryl; R¹⁰ can be an optionally substituted N-linked α-amino acid or an optionally substituted N-linked α-amino acid ester derivative; R¹¹ and R¹² can be independently an optionally substituted N-linked α-amino acid or an optionally substituted N-linked α-amino acid ester derivative; R¹³, R¹⁴, R¹⁶ and R¹⁷ can be independently selected from hydrogen, an optionally substituted C₁₋₂₄ alkyl and an optionally substituted aryl; R¹⁵ and R¹⁸ can be independently selected from hydrogen, an optionally substituted C₁₋₂₄ alkyl, an optionally substituted aryl, an optionally substituted —O—C₁₋₂₄ alkyl and an optionally substituted —O-aryl; R¹⁹ can be selected from hydrogen, an optionally substituted C₁₋₂₄ alkyl and an optionally substituted aryl; R²⁰, R²¹ and R²² can be independently absent or hydrogen; R⁵ and R⁶ can be independently hydrogen or halogen; and m can be 0 or 1.

The orientation of the substituents attached to the cyclobutyl ring can vary. For example, the following Formulae (Ia) and (Ib) are each an example of an embodiment of a compound of Formula (I).

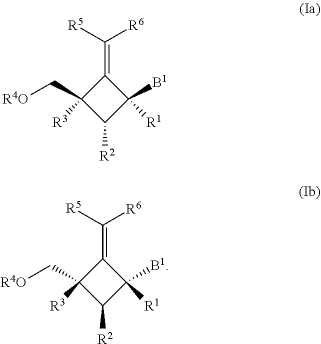

A variety of groups can be attached to the cyclobutyl ring. In some embodiments, R³ can be halogen. For example, R³ can be fluoro. In other embodiments, R³ can be cyano. In still other embodiments, R³ can be a substituted or unsubstituted, saturated or unsaturated hydrocarbon that includes 1 to 4 carbons. In some embodiments, R³ can be an optionally substituted C₁₋₄ alkyl, wherein when the C₁₋₄ alkyl is substituted, the C₁₋₄ alkyl can be substituted with at least one halogen. Examples of suitable C₁₋₄ alkyls include methyl, ethyl, n-propyl, isopropyl, n-butyl, sec-butyl, iso-butyl and tert-butyl. In some embodiments, R³ can be an unsubstituted C₁₋₄ alkyl, such as those described herein. In other embodiments, R³ can be a substituted C₁₋₄ alkyl, wherein the C₁₋₄ alkyl can be substituted with at least one halogen. For example, R³ can be a C₁₋₄ alkyl substituted with 1, 2 or 3 halogens, such as fluoro or chloro. When R³ is substituted with one halogen (for example, F or Cl), R³ can be a mono-substituted-halogenated C₁₋₄ alkyl. In some embodiments, R³ can be a fluoro-substituted C₁₋₄ alkyl. In other embodiments, R³ can be a chloro-substituted C₁₋₄ alkyl. A non-limiting list of halogen-substituted C₁₋₄ alkyls include —CH₂F or —CH₂Cl. In some embodiments, the hydrocarbon at R³ can include a double and/or a triple bond(s). For example, in some embodiments, R³ can be an optionally substituted C₂₋₄ alkenyl, wherein when the C₂₋₄ alkenyl is substituted, the C₂₋₄ alkenyl can be substituted with a halogen. As when a substituted C₁₋₄ alkyl group is present at R³, a substituted C₂₋₄ alkenyl can be substituted with 1, 2 or 3 halogens, such as fluoro or chloro. For example, in some embodiments, R³ can be a fluoro-substituted C₂₋₄ alkenyl. In other embodiments, R³ can be a chloro-substituted C₂₋₄ alkenyl. In some embodiments, R³ can be an unsubstituted C₂₋₄ alkenyl. Exemplary C₂₋₄ alkenyls include ethenyl, 1-propenyl, 2-propenyl, 1-butenyl, 2-butenyl and 3-butenyl. In some embodiments, R³ can be hydrogen.

The position opposite of the C═CR⁵R⁶ of Formula (I), or a pharmaceutically acceptable salt thereof, in the cyclobutyl ring, R², can be substituted or unsubstituted. In some embodiments, R² can be halogen. The halogen can be F, Cl, Br or I. In some embodiments, R² can be F. In other embodiments, R² can be Cl. In some embodiments, R² can be hydroxy (—OH). In other embodiments, R² can be cyano (—CN). In still other embodiments, R² can be an optionally substituted C₁₋₄ alkyl, wherein when the C₁₋₄ alkyl is substituted, the C₁₋₄ alkyl can be substituted with a hydroxy or at least one halogen. In some embodiments, R² can be an unsubstituted C₁₋₄ alkyl (such as methyl, ethyl, n-propyl, iso-propyl, n-butyl, iso-butyl, sec-butyl and tert-butyl). In other embodiments, R² can be a substituted C₁₋₄ alkyl, wherein the C₁₋₄ alkyl can be substituted with at least one halogen. For example, R² can be a C₁₋₄ alkyl substituted with 1, 2 or 3 halogens, such as fluoro or chloro. When R² is substituted with one halogen (for example, F or Cl), R² can be a mono-substituted-halogenated C₁₋₄ alkyl. In some embodiments, R² can be a fluoro-substituted C₁₋₄ alkyl. In other embodiments, R² can be a chloro-substituted C₁₋₄ alkyl. In various embodiments, the fluoro-substituted C₁₋₄ alkyl can be a mono-substituted, fluoro-substituted C₁₋₄ alkyl, such as CH₂F. In various other embodiments, the chloro-substituted C₁₋₄ alkyl can be a mono-substituted, chloro-substituted C₁₋₄ alkyl, such as CH₂Cl. In some embodiments, R² can be a C₁₋₄ alkyl substituted with one or more hydroxy groups. As an example, R² can be a mono-substituted with hydroxy. In various embodiments, R² can be —CH₂OH. In some embodiments, R² can be a C₁₋₄ alkyl substituted with 1 or 2 hydroxy groups and 1 or 2 halogens (such as F or Cl). In other embodiments, the position opposite of the C═CR⁵R⁶ of Formula (I), or a pharmaceutically acceptable salt thereof, in the cyclobutyl ring can be unsubstituted such that R² can be hydrogen.

As with other positions on the cyclobutyl ring, the carbon on which B¹ is attached can be further substituted or unsubstituted. In some embodiments, R¹ can be hydrogen. In other embodiments, R¹ can be halogen. Suitable halogens are described herein. For example, R¹ can be fluoro. In still other embodiments, R¹ can be cyano. In yet still other embodiments, R¹ can be an optionally substituted C₁₋₆ alkyl, wherein when the C₁₋₆ alkyl is substituted, the C₁₋₆ alkyl can be substituted with at least one halogen. When R¹ is an unsubstituted C₁₋₆ alkyl, R¹ can be methyl, ethyl, n-propyl, iso-propyl, n-butyl, iso-butyl, sec-butyl, tert-butyl, pentyl (branched or straight-chained) or hexyl (branched or straight-chained) in various embodiments described herein. In various embodiments, when R¹ is substituted, the C₁₋₆ alkyl can be substituted with one or more halogens (such as 1, 2, 3, 4, 5 or 6 halogens). Examples of suitable halogens are described herein. In some embodiments, R¹ can be a mono-halogenated C₁₋₆ alkyl. In other embodiments, R¹ can be a per-halogenated C₁₋₆ alkyl. Exemplary halogenated C₁₋₆ alkyls for R¹ include —CH₂F, —CH₂C₁, —CHF₂, —CHCl₂, —CF₃, —CCl₃, —CH₂CH₂F, CH₂CF₃, —CH₂CHClF, —CHFCH₂F and —CHClCH₂F. In some embodiments, R¹ can be an unsubstituted C₂₋₆ alkenyl. In other embodiments, R¹ can be an unsubstituted C₂₋₆ alkynyl. When R¹ is an unsaturated C₂₋₆ hydrocarbon, in various embodiments, R¹ can be ethenyl, ethynyl or —CH₂—CH═CH₂.

As described herein, R⁵ and R⁶ can be independently hydrogen or halogen. In some embodiments, R⁵ and R⁶ can be each hydrogen such that substituent attached to the cyclobutyl ring is ═CH₂. In other embodiments, R⁵ and R⁶ can be each halogen. When R⁵ and R⁶ are each halogen, the halogens can be the same or different. For example, R⁵ and R⁶ can be each fluoro, or one of R⁵ and R⁶ can be fluoro and the other of R⁵ and R⁶ can be chloro. In still other embodiments, one of R⁵ and R⁶ can be hydrogen, and the other of R⁵ and R⁶ can be halogen. In various embodiments, when one or both of R⁵ and R⁶ are halogen, the halogen(s) can be fluoro. Examples of substituents attached to the cyclobutyl ring that include a halogen include, but are not limited to, the following: ═CF₂, ═CCl₂, ═CFH, ═CClH and ═CClF.

Compounds of Formula (I), or a pharmaceutically acceptable salt thereof, can be referred to as cyclobutyl nucleoside analogs. In some embodiments, R⁴ can be hydrogen. When R⁴ is hydrogen, a compound of Formula (I), or a pharmaceutically acceptable salt thereof, can be a cyclobutyl nucleoside.

In some embodiments, R⁴ can be

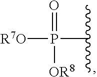

wherein m can be 0 or 1; and R⁷, R⁸, R²⁰, R²¹ and R²² can be independently absent or hydrogen. When R⁴ is

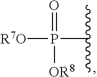

wherein m can be 0 or 1; R⁷ can be

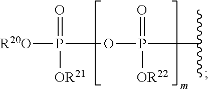

and R⁸, R²⁰, R²¹ and R²² can be independently absent or hydrogen, a compound of Formula (I), or a pharmaceutically acceptable salt thereof, can be a cyclobutyl nucleotide mono-, di- and/or tri-phosphate. Those skilled in the art understand that when R⁴ is

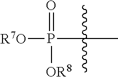

and R⁷ and R⁸ are independently absent or hydrogen, a compound of Formula (I), or a pharmaceutically acceptable salt thereof, can be a mono-phosphate. Those skilled in the art also understand that when R⁴ is

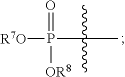

### R⁷ is

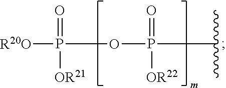

R⁸, R²⁰, R²¹ and R²² can be independently absent or hydrogen; and m is 0 or 1, a compound of Formula (I), or a pharmaceutically acceptable salt thereof, can be a di-phosphate (m is 0) or tri-phosphate (m is 1). When any one of R⁷, R⁸, R²⁰, R²¹ and R²² are absent, those skilled in the art understand that the respective oxygen to which R⁷, R⁸, R²⁰, R²¹ and R²² are shown attached will have an associated negative charge. For example, when R⁷ and R⁸ are each absent, R⁴ can be

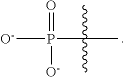 [SMILES: *P(=O)([O-])[O-]]

As further examples, when R⁴ is

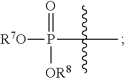

### R⁷ is

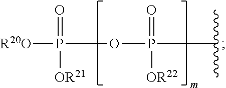

R⁸, R²⁰, R²¹ and R²² are absent; and m is 0 or 1, a compound of Formula (I), or a pharmaceutically acceptable salt thereof, R⁴ can have the following structures:

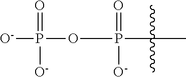 [SMILES: *P(=O)([O-])OP(=O)([O-])[O-]]

(m is 0) and

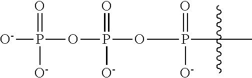 [SMILES: *P(=O)([O-])OP(=O)([O-])OP(=O)([O-])[O-]]

(m is 1).

Compounds of Formula (I), or a pharmaceutically acceptable salt thereof, can include prodrug group(s). The prodrug group(s) can be present at the position equivalent to R⁴. In some embodiments, R⁴ can be an optionally substituted acyl. In some embodiments, the acyl can be unsubstituted. In other embodiments, the acyl can be substituted. An example structure of the optionally substituted acyl can be —C(═O)R²³, wherein R²³ can be an optionally substituted C₁₋₁₂ alkyl, an optionally substituted monocyclic C₃₋₈ cycloalkyl or an optionally substituted phenyl. In some embodiments, R²³ can be an unsubstituted C₁₋₁₂ alkyl. In other embodiments, R²³ can be an unsubstituted monocyclic C₃₋₈ cycloalkyl. In still other embodiments, R²³ can be an unsubstituted phenyl. In some embodiments, R⁴ can be —C(═O)R²³, wherein R²³ can be an unsubstituted C₁₋₆ alkyl.

In some embodiments, R⁴ can be an optionally substituted O-linked α-amino acid. Examples of O-linked α-amino acids include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In various embodiments, the O-linked α-amino acid can be unsubstituted. In various other embodiments, the O-linked α-amino acid can be substituted. In some embodiments, R⁴ can be selected from unsubstituted O-linked alanine, unsubstituted O-linked valine, unsubstituted O-linked leucine and unsubstituted O-linked glycine. The α-amino acid can be a natural α-amino acid. Examples of suitable optionally substituted O-linked α-amino acids include the following:

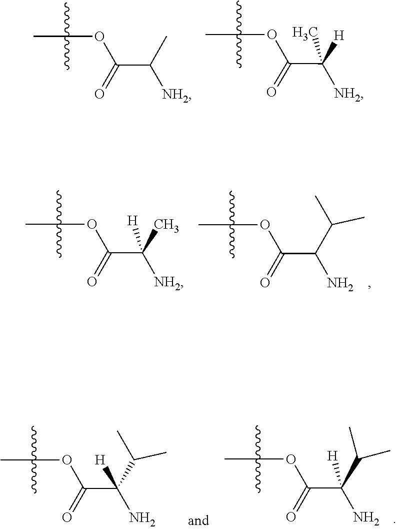

In some embodiments, R⁴ can be

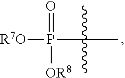

wherein one of R⁷ and R⁸ can be absent, hydrogen or

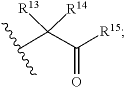

the other of R⁷ and R⁸ can be

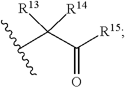

R¹³ and R¹⁴ can be independently selected from hydrogen, an optionally substituted C₁₋₂₄ alkyl and an optionally substituted aryl; and R¹⁵ can be selected from hydrogen, an optionally substituted C₁₋₂₄ alkyl, an optionally substituted aryl, an optionally substituted —O—C₁₋₂₄ alkyl and an optionally substituted —O-aryl. In other embodiments, R⁴ can be

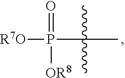

wherein R⁷ and R⁸ can be each

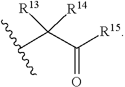

In various embodiments, when one or both of R⁷ and R⁸ are

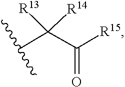

R¹³ and R¹⁴ can be each hydrogen; and R¹⁵ can be an unsubstituted C₁₋₂₄ alkyl. In other embodiments, at least one of R¹³ and R¹⁴ can be an optionally substituted C₁₋₂₄ alkyl or an optionally substituted aryl. In some embodiments, R¹⁵ can be an optionally substituted C₁₋₂₄ alkyl. In some embodiments, R¹⁵ can be an unsubstituted C₁₋₄ alkyl. In other embodiments, R¹⁵ can be an optionally substituted aryl. In still other embodiments, R¹⁵ can be an optionally substituted —O—C₁₋₂₄ alkyl, an optionally substituted —O-aryl, an optionally substituted —O-heteroaryl or an optionally substituted —O-monocyclic heterocyclyl. In some embodiments, R¹⁵ can be an unsubstituted —O—C₁₋₄ alkyl.

In some embodiments, R⁴ can be

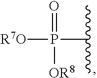

wherein one of R⁷ and R⁸ can be absent, hydrogen or

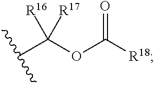

the other of R⁷ and R⁸ can be

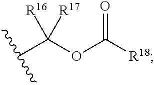

R¹⁶ and R¹⁷ can be independently selected from hydrogen, an optionally substituted C₁₋₂₄ alkyl and an optionally substituted aryl; and R¹⁸ can be selected from hydrogen, an optionally substituted C₁₋₂₄ alkyl, an optionally substituted aryl, an optionally substituted —O—C₁₋₂₄ alkyl and an optionally substituted —O-aryl. In other embodiments, R⁴ can be

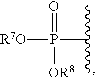

wherein R⁷ and R⁸ can be each

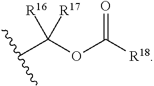

In various embodiments, when one or both of R⁷ and R⁸ are

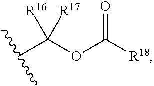

R¹⁶ and R¹⁷ can be each hydrogen; and R¹⁸ can be an unsubstituted C₁₋₂₄ alkyl. In various other embodiments, when one or both of R⁷ and R⁸ are

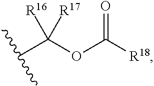

R¹⁶ and R¹⁷ can be each hydrogen; and R¹⁸ can be an unsubstituted —O—C₁₋₂₄ alkyl. In some embodiments, R¹⁶ and R¹⁷ can be hydrogen. In other embodiments, at least one of R¹⁶ and R¹⁷ can be an optionally substituted C₁₋₂₄ alkyl or an optionally substituted aryl. In some embodiments, R¹⁸ can be an optionally substituted C₁₋₂₄ alkyl. In some embodiments, R¹⁸ can be an unsubstituted C₁₋₄ alkyl. In other embodiments, R¹⁸ can be an optionally substituted aryl. In still other embodiments, R¹⁸ can be an optionally substituted —O—C₁₋₂₄ alkyl, an optionally substituted —O-aryl, an optionally substituted —O-heteroaryl or an optionally substituted —O-monocyclic heterocyclyl. In some embodiments, R¹⁸ can be an unsubstituted —O—C₁₋₄ alkyl. In some embodiments, one or both of R⁷ and R⁸ can be a pivaloyloxymethyl (POM) group. In some embodiments, R⁷ and R⁸ can be each a pivaloyloxymethyl (POM) group, and form a bis(pivaloyloxymethyl) (bis(POM)) prodrug. In some embodiments, one or both of R⁷ and R⁸ can be an isopropyloxycarbonyloxymethyl (POC) group. In some embodiments, R⁷ and R⁸ each can be an isopropyloxycarbonyloxymethyl (POC) group, and form a bis(isopropyloxycarbonyloxymethyl) (bis(POC)) prodrug.

In some embodiments, R⁴ can be

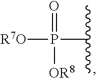

wherein one of R⁷ and R⁸ can be absent, hydrogen or

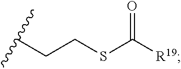

the other of R⁷ and R⁸ can be

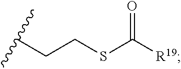

and R¹⁹ can be selected from hydrogen, an optionally substituted C₁₋₂₄ alkyl and an optionally substituted aryl. In other embodiments, R⁴ can be

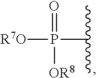

wherein R⁷ and R⁸ can be each

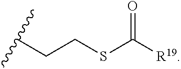

In various embodiments, R¹⁹ can be a substituted C₁₋₂₄ alkyl. In various other embodiments, R¹⁹ can be an unsubstituted C₁₋₂₄ alkyl. In still various other embodiments, R¹⁹ can be an unsubstituted C₁₋₄ alkyl. In some embodiments, R⁷ and R⁸ can be each a S-acylthioethyl (SATE) group and form a SATE ester prodrug. In some embodiments, R⁷ and R⁸ can be each

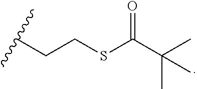 [SMILES: *CCSC(=O)C(C)(C)C]

In some embodiments, R⁴ can be

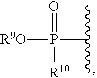

wherein R⁹ can be absent, hydrogen, an optionally substituted aryl or an optionally substituted heteroaryl; and R¹⁰ can be an optionally substituted N-linked α-amino acid or an optionally substituted N-linked α-amino acid ester derivative. In some embodiments, R⁹ can be an optionally substituted phenyl. In other embodiments, R⁹ can be an optionally substituted naphthyl. In still other embodiments, R⁹ can be an unsubstituted phenyl. In yet still other embodiments, R⁹ can be an unsubstituted naphthyl. In some embodiments, R⁹ can be an optionally substituted heteroaryl, such as an optionally substituted monocyclic heteroaryl.

In some embodiments, R¹⁰ can be an optionally substituted N-linked α-amino acid. In some embodiments, R¹⁰ can be an optionally substituted N-linked α-amino acid ester derivative. Various α-amino acids are known to those skilled in the art, and include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. The ester derivatives of the N-linked α-amino acid ester derivative can have one of the following structures: C₁₋₆ alkyl-O—C(═O)—, C₃₋₆ cycloalkyl-O—C(═O)—, phenyl-O—C(═O)—, naphthyl-O—C(═O)— and benzyl-O—C(═O)—. In some embodiments, the N-linked α-amino acid ester derivative can be a C₁₋₆ alkyl, C₃₋₆ cycloalkyl, phenyl, naphthyl or benzyl ester of alanine, asparagine, aspartate, cysteine, glutamate, glutamine, glycine, proline, serine, tyrosine, arginine, histidine, isoleucine, leucine, lysine, methionine, phenylalanine, threonine, tryptophan or valine. In some embodiments, R¹⁰ can be N-linked alanine, N-linked alanine isopropyl ester, N-linked alanine cyclohexyl ester or N-linked alanine neopentyl ester. In some embodiments, R⁹ can be an unsubstituted phenyl; and R¹⁰ can be C₁₋₆ alkyl-O—C(═O)—, C₃₋₆ cycloalkyl-O—C(═O)—, phenyl-O—C(═O)—, naphthyl-O—C(═O)— or benzyl-O—C(═O)— ester of N-linked alanine, N-linked glycine, N-valine, N-linked leucine or N-linked isoleucine. In some embodiments, when R⁴ is

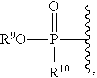

a compound of Formula (I), or a pharmaceutically acceptable salt thereof, can be a phosphoramidate prodrug, such as an aryl phosphoramidate prodrug.

In some embodiments, R⁴ can be

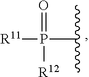

wherein R¹¹ and R¹² can be independently an optionally substituted N-linked α-amino acid ester derivative. In various embodiments, the α-amino acid portion of the optionally substituted N-linked α-amino acid ester derivative can be selected from alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. As described herein, the ester portion of the α-amino acid ester derivative can have various structures. In some embodiments, the ester portion of the N-linked α-amino acid ester derivative can have one of the following structures: C₁₋₆ alkyl-O—C(═O)—, C₃₋₆ cycloalkyl-O—C(═O)—, phenyl-O—C(═O)—, naphthyl-O—C(═O)— and benzyl-O—C(═O)—. In some embodiments, R¹¹ and R¹² can be independently selected from N-linked alanine, N-linked alanine isopropyl ester, N-linked alanine cyclohexyl ester or N-linked alanine neopentyl ester. In some embodiments, R¹¹ and R¹² can be each independently C₁₋₆ alkyl-O—C(═O)—, C₃₋₆ cycloalkyl-O—C(═O)—, phenyl-O—C(═O)—, naphthyl-O—C(═O)— or benzyl-O—C(═O)— ester of N-linked alanine, N-linked glycine, N-valine, N-linked leucine or N-linked isoleucine. In some embodiments, R¹¹ and R¹² can be the same. In other embodiments, R¹¹ and R¹² can be different. In some embodiments, when R⁴ can be

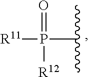

a compound of Formula (I), or a pharmaceutically acceptable salt thereof, can be an optionally substituted phosphonic diamide prodrug.

Examples of suitable N-linked α-amino acid ester derivative groups that can present at R¹⁰, R¹¹ and/or R¹² include the following:

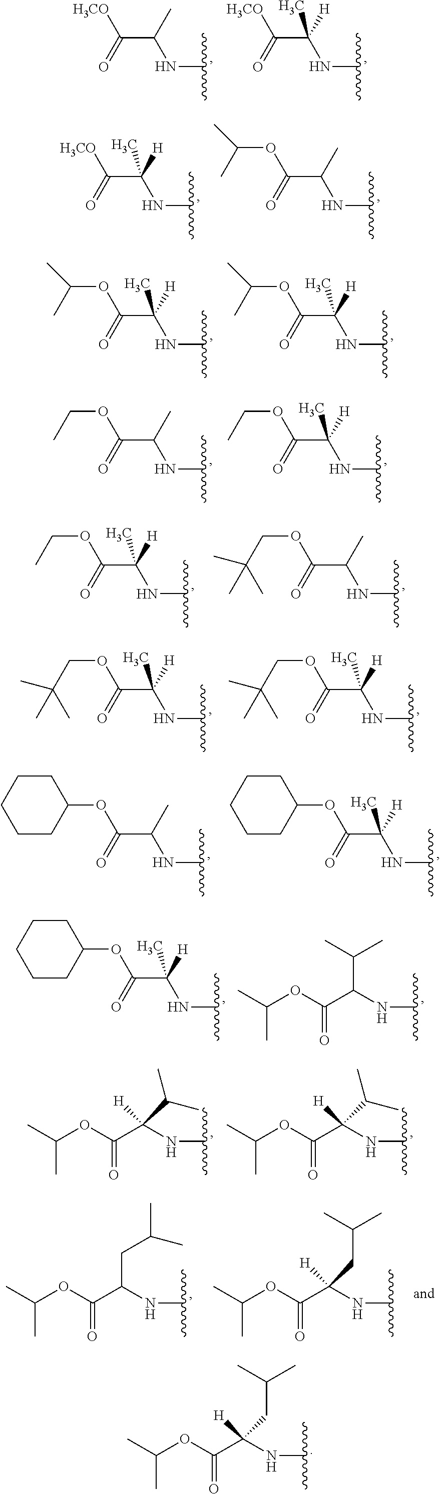

The heterocyclic base, B¹, present on a compound of Formula (I), or a pharmaceutically acceptable salt thereof, can be attached through a nitrogen (an optionally substituted N-linked heterocyclic base) or a carbon (an optionally substituted C-linked heterocyclic base). In some embodiments, B¹ can be an optionally substituted N-linked heterocyclic base. In some embodiments, B¹ can be an optionally substituted C-linked heterocyclic base.

When B¹ is an optionally substituted N-linked heterocyclic base, B¹ can be in various embodiments, an optionally substituted purine. In other various embodiments, B¹ can be an optionally substituted pyrimidine. In some embodiments, B¹ can be a substituted guanine, a substituted adenine, a substituted thymine, a substituted cytosine or a substituted uracil. In other embodiments, B¹ can be an unsubstituted guanine, an unsubstituted adenine, an unsubstituted thymine, an unsubstituted cytosine or an unsubstituted uracil.

In some embodiments, B¹ can be selected from:

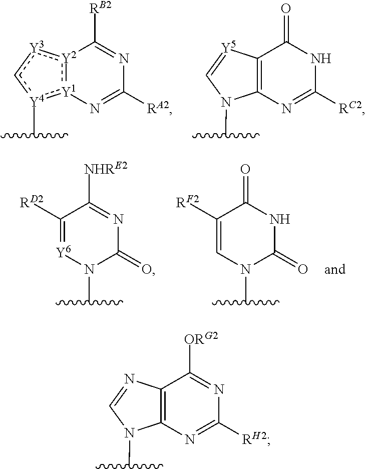

wherein: R^{A2} can be selected from hydrogen, halogen and NHR^{J2}, wherein R^{J2} can be selected from hydrogen, —C(═O)R^{K2} and —C(═O)OR^{L2}; R^{B2} can be halogen or NHR^{W2}, wherein R^{W2} can be selected from hydrogen, an optionally substituted C₁₋₆ alkyl, an optionally substituted C₂₋₆ alkenyl, an optionally substituted C₃₋₈ cycloalkyl, —C(═O)R^{M2} and —C(═O)OR^{N2}; R^{C2} can be hydrogen or NHR^{O2}, wherein R^{O2} can be selected from hydrogen, —C(═O)R^{P2} and —C(═O)OR^{Q2}; R^{D2} can be selected from hydrogen, deuterium, halogen, an optionally substituted C₁₋₆ alkyl, an optionally substituted C₂₋₆ alkenyl and an optionally substituted C₂₋₆ alkynyl; R^{E2} can be selected from hydrogen, hydroxy, an optionally substituted C₁₋₆ alkyl, an optionally substituted C₃₋₈ cycloalkyl, —C(═O)R^{R2} and —C(═O)OR^{S2}; R^{F2} can be selected from hydrogen, halogen, an optionally substituted C₁₋₆ alkyl, an optionally substituted C₂₋₆ alkenyl and an optionally substituted C₂₋₆ alkynyl; Y¹, Y² and Y⁴ can be independently N (nitrogen) or C (carbon), provided that at least one of Y¹, Y² and Y⁴ is N; Y³ can be N (nitrogen) or CR^{I2}, wherein R^{I2} can be selected from hydrogen, halogen, an unsubstituted C₁₋₆-alkyl, an unsubstituted C₂₋₆-alkenyl and an unsubstituted C₂₋₆-alkynyl; Y⁵ and Y⁶ can be independently N (nitrogen) or CH; each [symbol] can be independently a single or double bond, provided that the single bonds and the double bonds are situated in the ring so that each ring is aromatic; R^{G2} can be an optionally substituted C₁₋₆ alkyl; R^{H2} can be hydrogen or NHR^{T2}, wherein R^{T2} can be independently selected from hydrogen, —C(═O)R^{U2} and —C(═O)OR^{V2}; and R^{K2}, R^{L2}, R^{M2}, R^{N2}, R^{P2}, R^{Q2}, R^{R2}, R^{S2}, R^{U2} and R^{V2} can be independently selected from an unsubstituted C₁₋₆ alkyl, an unsubstituted C₂₋₆ alkenyl, an unsubstituted C₂₋₆ alkynyl, an optionally substituted C₃₋₆ cycloalkyl, an optionally substituted C₃₋₆ cycloalkenyl, an optionally substituted C₆₋₁₀ aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl).

Examples of suitable B¹ groups include the following:

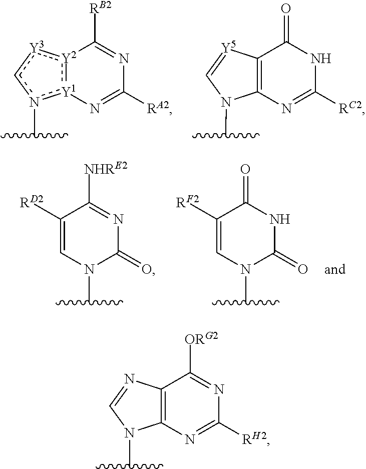

wherein R^{A2}, R^{B2}, R^{C2}, R^{D2}, R^{E2}, R^{F2}, R^{G2}, R^{H2}, Y¹, Y², Y³ and Y⁵ are provided herein. In some embodiments, B¹ can be

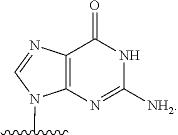 [SMILES: *n1cnc2c(=O)[nH]c(N)nc21]

In other embodiments, B¹ can be

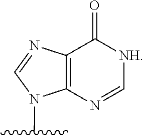 [SMILES: *n1cnc2c(=O)[nH]cnc21]

In still other embodiments, B¹ can be

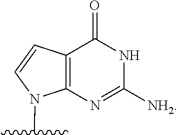 [SMILES: *n1ccc2c(=O)[nH]c(N)nc21]

In yet still other embodiments, B¹ can be

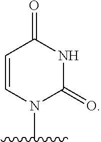 [SMILES: *n1ccc(=O)[nH]c1=O]

In some embodiments, B¹ can be

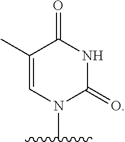 [SMILES: *n1cc(C)c(=O)[nH]c1=O]

In other embodiments, B¹ can be

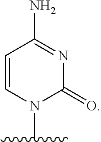 [SMILES: *n1ccc(N)nc1=O]

In still other embodiments, B¹ can be

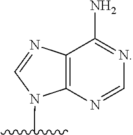 [SMILES: *n1cnc2c(N)ncnc21]

In yet still other embodiments, B¹ can be

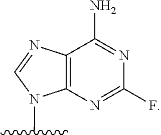 [SMILES: *n1cnc2c(N)nc(F)nc21]

In some embodiments, B¹ can be

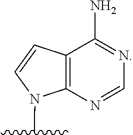 [SMILES: *n1ccc2c(N)ncnc21]

In other embodiments, B¹ can be

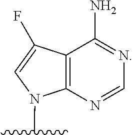 [SMILES: *n1cc(F)c2c(N)ncnc21]

In still other embodiments, B¹ can be

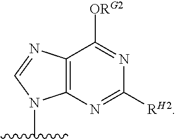

### When B¹ is

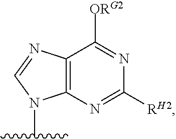

in various embodiments, R^{G2} can be an unsubstituted ethyl and R^{H2} can be NH₂.

When B¹ is an optionally substituted C-linked heterocyclic base, in various embodiments, B¹ can have the structure

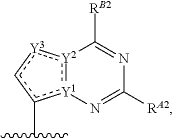

In some embodiments, B¹ can be selected from

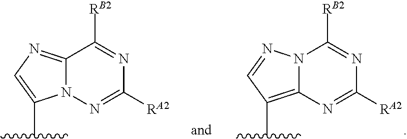

For example, B¹ can be

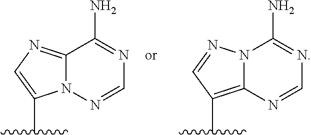 [SMILES: *c1cnc2c(N)ncnn12 ; *c1cnn2c(N)ncnc12]

In some embodiments, a compound of Formula (I), or a pharmaceutically acceptable salt thereof, can have a structure selected from:

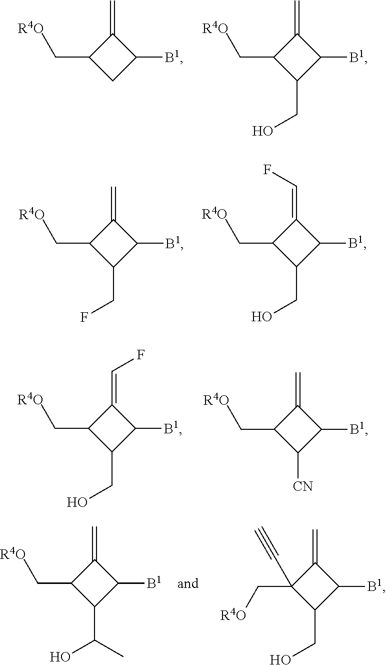

or a pharmaceutically acceptable salt of any of the foregoing. In some embodiments of this paragraph, B¹ can be an optionally substituted N-linked heterocyclic base. In some embodiments of this paragraph, B¹ can be an optionally substituted C-linked heterocyclic base. In some embodiments of this paragraph, B¹ can be an optionally substituted purine base. In other embodiments of this paragraph, B¹ can be an optionally substituted pyrimidine base. In some embodiments of this paragraph, B¹ can be guanine. In other embodiments of this paragraph, B¹ can be thymine. In still other embodiments of this paragraph, B¹ can be cytosine. In yet still other embodiments of this paragraph, B¹ can be uracil. In some embodiments of this paragraph, B¹ can be adenine. In some embodiments of this paragraph, R⁴ can be hydrogen. In other embodiments of this paragraph, R⁴ can be an optionally substituted acyl. In still other embodiments of this paragraph, R⁴ can be mono-, di- or tri-phosphate. In yet other embodiments of this paragraph, R⁴ can be phosphoramidate prodrug, such as an aryl phosphoramidate prodrug. In some embodiments of this paragraph, R⁴ can be an acyloxyalkyl ester phosphate prodrug. In other embodiments of this paragraph, R⁴ can be a S-acylthioethyl (SATE) prodrug. In still other embodiments, R⁴ can be a phosphonic diamide prodrug. In some embodiments of this paragraph, R⁴ can be an optionally substituted O-linked α-amino acid, such as one of those described herein.

In some embodiments, when R¹ is hydrogen; R² is hydroxy; R⁵ and R⁶ are each hydrogen; and B¹ is adenine; then R³ is not hydrogen. In some embodiments, when R¹ is hydrogen; R² is —CH₂OH; R⁵ and R⁶ are each hydrogen; and B¹ is adenine or guanine; then R³ is not hydrogen. In some embodiments, R² is not hydroxy. In some embodiments, R² is not CH₂OH. In some embodiments, R² is not H. In some embodiments, at least one of R⁵ and R⁶ is halogen. In some embodiments, R³ is not hydrogen. In some embodiments, R³ is halogen (such as, F), hydroxy, cyano, an unsubstituted or a substituted C₁₋₄ alkyl, an unsubstituted or a substituted C₂₋₄ alkenyl or an unsubstituted or a substituted C₁₋₄ alkynyl. In some embodiments, B¹ is not an unsubstituted adenine. In some embodiments, B¹ is not an unsubstituted guanine. In some embodiments, R⁴ is not hydrogen. In some embodiments, B¹ is not an unsubstituted purine. In some embodiments, B¹ is not an optionally substituted purine, such as an optionally substituted adenine or an optionally substituted guanine.

Examples of suitable compounds of Formula (I), or a pharmaceutically acceptable salt thereof, include, but are not limited to the following:

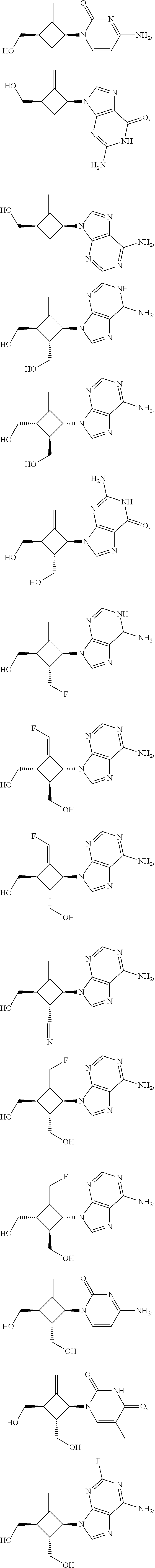

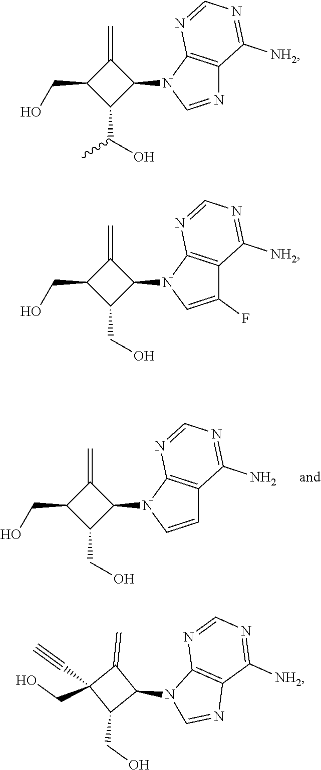 [SMILES: C=C1[C@@H](n2cnc3c(N)ncnc32)[C@H](C(C)O)[C@H]1CO ; C=C1[C@@H](n2cc(F)c3c(N)ncnc32)[C@H](CO)[C@H]1CO ; C=C1[C@@H](n2ccc3c(N)ncnc32)[C@H](CO)[C@H]1CO ; "and" ; C#C[C@]1(CO)C(=C)[C@@H](n2cnc3c(N)ncnc32)[C@@H]1CO]

or a pharmaceutically acceptable salt of any of the foregoing.

Additional examples of suitable compounds of Formula (I) include, but are not limited to the following:

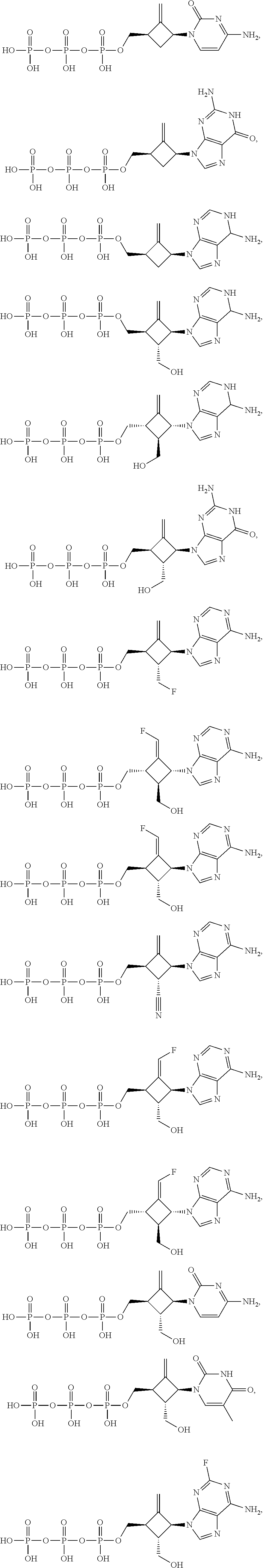

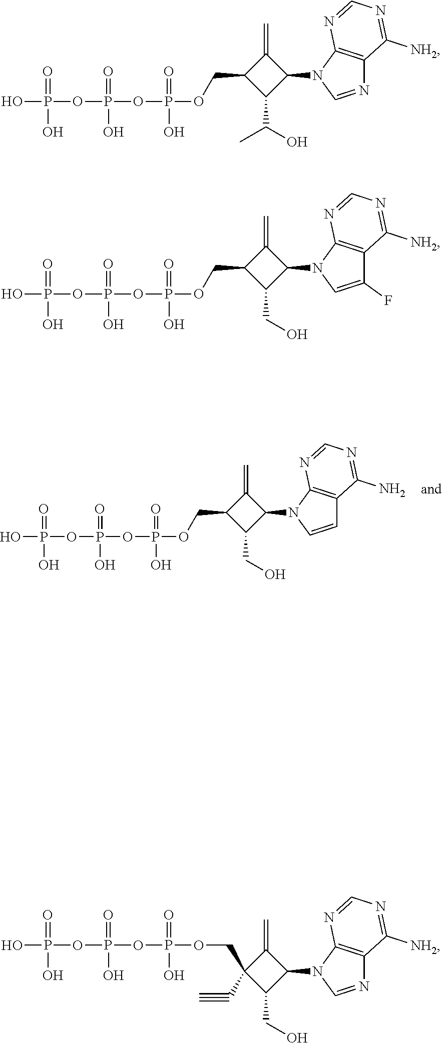 [SMILES: C=C1[C@@H](n2cnc3c(N)ncnc32)[C@H](C(C)O)[C@H]1COP(=O)(O)OP(=O)(O)OP(=O)(O)O ; C=C1[C@@H](n2cc(F)c3c(N)ncnc32)[C@H](CO)[C@H]1COP(=O)(O)OP(=O)(O)OP(=O)(O)O ; C=C1[C@@H](n2ccc3c(N)ncnc32)[C@H](CO)[C@H]1COP(=O)(O)OP(=O)(O)OP(=O)(O)O ; "and" ; C#C[C@]1(COP(=O)(O)OP(=O)(O)OP(=O)(O)O)C(=C)[C@@H](n2cnc3c(N)ncnc32)[C@@H]1CO]

or a pharmaceutically acceptable salt of any of the foregoing.

Even further examples of suitable compounds of Formula (I) include, but are not limited to the following:

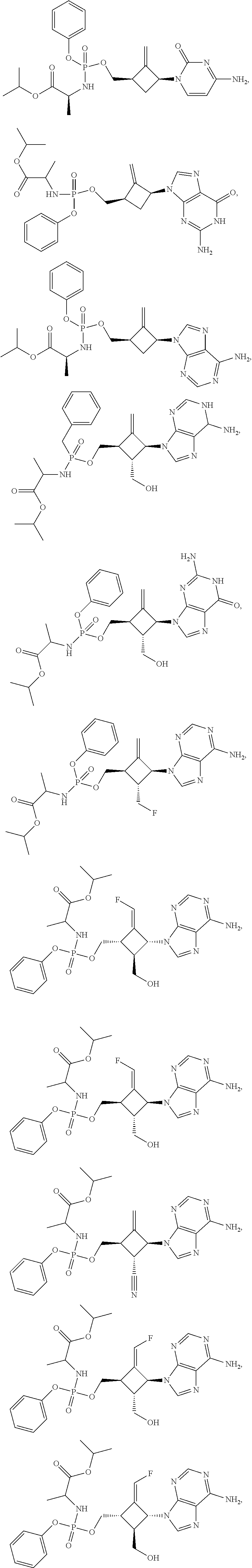

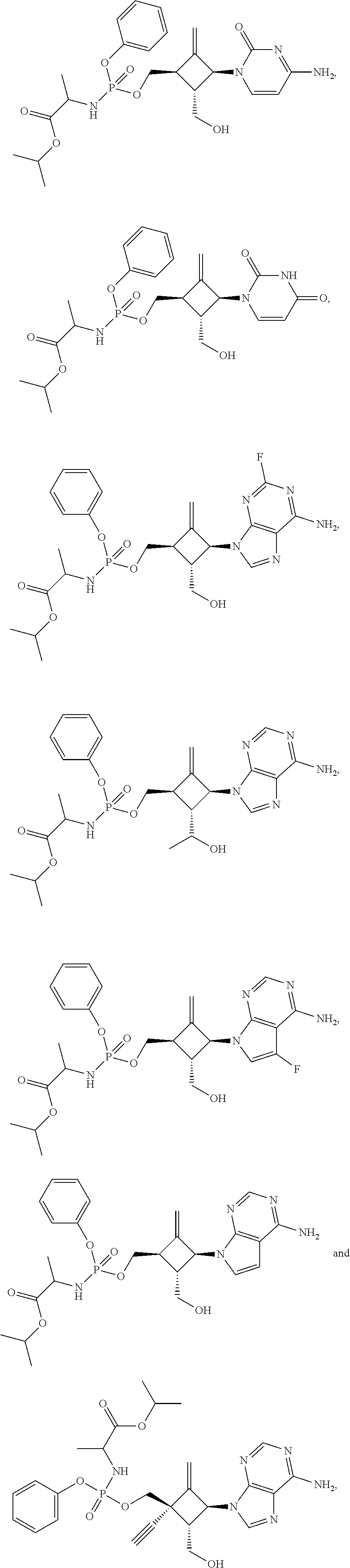

or a pharmaceutically acceptable salt of any of the foregoing.

### Synthesis

Exemplary compounds useful in methods provided herein are described by reference to the illustrative synthetic schemes for their general preparation below and the specific examples that follow. One skilled in the art will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Exemplary compounds useful in methods provided herein are described by reference to the illustrative synthetic schemes for their general preparation below and the specific examples to follow.

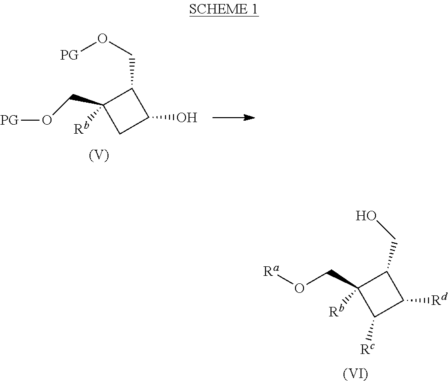

According to SCHEME 1, a commercially available or synthetically accessible compound of formula (V), where PG is benzoyl (Bz), is deprotected employing conditions known to one skilled in the art, to provide a compound of formula (VI), where R^{a} is H, R^{b} is H, and R^{d} is OH. For example ((1R,2R,3R)-3-hydroxycyclobutane-1,2-diyl)bis(methylene) dibenzoate, is reacted with ammonia-methylamine (AMA; 30% MeNH₂), in a suitable solvent such as MeOH, and the like, to provide ((1R,2R,3R)-3-hydroxycyclobutane-1,2-diyl)dimethanol.

A series of protection and deprotection steps afford a compound of formula (VI), where R^{d} is OBn; R^{c} is H; R^{b} is H; and R^{a} is TBDPS. ((1R,2R,3R)-3-Hydroxycyclobutane-1,2-diyl)dimethanol is reacted with cyclohexanone, p-toluenesulfonic acid (TsOH) and MgSO₄ to provide (1R,6R,7R)-2,4-dioxaspiro[bicyclo[4.2.0]octane-3,1′-cyclohexan]-7-ylmethanol. (1R,6R,7R)-2,4-Dioxaspiro[bicyclo[4.2.0]octane-3,1′-cyclohexan]-7-ylmethanol is protected as the silyl ether under conditions known to one skilled in the art. For example, the alcohol compound (1R,6R,7R)-2,4-dioxaspiro[bicyclo[4.2.0]octane-3,1′-cyclohexan]-7-ylmethanol is reacted with with tert-butyldimethylsilyl chloride, a suitable base such as using imidazole, dimethylaminopyridine, and the like, in a solvent such as DMF, at temperatures ranging from 0° C. to r.t., to afford ((1R,6R,7R)-2,4-dioxaspiro[bicyclo[4.2.0]octane-3,1′-cyclohexan]-7-ylmethoxy)(tert-butyl)diphenylsilane. The spirohexane carbocyclic compound ((1R,6R,7R)-2,4-dioxaspiro[bicyclo[4.2.0]octane-3,1′-cyclohexan]-7-ylmethoxy)(tert-butyl)diphenylsilane was hydrolyzed with pyridinium p-toluenesulfonate (PPTS) in a solvent such as MeOH, to afford (1R,2R,3R)-3-(((tert-butyldiphenylsilyl)oxy)methyl)-2-(hydroxymethyl)cyclobutanol. N-Triphenylmethyl (Trityl, Tr, or Trt) protection of the hydroxy compound (1R,2R,3R)-3-(((tert-butyldiphenylsilyl)oxy)methyl)-2-(hydroxymethyl)cyclobutanol, employing conditions known to one skilled in the art, provides (1R,2R,3R)-3-(((tert-butyldiphenylsilyl)oxy)methyl)-2-((trityloxy)methyl)cyclobutanol. For example, reaction of the alcohol compound (1R,2R,3R)-3-(((tert-butyldiphenylsilyl)oxy)methyl)-2-(hydroxymethyl)cyclobutanol with trityl chloride (TrtCl), in a suitable organic base, such as pyridine, dimethylaminopyridine (DMAP), 2,4,6-tri-t-butyl pyridine, 2,4,6-collidine, triethylamine (TEA), and 1,8-diazabicyclo[5.4.0]-undec-7-ene (DBU), preferably pyridine, affords (1R,2R,3R)-3-(((tert-butyldiphenylsilyl)oxy)methyl)-2-((trityloxy)methyl)cyclobutanol. Benzyl protection of (1R,2R,3R)-3-(((tert-butyldiphenylsilyl)oxy)methyl)-2-((trityloxy)methyl)cyclobutanol, employing benzyl bromide (BnBr), a suitable base such as NaH, and the like, in a suitable solvent such as THF, DMF, and the like, at temperatures ranging from 0° C. to r.t., affords (((1R,2R,3R)-3-(benzyloxy)-2-((trityloxy)methyl)cyclobutyl)methoxy)(tert-butyl)diphenylsilane. Trity deprotection, employing conditions known to one skilled in the art, for example mild acidic conditions such as TsOH in a solvent such as MeOH, affords an alcohol compound of formula (VI), where R^{a} is t-butyldiphenylsilyl (TBDPS), R^{b} is H; R^{c} is H; and R^{d} is O-Bn.

In an alternate method an alcohol compound of formula (VI), where R^{a} is t-butyldiphenylsilyl (TBDPS), R^{b} is H; R^{c} is H; and R^{d} is O-Bn is prepared in three steps from a compound of formula (V), where PG is benzoyl (Bz). In a first step, reaction of ((1R,2R,3R)-3-hydroxycyclobutane-1,2-diyl)bis(methylene) dibenzoate with benzyl 2,2,2-trichloroacetimidate, cyclohexane, benzyl 2,2,2-trichloroacetimidate, trifluoromethanesulfonic acid (TFMSA), in a suitable solvent such as DCM, afforded ((1R,2R,3R)-3-(benzyloxy)cyclobutane-1,2-diyl)bis(methylene) dibenzoate. Removal of the Bz protecting group employing conditions previously described provides ((1R,2R,3R)-3-(benzyloxy)cyclobutane-1,2-diyl)dimethanol. Protection of ((1R,2R,3R)-3-(benzyloxy)cyclobutane-1,2-diyl)dimethanol as the silyl ether, employing conditions previously described, provides an alcohol compound of formula (VI), where R^{a} is t-butyldiphenylsilyl (TBDPS), R^{b} is H; R^{c} is H; and R^{d} is O-Bn.

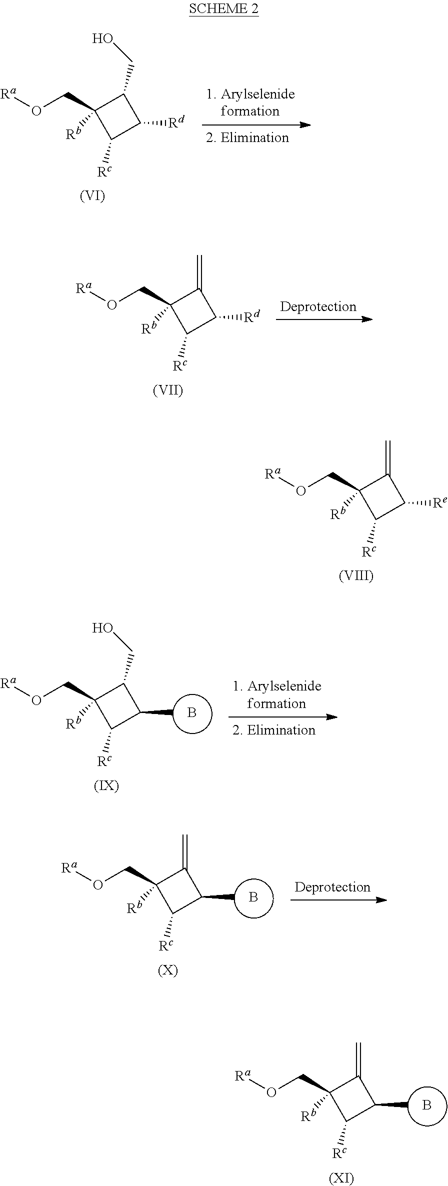

According to SCHEME 2, an alcohol compound of formula (VI) undergoes an elimination according to Grieco, to afford an alkene of formula (VII). For example, an alcohol of formula (VI), where R^{a} is a suitable protecting group such as t-butyldiphenylsilyl (TBDPS), benzyl (Bn), 4,4-dimethoxytrityl (DMTr), and the like; R^{b} is H; R^{c} is H or CH₂OBn; and R^{d} is O-Bn, or OAcetyl (OAc); is first converted to the arylselenide, followed by oxidation to selenoxide using H₂O₂ or m-CPBA, which then undergoes syn elimination to afford an olefin compound of formula (VII).

Debenzylation of an aryl benzyl ether compound of formula (VII) bearing an acid-labile silyl ether where R^{a} is TBDPS, R^{b} is H; employing conditions known to one skilled in the art, provides a compound of formula (VIII) where R^{e} is OH. For example, debenzylation was achieved employing BCl₃, a base such as aq. Na₂CO₃, and the like; in a suitable solvent such as DCM, THF, H₂O, or a mixture thereof; provides a compound of formula (VIII), where R^{a} is TBDPS, R^{b} is H, and R^{e} is OH.

Selective O-deacylation of a compound of formula (VII), where R^{a} is Bn, R^{b} is H, R^{c} is CH₂OBn; and R^{d} is OAc; is achieved employing K₂CO₃, MeOH, to provide a compound of formula (VIII), where R^{e} is OH.

In a similar Grieco elimination, a compound of formula (X) is prepared from a compound of formula (IX), where R^{a} is a suitable protecting group such as 4,4-dimethoxytrityl (DMTr) or benzyl (Bn); R^{b} is H or C≡C; R^{c} is CH₂OBn, or CH₂—O-monomethoxytrityl (MMtr); and ring B is a suitably protected nitrogen linked base such as tert-butyl (6-chloro-9H-purin-2-yl)carbamate, or N-(9H-purin-6-yl)benzamide.

Mild detritylation of a monomethoxytrityl (MMtr) and 4,4-dimethoxytrityl (DMTr) compound of formula (X), where R^{a} is 4,4-dimethoxytrityl (DMTr); R^{b} is C≡CH; R^{c} is CH₂—O-MMtr; and ring B is tert-butyl (6-chloro-9H-purin-2-yl)carbamate; is achieved under conditions known to one skilled in the art. For example, employing an acid such as trichloroacetic acid (TCA), and the like, in a suitable solvent such as DCM, and the like, at r.t., for a period of 1-3 h, provides a hydroxy compound of formula (XI), where R^{a} is H; R^{b} is C≡CH; R^{c} is CH₂—OH; and ring B is a base such as tert-butyl (6-chloro-9H-purin-2-yl)carbamate.

The optional protecting groups in the protected compound of formulas (XI) are then cleaved following established deprotection methodologies, such as those described in T. W. Greene and P. G. M. Wuts, “Protective Groups in Organic Synthesis,” 3 ed., John Wiley & Sons, 1999.

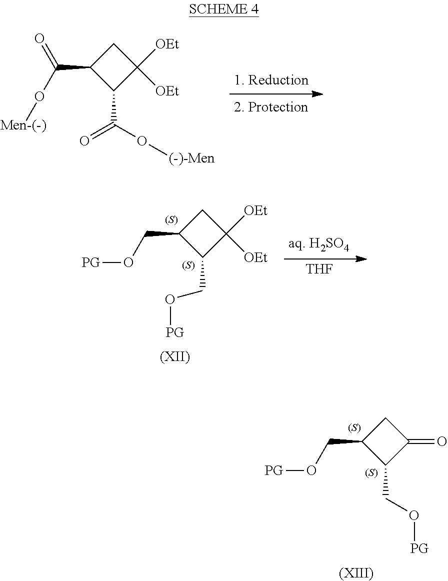

According to SCHEME 4, (1S,2R)-dimenthyl 3,3-diethoxycyclobutane-1,2-dicarboxylate (prepared according to methods as described in U.S. Pat. No. 6,025,519) is reduced employing lithium aluminum hydride (LAH), in a suitable solvent such as THF, and the like, to provide the corresponding diol ((1S,2S)-3,3-diethoxycyclobutane-1,2-diyl)dimethanol. The diol was converted to the corresponding bis-benzyl ether compound of formula (XII), where PG is Bn, under conditions previously described. Removal of the acetal provided the cyclobutanone compound of formula (XIII), where PG is Bn.

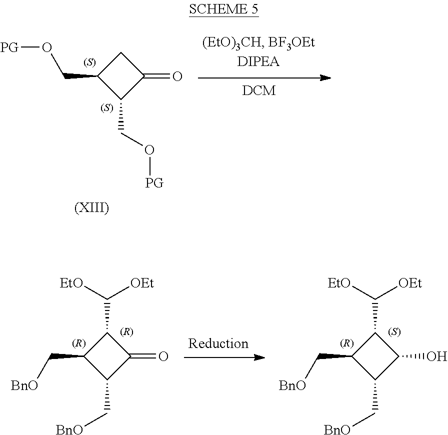

According to SCHEME 5, triethyl orthoformate, was reacted with BF₃.OEt₂, in a suitable solvent such as DCM, at temperatures ranging from −30° C. to 0° C.; (2S,3S)-2,3-bis((benzyloxy)methyl)cyclobutanone (XIII) is reacted with the resulting solution at −78° C., and a base such as DIPEA, to provide (2S,3R,4R)-2,3-bis((benzyloxy)methyl)-4-(diethoxymethyl)cyclobutanone. Reduction of (2S,3R,4R)-2,3-bis((benzyloxy)methyl)-4-(diethoxymethyl)cyclobutanone with L-Selectride provides (1R,2S,3R,4S)-2,3-bis((benzyloxy)methyl)-4-(diethoxymethyl)cyclobutanol.

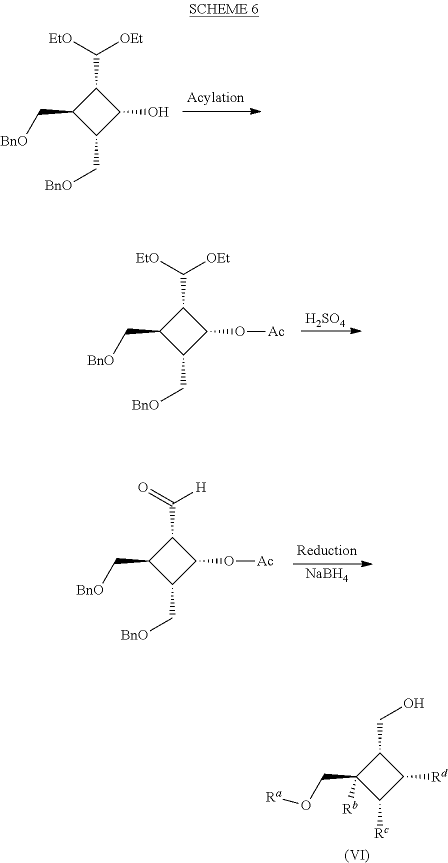

According to SCHEME 6, (1R,2S,3R,4S)-2,3-bis((benzyloxy)methyl)-4-(diethoxymethyl)cyclobutanol is acylated under conditions known to one skilled in the art. For example, reaction of (1R,2S,3R,4S)-2,3-bis((benzyloxy)methyl)-4-(diethoxymethyl)cyclobutanol with acetic anhydride, a catalyst such as 4-DMAP or pyridine, or a mixture thereof, to provide (1R,2S,3R,4S)-2,3-bis((benzyloxy)methyl)-4-(diethoxymethyl)cyclobutyl acetate. Deprotection of the diethyl acetal under conditions known to one skilled in the art provides (1R,2S,3R,4S)-2,3-bis((benzyloxy)methyl)-4-formylcyclobutyl acetate, preferably deprotection with a suitable acid such as H₂SO₄, in a solvent such as CN₃CN. Reduction of the aldehyde compound (1R,2S,3R,4S)-2,3-Bis((benzyloxy)methyl)-4-formylcyclobutyl acetate with a reducing agent such as NaBH₄, in a suitable solvent such as THF, and the like, provides a compound of formula (VI), where R^{a} is Bn, R^{b} is H, R^{c} is CH₂OBn, and R^{d} is OAc.

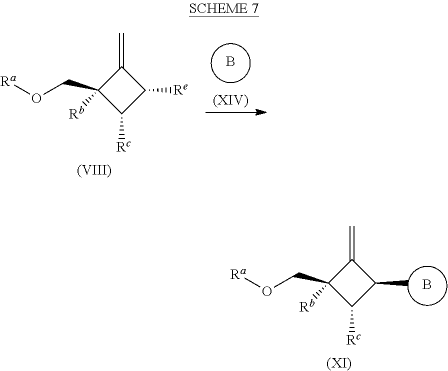

According to SCHEME 7, a compound of formula (VIII) is reacted under Mitsonobu conditions with 6-10 membered heterocyclic or heteroaromatic monocyclic or bicyclic ring of formula (XIV), to afford a compound of formula (XI), where R^{a} is TBDPS or Bn, R^{b} is H, and ring B is a 6-10 membered heterocyclic or heteroaromatic monocyclic or bicyclic ring. For example, a compound of formula (XIV), such as 3-benzoylpyrimidine-2,4(1H,3H)-dione, tert-butyl (6-chloro-9H-purin-2-yl) carbamate, N,N-diboc-9H-purin-6-amine, N,N-di-Boc-2-fluoro-9H-purin-6-amine, 3-benzoyl-5-methylpyrimidine-2,4(1H,3H)-dione, 4-chloro-7H-pyrrolo[2,3-d]pyrimidine, 4-chloro-5-fluoro-7H-pyrrolo[2,3-d]pyrimidine, or tert-butyl (6-chloro-9H-purin-2-yl)carbamate; is reacted with a compound of formula (VIII) such as (1R,3R)-3-(((tert-butyldiphenylsilyl)oxy)methyl)-2-methylenecyclobutanol, or (1R,2S,3R)-2,3-bis((benzyloxy)methyl)-4-methylenecyclobutanol; PPh₃; a reagent such as diethyl azodicarboxylate (DEAD), diisopropyl azodicarboxylate (DIAD), and the like; in a suitable solvent such as THF, ACN, and the like; at temperatures between 0° C. to 55° C.; for a period of about 24-36 h, to afford a compound of formula (XI) (the coupling reaction proceeds with inversion of the stereochemistry at the carbon bonded to the hydroxyl group of the alcohol reactant).

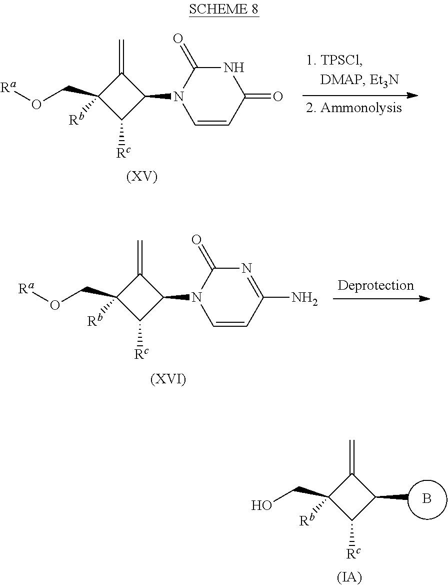

According to SCHEME 8, a compound of formula (XVI), where R^{a} is Bn, or TBDPS, R^{b} is H, and R^{c} is H or CH₂—O-Bn, is prepared from a compound of formula (XV) in two steps by first reaction with 2,4,6-triisopropylbenzenesulfonyl chloride (TPSCl) in the presence of dimethylaminopyridine (DMAP); triethylamine; in a suitable solvent such as acetonitrile and the like; followed by ammonolysis with NH₄OH. Deprotection of the TBDPS group on a compound of formula (XVI), where R^{a} is TBDPS, R^{b} is H, R^{c} is H or CH₂—O-Bn, is achieved with an acid such as HCl, in a solvent such as THF. The optional protecting groups in the protected compound of formulas (XVI) are then cleaved following established deprotection methodologies, such as those described in T. W. Greene and P. G. M. Wuts, “Protective Groups in Organic Synthesis,” 3 ed., John Wiley & Sons, 1999, to provide a compound of formula (IA), where R^{b} is H, R^{c} is H or CH₂OH, and ring B is

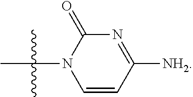 [SMILES: *n1ccc(N)nc1=O]

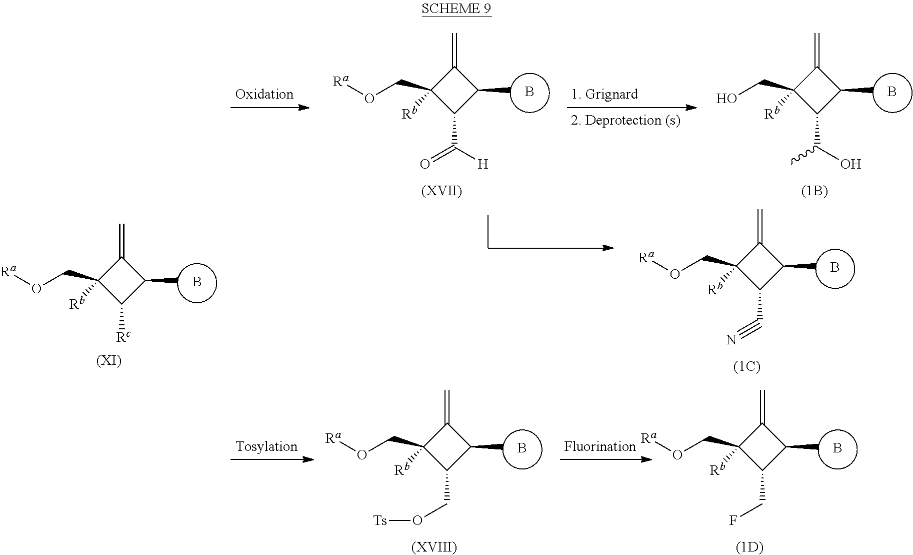

According to SCHEME 9, a compound of formula (XI), where R^{a} is TBDPS; R^{b} is H; R^{c} is CH₂—OH; and ring B is a nitrogen linked heterorcycle or heteroaryl such as N-(9H-purin-6-yl)benzamide; is oxidized with a suitable oxidizing agent, such as a chromium trioxide or chromate reagent, Dess-Martin periodinane, or by Swern oxidation. In a preferred embodiment, a compound of formula (XI) is treated with Dess-Martin periodinane, in a suitable solvent such as dichloromethane, and the like, at temperatures ranging from about 0° C. to about 25° C., for a period of approximately 0.5 to 4 h, to produce a compound of formula (XVII). Addition of a Grignard reagent, such as an alkyl, alkenyl, or alkynyl magnesium halide (for example, MeMgBr, EtMgBr, vinylMgBr, allylMgBr, and ethynylMgBr) or an alkyl, alkenyl, or alkynyl lithium, such as MeLi, to an aldehyde compound of formula (XVII), in a suitable organic solvent, such as tetrahydrofuran (THF), diethyl ether, and the like, affords an alcohol compound where R^{b} is H and R^{a} is TBDPS. The optional protecting groups in the protected alcohol compound are then cleaved following established deprotection methodologies, such as those described in T. W. Greene and P. G. M. Wuts, “Protective Groups in Organic Synthesis,” 3 ed., John Wiley & Sons, 1999, to provide a compound of Formula (IB).

Treatment of an aldehyde compound of formula (XVII) with hydroxylamine hydrochloride and pyridine gives a hydroxyimine intermediate which is converted to the cyano function using methanesulfonyl chloride and pyridine, to provide a cyano compound, where R^{a} is Bz, R^{b} is H, and ring B is N-(9H-purin-6-yl)benzamide. The optional protecting groups in the protected cyano compound are then cleaved following established deprotection methodologies, such as those described in T. W. Greene and P. G. M. Wuts, “Protective Groups in Organic Synthesis,” 3 ed., John Wiley & Sons, 1999, to provide a compound of Formula (IC).

Conversion of the hydroxy substituent to a leaving group such as halo or sulphonate allows displacement using nucleophilic reagents such as tetrabutylammonium fluoride. For example, the hydroxyl group of a compound of formula (XII), where R^{c} is CH₂OH is converted to a leaving group (mesylate, tosylate, and the like), employing conditions known to one skilled in the art. In a preferred embodiment, a sulfonate (p-toluenesulfonate (—OTs)) compound of formula (XVIII) is prepared using paratoluensulfonyl chloride, DMAP, TEA, in a suitable solvent such as DCM, and the like. Subsequent fluorination using TBAF, in a suitable solvent such as THF, and the like, at temperatures ranging from rt to about 50° C., for a period of about 16 h, affords a tosylate compound where R^{a} is MMtr, and ring B is N-(9l²-purin-6-yl)benzamide. The optional protecting groups in the fluoro compound are then cleaved following established deprotection methodologies, such as those described in T. W. Greene and P. G. M. Wuts, “Protective Groups in Organic Synthesis,” 3 ed., John Wiley & Sons, 1999, to provide a compound of Formula (ID).

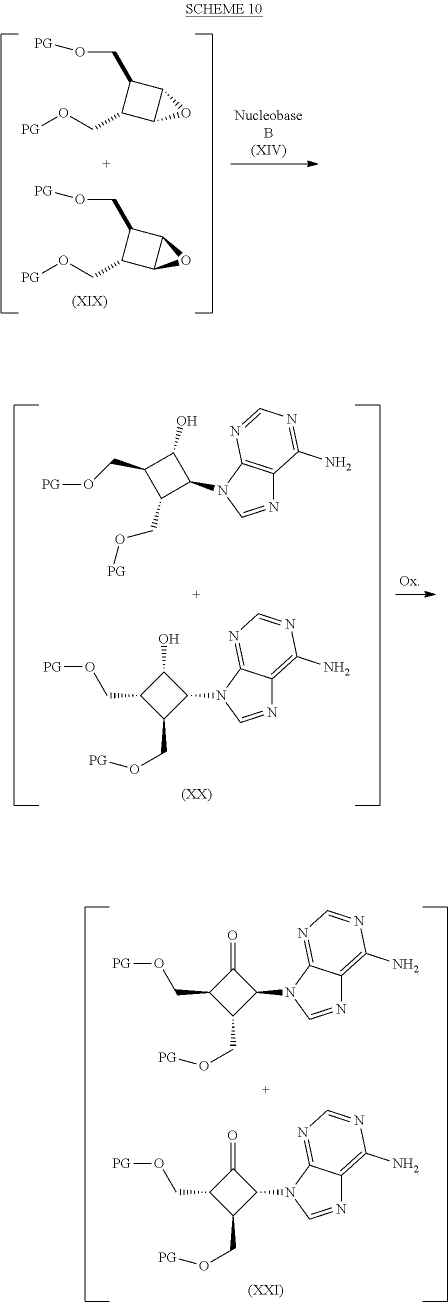

Epoxide mixture of formula (XIX), where PG is Bn, is prepared according to the methods as described in Tetrahedron (1994), 50(46), 13145-54. According to SCHEME 10 reaction of epoxide mixture (XIX) with a base such as NaH, and the like, in a suitable solvent such as DMF, and the like, with the appropriate nucleobase B (XIV) nucleophile such as adenine, at temperatures ranging from 80 to 100° C., for a period of 48-52 h, provides a mixture of compounds of formula (XX). A compound mixture of formula (XX) is oxidized employing conditions previously described to provide a compound mixture of formula (XXI).

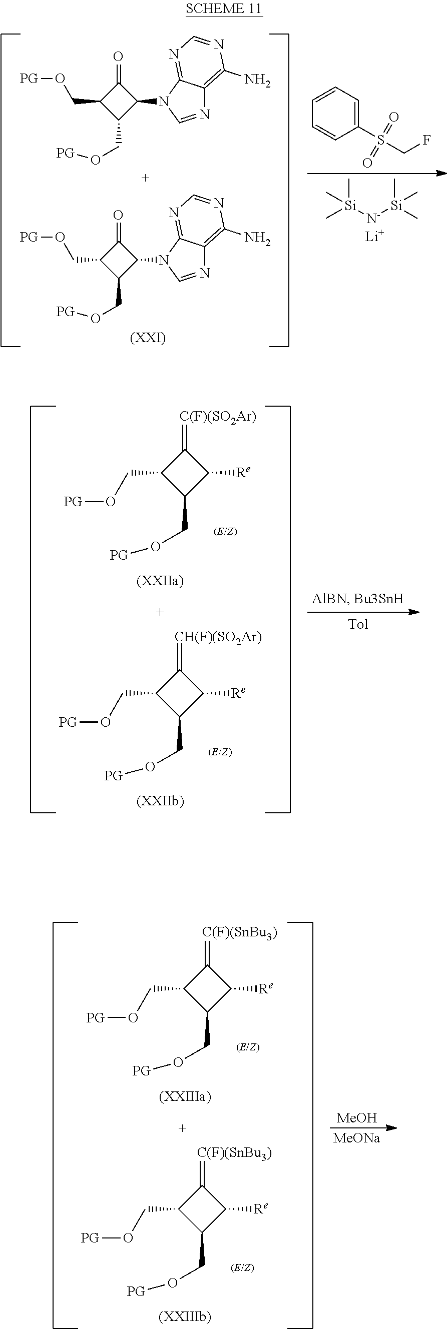

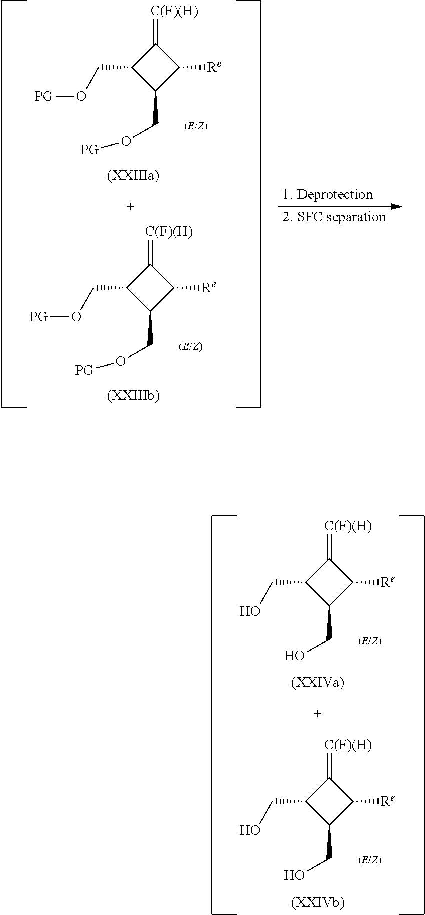

According to SCHEME 11, a compound mixture of formula (XXI) is reacted with fluoromethyl phenyl sulfone, diethyl chlorophosphite, and lithium bis(trimethylsilyl)amide, in a suitable solvent such as THF, and the like, to provide compounds of formula (XXIIa) and (XXIIb) as a mixture of E and Z isomers. It is clear that to one skilled in the art, the halomethylidene derivative represented by the compound of formula (XXIIa) and (XXIIb) exists as two geometric isomers which can be referred to as the (Z) and the (E) isomers.

The 2-arylsulfonylhalomethylidene mixture of compounds of formula (XXIIa and XXIIb) are converted to the corresponding 2-tributyl-tin-halomethylidene derivatives of formula (XXIIIa and XXIIIb). For example, reaction of a mixture of compounds of formula (XXIIa and XXIIb) with tributyl-tin hydride (HSnBu₃) in the presence of 2,2′-azobisisobutyronitrile (AIBN) in a suitable solvent such as benzene. The geometric isomers of the 2-tributyl-tin-halomethylidene derivative of formula (XXIIIa and XXIIIb) can optionally be isolated using procedures and techniques which are well known and appreciated in the art.

The tributyl-tin moiety of a compound mixture of formulas (XXIIa and XXIIb) is removed and replaced by a hydrogen atom to provide the corresponding 2-halomethylidene compound mixture of formulas (XXIIIa and XXIIIb). This is accomplished by procedures that are well known and appreciated in the art such as reaction with dilute acetic acid, ammonia in methanol or sodium methoxide. Compound mixture of formulas (XXIIa and XXIIIb) undergo protection and deprotection and separated by using conventional separation techniques well known to one skilled in the art, and as previously described, to provide isolated pure compounds of formula (XXIVa) and (XXIVb).

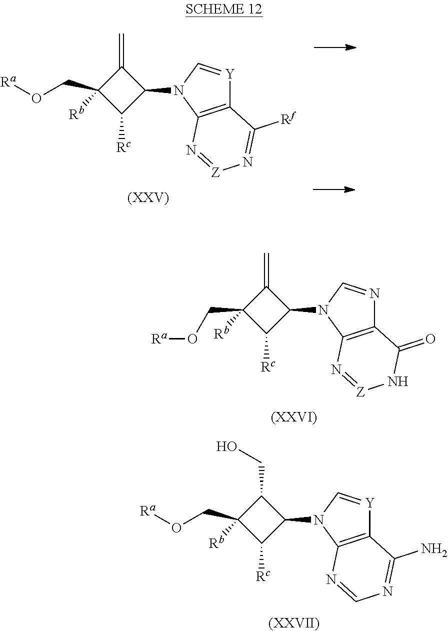

According to SCHEME 12, a chloropurine compound of formula (XXV), where R^{f} is Cl; R^{c} is H; R^{b} is H; R^{a} is TBDPS; Y is N; and Z is NH(BOC); is converted to a carbonyl compound of formula (XXVI) by treatment with the alkoxide of 3-hydroxypropionitrile. For example, reaction of 3-hydroxypropionitrile; in a suitable solvent such as THF, and the like; with a base such as NaH, and the like; tert-butyl (9-((1S,3R)-3-(((tert-butyldiphenylsilyl)oxy)methyl)-2-methylenecyclobutyl)-6-chloro-9H-purin-2-yl)carbamate; at a temperature of about 0° C.; affords tert-butyl (9-((1S,3R)-3-(((tert-butyldiphenylsilyl)oxy)methyl)-2-methylenecyclobutyl)-6-oxo-6,9-dihydro-1H-purin-2-yl)carbamate. In an alternate method, hydrolysis a chloropurine compound of formula (XXV), where R^{f} is Cl; R^{c} is CH₂O(Bn); R^{b} is H; R^{a} is Bn; Y is N; and Z is NH(PG); is achieved employing 75% aq. CF₃COOH at r.t.

Ammonolysis of a compound of formula (XXV), where R^{f} is Cl; R^{c} is CH₂O(MMtr); R^{b} is H; R^{a} is H; Y is CH, or CF; and Z is CH; is achieved by reaction with 25% strength ammonia solution; in a suitable solvent such as dioxane, and the like; at elevated pressure; at elevated temperature, preferably at 90 to 120° C.; to afford a compound of formula (XXVII), where R^{f} is NH₂.

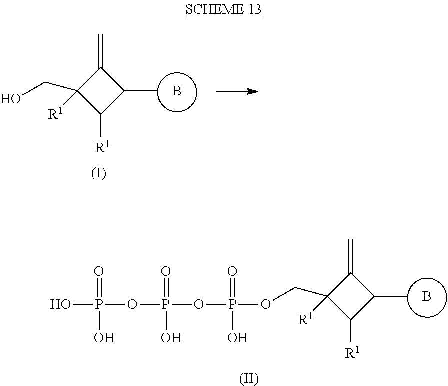

According to SCHEME 13, a nucleoside triphosphate compound of Formula (II), is prepared from a nucleoside compound of Formula (I), employing conditions known to one skilled in the art. For example, reaction of the nucleoside of Formula (I), with trimethyl phosphate, triethyl phosphate, and the like; phosphoryl chloride; and N-methylimidazole to provide the corresponding nucleoside monophosphate intermediate. Subsequent reaction of the nucleoside monophosphate with the tetrabutylammonium salt of pyrophosphate, in a suitable solvent such as DMF, and the like, provides the triphosphate of Formula (II).

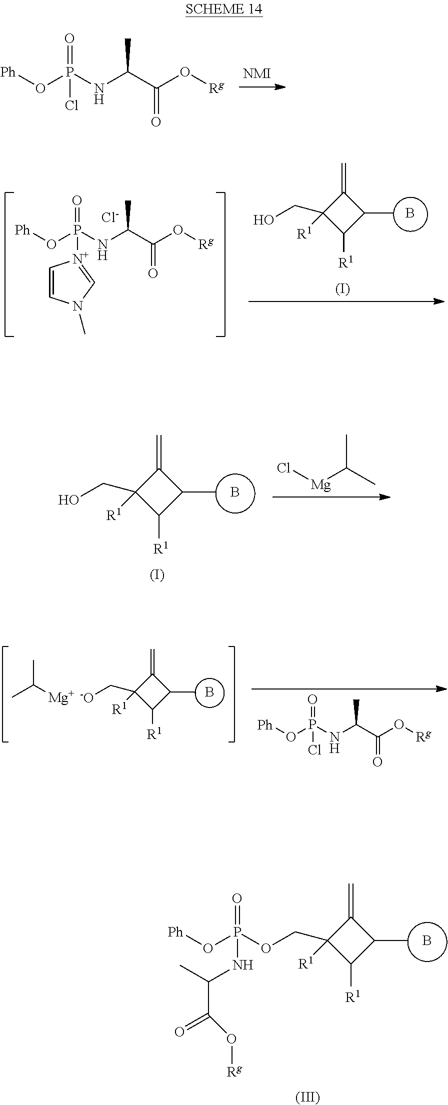

According to SCHEME 14, aryloxyphosphoramidate nucleoside prodrug compounds of Formula (III) are prepared by coupling of nucleosides compounds of Formula (I) with phosphorochloridate by either activation of the imidazolium intermediate with NMI (N-methylimidazole) or by 5′-deprotonation of the nucleoside with isoPrMgCl, t-BuMgCl, and the like, and subsequent substitution with the chlorophosphoramidate. It is noteworthy that these different synthetic approaches generally lead to approximate 1:1 mixtures of compounds of Formula (III) as diastereoisomers at the phosphorus center (Sₚ and Rₚ isomers).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Compounds prepared according to the schemes described herein may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

In some embodiments, varying the substituents on a compound described herein, such as a compound of Formula (I), or a pharmaceutically acceptable salt thereof, can result in the phosphorous being a chiral center. In some embodiments, the phosphorous can be in the (R)-configuration. In some embodiments, the phosphorous can be in the (S)-configuration. Examples of the two configurations are:

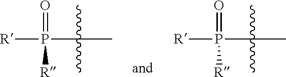

In some embodiments, a compound of Formula (I), or a pharmaceutically acceptable salt thereof, can be enriched in (R) or (S) configuration with respect to the phosphorous. For example, one of the (R) and (S) configuration with respect to the phosphorous atom can be present in an amount >50%, ≥75%, ≥90%, ≥95% or ≥99% compared to the amount of the other of the (R) or (S) configuration with respect to the phosphorous atom.

By neutralizing the charge on the phosphonate moiety of Formula (I), or a pharmaceutically acceptable salt thereof, penetration of the cell membrane may be facilitated as a result of the increased lipophilicity of the compound. Once absorbed and taken inside the cell, the groups attached to the phosphorus can be easily removed by esterases, proteases and/or other enzymes. In some embodiments, the groups attached to the phosphorus can be removed by simple hydrolysis. Inside the cell, the phosphonate thus released may then be metabolized by cellular enzymes to the monophosphonate or the active diphosphonate (for example, a phosphono diphosphate). Furthermore, in some embodiments, varying the substituents on a compound described herein, such as a compound of Formula (I), or a pharmaceutically acceptable salt thereof, can help maintain the efficacy of the compound by reducing undesirable effects.

In some embodiments, a compound of Formula (I), or a pharmaceutically acceptable salt thereof, can act as a chain terminator of a virus and inhibit the virus' replication, wherein the virus can be HBV, HDV and/or HIV. For example, compounds of Formula (I), or a pharmaceutically acceptable salt thereof, can be incorporated into a DNA chain of the virus (such as HBV, HDV and/or HIV) and then no further elongation is observed to occur.

In some embodiments, a compound of Formula (I), or a pharmaceutically acceptable salt thereof, can have increased metabolic and/or plasma stability. In some embodiments, a compound of Formula (I), or a pharmaceutically acceptable salt thereof, can be more resistant to hydrolysis and/or more resistant to enzymatic transformations. For example, a compound of Formula (I), or a pharmaceutically acceptable salt thereof, can have increased metabolic stability, increased plasma stability, and/or can be more resistant to hydrolysis. In some embodiments, a compound of Formula (I), or a pharmaceutically acceptable salt thereof, can have improved properties. A non-limiting list of example properties include, but are not limited to, increased biological half-life, increased bioavailability, increase potency, a sustained in vivo response, increased dosing intervals, decreased dosing amounts, decreased cytotoxicity, reduction in required amounts for treating disease conditions, reduction in viral load, reduction in plasma viral load, increase CD4+T lymphocyte counts, reduction in time to seroconversion (i.e., the virus becomes undetectable in patient serum), increased sustained viral response, a reduction of morbidity or mortality in clinical outcomes, decrease in or prevention of opportunistic infections, increased subject compliance, and compatibility with other medications. In some embodiments, a compound of Formula (I), or a pharmaceutically acceptable salt thereof, can have a biological half-life of greater than 24 hours. In some embodiments, a compound of Formula (I), or a pharmaceutically acceptable salt thereof, can have more potent antiviral activity (for example, a lower EC₅₀ in an HIV, HBV and/or HDV replicon assay) as compared to the current standard of care.

### Pharmaceutical Compositions

Some embodiments described herein relate to a pharmaceutical composition, that can include an effective amount of one or more compounds described herein (e.g., a compound of Formula (I), or a pharmaceutically acceptable salt thereof) and a pharmaceutically acceptable carrier, diluent, excipient or combination thereof.

In some embodiments, the pharmaceutical composition can include a single diastereomer of a compound of Formula (I), or a pharmaceutically acceptable salt thereof, (for example, a single diastereomer is present in the pharmaceutical composition at a concentration of greater than 99% compared to the total concentration of the other diastereomers). In other embodiments, the pharmaceutical composition can include a mixture of diastereomers of a compound of Formula (I), or a pharmaceutically acceptable salt thereof. For example, the pharmaceutical composition can include a concentration of one diastereomer of >50%, ≥60%, ≥70%, ≥80%, ≥90%, ≥95%, or ≥98%, as compared to the total concentration of the other diastereomers. In some embodiments, the pharmaceutical composition includes a 1:1 mixture of two diastereomers of a compound of Formula (I), or a pharmaceutically acceptable salt thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration. A pharmaceutical composition is suitable for human and/or veterinary applications.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

Some embodiments disclosed herein relate to a method of treating and/or ameliorating a disease or condition that can include administering to a subject an effective amount of one or more compounds described herein, such as a compound of Formula (I), or a pharmaceutically acceptable salt thereof, or a pharmaceutical composition that includes a compound described herein, or a pharmaceutically acceptable salt thereof. Other embodiments disclosed herein relate to a method of treating and/or ameliorating a disease or condition that can include administering to a subject identified as suffering from the disease or condition an effective amount of one or more compounds described herein, such as a compound of Formula (I), or a pharmaceutically acceptable salt thereof, or a pharmaceutical composition that includes a compound described herein, or a pharmaceutically acceptable salt thereof.

Some embodiments described herein relate to a method of treating a HBV and/or HDV infection that can include administering to a subject identified as suffering from the HBV and/or HDV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof) in the manufacture of a medicament for treating a HBV and/or HDV infection. Still other embodiments described herein relate to the use of a compound described herein (for example, a compound of Formula (I), or a pharmaceutically acceptable salt thereof), or a pharmaceutical composition that includes a compound described herein (such as, a compound of Formula (I), or a pharmaceutically acceptable salt thereof) for treating a HBV and/or HDV infection.

Some embodiments disclosed herein relate to a method of treating a HBV and/or HDV infection that can include contacting a cell infected with the HBV and/or HDV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HBV and/or HDV infection that can include contacting a cell infected with the HBV and/or HDV with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating a HBV and/or HDV infection, wherein the use includes contacting a cell infected with the HBV and/or HDV with an effective amount of said compound(s) and/or pharmaceutical composition described herein.

Some embodiments disclosed herein relate to a method of inhibiting replication of HBV and/or HDV that can include contacting a cell infected with the HBV and/or HDV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HBV and/or HDV that can include contacting a cell infected with HBV and/or HDV with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HBV and/or HDV, wherein the use includes contacting a cell infected with the HBV and/or HDV with an effective amount of said compound(s) and/or pharmaceutical composition described herein.

In some embodiments, the HBV infection can be an acute HBV infection. In some embodiments, the HBV infection can be a chronic HBV infection.

Some embodiments disclosed herein relate to a method of treating liver cirrohosis that is developed because of a HBV and/or HDV infection that can include administering to a subject suffering from liver cirrhosis and/or contacting a cell infected with the HBV and/or HDV in a subject suffering from liver cirrhosis with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver cirrhosis with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver cirrhosis.

Some embodiments disclosed herein relate to a method of treating liver cancer (such as hepatocellular carcinoma) that is developed because of a HBV and/or HDV infection that can include administering to a subject suffering from liver cancer and/or contacting a cell infected with the HBV and/or HDV in a subject suffering from liver cancer with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver cancer (such as hepatocellular carcinoma) with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver cancer (such as hepatocellular carcinoma).

Some embodiments disclosed herein relate to a method of treating liver failure that is developed because of a HBV and/or HDV infection that can include administering to a subject suffering from liver failure and/or contacting a cell infected with the HBV and/or HDV in a subject suffering from liver failure with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver failure with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver failure.

Various indicators for determining the effectiveness of a method for treating an HBV and/or HDV infection are also known to those skilled in the art. Examples of suitable indicators include, but are not limited to, a reduction in viral load indicated by reduction in HBV DNA (or load), HBV surface antigen (HBsAg) and HBV e-antigen (HBeAg), a reduction in plasma viral load, a reduction in viral replication, a reduction in time to seroconversion (virus undetectable in patient serum), an increase in the rate of sustained viral response to therapy, an improvement in hepatic function, and/or a reduction of morbidity or mortality in clinical outcomes.

In some embodiments, an effective amount of a compound of Formula (I), or a pharmaceutically acceptable salt thereof, is an amount that is effective to reduce HBV and/or HDV viral load to undetectable levels, for example, to about 10 to about 50, or to about 15 to about 25 international units/mL serum, or to less than about 20 international units/mL serum. In some embodiments, an effective amount of a compound of Formula (I), or a pharmaceutically acceptable salt thereof, is an amount that is effective to reduce HBV and/or HDV viral load compared to the HBV and/or HDV viral load before being provided the compound of Formula (I), or a pharmaceutically acceptable salt thereof. In some embodiments, an effective amount of a compound of Formula (I), or a pharmaceutically acceptable salt thereof, can be an amount that is effective to reduce HBV and/or HDV viral load to lower than about 20 international units/mL serum. In some embodiments, an effective amount of a compound of Formula (I), or a pharmaceutically acceptable salt thereof, is an amount that is effective to achieve a reduction in HBV and/or HDV viral load in the serum of the subject to an undetectable level and/or in the range of about 1.5-log to about a 2.5-log reduction, about a 3-log to about a 4-log reduction, or a greater than about 5-log reduction compared to the viral load before being provided the compound of Formula (I), or a pharmaceutically acceptable salt thereof. For example, the HBV and/or HDV viral load can be measured before being provided the compound of Formula (I), or a pharmaceutically acceptable salt thereof, and again after completion of at least a portion of the treatment regime with the compound of Formula (I), or a pharmaceutically acceptable salt thereof (for example, 1 month after initiation or completion).

In some embodiments, a compound of Formula (I), or a pharmaceutically acceptable salt thereof, can result in at least a 1, 2, 3, 4, 5, 10, 15, 20, 25, 50, 75, 100-fold or more reduction in the replication of HBV and/or HDV relative to pre-treatment levels in a subject, as determined after completion of, or completion of at least a portion of, the treatment regime (for example, 1 month after initiation or completion). In some embodiments, a compound of Formula (I), or a pharmaceutically acceptable salt thereof, can result in a reduction of the replication of HBV and/or HDV relative to pre-treatment levels in the range of more than 1 fold, about 2 to about 5 fold, about 10 to about 20 fold, about 15 to about 40 fold, or about 50 to about 100 fold. In some embodiments, a compound of Formula (I), or a pharmaceutically acceptable salt thereof, can result in a reduction of HBV and/or HDV replication in the range of more than 0.5 log, 1 to 1.5 log, 1.5 log to 2 log, 2 log to 2.5 log, 2.5 to 3 log, 3 log to 3.5 log or 3.5 to 4 log more reduction of HBV and/or HDV replication compared to the reduction of HBV and/or HDV replication achieved by the standard of care of HBV and/or HDV, administered according to the standard of care, or may achieve the same reduction as that standard of care therapy in a shorter period of time, for example, in one month, two months, or three months, as compared to the reduction achieved after six months of standard of care therapy.

In some embodiments, an effective amount of a compound of Formula (I), or a pharmaceutically acceptable salt thereof, is an amount that is effective to achieve a sustained virologic response, for example, non-detectable or substantially non-detectable HBV and/or HDV DNA load (e.g., less than about 25, or less than about 15 international units per milliliter serum) is found in the subject's serum for a period of at least about one month, at least about two months, at least about three months, at least about four months, at least about five months, or at least about six months following cessation of therapy.

In some embodiments, an effective amount of a compound of Formula (I), or a pharmaceutically acceptable salt thereof, can reduce the HBV and/or HDV viral load by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the viral load a subject treated with standard of care, in an untreated subject or a placebo-treated subject. Methods of detecting HBV and/or HDV viral load are known to those skilled in the art and include immunological-based methods, e.g., enzyme-linked immunosorbent assays (ELISA), radioimmunoassays, and the like, that detect HBV and/or HDV antibodies and other markers indicative of HBV and/or HDV viral load, and combinations thereof.

Some embodiments described herein relate to a method of inhibiting HIV activity that can include contacting a cell infected with HIV with an effective amount of a compound of Formula (I), or a pharmaceutically acceptable salt thereof. Some embodiments described herein relate to a method of inhibiting HIV activity that can include administering to a subject infected with HIV an effective amount of a compound of Formula (I), or a pharmaceutically acceptable salt thereof. In some embodiments, a compound of Formula (I), or a pharmaceutically acceptable salt thereof, can inhibit a viral reverse transcriptase, and thus, inhibit the transcription of HIV RNA to DNA. In some embodiments, a compound of Formula (I), or a pharmaceutically acceptable salt thereof, can inhibit an HIV integrase. In some embodiments, a compound of Formula (I), or a pharmaceutically acceptable salt thereof, can inhibit viral envelop glycoprotein 120 (gp 120).

Some embodiments described herein relate to a method of treating a HIV infection that can include administering to a subject identified as suffering from the HIV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof), in the manufacture of a medicament for treating a HIV infection. Still other embodiments described herein relate to the use of a compound described herein (such as, a compound of Formula (I), or a pharmaceutically acceptable salt thereof), or a pharmaceutical composition that includes an effective amount of a compound described herein (for example, a compound of Formula (I), or a pharmaceutically acceptable salt thereof) for treating a HIV infection.

Some embodiments disclosed herein relate to a method of treating a HIV infection that can include contacting a cell infected with the HIV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HIV infection that can include contacting a cell infected with the HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HIV infection, wherein the use includes contacting a cell infected with the HIV with an effective amount of said compound(s) and/or pharmaceutical composition.

Some embodiments disclosed herein relate to a method of inhibiting replication of HIV that can include contacting a cell infected with the HIV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HIV that can include contacting a cell infected with HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HIV, wherein the use includes contacting a cell infected with the HIV with an effective amount of said compound(s) and/or pharmaceutical composition.

In some embodiments described herein, when the infection is caused by HIV, and/or the virus is HIV, the subject suffers from an opportunistic infection (OI). OIs take advantage of the subjects weakened immune system. In some embodiments described herein, a subject having a CD4+T lymphocyte count of less than about 200 cells/mL is an at increased risk of developing an OI. In some embodiments, OIs occur when the CD4+ T lymphocyte count is less than about 500 cells/mL. In some embodiments, an OI occurs when an HIV viral load is greater than about 100,000 copies/mL. In some embodiments, HIV viral loads and/or CD4+ T lymphocyte counts can be determined by conventional standard of care methodologies, for example, through HIV immunoassay detection assays for the detection of HIV antibodies and/or HIV p24 antigen.

Some embodiments described herein relate to a method of treating an OI related to a HIV infection selected from candidiasis, bronchitis, pneumonitis, esophagitis, invasive cervical cancer, coccidioidomycosis, cryptococcosis, chronic intestinal cryptosporidiosis, cytomegalovirus disease, encephalopathy, herpes simplex, histoplasmosis, chronic intestinal isosporiasis, Kaposi's sarcoma, lymphoma, Mycobacterium avium complex, tuberculosis, Pneumocystis carinii pneumonia, progressive multifocal leukoencephalopathy, Salmonella septicemia, toxoplasmosis of brain, and wasting syndrome in a subject suffering from one or more of the aforementioned conditions that can include providing to the subject an effective amount of a compound or a pharmaceutical composition described herein (for example, a compound of Formula (I), or a pharmaceutically acceptable salt thereof). Some embodiments described herein relate to a method of preventing and/or treating one or more OI in a subject having a HIV infection that can include providing to the subject an effective amount of a compound or a pharmaceutical composition described herein (for example, a compound of Formula (I), or a pharmaceutically acceptable salt thereof). Also contemplated is a method for reducing or eliminating one or more OI in a subject having an HIV infection by providing to the subject an effective amount of a compound or a pharmaceutical composition described herein (for example, a compound of Formula (I), or a pharmaceutically acceptable salt thereof). In some embodiments, this method can include slowing or halting the progression of an OI. In other embodiments, the course of the OI can be reversed, and stasis or improvement in the infection is contemplated. In some embodiments, one or more of candidiasis, bronchitis, pneumonitis, esophagitis, invasive cervical cancer, coccidioidomycosis, cryptococcosis, chronic intestinal cryptosporidiosis, cytomegalovirus disease, encephalopathy, herpes simplex, histoplasmosis, chronic intestinal isosporiasis, Kaposi's sarcoma, lymphoma, Mycobacterium avium complex, tuberculosis, Pneumocystis carinii pneumonia, progressive multifocal leukoencephalopathy, Salmonella septicemia, toxoplasmosis of brain, and wasting syndrome can be treated by contacting a cell infected with HIV with an effective amount of a compound described herein (for example, a compound of Formula (I), or a pharmaceutically acceptable salt thereof.)

Two types of HIV have been characterized, HIV-1 and HIV-2. HIV-1 is more virulent and more infective, and has a global prevalence, whereas HIV-2 is less virulent and is geographically confined. In some embodiments, an effective amount of a compound of Formula (I), or a pharmaceutically acceptable salt thereof, or a pharmaceutical composition that includes an effective amount of a compound of Formula (I), or a pharmaceutically acceptable salt thereof, can be effective to treat HIV-1. In some embodiments, an effective amount of a compound of Formula (I), or a pharmaceutically acceptable salt thereof, or a pharmaceutical composition that includes an effective amount of a compound of Formula (I), or a pharmaceutically acceptable salt thereof, can be effective to treat HIV-2. In some embodiments, a compound described herein (for example, a compound of Formula (I), or a pharmaceutically acceptable salt thereof) can be effective to treat both genotypes of HIV (HIV-1 and HIV-2).

Various indicators for determining the effectiveness of a method for treating an HIV infection are known to those skilled in the art. Examples of suitable indicators include, but are not limited to, a reduction in viral load, a reduction in plasma viral load, an increase CD4+T lymphocyte counts, a reduction in viral replication, a reduction in time to seroconversion (virus undetectable in patient serum), an increase in the rate of sustained viral response to therapy, a reduction of morbidity or mortality in clinical outcomes and/or a reduction in the rate of opportunistic infections. Similarly, successful therapy with an effective amount of a compound or a pharmaceutical composition described herein (for example, a compound of Formula (I), or a pharmaceutically acceptable salt thereof) can reduce the incidence of opportunistic infections in HIV infected subjects.

In some embodiments, an effective amount of a compound of Formula (I), or a pharmaceutically acceptable salt thereof, is an amount that is effective to reduce HIV viral titers to undetectable levels, for example, to about 10 to about 50, or to about 15 to about 25 international units/mL serum, or to less than about 20 international units/mL serum. In some embodiments, an effective amount of a compound of Formula (I), or a pharmaceutically acceptable salt thereof, is an amount that is effective to reduce HIV viral load compared to the HIV viral load before being provided the compound of Formula (I), or a pharmaceutically acceptable salt thereof. In some embodiments, an effective amount of a compound of Formula (I), or a pharmaceutically acceptable salt thereof, can be an amount that is effective to reduce HIV viral load to lower than about 20 international units/mL serum. In some embodiments, an effective amount of a compound of Formula (I), or a pharmaceutically acceptable salt thereof, is an amount that is effective to achieve a reduction in HIV viral titer in the serum of the subject in the range of about 1.5-log to about a 2.5-log reduction, about a 3-log to about a 4-log reduction, or a greater than about 5-log reduction compared to the viral load before being provided the compound of Formula (I), or a pharmaceutically acceptable salt thereof. For example, the HIV viral load can be measured before being provided the compound of Formula (I), or a pharmaceutically acceptable salt thereof, and again after completion of the treatment regime with the compound of Formula (I), or a pharmaceutically acceptable salt thereof (for example, 1 month after completion).

In some embodiments, an effective amount of a compound of Formula (I), or a pharmaceutically acceptable salt thereof, is an amount that is effective to increase CD4+T lymphocyte counts from less than about 200 cells/mL to greater than about 1,200 cells/mL. In some embodiments, an effective amount of a compound of Formula (I), or a pharmaceutically acceptable salt thereof, is an amount that is effective to increase CD4+T lymphocyte counts from less than about 200 cells/mL to greater than about 500 cells/mL.

In some embodiments, a compound of Formula (I), or a pharmaceutically acceptable salt thereof, can result in at least a 1, 2, 3, 4, 5, 10, 15, 20, 25, 50, 75, 100-fold or more reduction in the replication of the human immunodeficiency virus relative to pre-treatment levels in a subject, as determined after completion of the treatment regime (for example, 1 month after completion). In some embodiments, a compound of Formula (I), or a pharmaceutically acceptable salt thereof, can result in a reduction of the replication of the human immunodeficiency virus relative to pre-treatment levels in the range of about 2 to about 5 fold, about 10 to about 20 fold, about 15 to about 40 fold, or about 50 to about 100 fold. In some embodiments, a compound of Formula (I), or a pharmaceutically acceptable salt thereof, can result in a reduction of the human immunodeficiency virus replication in the range of 1 to 1.5 log, 1.5 log to 2 log, 2 log to 2.5 log, 2.5 to 3 log, 3 log to 3.5 log or 3.5 to 4 log more reduction of the human immunodeficiency virus replication compared to the reduction of the human immunodeficiency virus reduction achieved by standard of care therapy, such as therapy including ritonavir in combination with etravirine, or may achieve the same reduction as that standard of care therapy in a shorter period of time, for example, in one month, two months, or three months, as compared to the reduction achieved after six months of standard of care therapy.

In some embodiments, an effective amount of a compound of Formula (I), or a pharmaceutically acceptable salt thereof, is an amount that is effective to achieve a sustained viral response, for example, non-detectable or substantially non-detectable HIV RNA (e.g., less than about 25, or less than about 15 international units per milliliter serum) is found in the subject's serum for a period of at least about one month, at least about two months, at least about three months, at least about four months, at least about five months, or at least about six months following cessation of therapy.

In some embodiments, an effective amount of a compound of Formula (I), or a pharmaceutically acceptable salt thereof, can reduce the HIV viral load by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the viral load in an untreated subject, or to a placebo-treated subject. Methods of detecting HIV viral load are known to those skilled in the art and include immunological-based methods, e.g., enzyme-linked immunosorbent assays (ELISA), radioimmunoassays, and the like, that detect HIV-1 and/or HIV-2 antibodies, HIV-1 p24 antigen, and other markers indicative of HIV viral load, and combinations thereof.

Subjects who are clinically diagnosed with HBV, HDV and/or HIV infection include “naïve” subjects (e.g., subjects not previously treated for HBV, HDV and/or HIV, particularly those who have not previously received ART for HIV, including ritonavir-based therapy) and individuals who have failed prior treatment for HBV, HDV and/or HIV (“treatment failure” subjects). Treatment failure subjects include “non-responders” (for HIV, these are subjects in whom the HIV titer was not significantly or sufficiently reduced by a previous treatment for HIV (≤0.5 log IU/mL)), for example, a previous ART, including ritonavir or other therapy; and “relapsers” (for HIV, subjects who were previously treated for HIV, for example, who received a previous ART whose HIV titer decreased, and subsequently increased). Further examples of subjects include subjects with an acute HBV and/or HDV infection, subjects with a chronic HBV and/or HDV, and subjects who are asymptomatic.

In some embodiments, a compound of Formula (I), or a pharmaceutically acceptable salt thereof, can be provided to a treatment failure subject suffering from HBV, HDV and/or HIV. In some embodiments, a compound of Formula (I), or a pharmaceutically acceptable salt thereof, can be provided to a non-responder subject suffering from HBV, HDV and/or HIV. In some embodiments, a compound of Formula (I), or a pharmaceutically acceptable salt thereof, can be provided to a relapsed subject suffering from HBV, HDV and/or HIV. In some embodiments, the subject can be asymptomatic, for example, the subject can be infected with HBV and/or HDV but does not exhibit any symptoms of the viral infection. In some embodiments, the subject can be immunocompromised. In some embodiments, the subject is suffering from at least one of HIV, HBV and/or HDV.

In some embodiments, a compound of Formula (I), or a pharmaceutically acceptable salt thereof, can be provided to a subject suffering from chronic HBV and/or HDV. In some embodiments, a compound of Formula (I), or a pharmaceutically acceptable salt thereof, can be provided to a subject suffering from acute HBV and/or HDV.

After a period of time, infectious agents can develop resistance to one or more therapeutic agents. The term “resistance” as used herein refers to a viral strain displaying a delayed, lessened and/or null response to a therapeutic agent(s). In some instances, the virus sometimes mutates or produces variations that are resistant or partially resistant to certain drugs. For example, after treatment with an antiviral agent, the viral load of a subject infected with a resistant virus may be reduced to a lesser degree compared to the amount in viral load reduction exhibited by a subject infected with a non-resistant strain. In some embodiments, a compound of Formula (I), or a pharmaceutically acceptable salt thereof, can be provided to a subject infected with an HBV and/or HDV strain that is resistant to one or more different anti-HBV and/or anti-HDV agents (for example, an agent used in a conventional standard of care). In some embodiments, development of resistant HBV and/or HDV strains is delayed when a subject is treated with a compound of Formula (I), or a pharmaceutically acceptable salt thereof, compared to the development of HBV and/or HDV strains resistant to other HBV and/or HDV drugs (such as an agent used in a conventional standard of care). In some embodiments, a compound of Formula (I), or a pharmaceutically acceptable salt thereof, can be provided to a subject infected with an HIV strain that is resistant to one or more different anti-HIV agents (for example, an agent used in a conventional standard of care). In some embodiments, development of resistant HIV strains is delayed when a subject is treated with a compound of Formula (I), or a pharmaceutically acceptable salt thereof, compared to the development of HIV strains resistant to other HIV drugs (such as an agent used in a conventional standard of care).

In some embodiments, an effective amount of a compound of Formula (I), or a pharmaceutically acceptable salt thereof, can be provided to a subject for whom other anti-HBV, anti-HDV and/or anti-HIV medications are contraindicated. In some embodiments, a compound of Formula (I), or a pharmaceutically acceptable salt thereof, can be provided to a subject that is hypersensitive to an antiviral agent.

Some subjects being treated for HBV, HDV and/or HIV experience a viral load rebound. The term “viral load rebound” as used herein refers to a sustained increase of viral load (such as ≥0.5 log IU/Ml for HIV) above nadir before the end of treatment. For HIV, nadir is a ≥0.5 log IU/mL decrease from baseline. In some embodiments, a compound of Formula (I), or a pharmaceutically acceptable salt thereof, can be provided to a subject experiencing viral load rebound, or can prevent such viral load rebound when used to treat the subject.

The standard of care for treating HBV, HDV and HIV has been associated with several side effects (also referred to as adverse effects). In some embodiments, a compound of Formula (I), or a pharmaceutically acceptable salt thereof, can decrease the number and/or severity of side effects observed in subjects being treated with the standard of care for a specific virus, such as HBV, HDV and HIV. Examples of side effects for a subject being treated for HBV and/or HDV include, but are not limited to dyspepsia, neuropathy, cough, loss of appetite, lactic acidosis, lipodystrophy, diarrhea, fatigue, insomnia, rash, fever, malaise, tachycardia, chills, headache, arthralgias, myalgias, apathy, nausea, vomiting, cognitive changes, asthenia, and drowsiness. In some embodiments, a compound of Formula (I), or a pharmaceutically acceptable salt thereof, can decrease the number and/or severity of side effects. For example, the number and/or severity of side effects observed in HIV subjects being treated with an ART according to the standard of care. Examples of side effects for a subject being treated for HIV include, but are not limited to loss of appetite, lipodystrophy, diarrhea, fatigue, elevated cholesterol and triglycerides, rash, insomnia, fever, malaise, tachycardia, chills, headache, arthralgias, myalgias, apathy, nausea, vomiting, cognitive changes, asthenia, drowsiness, lack of initiative, irritability, confusion, depression, severe depression, suicidal ideation, anemia, low white blood cell counts, and thinning of hair. In some embodiments, a compound of Formula (I), or a pharmaceutically acceptable salt thereof, can be provided to a subject that discontinued a HBV, HDV and/or HIV therapy because of one or more adverse effects or side effects associated with one or more other anti-HBV, HDV and/or HIV agents (for example, an agent used in a conventional standard of care).

Table 1 provides some embodiments of the percentage improvement obtained using a compound of Formula (I), or a pharmaceutically acceptable salt thereof, as compared to the standard of care for HBV, HDV and/or HIV. Examples include the following: in some embodiments, a compound of Formula (I), or a pharmaceutically acceptable salt thereof, results in a percentage of non-responders that is 10% less than the percentage of non-responders receiving the standard of care. In some embodiments, a compound of Formula (I), or a pharmaceutically acceptable salt thereof, results in a number of side effects that is in the range of about 10% to about 30% less than compared to the number of side effects experienced by a subject receiving the standard of care; and in some embodiments, a compound of Formula (I), or a pharmaceutically acceptable salt thereof, results in a severity of a side effect (such as one of those described herein) that is 25% less than compared to the severity of the same side effect experienced by a subject receiving the standard of care. Methods of quantifying the severity of a side effect are known to those skilled in the art.

TABLE 1

| | | | Percentage | | |
|---|---|---|---|---|---|
| Percentage | Percentage | Percentage | of viral | Number | Severity |
| of non- | of | of | load | of side | of side |
| responders | relapsers | resistance | rebound | effects | effects |
| 10% less | 10% less | 10% less | 10% less | 10% less | 10% less |
| 25% less | 25% less | 25% less | 25% less | 25% less | 25% less |
| 40% less | 40% less | 40% less | 40% less | 40% less | 40% less |
| 50% less | 50% less | 50% less | 50% less | 50% less | 50% less |
| 60% less | 60% less | 60% less | 60% less | 60% less | 60% less |
| 70% less | 70% less | 70% less | 70% less | 70% less | 70% less |
| 80% less | 80% less | 80% less | 80% less | 80% less | 80% less |
| 90% less | 90% less | 90% less | 90% less | 90% less | 90% less |
| about 10% | about 10% | about 10% | about 10% | about 10% | about 10% |
| to about | to about | to about | to about | to about | to about |
| 30% less | 30% less | 30% less | 30% less | 30% less | 30% less |
| about 20% | about 20% | about 20% | about 20% | about 20% | about 20% |
| to about | to about | to about | to about | to about | to about |
| 50% less | 50% less | 50% less | 50% less | 50% less | 50% less |
| about 30% | about 30% | about 30% | about 30% | about 30% | about 30% |
| to about | to about | to about | to about | to about | to about |
| 70% less | 70% less | 70% less | 70% less | 70% less | 70% less |
| about 20% | about 20% | about 20% | about 20% | about 20% | about 20% |
| to about | to about | to about | to about | to about | to about |
| 80% less | 80% less | 80% less | 80% less | 80% less | 80% less |

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The terms “therapeutically effective amount” and “effective amount” are used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively, dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years. In some embodiments, a compound of Formula (I), or a pharmaceutically acceptable salt thereof, can be administered less frequently compared to the frequency of administration of an agent within the standard of care. In some embodiments, a compound of Formula (I), or a pharmaceutically acceptable salt thereof, can be administered one time per day. For example, a compound of Formula (I), or a pharmaceutically acceptable salt thereof, can be administered one time per day to a subject suffering from a HIV infection. In some embodiments, the total time of the treatment regime with a compound of Formula (I), or a pharmaceutically acceptable salt thereof, can be less compared to the total time of the treatment regime with the standard of care.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Combination Therapies

In some embodiments, the compounds disclosed herein, such as a compound of Formula (I), or a pharmaceutically acceptable salt thereof, or a pharmaceutical composition that includes a compound described herein, or a pharmaceutically acceptable salt thereof, can be used in combination with one or more additional agent(s). Examples of additional agents that can be used in combination with a compound of Formula (I), or a pharmaceutically acceptable salt thereof, or a pharmaceutical composition that includes a compound of Formula (I), or a pharmaceutically acceptable salt thereof, can be agents currently used in a conventional standard of care for treating HIV, HBV, and/or HDV. In some embodiments, a compound of Formula (I), or a pharmaceutically acceptable salt thereof, or a pharmaceutical composition that includes a compound of Formula (I), or a pharmaceutically acceptable salt thereof, can be used with one, two, three or more additional agents described herein.

In some embodiments, when the infection is caused by HBV, HBV and/or HIV, the additional therapeutic agent can be an antiretroviral therapy (ART) agent such as a non-nucleoside reverse transcriptase inhibitor (NNRTI), a nucleoside reverse transcriptase inhibitor (NRTI), a polymerase inhibitor, a protease inhibitor (PI), a fusion/entry inhibitor, an interferon, a viral maturation inhibitor, a capsid assembly modulator, a FXR agonist, a TNF/cyclophilin inhibitor, a TLR agonist, a vaccine, an siRNA or ASO covalently closed circular DNA (cccDNA) inhibitor, a gene silencing agent, an HBx inhibitor, a surface antigen (sAg) secretion inhibitor (for example, HBsAg), other HBV antiviral compound, other HDV antiviral compound and/or other HIV antiviral compound, or a pharmaceutically acceptable salt of any of the foregoing.

In some embodiments, a compound of Formula (I), or a pharmaceutically acceptable salt thereof, or a pharmaceutical composition that includes a compound of Formula (I), or a pharmaceutically acceptable salt thereof, can be used in combination with an agent(s) currently used in a conventional standard of care therapy. For example, for the treatment of HBV and/or HDV, a compound disclosed herein can be used in combination with an interferon therapy.

In some embodiments, a compound of Formula (I), or a pharmaceutically acceptable salt thereof, or a pharmaceutical composition that includes a compound of Formula (I), or a pharmaceutically acceptable salt thereof, can be substituted for an agent currently used in a conventional standard of care therapy. For example, for the treatment of HIV, a compound of Formula (I), or a pharmaceutically acceptable salt thereof, or a pharmaceutical composition that includes a compound of Formula (I), or a pharmaceutically acceptable salt thereof, can be used in place of a conventional ART inhibitor.

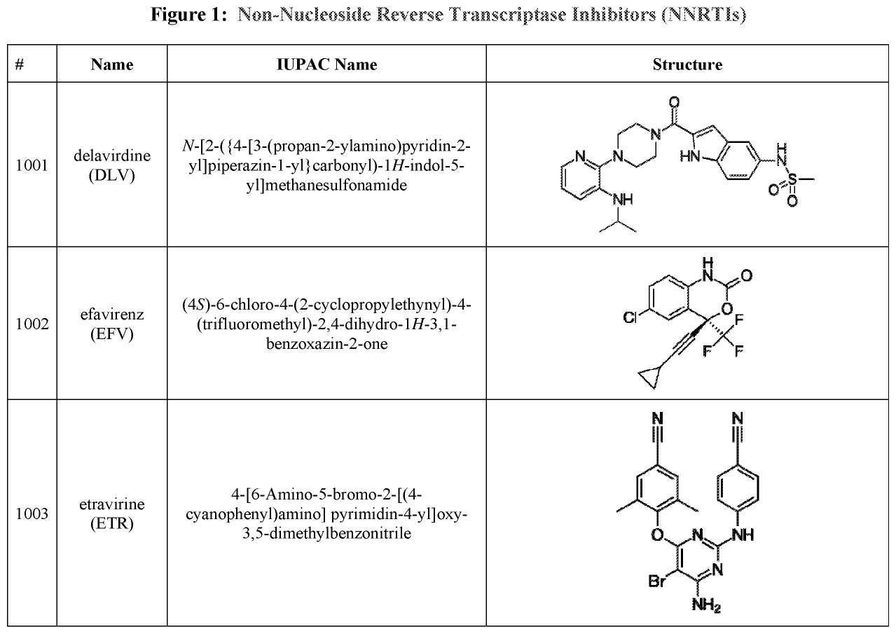

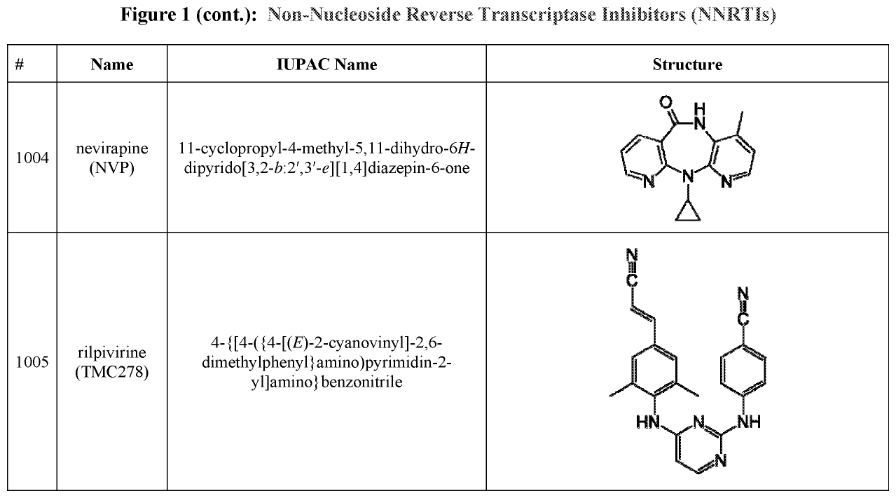

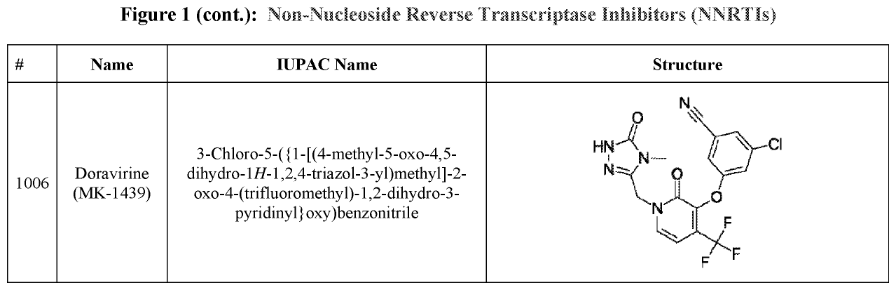

In some embodiments, a compound of Formula (I), or a pharmaceutically acceptable salt thereof, or a pharmaceutical composition that includes a compound of Formula (I), or a pharmaceutically acceptable salt thereof, can be used in combination with a non-nucleoside reverse transcriptase inhibitor (NNRTI). In some embodiments, the NNRTI can inhibit a HBV and/or HDV reverse transcriptase. Examples of suitable NNRTIs include, but are not limited to, delavirdine (Rescriptor®), efavirenz (Sustiva®), etravirine (Intelence®), nevirapine (Viramune®), rilpivirine (Edurant®), doravirine, and pharmaceutically acceptable salts of any of the foregoing, and/or a combination thereof. A non-limiting list of example NNRTIs includes compounds numbered 1001-1006 in FIG. 1.

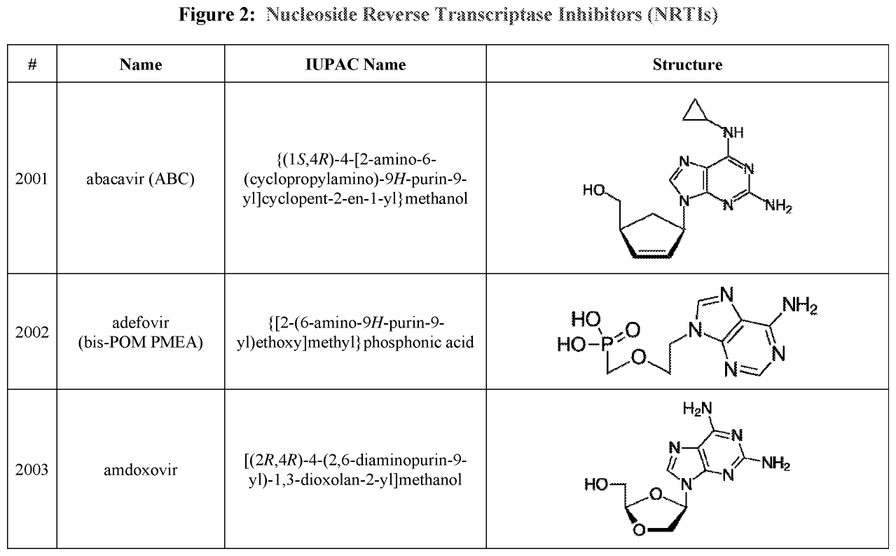

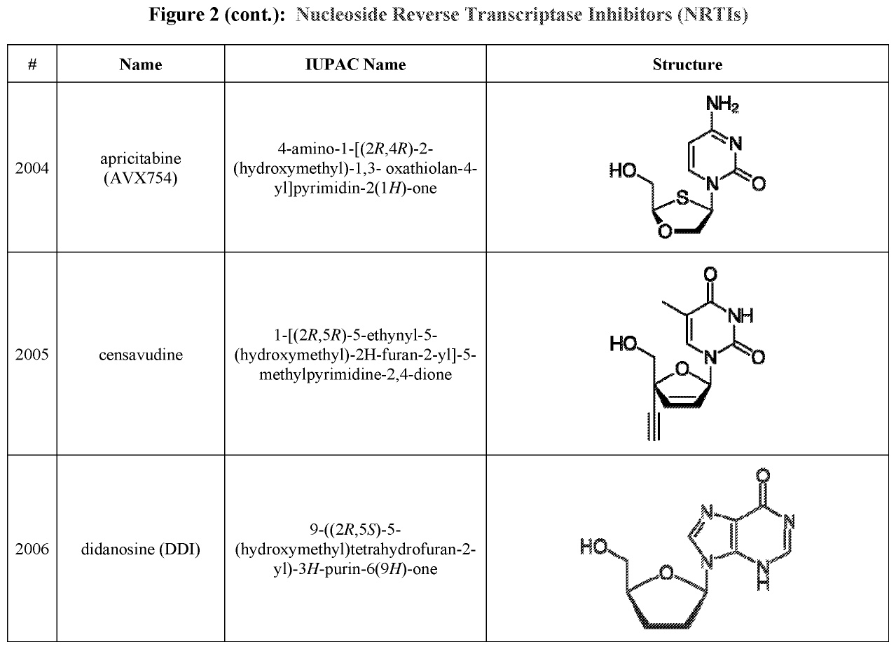

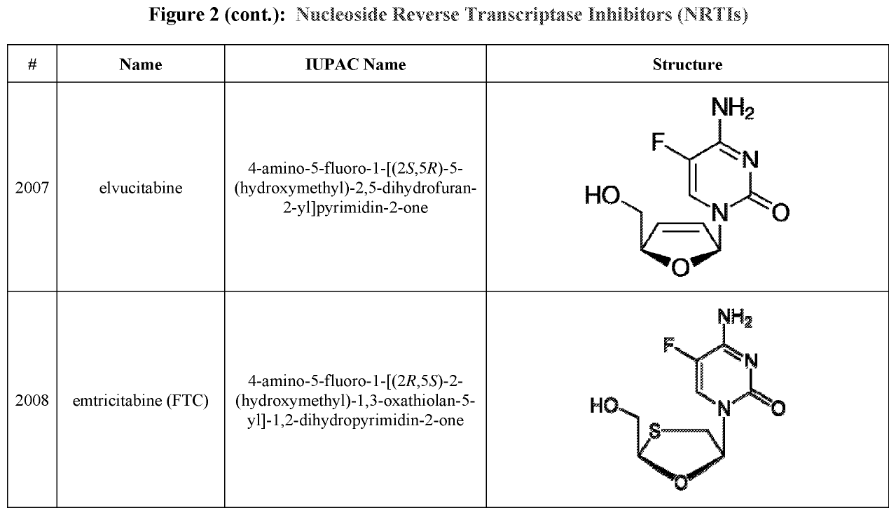

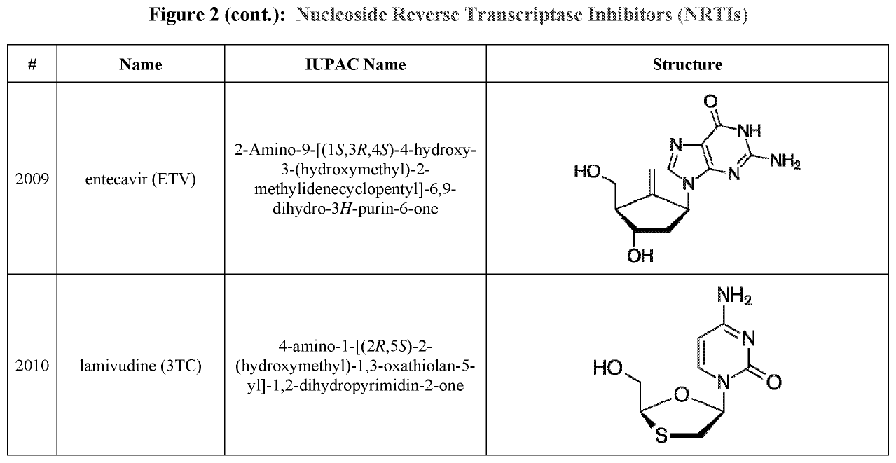

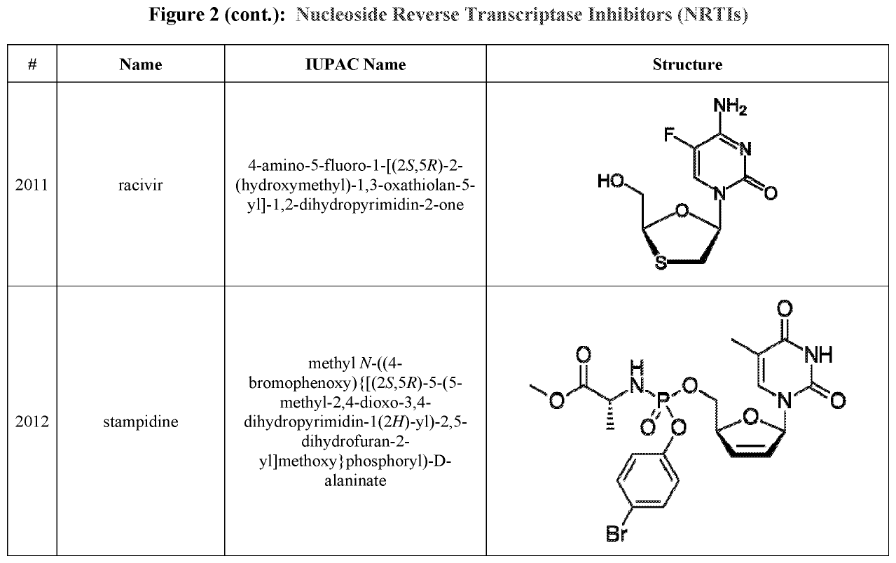

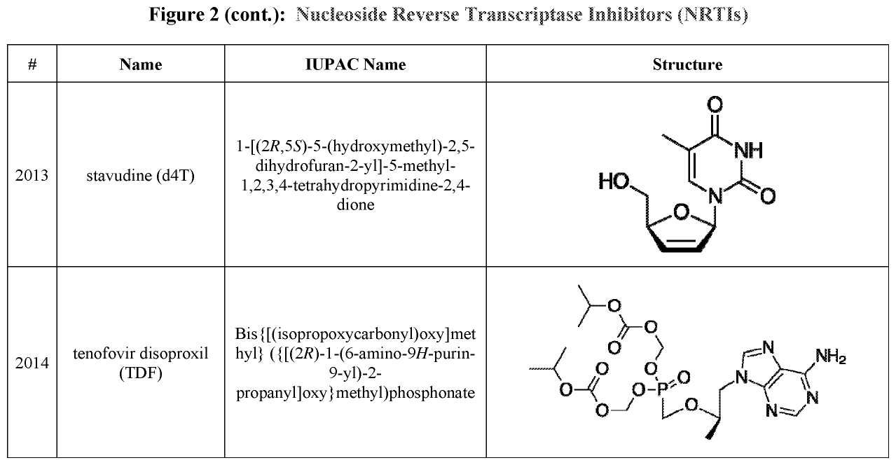

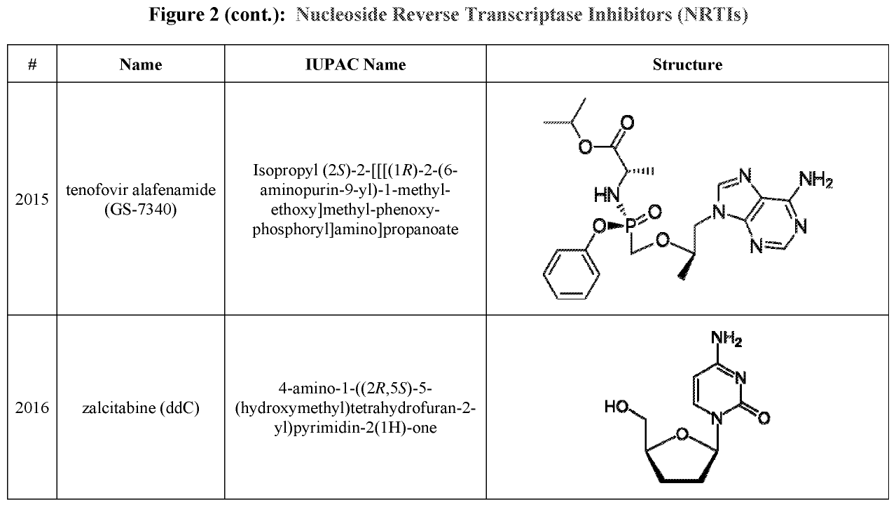

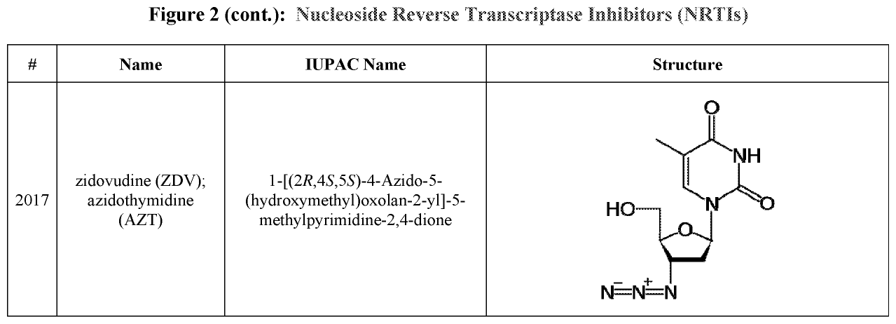

In some embodiments, a compound of Formula (I), or a pharmaceutically acceptable salt thereof, or a pharmaceutical composition that includes a compound of Formula (I), or a pharmaceutically acceptable salt thereof, can be used in combination with a nucleoside reverse transcriptase inhibitor (NRTI). In some embodiments, the NRTI can inhibit a HBV and/or HDV reverse transcriptase. Examples of suitable NRTIs include, but are not limited to, abacavir (Ziagen®), adefovir (Hepsera®), amdoxovir, apricitabine, censavudine, didanosine (Videx®), elvucitabine, emtricitabine (Emtriva®), entecavir (Baraclude®), lamivudine (Epivir®), racivir, stampidine, stavudine (Zerit®), tenofovir disoproxil (including Viread®), tenofovir alafenamide, zalcitabine (Hivid®), zidovudine (Retrovir®), and pharmaceutically acceptable salts of any of the foregoing, and/or a combination thereof. A non-limiting list of example NRTIs includes compounds numbered 2001-2017 in FIG. 2.

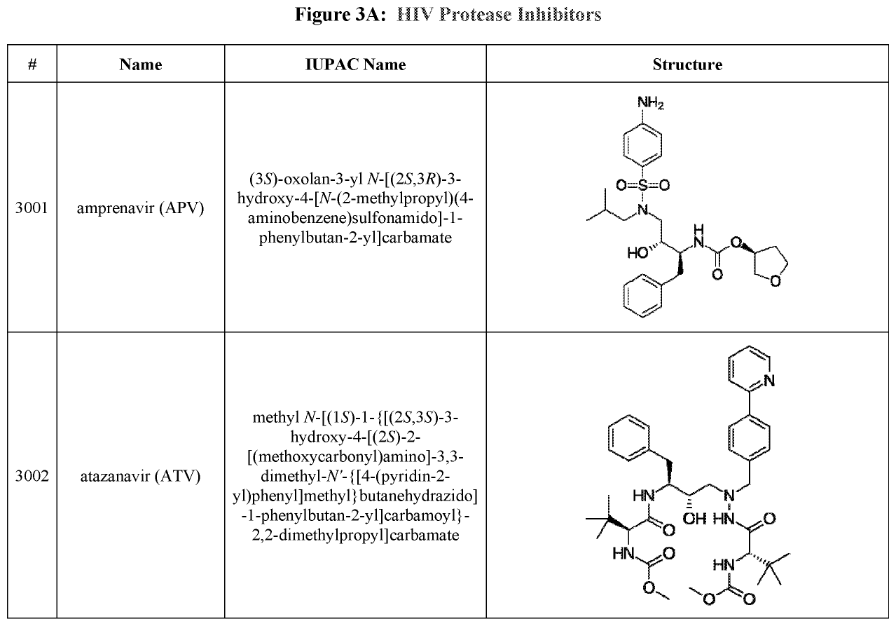

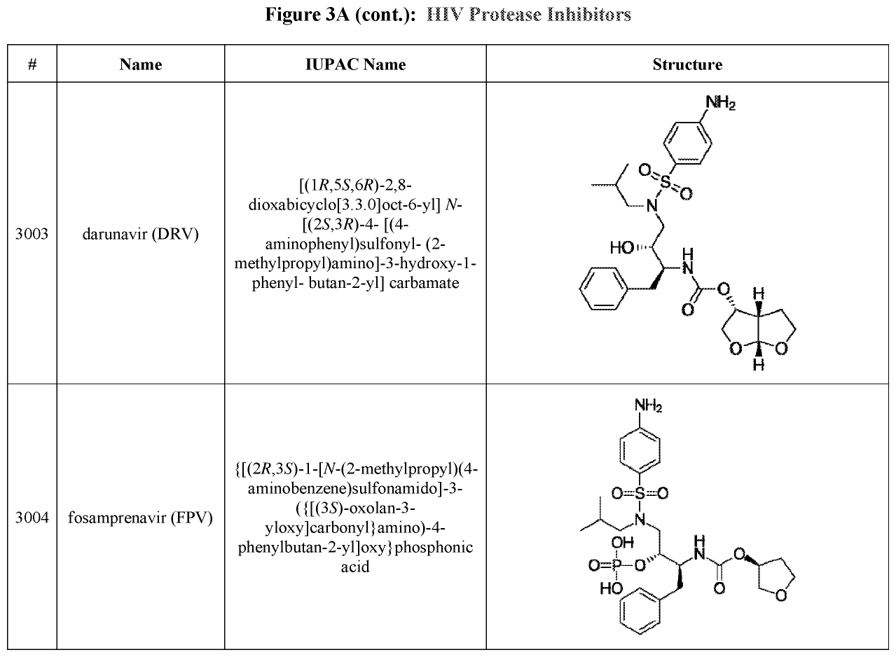

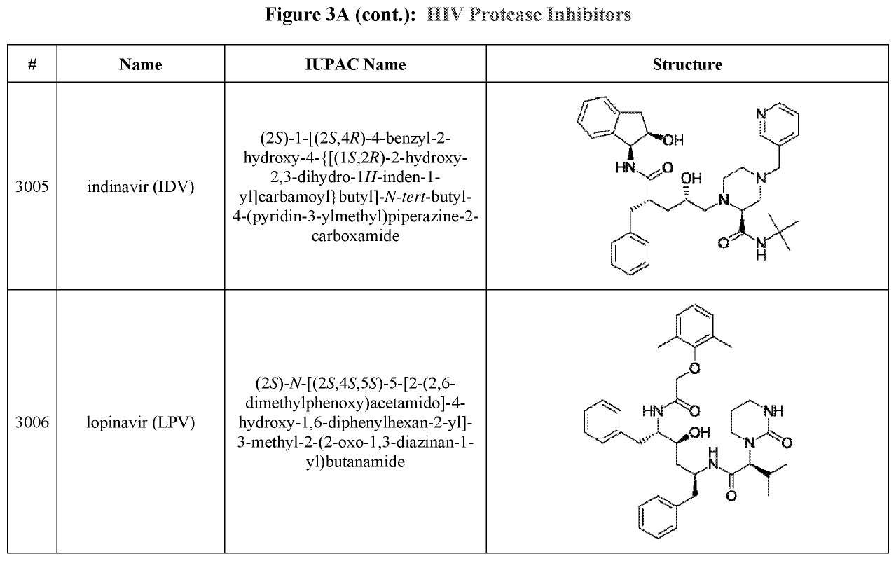

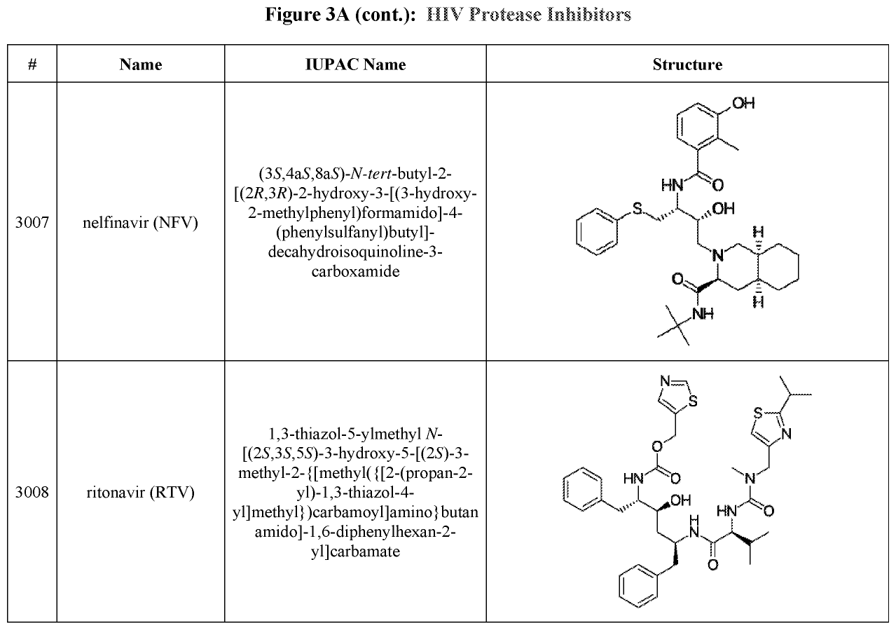

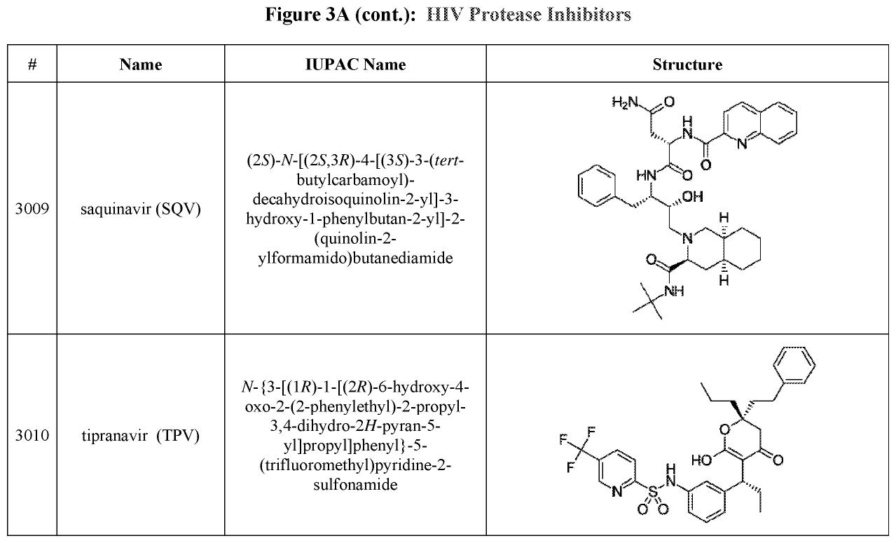

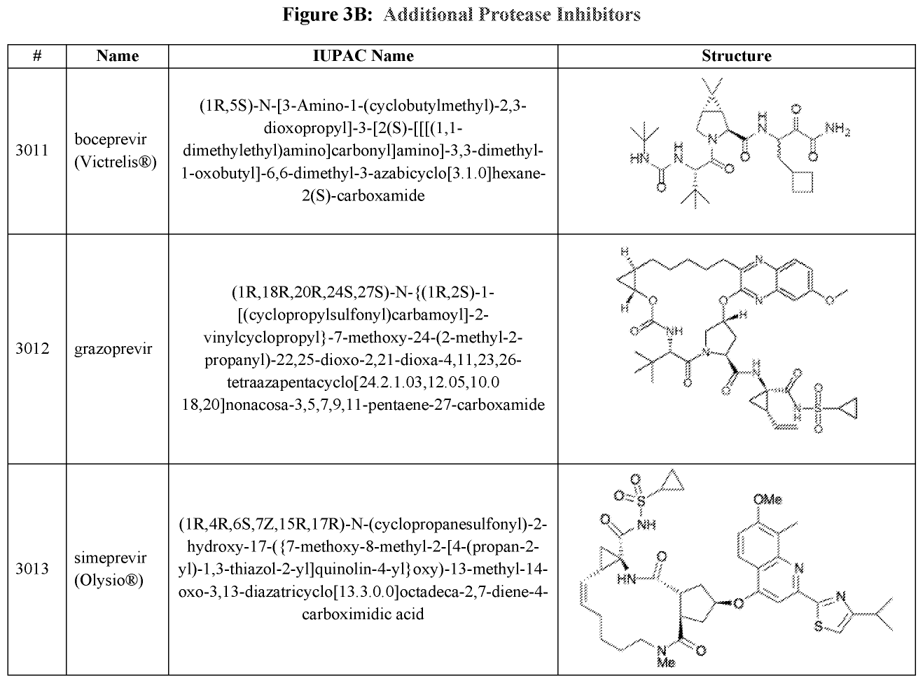

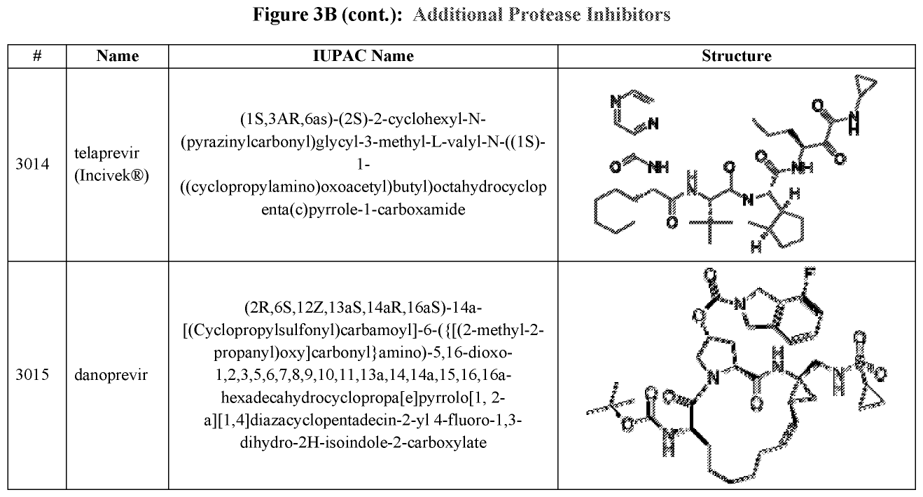

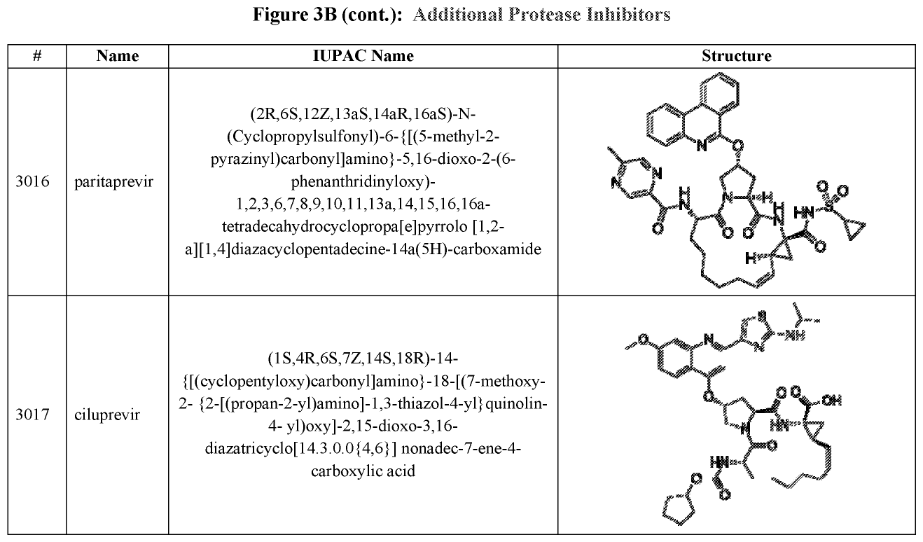

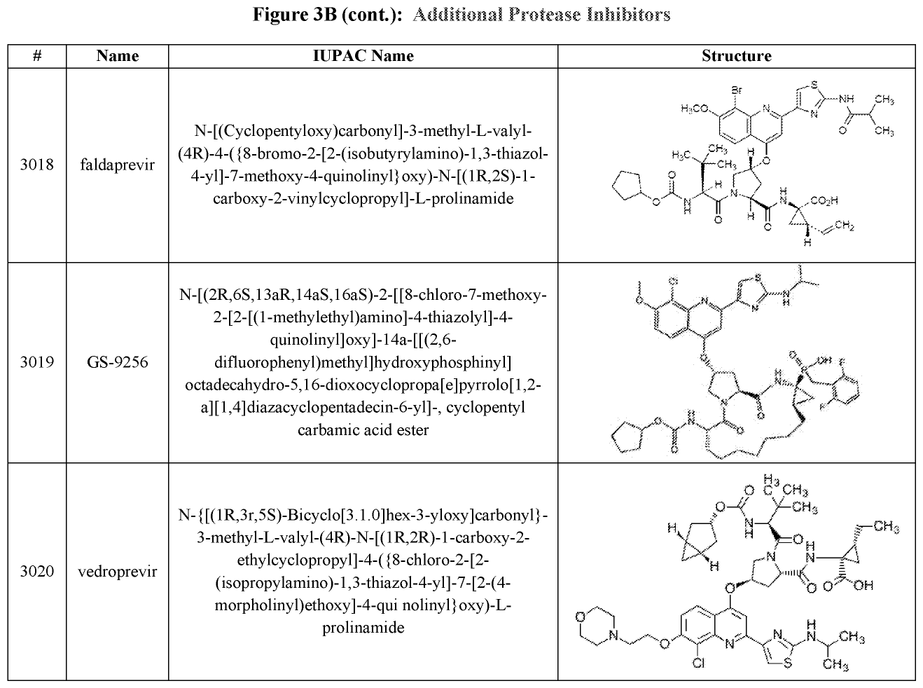

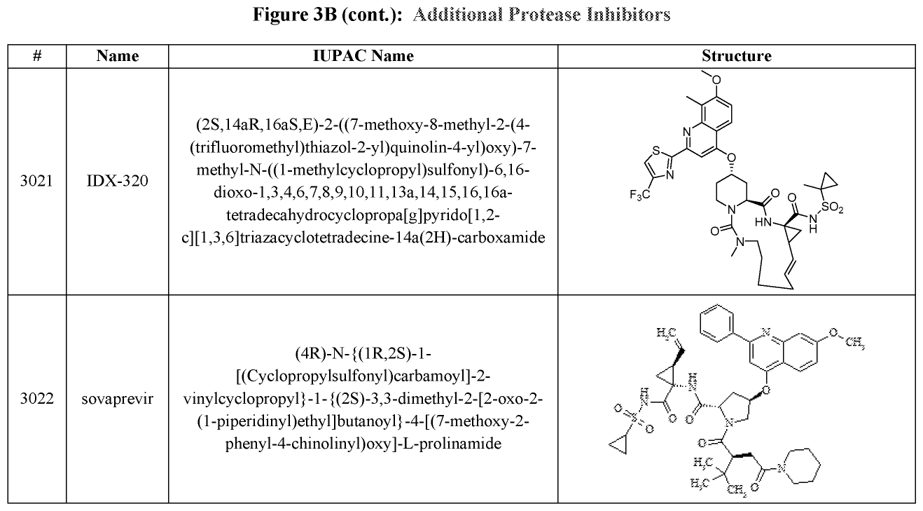

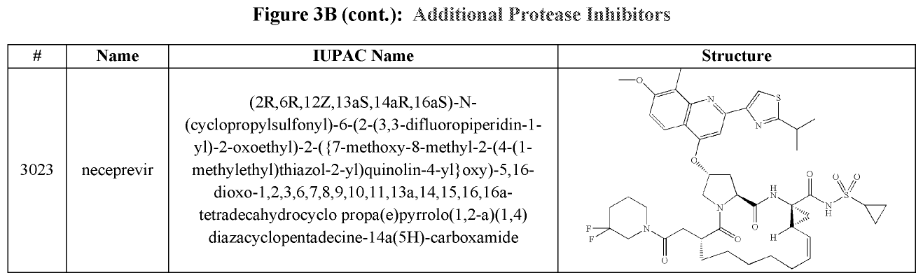

In some embodiments, a compound of Formula (I), or a pharmaceutically acceptable salt thereof, or a pharmaceutical composition that includes a compound of Formula (I), or a pharmaceutically acceptable salt thereof, can be used in combination with a protease inhibitor. In some embodiments, the protease inhibitor can inhibit a HBV and/or HDV protease, for example NS3/4A. A non-limiting list of example protease inhibitors include the following: amprenavir (Agenerase®), asunaprevir (Sunvepra®), atazanavir (Reyataz®), boceprevir (Victrelis®), darunavir (Prezista®), fosamprenavir (Lexiva®; Telzir®), grazoprevir, indinavir (Crixivan®), lopinavir (Kaletra®), nelfinavir (Viracept®), ritonavir (Norvir®), saquinavir (Fortovase®; Invirase®), simeprevir (Olysio®), telaprevir (Incivek®), danoprevir, tipranavir (Aptivus®), ABT-450 (paritaprevir), BILN-2061 (ciluprevir), BI-201335 (faldaprevir), GS-9256, vedroprevir (GS-9451), IDX-320, ACH-1625 (sovaprevir), ACH-2684, and pharmaceutically acceptable salts of any of the foregoing, and/or combinations thereof. A non-limiting list of example protease inhibitors includes compounds numbered 3001-3010 in FIG. 3A and 3011-3023 in FIG. 3B.

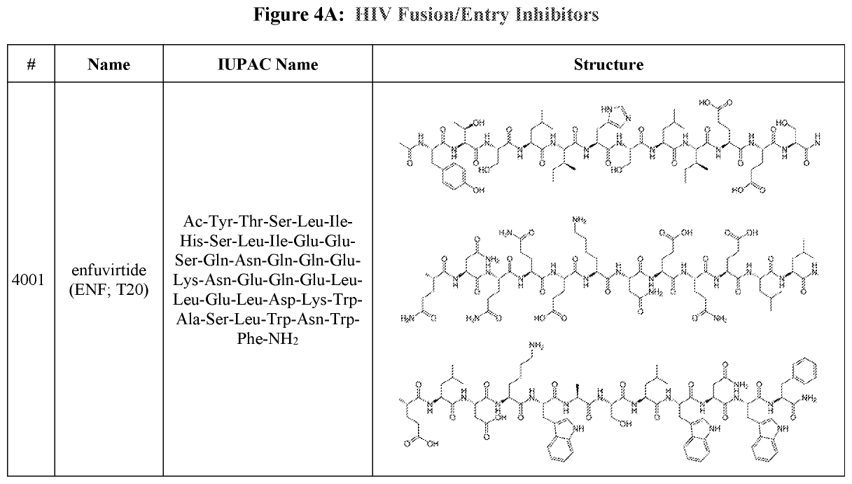

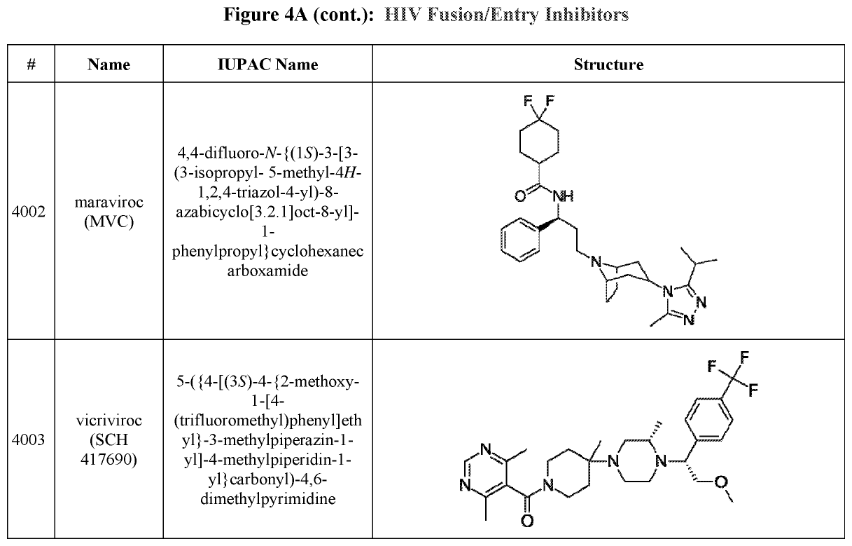

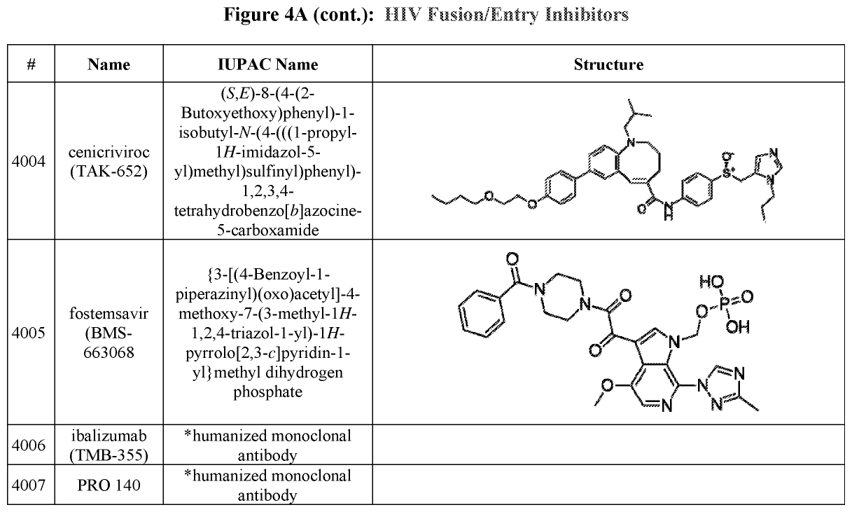

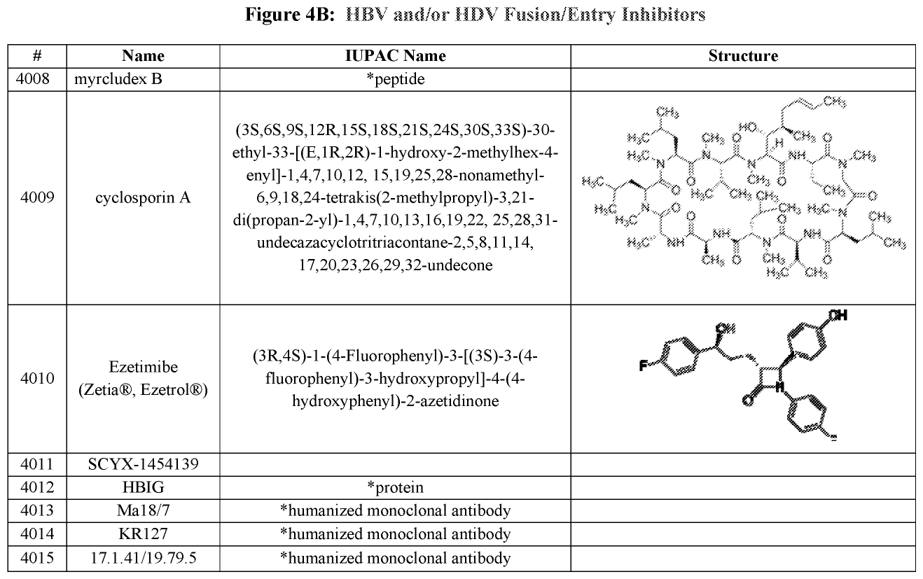

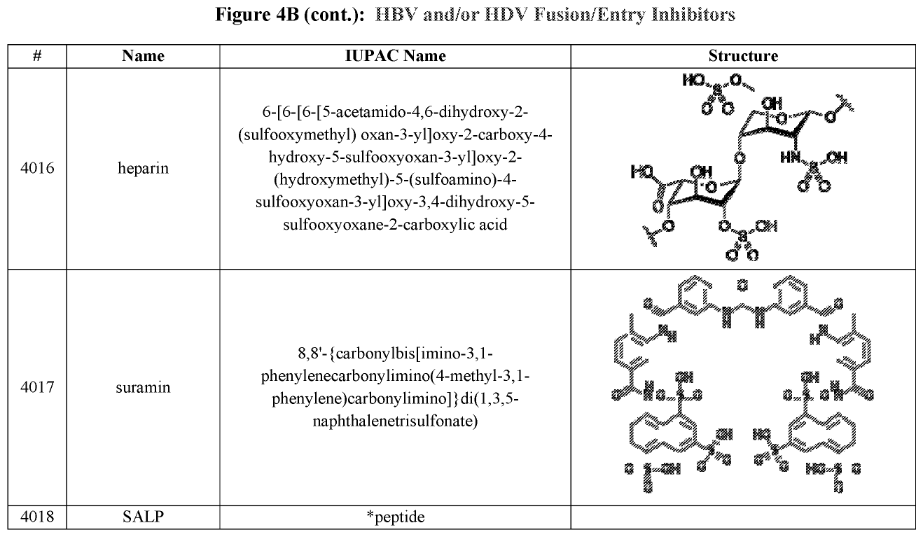

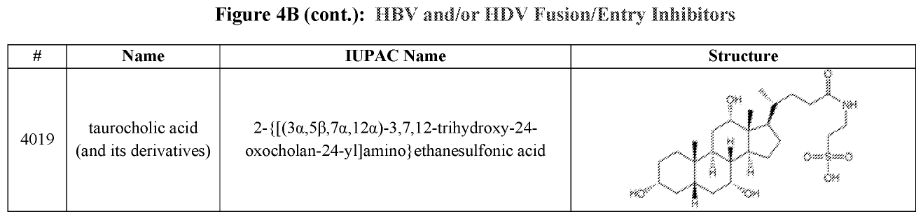

In some embodiments, a compound of Formula (I), or a pharmaceutically acceptable salt thereof, or a pharmaceutical composition that includes a compound of Formula (I), or a pharmaceutically acceptable salt thereof, can be used in combination with an HIV fusion/entry inhibitor. In some embodiments, the HIV fusion/entry inhibitors can block HIV from entering the CD4+ T lymphocytes. In some embodiments, the fusion/entry inhibitors, which are also known as CCR5 antagonists, can block proteins on the CD4+T lymphocyte cells that are required for HIV cellular entry. Examples of suitable fusion/entry inhibitors include, but are not limited to, enfuvirtide (Fuzeon®), maraviroc (Selzentry®), vicriviroc, cenicriviroc, fostemsavir, ibalizumab, PRO 140, and pharmaceutically acceptable salts of any of the foregoing, and/or combinations thereof. A non-limiting list of example HIV fusion/entry inhibitors includes compounds numbered 4001-4007 in FIG. 4A.

In some embodiments, a compound of Formula (I), or a pharmaceutically acceptable salt thereof, or a pharmaceutical composition that includes a compound of Formula (I), or a pharmaceutically acceptable salt thereof, can be used in combination with a HBV and/or HDV fusion/entry inhibitor. In some embodiments, the fusion/entry inhibitors can block HBV and/or HDV from entering hepatocytes. In some embodiments, the HBV and/or HDV fusion/entry inhibitors can block proteins on the hepatocytes that are required for HBV and/or HDV cellular entry. In some embodiments, the HBV and/or HDV fusion/entry inhibitors can bind to sodium-taurocholate cotransporting polypeptides. Examples of suitable HBV and/or HDV fusion/entry inhibitors include, but are not limited to, myrcludex B, cyclosporin A, ezetimibe, and SCYX1454139, HBIG, Ma18/7, KR127, 17.1.41/19.79.5, heparin, suramin, SALP, taurocholic acid derivatives, and pharmaceutically acceptable salts of any of the foregoing, and/or combinations thereof. A non-limiting list of example HBV and/or HDV fusion/entry inhibitors includes compounds numbered 4008-4019 in FIG. 4B.

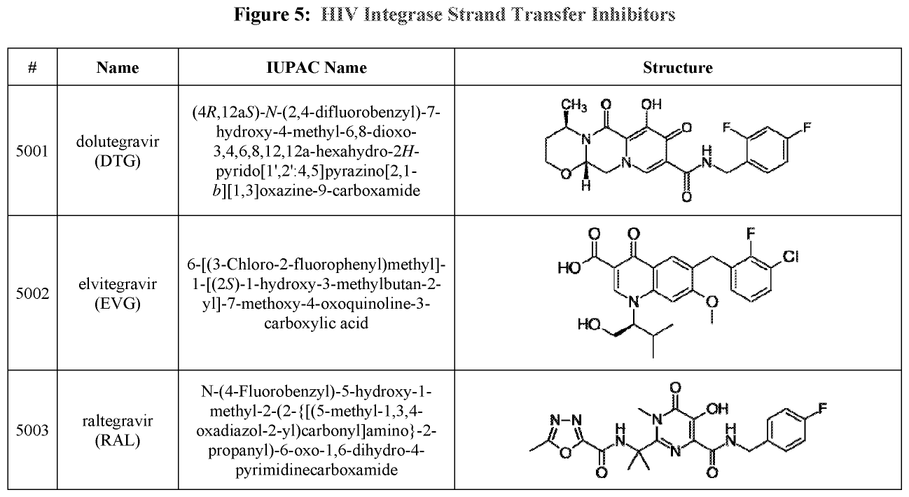

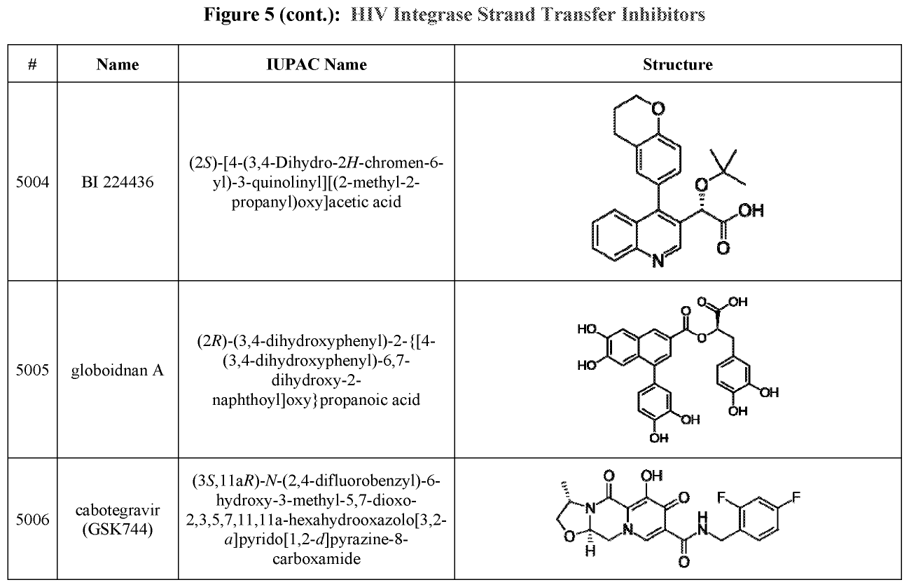

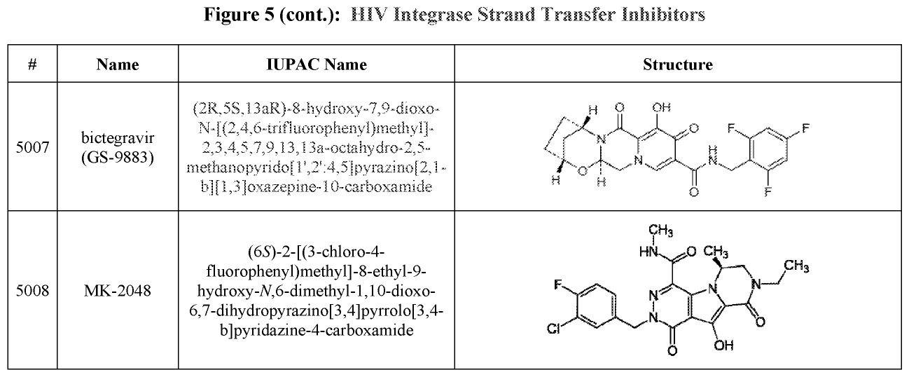

In some embodiments, a compound of Formula (I), or a pharmaceutically acceptable salt thereof, or a pharmaceutical composition that includes a compound of Formula (I), or a pharmaceutically acceptable salt thereof, can be used in combination with an HIV integrase strand transfer inhibitor (INSTI). In some embodiments, the INSTI can block HIV integrase. Examples of INSTIs include, but are not limited to, dolutegravir (Tivicay®), elvitegravir (Strivild®; Vitekta®), raltegravir (Isentress®), BI 224436, globoidnan A, cabotegravir, bictegravir, MK-2048, and pharmaceutically acceptable salts of any of the foregoing, and/or a combination thereof. A non-limiting list of example HIV INSTIs includes compounds numbered 5001-5008 in FIG. 5.

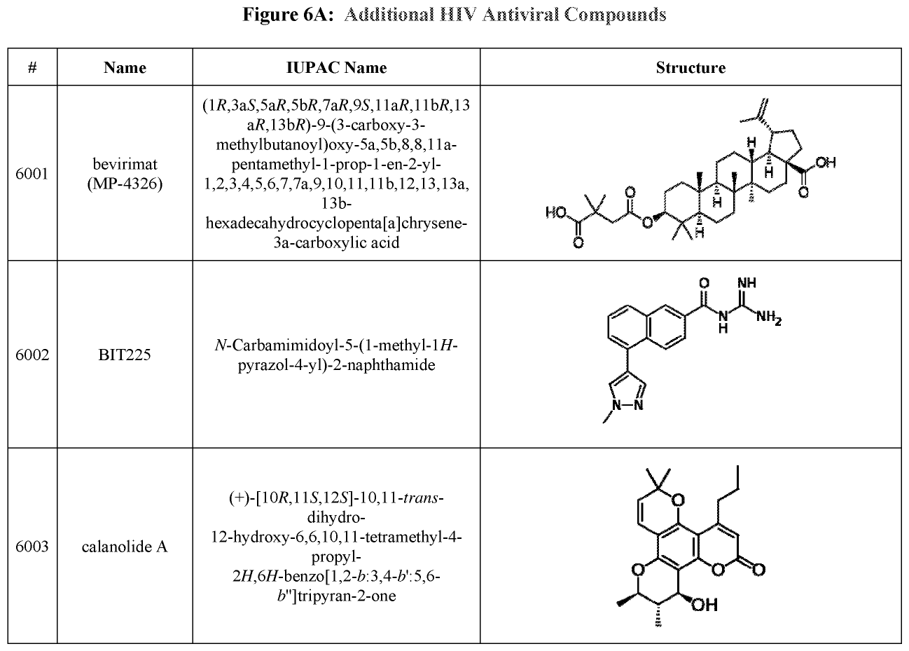

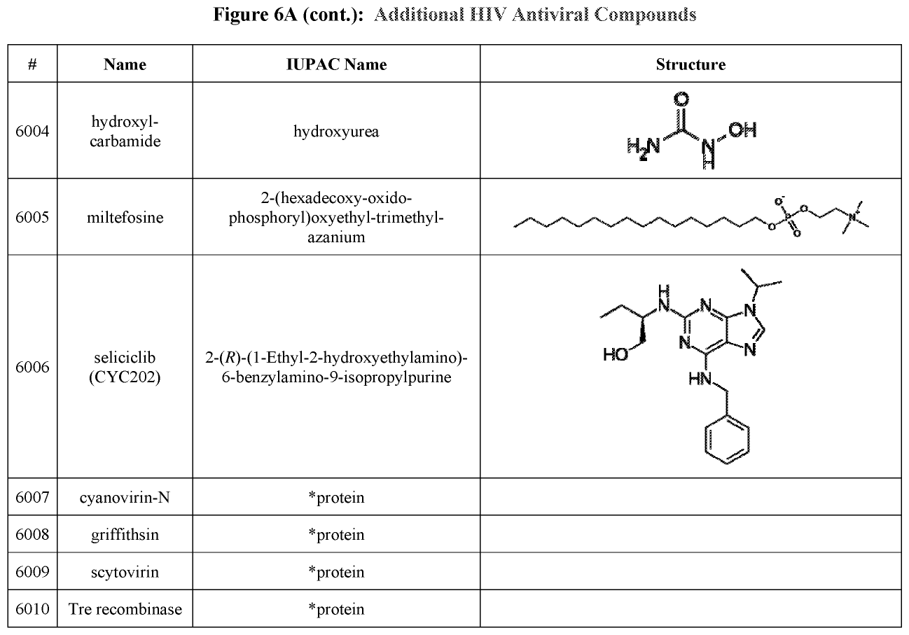

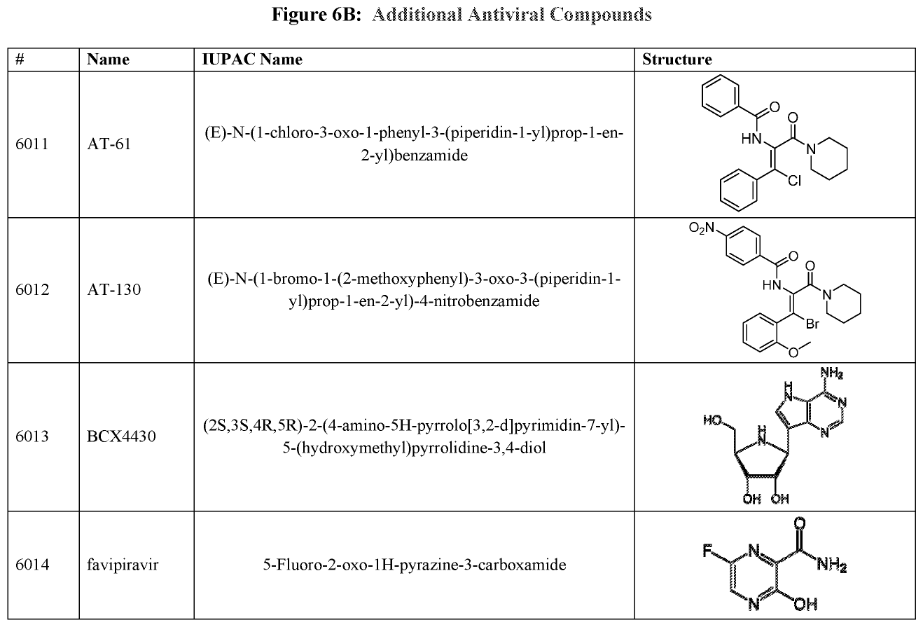

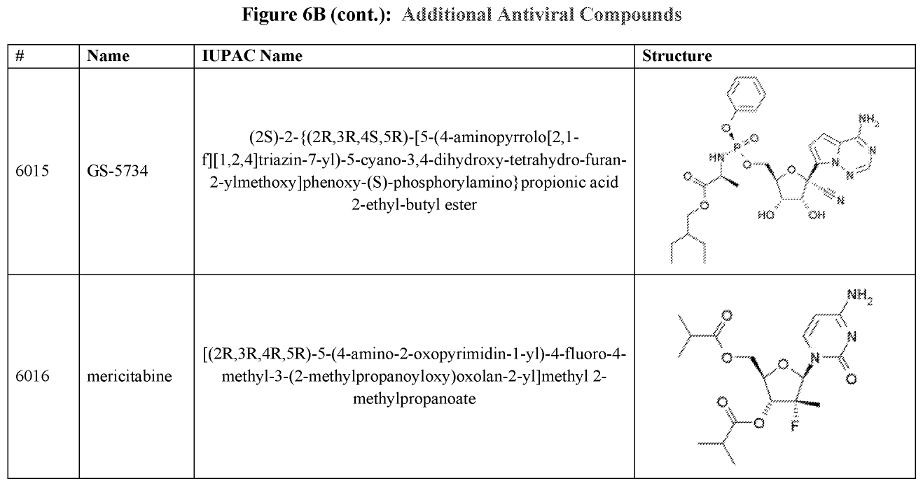

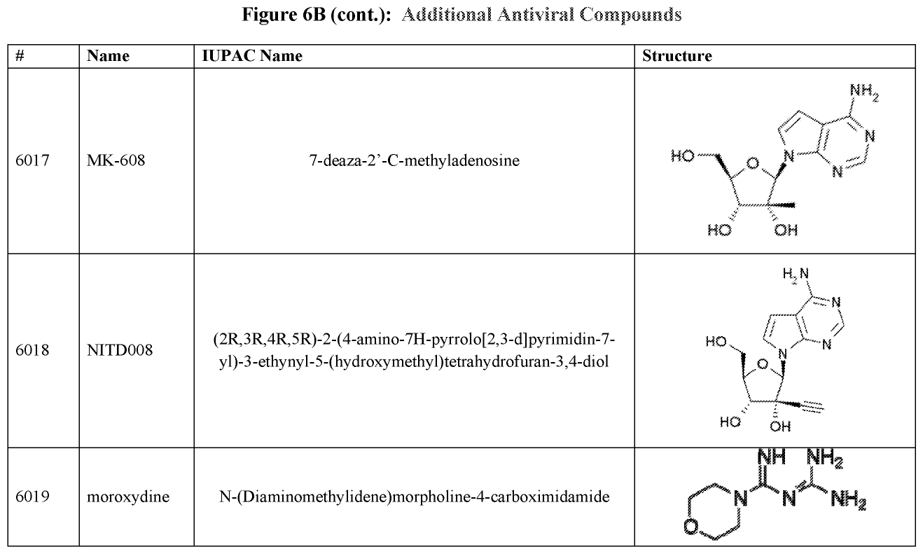

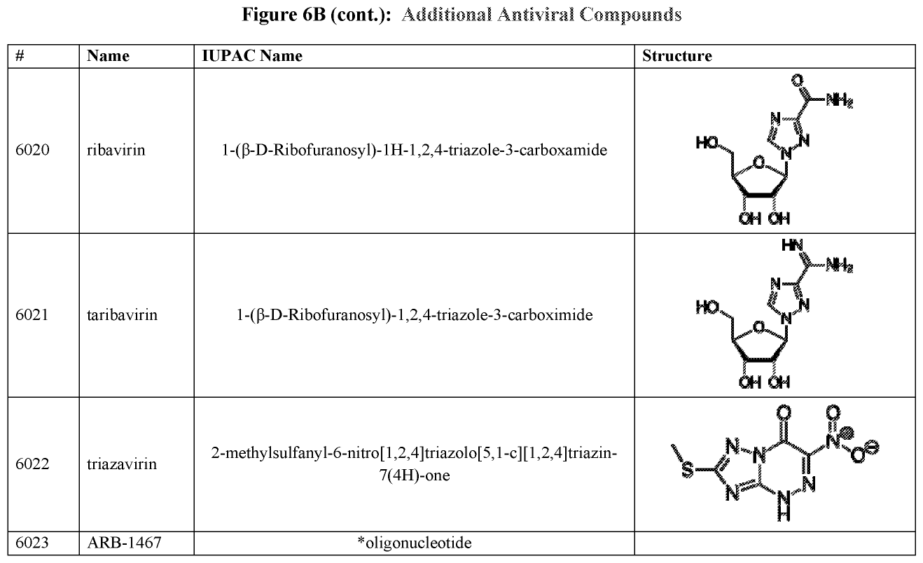

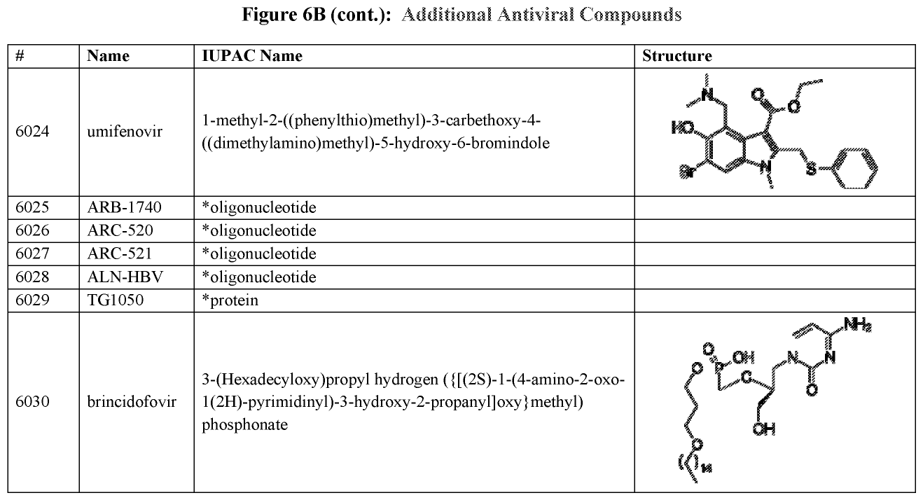

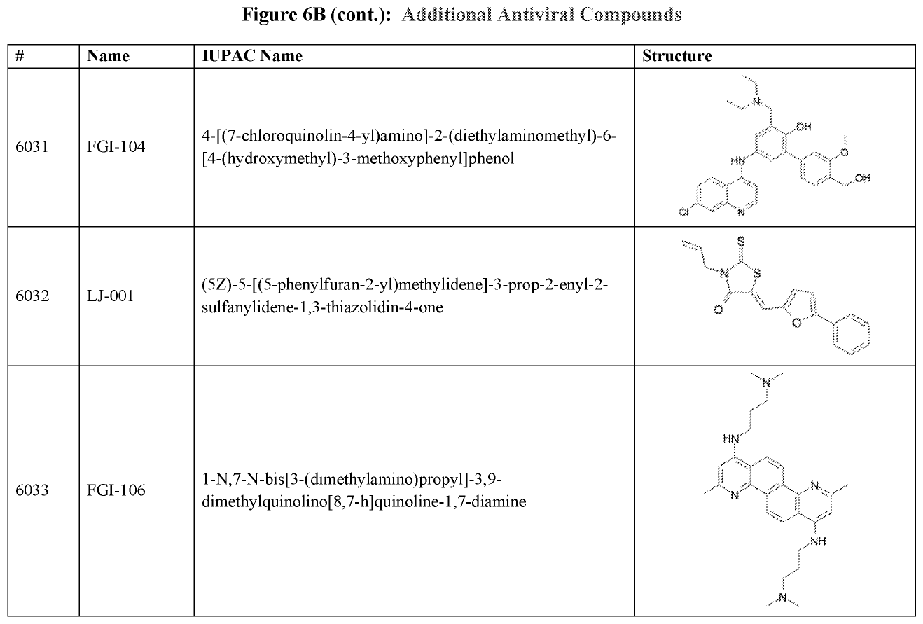

In some embodiments, a compound of Formula (I), or a pharmaceutically acceptable salt thereof, or a pharmaceutical composition that includes a compound of Formula (I), or a pharmaceutically acceptable salt thereof, can be used in combination with other antiviral compounds. Examples of other antiviral compounds include, but are not limited to, bevirimat, BIT225, calanolide A, hydroxycarbamide, miltefosine, seliciclib, cyanovirin-N, griffithsin, scytovirin, BCX4430, favipiravir, GS-5734, mericitabine, MK-608 (7-deaza-2′-C-methyladenosine), NITD008, moroxydine, ribavirin, taribavirin, triazavirin, ARB-1467, ARB-1740, ARC-520, ARC-521, ALN-HBV, TG1050, Tre recombinase, AT-61, AT-130, BCX4430, favipiravir, umifenovir, brincidofovir, FGI-104, LJ-001, FGI-106, and pharmaceutically acceptable salts of any of the foregoing, and/or combinations thereof. A non-limiting list of example other antiviral compounds includes the compounds numbered 6001-6010 in FIG. 6A and 6011-6033 in FIG. 6B. Additional examples of other antiviral compounds include, but are not limited to, an abzyme, an enzyme, a protein, or an antibody. Additional examples of other antiviral compounds include, but are not limited to, ceragenins, including CSA-54, diarylpyrimidines, synergistic enhancers, and zinc finger protein transcription factors, and pharmaceutically acceptable salts of any of the foregoing, and/or combinations thereof.

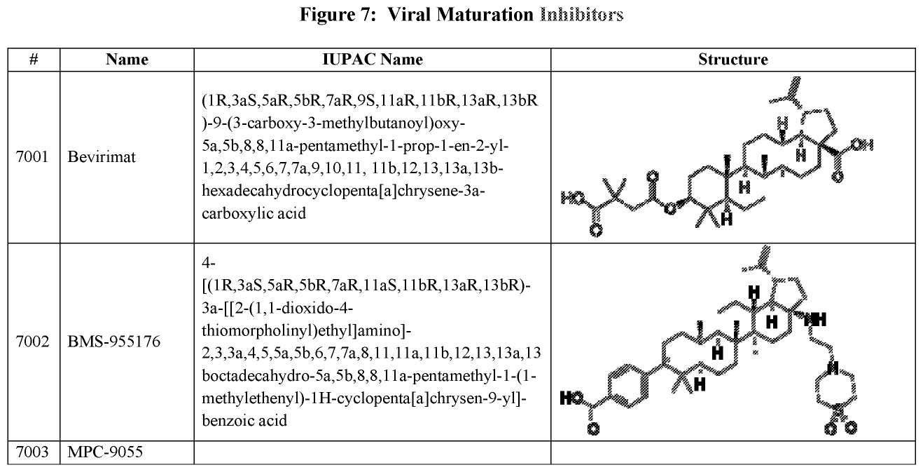

In some embodiments, a compound of Formula (I), or a pharmaceutically acceptable salt thereof, or a pharmaceutical composition that includes a compound of Formula (I), or a pharmaceutically acceptable salt thereof, can be used in combination with a viral maturation inhibitor. In some embodiments, the viral maturation inhibitor can inhibit maturation of HBV and/or HDV. Examples of viral maturation inhibitors include, but are not limited to bevimirat, BMS-955176, MPC-9055, and pharmaceutically acceptable salts of any of the foregoing, and/or combinations thereof. A non-limiting list of example viral maturation inhibitors includes the compounds numbered 7001-7003 in FIG. 7.

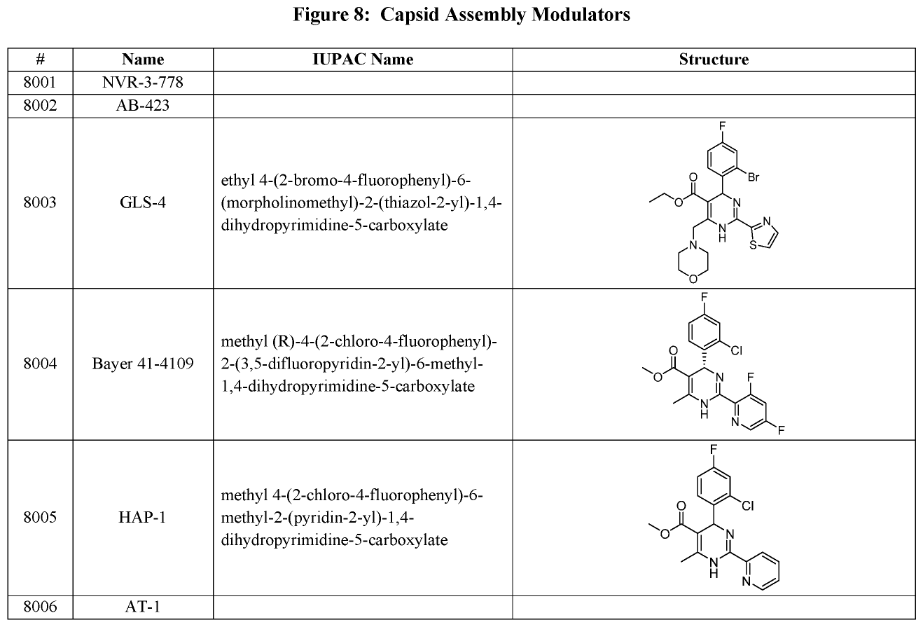

In some embodiments, a compound of Formula (I), or a pharmaceutically acceptable salt thereof, or a pharmaceutical composition that includes a compound of Formula (I), or a pharmaceutically acceptable salt thereof, can be used in combination with a capsid assembly modulator. In some embodiments, the capsid assembly modulator can stabilize the capsid. In some embodiments, the capsid assembly modulator can promote excess capsid assembly. In some embodiments, the capsid assembly modulator can induce formation of non-capsid polymers of capsid peptides. In some embodiments, the capsid assembly modulator can misdirect capsid assembly (e.g., decreasing capsid stability). In some embodiments, the capsid assembly modulator can bind to the HBV and/or HDV core protein. Examples of capsid assembly modulators include, but are not limited to NVR-3-778, AB-423, GLS-4, Bayer 41-4109, HAP-1, AT-1, and pharmaceutically acceptable salts of any of the foregoing, and/or combinations thereof. A non-limiting list of example capsid assembly modulators includes the compounds numbered 8001-8006 in FIG. 8.

In some embodiments, a compound of Formula (I), or a pharmaceutically acceptable salt thereof, or a pharmaceutical composition that includes a compound of Formula (I), or a pharmaceutically acceptable salt thereof, can be used in combination with a FXR agonist. Examples of FXR agonists include, but are not limited to cafestol; chenodeoxychoic acid; cholic acid; obeticholic acid; ursodeoxycholic acid; fexaramine;

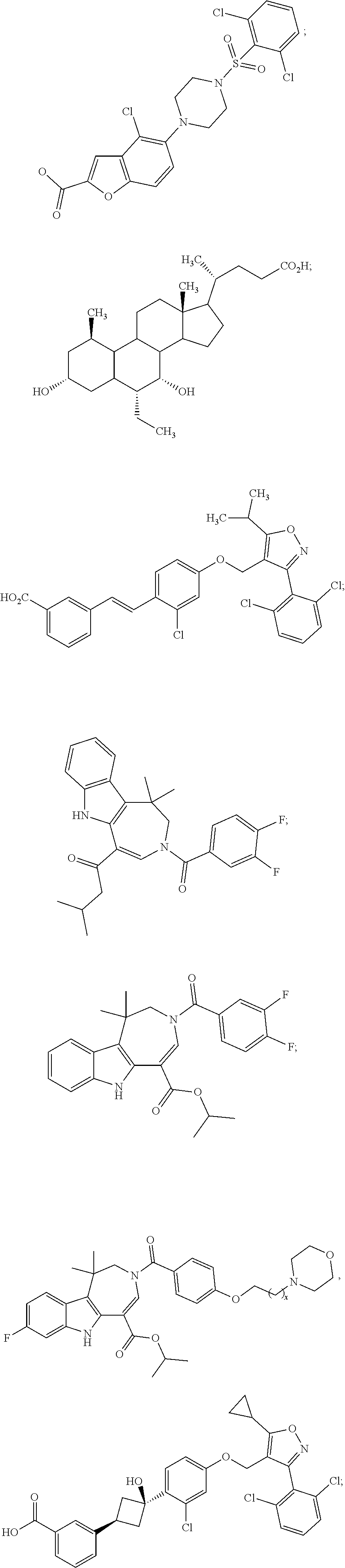

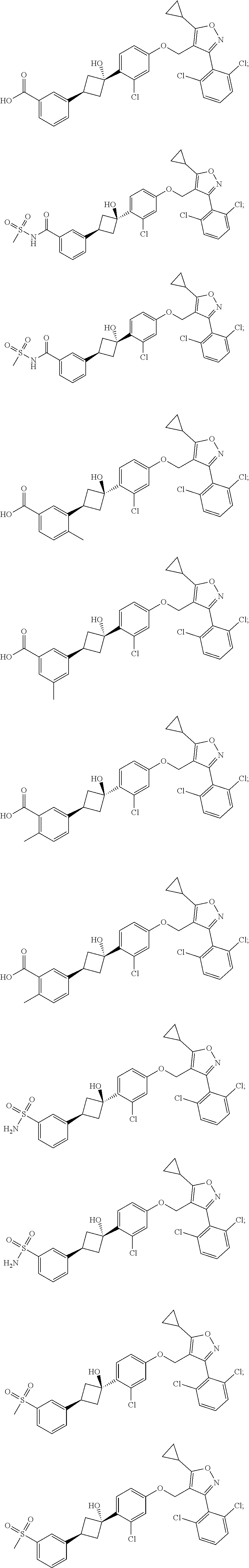

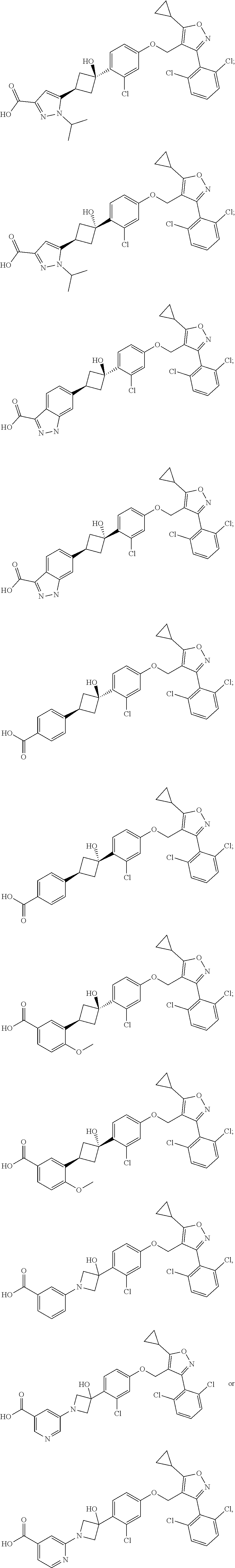

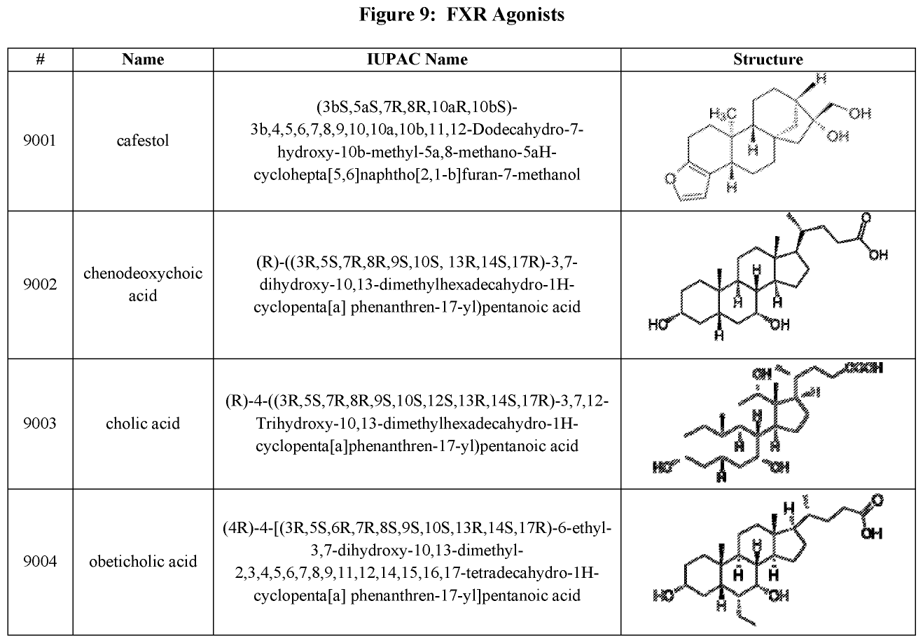

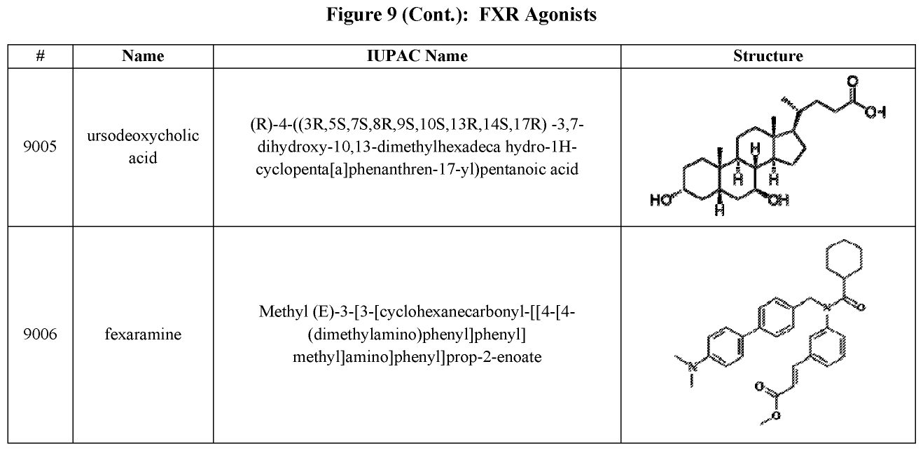

and pharmaceutically acceptable salts of any of the foregoing, and/or combinations thereof. A non-limiting list of additional example FXR agonists includes the compounds numbered 9001-9006 in FIG. 9.

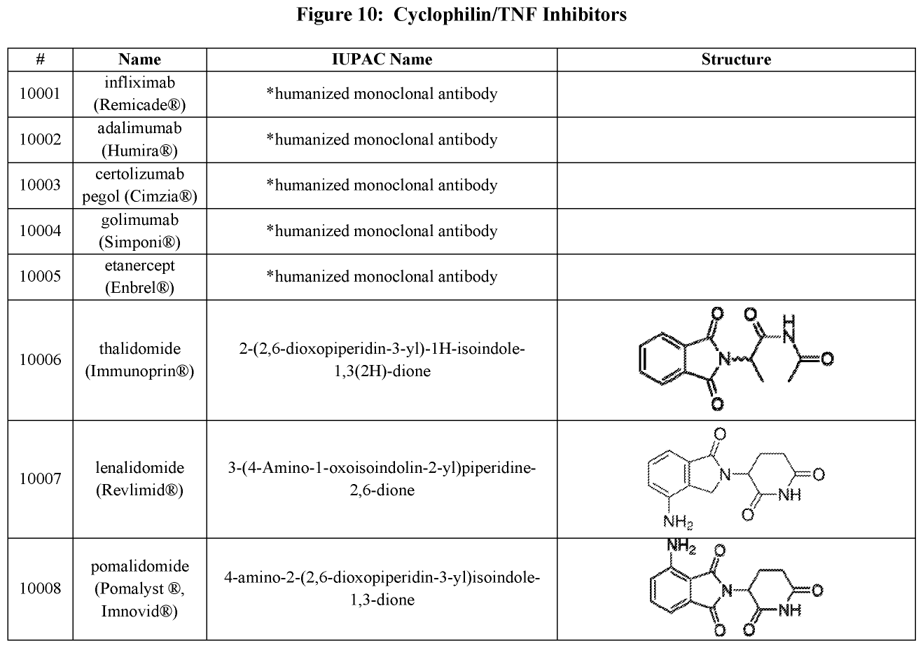

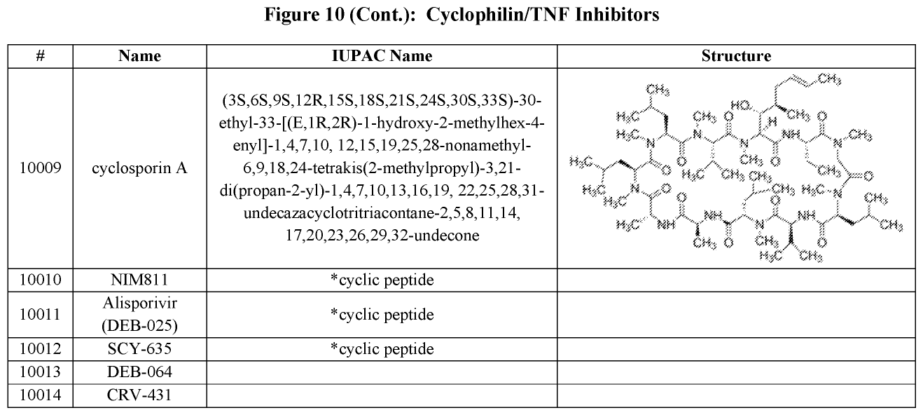

In some embodiments, a compound of Formula (I), or a pharmaceutically acceptable salt thereof, or a pharmaceutical composition that includes a compound of Formula (I), or a pharmaceutically acceptable salt thereof, can be used in combination with a cyclophilin/TNF inhibitor. Examples of cyclophilin/TNF inhibitors include, but are not limited to infliximab (Remicade®), adalimumab (Humira®), certolizumab pegol (Cimzia®), golimumab (Simponi®), etanercept (Enbrel®), thalidomide (Immunoprin®), lenalidomide (Revlimid®), pomalidomide (Pomalyst®, Imnovid®), cyclosporin A, NIM811, Alisporivir (DEB-025), SCY-635, DEB-064, CRV-431, and pharmaceutically acceptable salts of any of the foregoing, and/or combinations thereof. A non-limiting list of example TNF/cyclophilin inhibitors includes the compounds numbered 10001-10014 in FIG. 10.

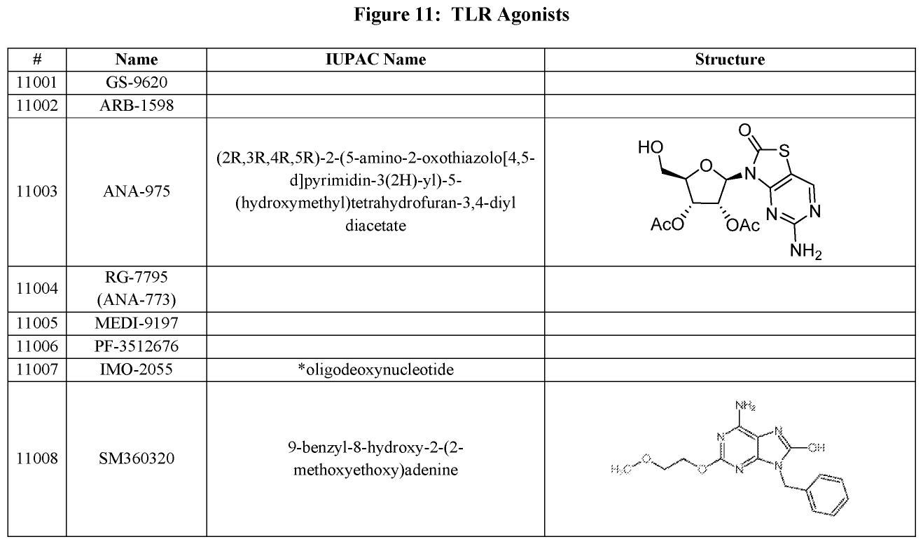

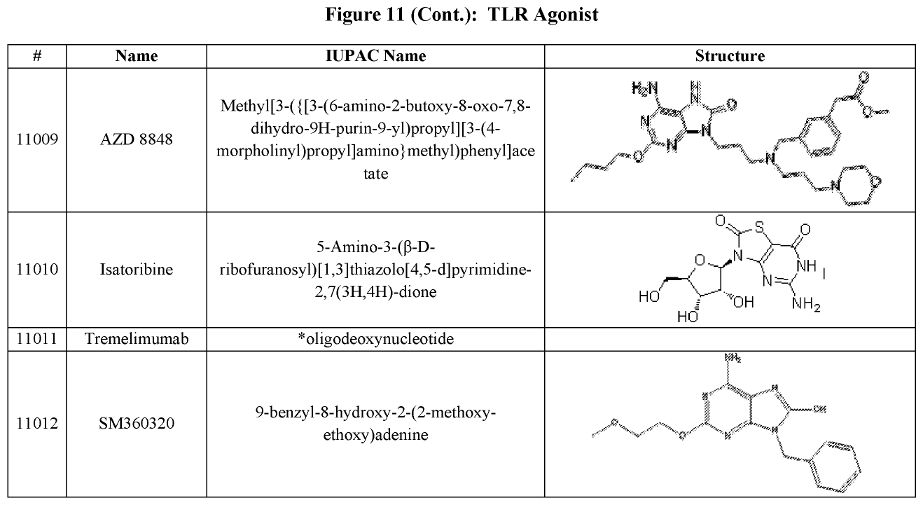

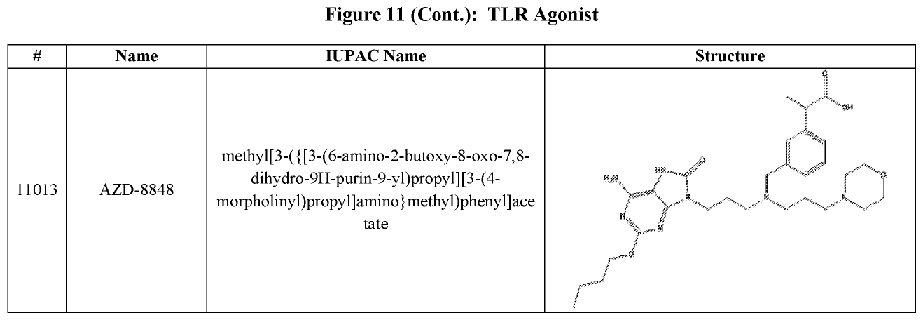

In some embodiments, a compound of Formula (I), or a pharmaceutically acceptable salt thereof, or a pharmaceutical composition that includes a compound of Formula (I), or a pharmaceutically acceptable salt thereof, can be used in combination with a TLR agonist. Examples of TLR agonists include, but are not limited to GS-9620, ARB-1598, ANA-975, RG-7795 (ANA-773), MEDI-9197, PF-3512676, IMO-2055, isatoribine, tremelimumab, SM360320, AZD-8848, and pharmaceutically acceptable salts of any of the foregoing, and/or combinations thereof. A non-limiting list of example TLR agonists includes the compounds numbered 11001-11013 in FIG. 11.

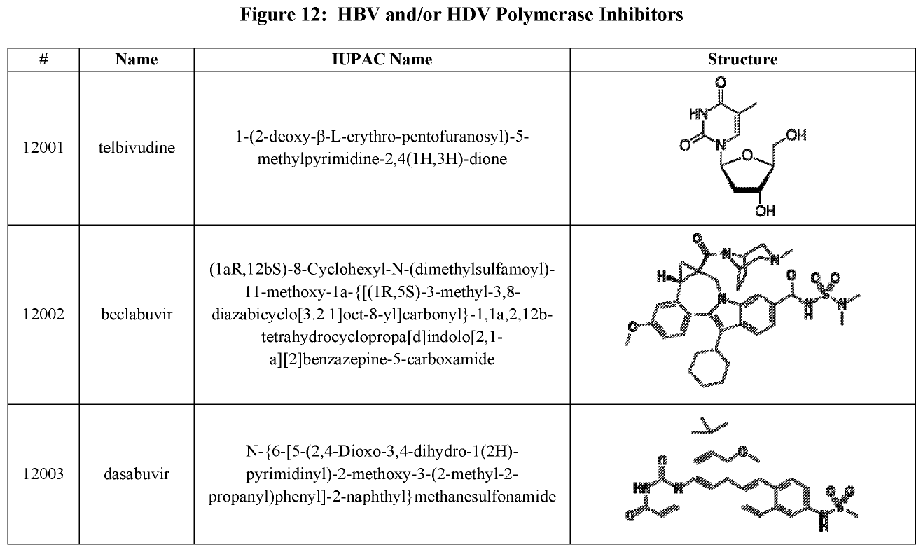

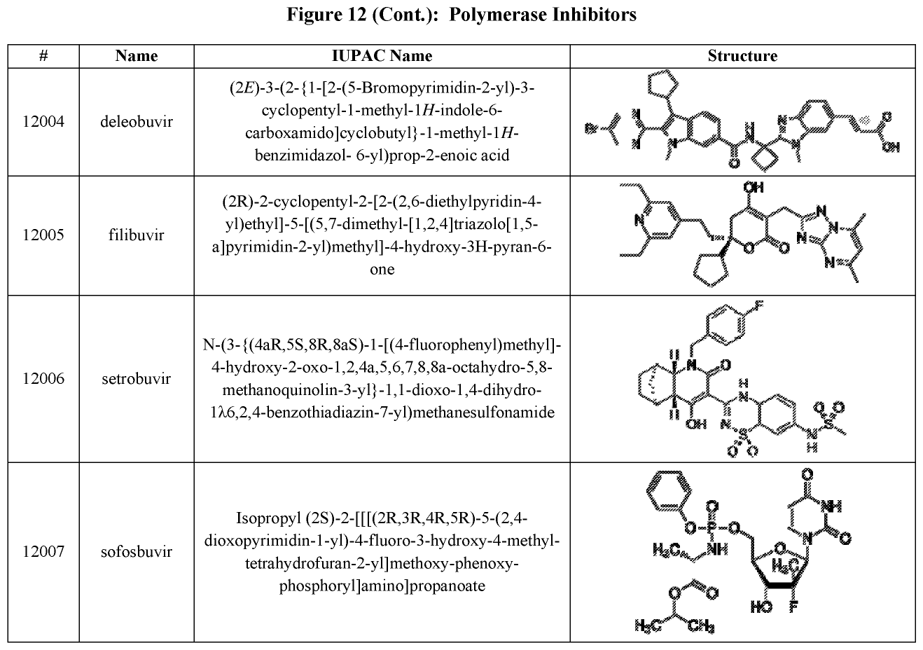

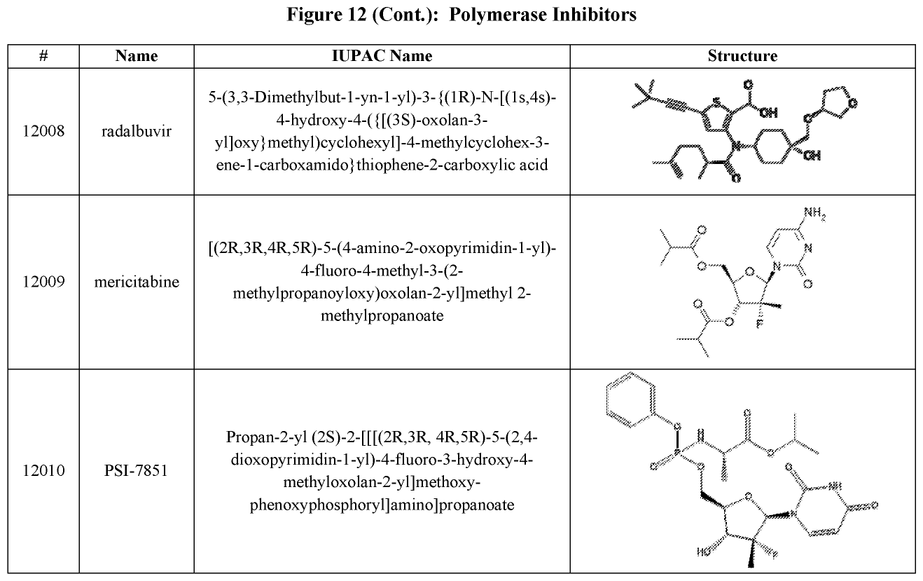

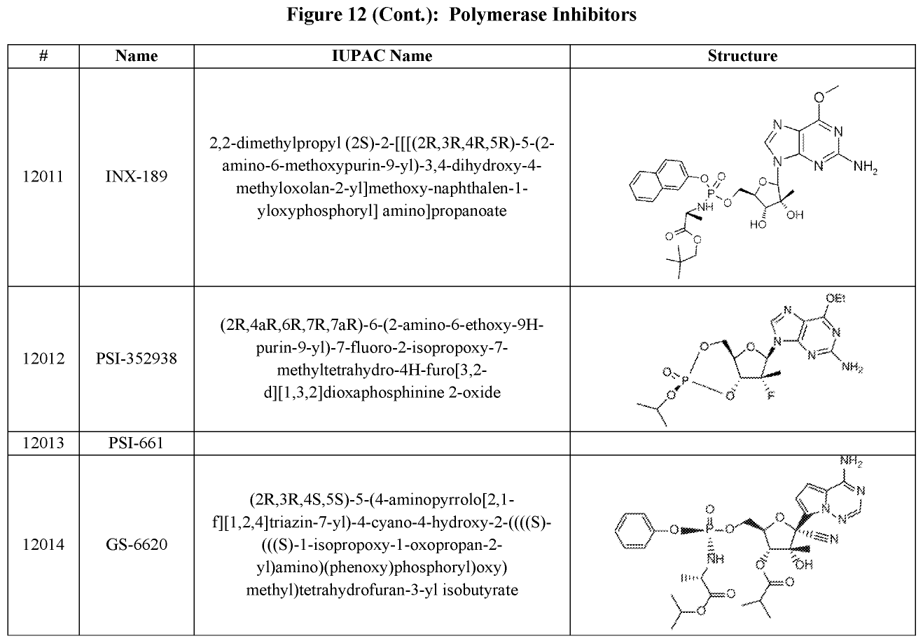

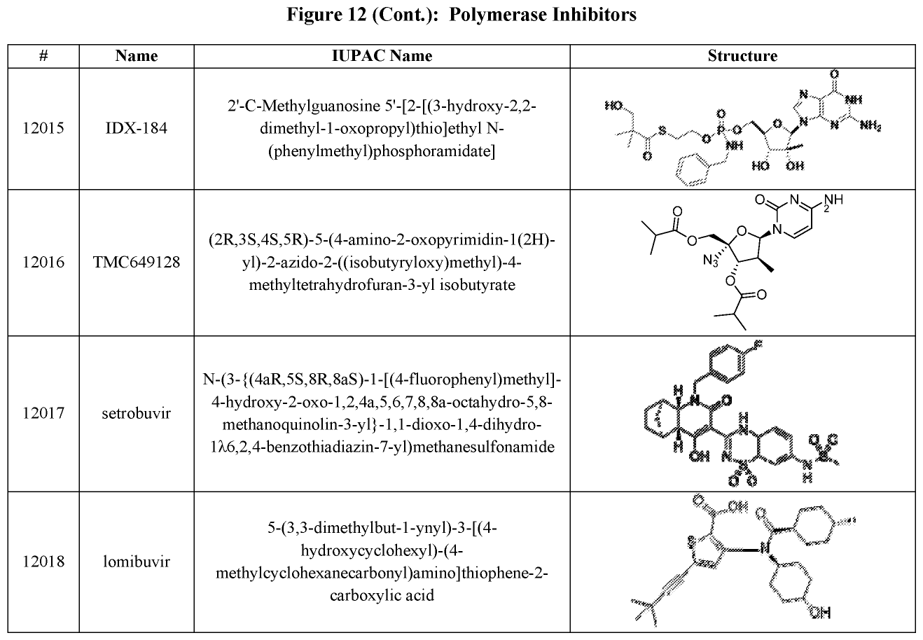

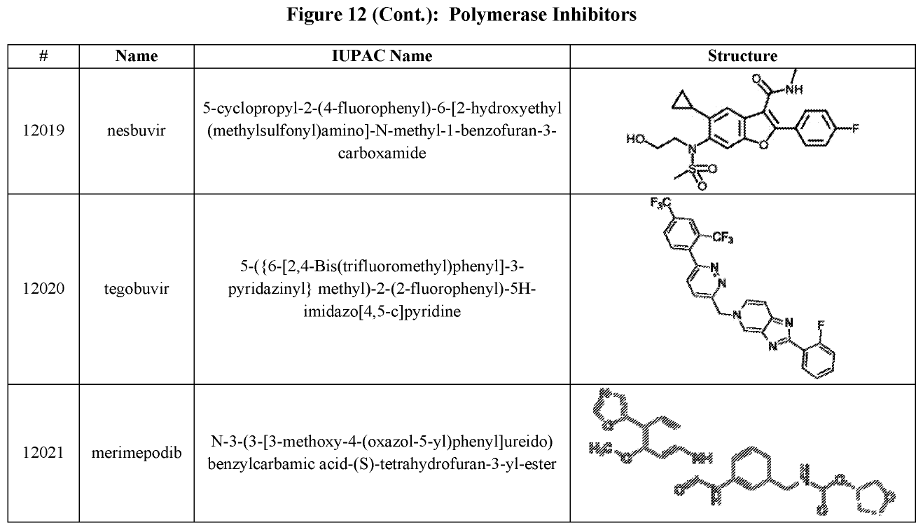

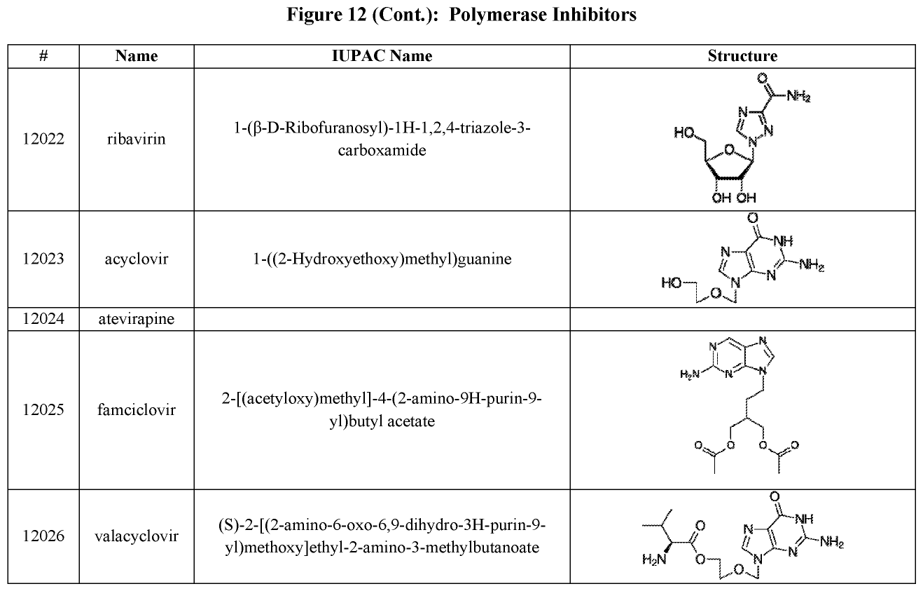

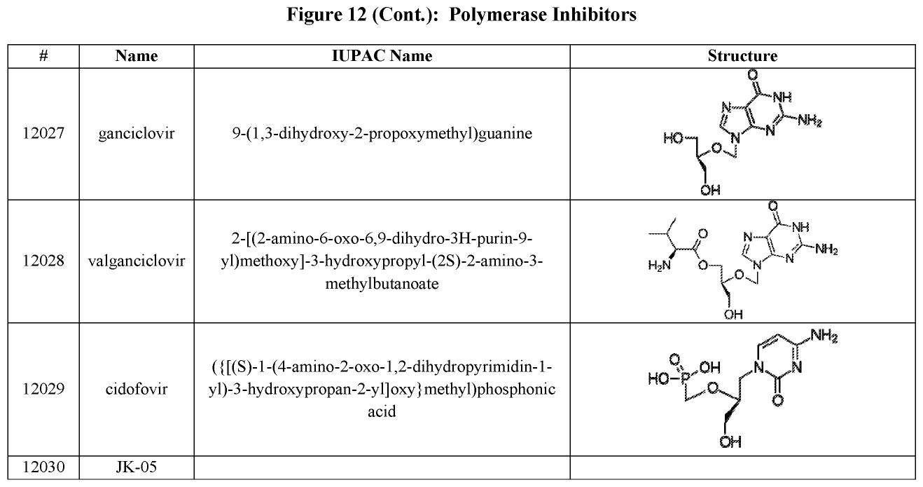

In some embodiments, a compound of Formula (I), or a pharmaceutically acceptable salt thereof, or a pharmaceutical composition that includes a compound of Formula (I), or a pharmaceutically acceptable salt thereof, can be used in combination with a polymerase inhibitor. Examples of polymerase inhibitors include, but are not limited to telbivudine, beclabuvir, dasabuvir, deleobuvir, filibuvir, setrobuvir, sofosbuvir, radalbuvir, RG7128 (mericitabine), PSI-7851, INX-189, PSI-352938, PSI-661, GS-6620, IDX-184, TMC649128, setrobuvir, lomibuvir, nesbuvir, GS-9190 (tegobuvir), VX-497 (merimepodib), ribavirin, acyclovir, atevirapine, famciclovir, valacyclovir, ganciclovir, valganciclovir, cidofovir, JK-05, and pharmaceutically acceptable salts of any of the foregoing, and/or combinations thereof. A non-limiting list of example polymerase inhibitors includes the compounds numbered 12001-12030 in FIG. 12.

In some embodiments, a compound of Formula (I), or a pharmaceutically acceptable salt thereof, or a pharmaceutical composition that includes a compound of Formula (I), or a pharmaceutically acceptable salt thereof, can be used in combination with a vaccine. Examples of vaccines include, but are not limited to Heplislav®, ABX-203, INO-1800, and pharmaceutically acceptable salts of any of the foregoing, and/or combinations thereof. A non-limiting list of example vaccines includes those numbered 13001-13003 in FIG. 13.

In some embodiments, a compound of Formula (I), or a pharmaceutically acceptable salt thereof, or a pharmaceutical composition that includes a compound of Formula (I), or a pharmaceutically acceptable salt thereof, can be used in combination with an interferon. Examples of interferons include, but are not limited to alpha-interferons, beta-interferons, delta-interferons, omega-interferons, tau-interferons, x-interferons, consensus interferons, and asialo-interferons. Specific non-limiting examples include: interferon alpha 1A, interferon alpha 1B, interferon alpha 2A, interferon alpha 2B, pegylated-interferon alpha 2a (PEGASYS®, Roche), recombinant interferon alpha 2a (ROFERON®, Roche), inhaled interferon alpha 2b (AERX®, Aradigm), pegylated-interferon alpha 2b (ALBUFERON®, Human Genome Sciences/Novartis, PEGINTRON®, Schering), recombinant interferon alpha 2b (INTRON A®, Schering), pegylated interferon alpha 2b (PEG-INTRON®, Schering, VIRAFERONPEG®, Schering), interferon beta-1a (REBIF®, Serono, Inc. and Pfizer), consensus interferon alpha (INFERGEN®, Valeant Pharmaceutical).

In some embodiments, a compound of Formula (I), or a pharmaceutically acceptable salt thereof, or a pharmaceutical composition that includes a compound of Formula (I), or a pharmaceutically acceptable salt thereof, can be used in combination with an siRNA or ASO cccDNA inhibitor. In some embodiments, the an siRNA or ASO cccDNA inhibitor can prevent cccDNA formation, eliminate existing cccDNA, destabilizing existing cccDNA, and/or silence cccDNA transcription.

In some embodiments, a compound of Formula (I), or a pharmaceutically acceptable salt thereof, or a pharmaceutical composition that includes a compound of Formula (I), or a pharmaceutically acceptable salt thereof, can be used in combination with a gene silencing agent. In some embodiments, the gene silencing agent decreases transcription of a target gene or genes. In some embodiments, the gene silencing agent decreases translation of a target gene or genes. In some embodiments, the gene silencing agent can be an oligodeoxynucleotide, a ribozyme, siRNA, a morpholino, or a combination of any of the foregoing.

In some embodiments, a compound of Formula (I), or a pharmaceutically acceptable salt thereof, or a pharmaceutical composition that includes a compound of Formula (I), or a pharmaceutically acceptable salt thereof, can be used in combination with an HBx inhibitor. HBx is a polypeptide encoded by hepadnaviruses that contributes to viral infectivity. In some embodiments, the HBx inhibitor decreases HBx transactivation activity. In some embodiments, the HBx inhibitor blocks or decreases HBx binding to mammalian cellular proteins. In some embodiments, the HBx inhibitor decreases HBx blocks or decreases recruitment of kinases.

In some embodiments, a compound of Formula (I), or a pharmaceutically acceptable salt thereof, or a pharmaceutical composition that includes a compound of Formula (I), or a pharmaceutically acceptable salt thereof, can be used in combination with an HBsAg secretion inhibitor. HBV and HDV surface antigens are proteins found on both new HBV particle and subviral particles. The subviral particles are non-infectious and are secreted in significant excess to infectious virus, potentially exhausting a subject's immune system. In some embodiments, the HBsAg secretion inhibitor can reduce a subject's immune exhaustion due to the surface antigen. In some embodiments, the HBsAg secretion inhibitor can promote a subject's immune response to HBV and/or HDV.

In some embodiments, a compound of Formula (I), or a pharmaceutically acceptable salt thereof, or a pharmaceutical composition that includes a compound of Formula (I), or a pharmaceutically acceptable salt thereof, can be used in combination with a covalently closed circular DNA (cccDNA) inhibitor. In some embodiments, the cccDNA inhibitor can directly bind cccDNA, can inhibit conversion of relaxed circular DNA (rcDNA) to cccDNA, can reduce or silence transcription of cccDNA, and/or can promote elimination of existing cccDNA.

In some embodiments, a compound of Formula (I), or a pharmaceutically acceptable salt thereof, or a pharmaceutical composition that includes a compound of Formula (I), or a pharmaceutically acceptable salt thereof, can be used in combination with a compound of Formula (I), or a pharmaceutically acceptable salt thereof, described in PCT Publication No. WO 2017/156262, filed Mar. 9, 2017.

Some embodiments described herein relate to a method of treating a HBV and/or HDV infection that can include contacting a cell infected with the HBV and/or HDV infection with an effective amount of a compound of Formula (I), or a pharmaceutically acceptable salt thereof, in combination with one or more additional agents, such as those described herein. Other embodiments described herein relate to a method of treating a HBV and/or HDV infection that can include administering to a subject suffering from the HBV and/or HDV infection an effective amount of a compound of Formula (I), or a pharmaceutically acceptable salt thereof, in combination with one or more additional agents, such as those described herein. Still other embodiments described herein relate to a method of inhibiting the replication of a HBV and/or HDV that can include contacting a cell infected with the HBV and/or HDV with an effective amount of a compound of Formula (I), or a pharmaceutically acceptable salt thereof, in combination with one or more additional agents, such as those described herein. Yet still other embodiments described herein relate to a method of inhibiting the replication of a HBV and/or HDV that can include administering to a subject infected with the HBV and/or HDV an effective amount of a compound of Formula (I), or a pharmaceutically acceptable salt thereof, in combination with one or more additional agents, such as those described herein. Examples of additional agents include those described herein, such as, a polymerase inhibitor, a protease inhibitor (PI), a fusion/entry inhibitor, an interferon, a FXR agonist, a TLR agonist, a viral maturation inhibitor, a capsid assembly modulator, a cyclophilin/TNF inhibitor, a vaccine, an siRNA or ASO cccDNA inhibitor, a gene silencing agent, an HBx inhibitor, an HBsAg secretion inhibitor, and another antiviral compound, or a pharmaceutically acceptable salt of any of the foregoing.

Some embodiments described herein relate to a method of treating a HIV infection that can include contacting a cell infected with the HIV infection with an effective amount of a compound of Formula (I), or a pharmaceutically acceptable salt thereof, in combination with one or more additional agents, such as those described herein. Other embodiments described herein relate to a method of treating a HIV infection that can include administering to a subject suffering from the HIV infection an effective amount of a compound of Formula (I), or a pharmaceutically acceptable salt thereof, in combination with one or more additional agents, such as those described herein. Still other embodiments described herein relate to a method of inhibiting the replication of a HIV that can include contacting a cell infected with the HIV with an effective amount of a compound of Formula (I), or a pharmaceutically acceptable salt thereof, in combination with one or more additional agents, such as those described herein. Yet still other embodiments described herein relate to a method of inhibiting the replication of a HIV that can include administering to a subject infected with the HIV an effective amount of a compound of Formula (I), or a pharmaceutically acceptable salt thereof, in combination with one or more additional agents, such as those described herein. Examples of additional agents include those described herein, such as, antiretroviral therapy (ART) agents, such as a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy compound, or a pharmaceutically acceptable salt of any of the foregoing.

In some embodiments, a compound of Formula (I), or a pharmaceutically acceptable salt thereof, can be administered with one or more additional agent(s) together in a single pharmaceutical composition. In some embodiments, a compound of Formula (I), or a pharmaceutically acceptable salt the thereof, can be administered with one or more additional agent(s) as two or more separate pharmaceutical compositions. For example, a compound of Formula (I), or a pharmaceutically acceptable salt thereof, can be administered in one pharmaceutical composition, and at least one of the additional agents can be administered in a second pharmaceutical composition. If there are at least two additional agents, one or more of the additional agents can be in a first pharmaceutical composition that includes a compound of Formula (I), or a pharmaceutically acceptable salt thereof, and at least one of the other additional agent(s) can be in a second pharmaceutical composition.

The dosing amount(s) and dosing schedule(s) when using a compound of Formula (I), or a pharmaceutically acceptable salt thereof, or a pharmaceutical composition that includes a compound of Formula (I), or a pharmaceutically acceptable salt thereof, and one or more additional agents are within the knowledge of those skilled in the art. For example, when performing a conventional standard of care therapy using art-recognized dosing amounts and dosing schedules, a compound of Formula (I), or a pharmaceutically acceptable salt thereof, or a pharmaceutical composition that includes a compound of Formula (I), or a pharmaceutically acceptable salt thereof, can be administered in addition to that therapy, or in place of one of the agents of a combination therapy, using effective amounts and dosing protocols as described herein.

The order of administration of a compound of Formula (I), or a pharmaceutically acceptable salt thereof, with one or more additional agent(s) can vary. In some embodiments, a compound of Formula (I), or a pharmaceutically acceptable salt thereof, can be administered prior to all additional agents. In other embodiments, a compound of Formula (I), or a pharmaceutically acceptable salt thereof, can be administered prior to at least one additional agent. In still other embodiments, a compound of Formula (I), or a pharmaceutically acceptable salt thereof, can be administered concomitantly with one or more additional agent(s). In yet still other embodiments, a compound of Formula (I), or a pharmaceutically acceptable salt thereof, can be administered subsequent to the administration of at least one additional agent. In some embodiments, a compound of Formula (I), or a pharmaceutically acceptable salt thereof, can be administered subsequent to the administration of all additional agents.

In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in an additive effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in a synergistic effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in a strongly synergistic effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) is not antagonistic.

As used herein, the term “antagonistic” means that the activity of the combination of compounds is less compared to the sum of the activities of the compounds in combination when the activity of each compound is determined individually (i.e. as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compound in the combination when the activity of each compound is determined individually.

A potential advantage of utilizing a compound of Formula (I), or a pharmaceutically acceptable salt thereof, in combination with one or more additional agent(s) in FIGS. 1-13 (including pharmaceutically acceptable salts of any of the foregoing) may be a reduction in the required amount(s) of one or more compounds of FIGS. 1-13 (including pharmaceutically acceptable salts of any of the foregoing) that is effective in treating a disease condition disclosed herein (for example, HBV, HDV and/or HIV), as compared to the amount required to achieve same therapeutic result when one or more compounds of FIGS. 1-13 (including pharmaceutically acceptable salts of any of the foregoing) are administered without a compound of Formula (I), or a pharmaceutically acceptable salt thereof. For example, the amount of a compound in FIGS. 1-13 (including a pharmaceutically acceptable salt of any of the foregoing), can be less compared to the amount of the compound in FIGS. 1-13 (including a pharmaceutically acceptable salt of any of the foregoing), needed to achieve the same viral load reduction when administered as a monotherapy. Another potential advantage of utilizing a compound of Formula (I), or a pharmaceutically acceptable salt thereof, in combination with one or more additional agent(s) in FIGS. 1-13 (including pharmaceutically acceptable salts of any of the foregoing) is that the use of two or more compounds having different mechanism of actions can create a higher barrier to the development of resistant viral strains compared to the barrier when a compound is administered as monotherapy.

Additional advantages of utilizing a compound of Formula (I), or a pharmaceutically acceptable salt thereof, in combination with one or more additional agent(s) in FIGS. 1-13 (including pharmaceutically acceptable salts of any of the foregoing) may include little to no cross resistance between a compound of Formula (I), or a pharmaceutically acceptable salt thereof, and one or more additional agent(s) in FIGS. 1-13 (including pharmaceutically acceptable salts of any of the foregoing) thereof; different routes for elimination of a compound of Formula (I), or a pharmaceutically acceptable salt thereof, and one or more additional agent(s) in FIGS. 1-13 (including pharmaceutically acceptable salts of any of the foregoing); little to no overlapping toxicities between a compound of Formula (I), or a pharmaceutically acceptable salt thereof, and one or more additional agent(s) in FIGS. 1-13 (including pharmaceutically acceptable salts of any of the foregoing); little to no significant effects on cytochrome P450; little to no pharmacokinetic interactions between a compound of Formula (I), or a pharmaceutically acceptable salt thereof, and one or more additional agent(s) in FIGS. 1-13 (including pharmaceutically acceptable salts of any of the foregoing); greater percentage of subjects achieving a sustained viral response compared to when a compound is administered as monotherapy and/or a decrease in treatment time to achieve a sustained viral response compared to when a compound is administered as monotherapy.

### Examples

The following specific examples are provided to further illustrate various embodiments described herein.

In obtaining the compounds described in the examples below and the corresponding analytical data, the following experimental and analytical protocols were followed unless otherwise indicated.

Unless otherwise stated, reaction mixtures were magnetically stirred at room temperature (r.t.) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on: a Gilson 281/215 HPLC with an Xtimate Prep RP18 column (5 μM, 25×150 mm) or an YMC-Actus Triart C18 column (5 μM, 30×100 mm), and a mobile phase of 1% ACN in 0.225% FA was held for 1 min, then a gradient of 1-23% ACN over 9 min, then held at 95% ACN for 2 min, with a flow rate of 25 mL/min.

Mass spectra (MS) were obtained on an Agilent G1969A LCMS-TOF. The mobile phase: 0.1% FA (formic acid) in water (solvent A) and 0.1% FA in ACN (solvent B); Elution Gradient: 0%-30% (solvent B) over 3 minutes and holding at 30% for 1 minute at a flow rate of 1 mL/minute; Column: Xbridge Shield RP 18 Sum, 2.1*50 mm Ion Source: ESI source; Ion Mode: Positive; Nebulization Gas: Nitrogen; Drying Gas (N2) Flow Rate: 5 L/min; Nebulizer Pressure: 30 psig; Gas Temperature: 325° C. Capillary Voltage: 3.5 KV; Fragmentor Voltage: 50 V.

Nuclear magnetic resonance (NMR) spectra were obtained on Bruker 400 MHz or Varian 400 MHz spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1. (1R,3R)-3-(((tert-Butyldiphenylsilyl)oxy)methyl)-2-methylenecyclobutanol

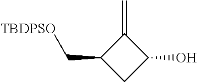

Step A. 1,1-Diethoxyethene. To a flask equipped with condenser was added t-BuOK (115.0 g, 1.02 mol) and 2-bromo-1,1-diethoxyethane (200.0 g, 1.01 mol) at r.t. The reaction was extremely exothermic and started to reflux. After the reflux stopped, the reaction was stirred for 1 h. t-BuOH was removed by distillation, the resulting mixture was distilled under reduced pressure to afford 1,1-diethoxyethene (82.0 g, 705.9 mmol, 69.9% yield) as a colorless liquid. ¹H NMR (400 MHz, CDCl₃) δ 4.27 (q, J=7.1 Hz, 4H), 2.05 (s, 2H), 1.33 (t, J=7.1 Hz, 6H).

Step B. (1R,2S)-Diethyl 3,3-diethoxycyclobutane-1,2-dicarboxylate. A mixture of toluene (100 mL) and 1,1-diethoxyethene (6.50 g, 37.75 mmol) was cooled to −45° C. under N₂ atmosphere. Diethylaluminum chloride (1 M, 113.25 mL) was added slowly by syringe. The reaction mixture was stirred for 10 min, then N,N-diisopropylethylamine (DIPEA) (1.95 g, 15.10 mmol) was added. After stirring at −45° C. for 10 min, diethyl fumarate (8.77 g, 75.50 mmol) was added by syringe, and the mixture was stirred at −45° C. for 3 h. The mixture was quenched with saturated aqueous sodium bicarbonate (200 mL), extracted with hexane (200 mL×2). The combined organic layer was washed with brine, dried with anhydrous sodium sulfate, filtered, and concentrated under reduced pressure to afford (1R,2S)-diethyl 3,3-diethoxycyclobutane-1,2-dicarboxylate. Purification (FCC, SiO₂, PE:EA=80:1) afforded the title compound (3.41 g, 42.3% yield) as a yellow oil. ¹H NMR (400 MHz, CDCl₃) δ 4.31-4.18 (m, 4H), 4.18-4.11 (m, 4H), 3.75-3.69 (m, 1H), 3.34 (m, J=10.2, 8.5 Hz, 1H), 2.59 (m, 1H), 2.31-2.21 (m, 1H), 1.33 (m, 6H), 1.20-1.07 (m, 6H).

Step C. ((1R,2R)-3,3-Diethoxycyclobutane-1,2-diyl)dimethanol. To cooled (0° C.) mixture of (1R,2S)-diethyl 3,3-diethoxycyclobutane-1,2-dicarboxylate (55.30 g, 191.79 mmol) and anhydrous tetrahydrofuran (THF) (300 mL), was added LiAlH₄ (21.84 g, 575.37 mmol) slowly. The reaction mixture was stirred at r.t. for 4 h. The reaction mixture was quenched with H₂O (21 mL) slowly at 0° C., followed by NaOH aq solution (63 mL, The reaction mixture was filtered, and filtrate was concentrated under reduced pressure. The title compound, ((1R,2R)-3,3-diethoxycyclobutane-1,2-diyl)dimethanol (33.40 g, 163.52 mmol, 85.3% yield) was used in the next step without further purification.

Step D. ((1R,2R)-3,3-Diethoxycyclobutane-1,2-diyl)bis(methylene) dibenzoate. To a solution of ((1R,2R)-3,3-diethoxycyclobutane-1,2-diyl)dimethanol (33.40 g, 163.52 mmol) in pyridine (300 mL), was added benzoyl chloride (BzCl) (91.94 g, 654.08 mmol) slowly by syringe under N₂ atmosphere at 0° C. The reaction mixture was warmed to r.t. and stirred for 3 h. The reaction mixture was concentrated under reduced pressure, then dichloromethane (DCM) (500 mL) was added. The resulting solution was washed with saturated NaHCO₃ aqueous solution, then brine, dried with anhydrous sodium sulfate, filtered, and concentrated under reduced pressure. Purification (FCC, SiO₂, PE:EA=20:1) afforded ((1R,2R)-3,3-diethoxycyclobutane-1,2-diyl)bis(methylene) dibenzoate (59.70 g, 88.5% yield) as a colorless oil.

Step E. ((1R,2R)-3-Oxocyclobutane-1,2-diyl)bis(methylene) dibenzoate. To a solution of ((1R,2R)-3,3-diethoxycyclobutane-1,2-diyl)bis(methylene) dibenzoate (59.70 g, 144.98 mmol) in tetrahydrofuran (THF) (350 mL) was added HCl (0.4 M, 362.45 mL). The mixture was stirred at r.t. overnight. NaHCO₃ (0.2N) was added to the reaction mixture until a pH=7. The reaction mixture was extracted with EA (30 mL×2), washed with brine, dried with anhydrous sodium sulfate, filtered and concentrated under reduced pressure. Purification (FCC, SiO₂, PE:EA=10:1) afforded ((1R,2R)-3-oxocyclobutane-1,2-diyl)bis(methylene) dibenzoate (26.10 g, 77.14 mmol, 53.2% yield) as a white solid. ¹H NMR (400 MHz, CDCl₃) δ 8.07-7.98 (m, 4H), 7.62-7.55 (m, 2H), 7.44 (m, 4H), 4.68-4.53 (m, 4H), 3.75-3.63 (m, 1H), 3.34-3.22 (m, 1H), 3.15-3.05 (m, 1H), 3.05-2.91 (m, 1H).

Step F. ((1R,2R,3R)-3-Hydroxycyclobutane-1,2-diyl)bis(methylene) dibenzoate. A solution of THF (300 mL) and ((1R,2R)-3-oxocyclobutane-1,2-diyl)bis(methylene) dibenzoate (26.10 g, 77.14 mmol) was stirred at −78° C. under N₂ atmosphere. LS-selectride (77.14 mmol) was added slowly to the reaction mixture by syringe. The reaction mixture was stirred at −78° C. for 3 h. The pH of the reaction mixture was adjusted to pH=7.0 with 0.1 N HCl and stirred for 30 min. The reaction mixture was extracted with ethyl acetate (EA) (300 mL×2), the combined organic layer was washed with brine, dried over anhydrous sodium sulfate, filtered and concentrated under reduced pressure. Purification (FCC, SiO₂, PE:EA=30:1) afforded ((1R,2R,3R)-3-hydroxycyclobutane-1,2-diyl)bis(methylene) dibenzoate (21.10 g, 61.99 mmol, 80.4% yield) as a colorless oil. ¹H NMR (400 MHz, CDCl₃) δ 8.06 (m, 4H), 7.63-7.56 (m, 2H), 7.46 (m, 4H), 4.87 (dd, J=11.5, 8.9 Hz, 1H), 4.49 (m, 1H), 4.46-4.34 (m, 2H), 4.28 (dd, J=11.6, 4.5 Hz, 1H), 4.14 (q, J=7.2 Hz, 1H), 2.79 (d, J=7.4 Hz, 1H), 2.70 (d, J=8.4 Hz, 1H), 2.28-2.11 (m, 2H).

Step G. ((1R,2R,3R)-3-Hydroxycyclobutane-1,2-diyl)dimethanol. To a solution of ((1R,2R,3R)-3-hydroxycyclobutane-1,2-diyl)bis(methylene) dibenzoate (21.10 g, 61.99 mmol) in methanol (MeOH) (100 mL) was added 30% MeNH₂ (61.99 mmol, 200 mL). The reaction mixture was stirred at r.t. overnight under N₂ atmosphere. The reaction mixture was concentrated under reduced pressure. Purification (FCC, SiO₂, DCM:MeOH=15:1) afforded ((1R,2R,3R)-3-hydroxycyclobutane-1,2-diyl)dimethanol (5.50 g, 41.62 mmol, 67.1% yield) as a yellow oil. ¹H NMR (400 MHz, DMSO-d₆) δ 4.69 (d, J=5.2 Hz, 1H), 4.45 (t, J=5.3 Hz, 1H), 4.26-4.18 (m, 1H), 4.12 (t, J=5.4 Hz, 1H), 3.66 (m, 1H), 3.46 (m, 1H), 3.37-3.29 (m, 2H), 2.17-2.09 (m, 1H), 2.09-1.98 (m, 1H), 1.93 (m, 1H), 1.80 (m, 1H).

Step H. (1R,6R,7R)-2,4-Dioxaspiro[bicyclo[4.2.0]octane-3,1′-cyclohexan]-7-ylmethanol. To a solution of ((1R,2R,3R)-3-hydroxycyclobutane-1,2-diyl)dimethanol (4.80 g, 36.32 mmol) in cyclohexanone (142.58 g, 1.45 mol) was added p-toluenesulfonic acid (TsOH) (8.29 g, 43.58 mmol) and MgSO₄ (20 g, 192.31 mmol). The reaction mixture was stirred at r.t. overnight. To the reaction mixture was added 0.5 mL triethylamine (0.5 mL). The reaction mixture was concentrated under reduced pressure. Purification (FCC, SiO₂, DCM:MeOH=30:1) afforded (1R,6R,7R)-2,4-dioxaspiro[bicyclo[4.2.0]octane-3,1′-cyclohexan]-7-ylmethanol (8.10 g, 38.16 mmol, 105.1% yield) as an oil.

Step I. ((1R,6R,7R)-2,4-Dioxaspiro[bicyclo[4.2.0]octane-3,1′-cyclohexan]-7-ylmethoxy)(tert-butyl)diphenylsilane. To a solution of (1R,6R,7R)-2,4-dioxaspiro[bicyclo[4.2.0]octane-3,1′-cyclohexan]-7-ylmethanol (9.90 g, 46.64 mmol) in DMF (250 mL) was added imidazole (9.53 g, 139.92 mmol) at 0° C. tert-Butyldiphenylchlorosilane (TBDPSCl) (10.85 g, 93.28 mmol), was added and the reaction mixture was stirred at r.t. overnight. The reaction mixture was poured into water (500 mL), and extracted with EA (400 mL×2). The combined organic layers were washed with brine, dried with anhydrous sodium sulfate, filtered concentrated under reduced pressure to afford ((1R,6R,7R)-2,4-dioxaspiro[bicyclo[4.2.0]octane-3,1′-cyclohexan]-7-ylmethoxy)(tert-butyl)diphenylsilane (20 g, 44.38 mmol, 95.2% yield), which was used directly in the next step without further purification.

Step J. (1R,2R,3R)-3-(((tert-Butyldiphenylsilyl)oxy)methyl)-2-(hydroxymethyl)cyclobutanol. To a solution of ((1R,6R,7R)-2,4-dioxaspiro[bicyclo[4.2.0]octane-3,1′-cyclohexan]-7-ylmethoxy)(tert-butyl)diphenylsilane (20 g, 44.60 mmol) in MeOH (200 mL) was added pyridinium p-toluenesulfonate (PPTS) (4.48 g, 17.84 mmol), the mixture was stirred at r.t. overnight. The mixture was quenched with saturated NaHCO₃ (100 mL), extracted with EA (200×2), washed with brine, dried over anhydrous Na₂SO₄, filtered, and under reduced pressure. Purification (FCC, SiO₂, DCM:MeOH=10:1) afforded (1R,2R,3R)-3-(((tert-butyldiphenylsilyl)oxy)methyl)-2-(hydroxymethyl)cyclobutanol (9.50 g, 25.6 mmol, 57.5% yield) as a colorless oil. ¹H NMR (400 MHz, CDCl₃) δ 7.68 (m, 4H), 7.51-7.36 (m, 6H), 4.57 (s, 1H), 3.99-3.85 (m, 2H), 3.71-3.56 (m, 3H), 2.55 (d, J=6.2 Hz, 1H), 2.39-2.31 (m, 2H), 2.20 (m, 1H), 1.08 (s, 9H).

Step K. (1R,2R,3R)-3-(((tert-Butyldiphenylsilyl)oxy)methyl)-2-((trityloxy)methyl)cyclobutanol. To a solution of (1R,2R,3R)-3-(((tert-butyldiphenylsilyl)oxy)methyl)-2-(hydroxymethyl)cyclobutanol (9.50 g, 25.64 mmol) in pyridine (200 mL) was added trityl chloride (TrtCl) (10.71 g, 38.46 mmol). The mixture was stirred at r.t. overnight. The reaction mixture was concentrated under reduced pressure. Purification (FCC, SiO₂, PE:EA=20:1) afforded (1R,2R,3R)-3-(((tert-butyldiphenylsilyl)oxy)methyl)-2-((trityloxy)methyl)cyclobutanol (12.50 g, 20.4 mmol, 79.6% yield) as a colorless oil. ¹H NMR (400 MHz, CDCl₃) δ 7.70-7.63 (m, 4H), 7.52-7.46 (m, 6H), 7.42-7.24 (m, 10H), 4.52-4.41 (m, 1H), 4.15 (q, J=7.2 Hz, 2H), 3.73-3.59 (m, 2H), 3.47-3.36 (m, 2H), 2.68 (dd, J=12.1, 6.3 Hz, 2H), 2.49-2.37 (m, 1H), 2.26-2.14 (m, 1H), 2.08 (s, 3H), 1.29 (t, J=7.2 Hz, 3H), 1.06 (s, 9H). ESI-LCMS: m/z 635.4 [M+Na]⁺.

Step L. (((1R,2R,3R)-3-(Benzyloxy)-2-((trityloxy)methyl)cyclobutyl)methoxy)(tert-butyl)diphenylsilane. To a solution of (1R,2R,3R)-3-(((tert-butyldiphenylsilyl)oxy)methyl)-2-((trityloxy)methyl)cyclobutanol (1.97 g, 3.21 mmol) in DMF (30 mL) was added NaH (115.56 mg, 4.81 mmol) at 0° C. under N₂ atmosphere. The mixture was stirred at 0° C. for 30 min. Benzyl bromide (BnBr) (415.05 mg, 3.85 mmol) was added, the mixture was stirred at r.t. overnight. Water (100 mL) was added and the reaction mixture was extracted with EA (100 mL×2), washed with brine, dried over anhydrous sodium sulfate, filtered and the concentrated under reduced pressure. Purification (FCC, SiO₂, PE:EA=30:1) afforded (((1R,2R,3R)-3-(benzyloxy)-2-((trityloxy)methyl)cyclobutyl)methoxy)(tert-butyl)diphenylsilane (941.0 mg, 1.3 mmol, 41.7% yield) as a yellow oil, ESI-LCMS: m/z 725.5 [M+Na]⁺.

Step M. ((1R,2R,4R)-2-(Benzyloxy)-4-(((tert-butyldiphenylsilyl)oxy)methyl)cyclobutyl)methanol. To a solution of (((1R,2R,3R)-3-(benzyloxy)-2-((trityloxy)methyl)cyclobutyl)methoxy)(tert-butyl)diphenylsilane (941 mg, 1.34 mmol) in MeOH (20 mL) was added TsOH (127.45 mg, 670 μmol) at 0° C. The reaction mixture was stirred at 0° C. for 30 min, then warmed to r.t. and stirred for 3 h. To the reaction mixture was added saturated aq Na₂CO₃ (50 mL). The reaction mixture was extracted with EA (50 mL×2), the combined layers were washed with brine, dried over anhydrous sodium sulfate, filtrated and concentrated under reduced pressure. Purification (FCC, SiO₂, PE:EA=20:1) afforded ((1R,2R,4R)-2-(benzyloxy)-4-(((tert-butyldiphenylsilyl)oxy)methyl)cyclobutyl)methanol (320.0 mg, 694.6 μmol, 51.8% yield) as a colorless oil. ESI LC-MS: m/z 483.3 [M+Na]⁺.

Step N. (((1R,2S,3R)-3-(Benzyloxy)-2-(((2-nitrophenyl)selanyl)methyl)cyclobutyl)methoxy)(tert-butyl)diphenylsilane. To a solution of ((1R,2R,4R)-2-(benzyloxy)-4-(((tert-butyldiphenylsilyl)oxy)methyl)cyclobutyl)methanol (320 mg, 694.63 μmol) in THF (7.50 mL) was added 1-nitro-2-selenocyanatobenzene (315.47 mg, 1.39 mmol), followed by tributylphosphine (PBu₃) (281.08 mg, 1.39 mmol). The reaction mixture was stirred at 55° C. overnight. The reaction mixture was concentrated under reduced pressure. Purification (FCC, SiO₂, PE:EA=40:1) afforded (((1R,2S,3R)-3-(benzyloxy)-2-(((2-nitrophenyl)selanyl)methyl)cyclobutyl)methoxy)(tert-butyl)diphenylsilane (460.0 mg, 713.5 μNmol, 102.7% yield) as a brown smelly solid. ESI LC-MS: m/z 668.3 [M+Na]⁺.

Step O. (((1R,3R)-3-(Benzyloxy)-2-methylenecyclobutyl)methoxy)(tert-butyl)diphenylsilane. To a solution of (((1R,2S,3R)-3-(benzyloxy)-2-(((2-nitrophenyl)selanyl)methyl)cyclobutyl)methoxy)(tert-butyl)diphenylsilane (460 mg, 713.48 μmol) in pyridine (15 mL) was added H₂O₂ (48.53 g, 1.43 mol, 1.62 mL). The reaction mixture was stirred at 55° C. overnight. To the reaction mixture was added H₂O (40 mL). The reaction mixture was extracted with EA (40 mL×2), and the combined organic layers were washed with brine, dried over anhydrous sodium sulfate, filtered, and concentrated under reduced pressure. Purification (FCC, SiO₂, PE:EA=30:1) afforded (((1R,3R)-3-(benzyloxy)-2-methylenecyclobutyl)methoxy)(tert-butyl)diphenylsilane (250.0 mg, 564.8 μmol, 79.2% yield) as a yellow smelly solid. ¹H NMR (400 MHz, CDCl₃) δ 7.68 (m, 4H), 7.49-7.34 (m, 11H), 5.19 (t, J=2.2 Hz, 1H), 5.04 (t, J=2.2 Hz, 1H), 4.57 (d, J=1.8 Hz, 2H), 3.70 (m, 2H), 3.09 (s, 1H), 2.16-2.10 (m, 2H), 1.06 (s, 9H). ESI-LCMS: m/z 443.3 [M+H]⁺.

Step P. (1R,3R)-3-(((tert-Butyldiphenylsilyl)oxy)methyl)-2-methylenecyclobutanol. A solution of (((1R,3R)-3-(benzyloxy)-2-methylenecyclobutyl)methoxy)(tert-butyl)diphenylsilane (250 mg, 564.77 μmol) in DCM (20 mL) was stirred at −75° C. BCl₃ (1 M, 847.16 μL) was added slowly. The mixture was stirred at −75° C. for 1 h. To the reaction mixture was added saturated aq Na₂CO₃ (4 mL) and H₂O (20 mL). The reaction mixture was extracted with DCM (20 mL×2), washed with brine, dried, and concentrated under reduced pressure. Purification (FCC, SiO₂, DCM:MeOH=25:1) afforded (1R,3R)-3-(((tert-butyldiphenylsilyl)oxy)methyl)-2-methylenecyclobutanol (104.0 mg, 295.0 μmol, 52.2% yield) as a colorless oil. ¹H NMR (400 MHz, DMSO-d₆) δ 7.67-7.57 (m, 4H), 7.51-7.41 (m, 6H), 5.30 (d, J=7.2 Hz, 1H), 5.02 (t, J=2.3 Hz, 1H), 4.87 (t, J=2.1 Hz, 1H), 4.58 (d, J=7.8 Hz, 1H), 3.74-3.59 (m, 2H), 2.96-2.83 (m, 1H), 2.07 (m, 1H), 1.87 (m, 1H), 1.26-1.16 (m, 1H), 1.01 (s, 9H). ESI-LCMS: m/z 376.3 [M+Na]⁺. See also Slusarchyk et al., Tetrahedron Letters (1989), 30(47), 6453-6456.

### Intermediate 2. (1R,3R)-3-(((tert-Butyldiphenylsilyl)oxy)methyl)-2-methylenecyclobutanol

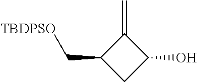

Step A. ((1R,2R,3R)-3-(Benzyloxy)cyclobutane-1,2-diyl)bis(methylene) dibenzoate. To a solution of ((1R,2R,3R)-3-hydroxycyclobutane-1,2-diyl)bis(methylene) dibenzoate (Intermediate 1, product from Step F, 9.40 g, 27.62 mmol) in DCM (29.70 mL) and cyclohexane (60.30 mL) was added benzyl 2,2,2-trichloroacetimidate (6.97 g, 27.62 mmol), followed by CF₃SO₃H (829.04 mg, 5.52 mmol. The reaction mixture was stirred at r.t. for 4 h. The reaction mixture was quenched with sat. aq NaHCO₃ (300 mL), extracted with EA (200 mL×3), washed with brine, dried with Na₂SO₄, concentrated under reduced pressure. Purification (FCC, SiO₂, DCM:MeOH=15:1) afforded ((1R,2R,3R)-3-(benzyloxy)cyclobutane-1,2-diyl)bis(methylene) dibenzoate (12.60 g, 29.27 mmol, 105.97% yield) as a yellow oil. ESI LC-MS: m/z 431.2 [M+H]⁺.

Step B. ((1R,2R,3R)-3-(Benzyloxy)cyclobutane-1,2-diyl)dimethanol. A solution of ((1R,2R,3R)-3-(benzyloxy)cyclobutane-1,2-diyl)bis(methylene) dibenzoate (12.40 g, 28.80 mmol) and 33% MeNH₂ (28.8 mmol, 200 mL) was stirred at 55° C. overnight. The reaction mixture was concentrated under reduced pressure. Purification (FCC, SiO₂, DCM:MeOH=10:1) afforded ((1R,2R,3R)-3-(benzyloxy)cyclobutane-1,2-diyl)dimethanol (3.95 g 17.79 mmol, 61.72% yield) as a colorless oil. ESI-LCMS: m/z 223.1 [M+H]⁺.

Step C. ((1R,2R,4R)-2-(Benzyloxy)-4-(((tert-butyldiphenylsilyl)oxy)methyl)cyclobutyl)methanol. To a solution of ((1R,2R,3R)-3-(benzyloxy)cyclobutane-1,2-diyl)dimethanol (3.45 g, 15.52 mmol) in DCM (120 mL) was added imidazole (3.17 g, 46.56 mmol), followed by TBDPSCl (1.81 g, 15.52 mmol) at 0° C. The reaction mixture was stirred at r.t. for 2 h. The reaction mixture was quenched with water (20 mL), extracted with DCM (20 mL×2), washed with brine, dried over anhydrous Na₂SO₄, filtered and concentrated under reduced pressure. Purification (FCC, SiO₂, PE:EA=20:1) afforded ((1R,2R,4R)-2-(benzyloxy)-4-(((tert-butyldiphenylsilyl)oxy)methyl)cyclobutyl)methanol (2.40 g, 5.21 mmol, 33.6% yield) as a colorless oil. ESI-LCMS: m/z 483.3 [M+Na]+. The double protected product 2.3 g, the recovered starting material 1.1 g, and ((1R,2R,3R)-3-(benzyloxy)-2-(((tert-butyldiphenylsilyl)oxy)methyl)cyclobutyl)methanol as byproduct (550 mg).

Step D. (((1R,2S,3R)-3-(Benzyloxy)-2-(((2-nitrophenyl)selanyl)methyl)cyclobutyl)methoxy)(tert-butyl)diphenylsilane. To a solution of ((1R,2R,4R)-2-(benzyloxy)-4-(((tert-butyldiphenylsilyl)oxy)methyl)cyclobutyl)methanol (2.70 g, 5.86 mmol) in THF (30 mL) was added PBu₃ (3.56 g, 17.58 mmol, 4.40 mL), 1-nitro-2-selenocyanatobenzene (3.99 g, 17.58 mmol) at r.t. To the reaction mixture was added PBu₃ (3.56 g, 17.58 mmol, 4.40 mL) at r.t. under N₂, and the reaction mixture was stirred at 55° C. overnight. The reaction mixture was concentrated under reduced pressure. Purification (FCC, SiO₂, PE:EA=40:1 afforded (((1R,2S,3R)-3-(benzyloxy)-2-(((2-nitrophenyl)selanyl)methyl)cyclobutyl)methoxy)(tert-butyl)diphenylsilane (4.50 g, 6.98 mmol, 119.1% yield) as a yellow solid. ESI-LCMS: m/z 668.3 [M+Na]⁺.

Step E. (((1R,3R)-3-(Benzyloxy)-2-methylenecyclobutyl)methoxy)(tert-butyl)diphenylsilane. To a solution of (((1R,2S,3R)-3-(benzyloxy)-2-(((2-nitrophenyl)selanyl)methyl)cyclobutyl)methoxy)(tert-butyl)diphenylsilane (3.78 g, 5.86 mmol) in pyridine (100 mL) was added 30% H₂O₂ (13.29 g, 117.26 mmol, 13.29 mL). The reaction mixture was stirred at 55° C. under N₂ for 4 h. To the reaction mixture was added water (500 mL). The reaction mixture was extracted with EA (500 mL×2), the combined organic layers were washed with brine, dried with anhydrous Na₂SO₄, concentrated under reduced pressure. Purification (FCC, SiO₂, PE:EA=30:1) afforded (((1R,3R)-3-(benzyloxy)-2-methylenecyclobutyl)methoxy)(tert-butyl)diphenylsilane (2.30 g, 5.20 mmol, 88.7% yield) as a colorless smelly oil. ESI-LCMS: m/z 443.3 [M+H]⁺.

Step F. (1R,3R)-3-(((tert-Butyldiphenylsilyl)oxy)methyl)-2-methylenecyclobutanol. To a solution of (((1R,3R)-3-(benzyloxy)-2-methylenecyclobutyl)methoxy)(tert-butyl)diphenylsilane (2.30 g, 5.20 mmol) in DCM (40 mL) was added BCl₃ (1 M, 10.40 mL) at −78° C., under N₂ atmosphere. The mixture was stirred at −78° C. for 30 min. The reaction mixture was quenched with MeOH and triethylamine (TEA) (1:2, 3 mL), and concentrated under reduced pressure. Purification (FCC, SiO₂, DCM:MeOH=25:1) afforded (1R,3R)-3-(((tert-butyldiphenylsilyl)oxy)methyl)-2-methylenecyclobutanol (1.01 g, 2.86 mmol, 55.1% yield) as a colorless oil. ESI LC-MS: m/z 376.3 [M+Na]⁺.

### Intermediate 3. (1R,2S,3S,4S)-2,3-Bis((benzyloxy)methyl)-5-oxabicyclo[2.1.0]pentane

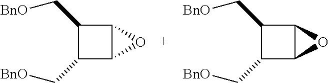 [SMILES: c1ccc(COC[C@@H]2[C@@H](COCc3ccccc3)[C@@H]3O[C@H]23)cc1 ; c1ccc(COC[C@@H]2[C@@H](COCc3ccccc3)[C@@H]3O[C@H]23)cc1]

Step A. (1R,5S)-3-Oxabicyclo[3.2.0]hept-6-ene-2,4-dione. Furan-2,5-dione (32.0 g, 326.3 mmol), acetophenone (75.1 mmol, 8.76 mL) and ethyl acetate (1.3 L) were placed in a 2.0 litre Pyrex vessel with a polypropylene lid, through which were fitted a triple-walled immersion-well lamp housing, a fitted gas bubbler, a low temperature thermometer and a gas outlet tube. With the supply of cooling ethanol to the lamp flowing and a continuous stream of nitrogen passing through the solution, the whole reactor was cooled to −40° C. to −70° C. Acetylene gas was then passed into the mixture at a high flow rate and irradiation was commenced. Exhausted gases was carefully ducted into a powerful extraction system. The reaction was monitored by evaporating 1 mL portions for examination by 41 NMR. After 72 h, 20% SM was still remained and no improvement on the ratio of SM:product, then the irradiation was stopped. The solvent was removed in vacuo. The residue was washed with petroleum ether (PE):CHCl₃=100:1 (500 mL) to afford (1R,5S)-3-oxabicyclo[3.2.0]hept-6-ene-2,4-dione (38.5 g, 315.3 mmol, 97% yield) as a light yellow solid. ¹H-NMR (400 MHz, DMSO-d₆): δ 5.09 (s, 2H), 4.08 (s, 2H). ESI-LCMS: m/z 407.1 [M+H]⁺.

Step B. (1R,4R)-4-(Methoxycarbonyl)cyclobut-2-enecarboxylic acid. To a suspension of (1R,5S)-3-oxabicyclo[3.2.0]hept-6-ene-2,4-dione (47.9 g, 386.0 mmol) in MeOH (500 mL) was added NaOMe (2.5 M, 1.24 L) at 0° C. over 1 h. The resulting mixture was stirred at r.t. for 6 days. The mixture was added to 4 M HCl (770 ml) at 0° C., then concentrated under reduced pressure. The resulting residue was diluted with EA (2 L), dried over anhydrous sodium sulfate, filtered, concentrated under reduced pressure to afford (1R,4R)-4-(methoxycarbonyl)cyclobut-2-enecarboxylic acid (57.0 g, 365.1 mmol, 95% yield) as a yellow oil which was used directly in the next step without purification.

Step C. (1S,2S)-Cyclobut-3-ene-1,2-diyldimethanol. To a suspension of LiAlH₄ (27.7 g, 730.1 mmol) in THF (1 L) was added a solution of (1R,4R)-4-(methoxycarbonyl)cyclobut-2-enecarboxylic acid (28.5 g, 182.5 mmol) in THF (100 mL) at 0° C. The resulting mixture was stirred at r.t. overnight. The mixture was quenched by water (27.8 mL) followed by 15% NaOH aq. (83.4 mL). The mixture was filtered and the filter cake was washed with DCM (1 L×4). The filtrate was concentrated under reduced pressure. Purification (FCC, SiO₂, PE:EA=1:1) afforded (1S,2S)-cyclobut-3-ene-1,2-diyldimethanol (13.3 g, 116.5 mmol, 64% yield) as a yellow oil.

Step D. (3S,4S)-3,4-Bis((benzyloxy)methyl)cyclobut-1-ene. To a stirred solution of (1S,2S)-cyclobut-3-ene-1,2-diyldimethanol (26.7 g, 233.9 mmol) in DMF (800 mL) was added NaH (28.1 g, 701.8 mmol, 60% purity) and BnBr (60.5 g, 561.4 mmol) sequentially at 0° C. The resulting mixture was stirred at r.t. for 1 h. The reaction was quenched with H₂O (500 mL) at 0° C. The reaction mixture was extracted with EA (300 ml×5, and the combined extracts were washed with brine, dried over anhydrous sodium sulfate, filtered, and concentrated under reduced pressure. Purification (FCC, SiO₂, PE:EA=30:1) afforded (3S,4S)-3,4-bis((benzyloxy)methyl)cyclobut-1-ene (41.9 g, 142.3 mmol, 61% yield) as yellow oil.

Step E. (1R,2S,3S,4S)-2,3-Bis((benzyloxy)methyl)-5-oxabicyclo[2.1.0]pentane. To a solution of (3S,4S)-3,4-bis((benzyloxy)methyl)cyclobut-1-ene (41.9 g, 142.3 mmol) in DCM (1.2 L) was added NaHCO₃ (4.8 g, 56.9 mmol) and meta-chloroperoxybenzoic acid (m-CPBA) (31.9 g, 185.0 mmol) at 0° C. The resulting mixture was stirred at r.t. for 16 h. The mixture was quenched by sat. aq. Na₂SO₃, then the mixture was basified by saturated aq. NaHCO₃ to pH=9, and extracted with DCM (1 L×2). The combined extracts were washed with brine, dried over anhydrous sodium sulfate, filtered, and concentrated under reduced pressure. Purification (FCC, SiO₂, PE:EA=20:1) afforded (1R,2S,3S,4S)-2,3-bis((benzyloxy)methyl)-5-oxabicyclo[2.1.0]pentane and its isomer as a mixture (20.8 g, 46.9 mmol, 33% yield, 70% purity) as a yellowish oil. ESI LC-MS: m/z 311 [M+H]⁺.

### Intermediate 4: (1R,2S,3R,4S)-2,3-Bis((benzyloxy)methyl)-4-(diethoxymethyl)cyclobutanol

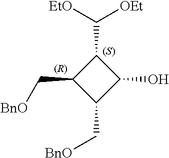

Step A. (−)-Dimenthyl fumarate. The title compound was prepared according to the procedure as described in WO 2007/008564, page 20. Int. Pub. Date 18 Jan. 2007.

Step B. (1S,2R)-Dimenthyl 3,3-diethoxycyclobutane-1,2-dicarboxylate. To a 3-neck flask was added toluene (500 mL), (−)-dimenthyl fumarate (100 g, 0.25 mol), the mixture was cooled to −45° C. under N₂ atmosphere, diethylaluminum chloride (1 M, 750 mL) was added slowly by syringe, the mixture was stirred for 10 min. DIPEA (11.7 g, 90 mmol) was added and the mixture was stirred at −45° C. for 10 min, diethyl fumarate (8.77 g, 75.50 mmol) was added by syringe, the mixture was keep at −45° C. for 3 h. The mixture was quenched with saturated aqueous sodium bicarbonate (200 mL), extracted with hexane (200 mL, ×2). The combined organic layer was washed with brine, dried over anhydrous sodium sulfate, filtered, and concentrated under reduced pressure. Purification (FCC, SiO₂, PE:EA=100:1) afforded (1S,2R)-dimenthyl 3,3-diethoxycyclobutane-1,2-dicarboxylate (3.41 g, 11.8 mmol, 42.3% yield) as a yellow oil. 41 NMR (400 MHz, CDCl₃) δ 4.31-4.18 (m, 4H), 4.18-4.11 (m, 4H), 3.75-3.69 (m, 1H), 3.34 (m, J=10.2, 8.5 Hz, 1H), 2.59 (m, 1H), 2.31-2.21 (m, 1H), 1.33 (m, Hz, 6H), 1.20-1.07 (m, 6H).

Step C. ((1S,2S)-3,3-Diethoxycyclobutane-1,2-diyl)dimethanol. To a cooled, 0° C., solution of (1S,2R)-dimenthyl 3,3-diethoxycyclobutane-1,2-dicarboxylate (300.5 g, 0.55 mol) dissolved in anhydrous THF (300 mL), was added LiAlH₄ (118.5 g, 3.12 mol) slowly. The mixture was stirred at r.t. for 4 h. The mixture was quenched with H₂O (100 mL) slowly at 0° C., followed by aq. NaOH (15%) (300 mL). The reaction mixture was filtered and the filtrate was concentrated in vacuo. Purification (FCC, SiO₂, PE:EA=1:1) afforded ((1S,2S)-3,3-diethoxycyclobutane-1,2-diyl)dimethanol (102.1 g, 0.50 mol, 90.9% yield) as a colorless oil.

Step D. (((((1S,2S)-3,3-Diethoxycyclobutane-1,2-diyl)bis(methylene))bis(oxy))bis(methylene))dibenzene. A cooled solution, 0° C., of ((1S,2S)-3,3-diethoxycyclobutane-1,2-diyl)dimethanol (102.1 g, 0.50 mol) dissolved in DMF (300 mL) was stirred under a N₂ atmosphere. NaH (100 g, 2.5 mmol, 60% in mineral oil) was added to the mixture, and the reaction mixture was stirred for 30 min at 0° C. BnBr (256.5 g, 1.5 mmol) was added slowly by syringe and the reaction mixture was allowed to warm to r.t. stirred for 3 h. The reaction mixture was quenched with ice water and concentrated in vacuo. The crude product was dissolved in EA (500 mL) and the resulting solution was washed with water, then brine, dried over anhydrous sodium sulfate, filtered, and concentrated under reduced pressure. Purification (FCC, SiO₂, PE:EA=30:1) afforded (441S,2S)-3,3-diethoxycyclobutane-1,2-diyl)bis(methylene))bis(oxy))bis(methylene))dibenzene (170.1 g, 0.44 mmol, 88.5% yield) as a colorless oil. LCMS m/z=385.2 [M+H]⁺.

Step E. (2S,3S)-2,3-Bis((benzyloxy)methyl)cyclobutanone. To a solution of (441S,2S)-3,3-diethoxycyclobutane-1,2-diyl)bis(methylene))bis(oxy))bis(methylene))dibenzene (170.1 g, 0.44 mmol) in CH₃CN (1.75 L) was added 0.5 N H₂SO₄ (660 mL). The reaction mixture was stirred at r.t. for 2 h, then diluted with EtOAc (5 L), washed with water (2×1 L), saturated sodium bicarbonate (1 L), water (2×1 L) and brine (1 L). The organic phase was separated, dried (Na₂SO₄), filtered, and concentrated in vacuo. Purification (FCC, SiO₂, PE:EA=30:1) afforded (2S,3S)-2,3-bis((benzyloxy)methyl)cyclobutanone (125.4 g, 0.41 mmol, 93.2% yield) as a colorless oil. LC-MS m/z=311.2 [M+H]⁺.

Step F. (2S,3R,4R)-2,3-Bis((benzyloxy)methyl)-4-(diethoxymethyl)cyclobutanone. To a solution of triethyl orthoformate (143.0 g, 0.96 mol) in DCM (100 mL) at −30° C. was added BF₃.OEt₂ (203.4 g, 1.44 mol) in a dropwise fashion. After 30 min the reaction mixture was warmed to 0° C. for 15 min and cooled back down to −78° C. To the reaction mixture was added a solution of (2S,3S)-2,3-bis((benzyloxy)methyl)cyclobutanone (150 g, 0.48 mol) in DCM (200 mL) and DIPEA (245.1 g, 1.9 mol) in a dropwise fashion. After 1 h at −78° C., the reaction mixture was quenched with sat. NaHCO₃ solution and diluted with DCM. The biphasic solution was separated, and the aqueous phase was extracted two more times with DCM. The organic phases were combined, backwashed with brine, dried over Na₂SO₄, filtered and concentrated under reduced pressure. Purification (FCC, SiO₂, PE:EA=10:1) afforded (2S,3R,4R)-2,3-bis((benzyloxy)methyl)-4-(diethoxymethyl)cyclobutanone (180.5 g, 0.44 mol, 45.8% yield) as a colorless oil. LCMS m/z=413.2 [M+H]⁺.

Step G. (1R,2S,3R,4S)-2,3-Bis((benzyloxy)methyl)-4-(diethoxymethyl)cyclobutanol. To a solution of (2S,3R,4R)-2,3-bis((benzyloxy)methyl)-4-(diethoxymethyl)cyclobutanone (80.1 g, 0.19 mol) in THF (100 mL) was added a 1M solution of L-Selectride in THF (290 mL, 0.29 mol) at −78° C. The reaction mixture was warmed up to r.t. over 30 min and cooled back down to 0° C. to be quenched with sat. NH₄Cl solution (100 mL). The solution was diluted with water and EtOAc, the biphasic solution was separated and the organic phase was washed three times with water, dried over Na₂SO₄, filtered, and concentrated under reduced pressure. Purification (FCC, SiO₂, PE:EA=5:1) afforded (1R,2S,3R,4S)-2,3-bis((benzyloxy)methyl)-4-(diethoxymethyl)cyclobutanol (40.2 g, 97 mmol, 51.1% yield) as a colorless oil. LCMS m/z=437.2 [M+H]⁺.

### Intermediate 5. (1R,2S,3R)-2,3-Bis((benzyloxy)methyl)-4-methylenecyclobutanol

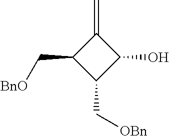 [SMILES: C=C1[C@H](O)[C@H](COCc2ccccc2)[C@H]1COCc1ccccc1]

Step A. (1R,2S,3R,4S)-2,3-Bis((benzyloxy)methyl)-4-fdiethoxymethyl)cyclobutyl acetate. To a solution of (1R,2S,3R,4S)-2,3-bis((benzyloxy)methyl)-4-(diethoxymethyl)cyclobutanol (Intermediate 4, 10.0 g, 24.0 mmol) in pyridine (100 mL) at r.t. was added acetic anhydride (Ac20) (7.4 g, 72.5 mmol) and DMAP (0.6 g, 5.0 mmol). The mixture was stirred at r.t. The reaction mixture was concentrated under reduced pressure. The resulting crude product dissolved with DCM, washed with H₂O, then washed with brine, dried over Na₂SO₄, filtered and concentrated under reduced pressure. Purification (FCC, SiO₂, PE:EA=5:1) afforded (1R,2S,3R,4S)-2,3-bis((benzyloxy)methyl)-4-(diethoxymethyl)cyclobutyl acetate (9.5 g, 86.3% yield) as a colorless oil. ESI LC-MS m/z=479.2 [M+H]+. ¹H-NMR (400 MHz, DMSO-d₆): δ 7.37-7.24 (m, 12H), 5.29 (t, J=7.3 Hz, 1H), 4.59 (d, J=8.5 Hz, 1H), 4.51-4.35 (m, 5H), 3.57-3.34 (m, 3H), 2.65-2.52 (m, 2H), 2.32-2.21 (m, 1H), 1.98-1.94 (m, 4H), 1.07-0.96 (m, 7H).

Step B. (1R,2S,3R,4S)-2,3-Bis((benzyloxy)methyl)-4-formylcyclobutyl acetate. To a solution of (1R,2S,3R,4S)-2,3-bis((benzyloxy)methyl)-4-(diethoxymethyl)cyclobutyl acetate (9.5 g, 20.8 mmol) in CH₃CN (200 mL) at r.t. was added 1N H₂SO₄ (187.2 mmol, 187.2 mL). The mixture was stirred at r.t. for 3 h. The reaction mixture was extracted with EtOAc and the organic layer was washed with saturated sodium bicarbonate, H₂O and brine, dried over Na₂SO₄, filtered and concentrated under reduced pressure. Purification (FCC, SiO₂, PE:EA=5:1) afforded (1R,2S,3R,4S)-2,3-bis((benzyloxy)methyl)-4-formylcyclobutyl acetate (7.6 g, 19.9 mmol, 95.5% yield) as a colorless oil. ESI LC-MS m/z=383.1 [M+H]⁺.

Step C. (1S,2S,3S,4R)-2,3-Bis((benzyloxy)methyl)-4-hydroxymethyl)cyclobutyl acetate. To a solution of (1R,2S,3R,4S)-2,3-bis((benzyloxy)methyl)-4-formylcyclobutyl acetate (7.6 g, 19.9 mmol) in THF (150 mL) at r.t. was added NaBH₄ (1.1 g, 29.8 mmol). The reaction mixture was stirred for 0.5 h. The reaction mixture was quenched with water and the resulted mixture was extracted with EtOAc. The combined organic layers were washed with H₂O, brine, dried over Na₂SO₄, filtered and concentrated under reduced pressure. Purification (FCC, SiO₂, PE:EA=2:1) afforded (1S,2S,3S,4R)-2,3-bis((benzyloxy)methyl)-4-(hydroxymethyl)cyclobutyl acetate (4.9 g, 12.7 mmol, 64.1% yield) as a colorless oil. ESI LC-MS m/z=385.1 [M+H]⁺. ¹H-NMR (400 MHz, DMSO-d₆): δ 7.39-7.23 (m, 10H), 5.24 (td, J=6.8, 0.8 Hz, 1H), 4.48 (s, 2H), 4.41 (d, J=4.7, 2H), 4.30 (t, J=5.4 Hz, 1H), 3.58-3.35 (m, 6H), 2.64-2.54 (m, 1H), 2.46-2.36 (m, 1H), 2.17-2.07 (m, 1H), 1.98 (s, 3H).

Step D. (1R,2S,3R)-2,3-Bis((benzyloxy)methyl)-4-methylenecyclobutyl acetate. To a solution of (1S,2S,3S,4R)-2,3-bis((benzyloxy)methyl)-4-(hydroxymethyl)cyclobutyl acetate (4.9 g, 12.7 mmol) in THF (100 mL) at r.t. was added phenylselenocyanate (4.6 g, 25.4 mmol) and (tBu)₃P (5.1 g, 25.4 mmol). The mixture was stirred at r.t. for 2 h, then H₂O₂ (100 mL) was added. The mixture was stirred at r.t. for 2 h and then stirred at 50° C. for another 2 h. The reaction mixture was quenched with Na₂SO₃ aqueous solution, and extracted with EtOAc. The organic layer was washed with H₂O, brine, dried over Na₂SO₄, filtered, and concentrated under reduced pressure. Purification (FCC, SiO₂, PE:EA=3:1) afforded (1R,2S,3R)-2,3-bis((benzyloxy)methyl)-4-methylenecyclobutyl acetate (2.9 g, 7.9 mmol, 62.1% yield) as a colorless oil. ESI LC-MS m/z=367.2 [M+H]⁺. ¹H-NMR (400 MHz, DMSO-d₆): δ 7.42-7.21 (m, 10H), 5.64-5.55 (m, 1H), 5.13 (t, J=2.3 Hz, 1H), 5.09 (t, J=2.3 Hz, 1H), 4.51 (s, 2H), 4.46 (s, 2H), 3.67-3.42 (m, 4H), 2.89-2.78 (m, 1H), 2.76-2.64 (m, 1H), 1.98 (s, 3H).

Step E. (1R,2S,3R)-2,3-Bis((benzyloxy)methyl)-4-methylenecyclobutanol. To a solution of (1R,2S,3R)-2,3-bis((benzyloxy)methyl)-4-methylenecyclobutyl acetate (2.9 g, 7.9 mmol) in MeOH (30 mL) at r.t. was added K₂CO₃ (3.3 g, 23.7 mmol). The mixture was stirred at r.t. for 1 h. The resulting solid was filtered from the solution and the liquid was concentrated under reduced pressure. Purification (FCC, SiO₂, PE:EA=2:1) afforded (1R,2S,3R)-2,3-bis((benzyloxy)methyl)-4-methylenecyclobutanol (2.1 g, 6.5 mmol, 81.8% yield) as a colorless oil. ESI LC-MS m/z=325.1 [M+H]⁺. ¹H-NMR (400 MHz, DMSO-d₆): δ 7.41-7.22 (m, 10H), 5.22 (d, J=6.4 Hz, 1H), 5.03 (t, J=2.2 Hz, 1H), 4.93 (t, J=2.2 Hz, 1H), 4.75-4.65 (m, 1H), 4.55-4.40 (m, 4H), 3.66 (dd, J=9.9, 6.1 Hz, 1H), 3.56-3.42 (m, 3H), 2.77 (t, J=3.4 Hz, 1H), 2.49-2.41 (m, 1H).

### Intermediate 6: ((1R,2R,3S)-3-(6-Amino-9H-purin-9-yl)-2-(((4-methoxyphenyl)diphenylmethoxy)methyl)-4-methylenecyclobutyl)methanol

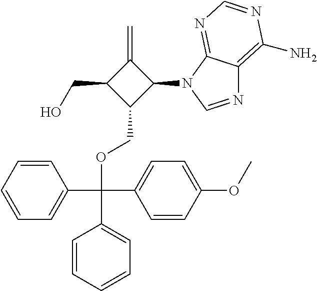 [SMILES: C=C1[C@@H](n2cnc3c(N)ncnc32)[C@H](COC(c2ccccc2)(c2ccccc2)c2ccc(OC)cc2)[C@H]1CO]

Step A. N-(9-((1S,2R,3R)-3-(Hydroxymethyl)-2-(((4-methoxyphenyl)diphenylmethoxy)methyl)-4-methylenecyclobutyl)-9H-purin-6-yl)benzamide. To a solution of N-(9-((1S,2R,3R)-2,3-bis(hydroxymethyl)-4-methylenecyclobutyl)-9H-purin-6-yl)benzamide (Example 8, Product from Step F, 225 mg, 615.8 μmol) in DCM (5 mL) was added pyridine (3.08 mmol, 250 μL), followed by 4-methoxytriphenylchloromethane (MMTrCl) (190 mg, 615.8 μmol) at rt. The resulting mixture was stirred at r.t. for 18 h. The reaction mixture was diluted with DCM, washed with citric acid aq., brine, dried over anhydrous sodium sulfate, filtered, concentrated under reduced pressure. Purification (FCC, SiO₂, DCM:MeOH=100:1 to 50:1) N-(9-((1S,2R,3R)-3-(hydroxymethyl)-2-(((4-methoxyphenyl)diphenylmethoxy)methyl)-4-methylenecyclobutyl)-9H-purin-6-yl)benzamide (145 mg, 227.4 μmol, 37% yield) as a white solid, ESI-LCMS: m/z 638 [M+H]⁺.

Step B. ((1R,2R,3S)-3-(6-Amino-9H-purin-9-yl)-2-(((4-methoxyphenyl)diphenylmethoxy)methyl)-4-methylenecyclobutyl)methanol. A solution of N-(9-((1S,2R,3R)-3-(hydroxymethyl)-2-(((4-methoxyphenyl)diphenylmethoxy)methyl)-4-methylenecyclobutyl)-9H-purin-6-yl)benzamide (112 mg, 175.6 μmol) in CH₃NH₂/EtOH (3 mL) was stirred at r.t. for 15 min. The reaction mixture was concentrated in vacuo. Purification (Flash-Prep-HPLC with the following conditions: Column, C18 silica gel (4 g); mobile phase, CH₃CN/H₂O=0/1 increasing to CH₃CN/H₂O (5 mM NH₄HCO₃)=1/0 within 15 min, the eluted product was collected at CH₃CN/H₂O=45/55; Detector, UV 254 nm) afforded ((1R,2R,3S)-3-(6-amino-9H-purin-9-yl)-2-(((4-methoxyphenyl)diphenylmethoxy)methyl)-4-methylenecyclobutyl)methanol (62 mg, 116.2 μmol, 66% yield) as a white solid. ¹H-NMR (400 MHz, DMSO-d₆): δ 8.29 (s, 1H), 8.17 (s, 1H), 7.22-7.29 (m, 12H), 7.13 (d, J=8.8 Hz, 2H), 6.83 (d, J=8.8 Hz, 2H), 5.44 (d, J=8.4 Hz, 1H), 5.09 (s, 1H), 4.79 (s, 1H), 4.72 (t, J=5.6 Hz, 1H), 3.73 (s, 3H), 3.62-3.71 (m, 2H), 3.19-3.21 (m, 2H), 2.96-3.04 (m, 1H), 2.84-2.86 (m, 1H). ESI-LCMS: m/z 534 [M+H]⁺.

### Intermediate 7: ((1S,2S,3R)-3-(6-Amino-9H-purin-9-yl)-2-(((4-methoxyphenyl)diphenylmethoxy)methyl)-4-methylenecyclobutyl)methanol

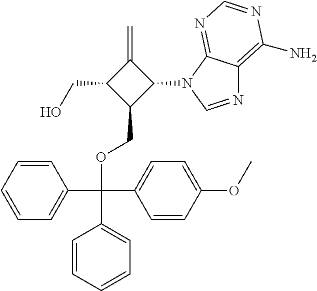 [SMILES: C=C1[C@@H](CO)[C@H](COC(c2ccccc2)(c2ccccc2)c2ccc(OC)cc2)[C@H]1n1cnc2c(N)ncnc21]

Step A. N-(9-((1R,2S,3S)-3-(Hydroxymethyl)-2-(((4-methoxyphenyl)diphenylmethoxy)methyl)-4-methylenecyclobutyl)-9H-purin-6-yl)benzamide. To a solution of N-(9-((1R,2S,3S)-2,3-bis(hydroxymethyl)-4-methylenecyclobutyl)-9H-purin-6-yl)benzamide (Example 8, product from Step E and F, 301 mg, 823.8 μmol) in DCM (6 mL) was added pyridine (4.12 mmol, 332 μL) followed by MMTrCl (254 mg, 823.8 μmol) at r.t. The resulting mixture was stirred at r.t. for 18 h. The mixture was diluted with DCM, washed with citric acid aq. solution, brine, dried over anhydrous sodium sulfate, filtered and concentrated under reduced pressure. Purification (FCC, SiO₂, DCM:MeOH=100:1 to 50:1) afforded N-(9-((1R,2S,3S)-3-(hydroxymethyl)-2-(((4-methoxyphenyl)diphenylmethoxy)methyl)-4-methylenecyclobutyl)-9H-purin-6-yl)benzamide (112 mg, 175.6 μmol, 21% yield) as a white solid, ESI LC-MS: m/z 638 [M+H]⁺ and N-(9-((1R,2S,3S)-2-(hydroxymethyl)-3-(((4-methoxyphenyl)diphenylmethoxy)methyl)-4-methylenecyclobutyl)-9H-purin-6-yl)benzamide (75 mg, 117.6 μmol, 14% yield) as a white solid, ESI LC-MS: m/z 638 [M+H]⁺.

Step B. ((1S,2S,3R)-3-(6-Amino-9H-purin-9-yl)-2-(((4-methoxyphenyl)diphenylmethoxy)methyl)-4-methylenecyclobutyl)methanol. A solution of N-(9-((1R,2S,3S)-3-(hydroxymethyl)-2-(((4-methoxyphenyl)diphenylmethoxy)methyl)-4-methylenecyclobutyl)-9H-purin-6-yl)benzamide (102 mg, 159.9 μmol) in CH₃NH₂/EtOH (3 mL) was stirred at r.t. for 15 min. Purification (Flash-Prep-HPLC with the following conditions: Column, C18 silica gel (4 g); mobile phase, CH₃CN/H₂O=0/1 increasing to CH₃CN/H₂O=1/0 within 15 min, the eluted product was collected at CH₃CN/H₂O=1/1; Detector, UV 254 nm) afforded ((1S,2S,3R)-3-(6-amino-9H-purin-9-yl)-2-(((4-methoxyphenyl)diphenylmethoxy)methyl)-4-methylenecyclobutyl)methanol (62 mg, 116.2 μmol, 73% yield) as a white solid. ¹H-NMR (400 MHz, DMSO-d₆): δ 8.29 (s, 1H), 8.17 (s, 1H), 7.22-7.29 (m, 12H), 7.13 (d, J=8.8 Hz, 2H), 6.83 (d, J=8.8 Hz, 2H), 5.44 (d, J=8.4 Hz, 1H), 5.09 (s, 1H), 4.79 (s, 1H), 4.72 (t, J=5.6 Hz, 1H), 3.73 (s, 3H), 3.62-3.71 (m, 2H), 3.19-3.21 (m, 2H), 2.96-3.04 (m, 1H), 2.84-2.86 (m, 1H). ESI-LCMS: m/z 534 [M+H]⁺.

### Example 1: 4-Amino-1-((1S,3R)-3-(hydroxymethyl)-2-methylenecyclobutyl)pyrimidin-2(1H)-one. #60107#

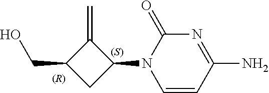

Step A. 3-Benzoyl-1-((1S,3R)-3-(((tert-butyldiphenylsilypoxy)methyl)-2-methylenecyclobutyl)pyrimidine-2,4(1H,3H)-dione. To a solution of (1R,3R)-3-(((tert-butyldiphenylsilyl)oxy)methyl)-2-methylenecyclobutanol (Intermediate 2, 387 mg, 1.1 mmol) in THF (8 mL) was added 3-benzoylpyrimidine-2,4(1H,3H)-dione (356 mg, 1.65 mmol), and PPh₃ (425 mg, 1.65 mmol). DIAD (445 mg, 2.2 mmol) was added dropwise under N₂ at r.t. The reaction mixture was stirred at r.t. under N₂ overnight. Water was added to the reaction mixture and the mixture was extracted with EA. The organic layer was washed by water, dried over Na₂SO₄, filtered, and concentrated under reduced pressure. Purification (FCC, SiO₂, PE:EA=1:1) afforded 3-benzoyl-1-((1S,3R)-3-(((tert-butyldiphenylsilyl)oxy)methyl)-2-methylenecyclobutyl)pyrimidine-2,4(1H,3H)-dione (650 mg). LC-MS: m/z=551.3 [M+H]⁺.

Step B. 1-((1S,3R)-3-(((tert-Butyldiphenylsilyl)oxy)methyl)-2-methylenecyclobutyl)pyrimidine-2,4(1H,3H)-dione. 3-Benzoyl-1-((1S,3R)-3-(((tert-butyldiphenylsilyl)oxy)methyl)-2-methylenecyclobutyl)pyrimidine-2,4(1H,3H)-dione (650 g, 1.1 mmol) was dissolved in 7M ammonia methanol solution (10 mL). The mixture was stirred at ambient temperature for 1.5 h. Solvent was removed under reduced pressure. Purification (FCC, SiO₂, DCM:MeOH=10:1) afforded 1-((1S,3R)-3-(((tert-butyldiphenylsilyl)oxy)methyl)-2-methylenecyclobutyl)pyrimidine-2,4(1H,3H)-dione (363 mg, 73.8% yield). LC-MS: m/z=447.2 [M+H]⁺.

Step C. 4-Amino-1-((1S,3R)-3-(((tert-butyldiphenylsilyl)oxy)methyl)-2-methylenecyclobutyl)pyrimidin-2(1H)-one. To a solution of 1-((1S,3R)-3-(((tert-butyldiphenylsilyl)oxy)methyl)-2-methylenecyclobutyl)pyrimidine-2,4(1H,3H)-dione (361 mg, 0.81 mmol) in THF (4.5 mL) was added TBDPSCl (491 mg, 1.62 mmol), DMAP (198 mg, 1.62 mmol) and triethylamine (TEA) (164 mg, 1.62 mmol). The reaction mixture was stirred at r.t. under N₂ for 3 h. 28% NH₃ aqueous solution (5 mL) was added. The reaction mixture was stirred at r.t. overnight. Solvent was removed by vacuo. The residue was extracted with EA and water. The organic layer was washed with water, dried over Na₂SO₄, filtered, and concentrated under reduced pressure. Purification (FCC, SiO₂, PE:EA=1:1) afforded 4-amino-1-((1S,3R)-3-(((tert-butyldiphenylsilyl)oxy)methyl)-2-methylenecyclobutyl)pyrimidin-2(1H)-one (290 mg, 80.5% yield). LC-MS: m/z=446.2 [M+H]⁺. ¹H NMR (400 MHz, DMSO-d₆) δ ppm 7.62-7.65 (m, 4H), 7.54 (d, J=7.6 Hz, 1H), 7.43-7.48 (m, 6H), 7.07 (d, J=16.0 Hz, 2H), 5.63 (d, J=7.2 Hz, 1H), 5.52 (m, 1H), 5.08 (t, J=4.4 Hz, 1H), 4.79 (t, J=4.4 Hz, 1H), 3.81 (d, J=5.6 Hz, 2H), 2.98-3.01 (m, 1H), 2.35-2.42 (m, 1H), 1.93-2.00 (m, 1H), 1.02 (s, 9H).

Step D. 4-Amino-1-((1S,3R)-3-(hydroxymethyl)-2-methylenecyclobutyl)pyrimidin-2(1H)-one. To a solution of 4-amino-1-((1S,3R)-3-(((tert-butyldiphenylsilyl)oxy)methyl)-2-methylenecyclobutyl)pyrimidin-2(1H)-one (290 mg, 0.65 mmol) in THF (4 mL) was added concentrated HCl solution (4 mL). The mixture was stirred at ambient temperature for 1.5 h. The water layer was washed by DCM several times and concentrated under reduced pressure. Purification (Flash-Prep-HPLC with the following conditions: Column, C18 silica gel (4 g); mobile phase, CH₃CN/H₂O (5 mM HCOOH)=0/1 increasing to CH₃CN/H₂O (5 mM HCOOH)=1/0 within 15 min, the eluted product was collected at CH₃CN/H₂O=23/77; Detector, UV 254 nm) afforded 4-amino-1-((1S,3R)-3-(hydroxymethyl)-2-methylenecyclobutyl)pyrimidin-2(1H)-one (80 mg, 59.2% yield). LCMS: m/z=208.1 [M+H]⁺. ¹H NMR (400 MHz, DMSO-d₆) δ ppm 7.59 (d, J=7.6 Hz, 1H), 7.04 (d, 2H), 5.71 (d, J=7.2 Hz, 1H), 5.50-5.54 (m, 1H), 5.04-5.05 (m, 1H), 4.72-4.73 (m, 1H), 4.66 (t, J=5.2 Hz, 1H), 2.83-2.87 (m, 1H), 2.33-2.41 (m, 1H), 1.87-1.94 (m, 1H); ¹H NMR (400 MHz, DMSO-d₆/D₂O) δ ppm 7.61 (d, J=7.6 Hz, 1H), 5.76 (d, J=7.2 Hz, 1H), 5.48 (t, J=2.4 Hz, 1H), 5.05 (t, 1H), 4.73 (m, 1H), 2.84-2.88 (m, 1H), 2.35-2.42 (m, 1H), 1.88-1.95 (m, 1H).

### Example 2: ((1R,3S)-3-(6-Amino-9H-purin-9-yl)-2-methylenecyclobutyl)methanol

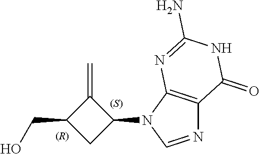

Step A. tert-Butyl (9-((1S,3R)-3-(((tert-butyldiphenylsilyl)oxy)methyl)-2-methylenecyclobutyl)-6-chloro-9H-purin-2-yl)carbamate. To a solution of (1R,3R)-3-(((tert-butyldiphenylsilyl)oxy)methyl)-2-methylenecyclobutanol (Intermediate 1, 380 mg, 858.45 μmol) in THF (5 mL) was added tert-butyl (6-chloro-9H-purin-2-yl) carbamate (347.27 mg, 1.29 mmol), and PPh₃ (337.74 mg, 1.29 mmol). The reaction mixture was stirred at 0° C. under N₂ atmosphere. Diethyl azodicarboxylate (DEAD) (299 mg, 1.72 mmol) was added slowly to the reaction mixture. The mixture was stirred at r.t. overnight. The reaction mixture was concentrated under reduced pressure. Purification (FCC, SiO₂, PE:EA=1:1) afforded tert-butyl (9-((1S,3R)-3-(((tert-butyldiphenylsilyl)oxy)methyl)-2-methylenecyclobutyl)-6-chloro-9H-purin-2-yl)carbamate (575 mg, 951.7 μmol, 110.9% yield) as a white solid. ¹H NMR (400 MHz, DMSO-d₆) δ 10.30 (s, 1H), 8.52 (s, 1H), 7.66 (m, 4H), 7.53-7.43 (m, 6H), 5.52 (s, 1H), 5.14 (d, J=2.8 Hz, 1H), 4.88 (d, J=2.7 Hz, 1H), 4.20 (d, J=7.1 Hz, 1H), 3.93 (m, 1H), 3.18 (s, 1H), 2.70-2.56 (m, 2H), 1.45 (s, 9H), 1.03 (s, 9H). ESI-LCMS: m/z 604.3 [M+H]⁺.

Step B. tert-Butyl (9-((1S,3R)-3-(((tert-butyldiphenylsilyl)oxy)methyl)-2-methylenecyclobutyl)-6-oxo-6,9-dihydro-1H-purin-2-yl)carbamate. To a solution of 3-hydroxypropionitrile (792.5 mg, 11.2 mmol) in THF (25 mL) was added NaH (374.64 mg, 15.61 mmol, 624.40 μL) at 0° C. under N₂ atmosphere. The mixture was stirred at 0° C. for 30 min, tert-butyl (9-((1S,3R)-3-(((tert-butyldiphenylsilyl)oxy)methyl)-2-methylenecyclobutyl)-6-chloro-9H-purin-2-yl)carbamate (1.35 g, 2.23 mmol) dissolved in THF (0.5 mL) was added dropwise at 0° C. The mixture was stirred at r.t. for 4 h. The reaction mixture was added H₂O (50 mL), extracted with EA (50 mL×3). The combined organic layers were washed with brine, dried with anhydrous sodium sulfate, filtered and concentrated under reduced pressure. Purification (FCC, SiO₂, DCM:MeOH=150:1) afforded tert-butyl (9-((1S,3R)-3-(((tert-butyldiphenylsilyl)oxy)methyl)-2-methylenecyclobutyl)-6-oxo-6,9-dihydro-1H-purin-2-yl)carbamate (850.0 mg, 1.45 mmol, 65.1% yield) as a white solid. ¹H NMR (400 MHz, DMSO-d₆) δ 11.41 (s, 1H), 11.12 (s, 1H), 8.02 (s, 1H), 7.66 (m, 4H), 7.54-7.42 (m, 6H), 5.33 (s, 1H), 5.12 (d, J=2.8 Hz, 1H), 4.85 (d, J=2.8 Hz, 1H), 3.95 (m 1H), 3.88 (m, 1H), 3.17 (s, 1H), 2.65-2.55 (m, 1H), 2.37 (d, J=11.0 Hz, 1H), 1.51 (s, 9H), 1.03 (s, 9H). ESI-LCMS: m/z 586.3 [M+H]⁺.

Step C. tert-Butyl (9-((1S,3R)-3-(hydroxymethyl)-2-methylenecyclobutyl)-6-oxo-6,9-dihydro-1H-purin-2-yl)carbamate. To a solution of tert-butyl (9-((1S,3R)-3-(((tert-butyldiphenylsilyl)oxy)methyl)-2-methylenecyclobutyl)-6-oxo-6,9-dihydro-1H-purin-2-yl)carbamate (325 mg, 554.83 μmol) in THF (5 mL) was added tetra-n-butylammonium fluoride (TBAF) (1 M, 5.55 mL). The reaction mixture was stirred at r.t. for 2 h. The reaction mixture was concentrated under reduced pressure. Purification (FCC, SiO₂, DCM:MeOH=40:1) afforded tert-butyl (9-((1S,3R)-3-(hydroxymethyl)-2-methylenecyclobutyl)-6-oxo-6,9-dihydro-1H-purin-2-yl)carbamate (150 mg, 388.6 μmol, 70.1% yield, 90% purity) as a white solid. ESI-LCMS: m/z 348.6 [M+H]⁺.

Step D. 2-Amino-9-((1S,3R)-3-(hydroxymethyl)-2-methylenecyclobutyl)-1H-purin-6(9H)-one. To a solution of tert-butyl (9-((1S,3R)-3-(hydroxymethyl)-2-methylenecyclobutyl)-6-oxo-6,9-dihydro-1H-purin-2-yl)carbamate (70 mg, 201.51 μmol) in THF (400 μL) was added HCl (6 M, 349.96 μL). The reaction mixture was stirred at r.t. for 2 h. Purification (Flash-Prep-HPLC with the following conditions: Column, C18 silica gel (4 g); mobile phase, CH₃CN/H₂O (5 mM HCOOH)=0/1 increasing to CH₃CN/H₂O (5 mM HCOOH)=1/0 within 25 min, the eluted product was collected at CH₃CN/H₂O (5 mM HCOOH)=21/79; Detector, UV 254 nm.) afforded 2-amino-9-((1S,3R)-3-(hydroxymethyl)-2-methylenecyclobutyl)-1H-purin-6(9H)-one (40 mg, 161.8 μmol, 80.3% yield) as a white powder. ¹H NMR (400 MHz, DMSO-d₆) δ 10.71 (s, 1H), 7.92 (s, 1H), 6.50 (d, J=18.1 Hz, 2H), 5.25 (dd, J=10.0, 7.5 Hz, 1H), 5.07 (t, J=2.6 Hz, 1H), 4.84-4.76 (m, 1H), 3.69-3.60 (m, 2H), 3.04-2.94 (m, 1H), 2.57 (m, 1H), 2.27 (m, 1H). ESI-LCMS: m/z 248.1 [M+H]⁺.

### Example 3: ((1R,3S)-3-(6-Amino-9H-purin-9-yl)-2-methylenecyclobutyl)methanol

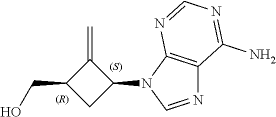

Step A. ((1R,3S)-3-(6-(N,N-DiBoc)amino-9H-purin-9-yl)-2-methylenecyclobutyl)methanol. To a solution of (1R,3R)-3-(((tert-butyldiphenylsilyl)oxy)methyl)-2-methylenecyclobutanol (Intermediate 2, 750 mg, 2.13 mmol) in THF (8 mL) was added N,N-diboc-9H-purin-6-amine (714.32 mg, 3.20 mmol), followed by PPh₃ (1.12 g, 6.39 mmol). The reaction mixture was stirred at 0° C. under N₂ atmosphere. DEAD (741.88 mg, 4.26 mmol) was added slowly to the reaction mixture via syringe. The mixture was stirred at 55° C. overnight. The reaction mixture was concentrated under reduced pressure. Purification (FCC, SiO₂, PE:EA=1:1) afforded ((1R,3S)-3-(6-(N,N-DiBoc)amino-9H-purin-9-yl)-2-methylenecyclobutyl)methanol (1.30 g, 1.94 mmol, 90.8% yield) as a yellow solid. ESI-LCMS: m/z 670.4 [M+H]⁺.

Step B. ((1R,3S)-3-(6-Amino-9H-purin-9-yl)-2-methylenecyclobutyl)methanol. To a solution of ((1R,3S)-3-(6-(N,N-DiBoc)amino-9H-purin-9-yl)-2-methylenecyclobutyl)methanol (1.30 g, 1.94 mmol) in THF (5 mL) was added HCl (12 M, 5 mL). The reaction mixture was stirred at r.t. for 2 h. Purification (Flash-Prep-HPLC with the following conditions: Column, C18 silica gel (4 g); mobile phase, CH₃CN/H₂O (5 mM HCOOH)=0/1 increasing to CH₃CN/H₂O (5 mM HCOOH)=1/0 within 25 min, the eluted product was collected at CH₃CN/H₂O (5 mM HCOOH)=23/77; Detector, UV 254 nm.) afforded ((1R,3S)-3-(6-amino-9H-purin-9-yl)-2-methylenecyclobutyl)methanol (400 mg, 1.73 mmol, 89.2% yield). 41 NMR (400 MHz, CD₃OD) δ 8.30 (s, 1H), 8.22 (s, 1H), 5.66-5.58 (m, 1H), 5.21 (td, J=2.6, 1.2 Hz, 1H), 4.93 (m, 1H), 3.92-3.78 (m, 2H), 3.24-3.13 (m, 1H), 2.78 (m, 1H), 2.49 (m, 1H). ESI-LCMS: m/z 232.1 [M+H]⁺.

### Example 4: 4-Amino-1-((1S,2R,3R)-2,3-bis(hydroxymethyl)-4-methylenecyclobutyl)pyrimidin-2(1H)-one

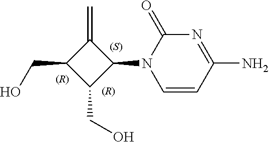

Step A. 3-Benzoyl-1-((1S,2R,3R)-2,3-bis((benzyloxy)methyl)-4-methylenecyclobutyl)pyrimidine-2,4(1H,3H)-dione. To a solution of (1R,2S,3R)-2,3-bis((benzyloxy)methyl)-4-methylenecyclobutanol (1.7 g, 5.24 mmol), 3-benzoylpyrimidine-2,4(1H,3H)-dione (1.70 g, 7.86 mmol) and triphenylphosphine (2.06 g, 7.86 mmol) in tetrahydrofuran (26 mL) was added diisopropyl azodicarboxylate (1.59 g, 7.86 mmol, 1.54 mL) dropwise at 0° C. The reaction mixture was stirred at 50° C. for 3 h. The reaction mixture was concentrated under reduced pressure. Purification (Flash-Prep-HPLC with the following conditions: Column, C18 silica gel (20 g); mobile phase, CH₃CN/H₂O=0/1 increasing to CH₃CN/H₂O=1/0 within 25 min, the eluted product was collected at CH₃CN/H₂O=35/65; Detector, UV 254 nm.) afforded 3-benzoyl-1-((1S,2R,3R)-2,3-bis((benzyloxy)methyl)-4-methylenecyclobutyl)pyrimidine-2,4(1H,3H)-dione (2.5 g, 4.31 mmol, 82.2% yield, 90% purity) as a white solid. ESI-LCMS m/z=523.4 [M+H]⁺.

Step B. 1-((1S,2R,3R)-2,3-Bis((benzyloxy)methyl)-4-methylenecyclobutyl)pyrimidine-2,4(1H,3H)-dione. A solution of 3-benzoyl-1-((1S,2R,3R)-2,3-bis((benzyloxy)methyl)-4-methylenecyclobutyl)pyrimidine-2,4(1H,3H)-dione (2.5 g, 4.78 mmol) in methylamine/ethanol (5 mL) was stirred at r.t. for 30 min. The reaction mixture was concentrated under reduced pressure. Purification (MPLC, C18 Flash Column, Agela Technologies, 20 g, 20 mL/min, ACN:H₂O=30:70) afforded 1-((1S,2R,3R)-2,3-bis((benzyloxy)methyl)-4-methylenecyclobutyl)pyrimidine-2,4(1H,3H)-dione (1.8 g, 4.09 mmol, 85.4% yield, 95% purity) as a white solid. ESI LC-MS m/z=419.2 [M+H]⁺. ¹H-NMR (400 MHz, DMSO-d₆): δ 11.32 (s, 1H), 7.62 (d, J=8.0 Hz, 1H), 7.36-7.26 (m, 10H), 5.52 (d, J=8.0 Hz, 1H), 5.38 (d, J=7.9 Hz, 1H), 5.10 (s, 1H), 4.90 (s, 1H), 4.49 (d, J=10.2 Hz, 4H), 3.67 (d, J=5.7 Hz, 2H), 3.59-3.57 (m, 2H), 2.89-2.87 (m, 1H), 2.72-2.65 (m, 1H).

Step C. 4-Amino-1-((1S,2R,3R)-2,3-bis((benzyloxy)methyl)-4-methylenecyclobutyl)pyrimidin-2(1H)-one. To a solution of 1-((1S,2R,3R)-2,3-bis((benzyloxy)methyl)-4-methylenecyclobutyl)pyrimidine-2,4(1H,3H)-dione (1.7 g, 4.06 mmol), DMAP (1.09 g, 8.94 mmol), TEA (1.03 g, 10.16 mmol, 1.42 mL) in acetonitrile (20 mL) was added 2,4,6-triisopropylbenzenesulfonyl chloride (2.71 g, 8.94 mmol) at 0° C. The reaction mixture was stirred at r.t. for 3 h. Ammonium hydroxide (7 mL) was added to the mixture at r.t., and the mixture was stirred for 16 h. The reaction mixture was poured into water and extracted with EA. The organic layer was washed with brine, and concentrated under reduced pressure. Purification (MPLC, C18 Flash Column, Agela Technologies, 20 g, 20 mL/min, ACN:H₂O=30:70) afforded 4-amino-1-((1S,2R,3R)-2,3-bis((benzyloxy)methyl)-4-methylenecyclobutyl)pyrimidin-2(1H)-one (1.6 g, 3.83 mmol, 94.3% yield) as a white solid. ESI-LCMS m/z=418.2 [M+H]⁺.

Step D. N-(1-((1S,2R,3R)-2,3-Bis((benzyloxy)methyl)-4-methylenecyclobutyl)-2-oxo-1,2-dihydropyrimidin-4-yl)benzamide. To a solution of 4-amino-1-((1S,2R,3R)-2,3-bis((benzyloxy)methyl)-4-methylenecyclobutyl)pyrimidin-2(1H)-one (1.5 g, 3.59 mmol) in pyridine (20 mL) was added benzoyl chloride (757.55 mg, 5.39 mmol, 626.07 μL) dropwise at 0° C. The mixture was stirred at r.t. for 6 h. The reaction mixture was quenched by NH₄OH and stirred for 20 min. The reaction mixture was concentrated under reduced pressure. Purification (MPLC, C18 Flash Column, Agela Technologies, 20 g, 20 mL/min, ACN:H₂O=40:60) afforded N-(1-((1S,2R,3R)-2,3-bis((benzyloxy)methyl)-4-methylenecyclobutyl)-2-oxo-1,2-dihydropyrimidin-4-yl)benzamide (1.8 g, 3.38 mmol, 94.1% yield, 98% purity) as a white solid. ESI-LCMS m/z=522.2 [M+H]⁺.

Step E. N-(1-((1S,2R,3R)-2,3-Bis(hydroxymethyl)-4-methylenecyclobutyl)-2-oxo-1,2-dihydropyrimidin-4-yl)benzamide. To a solution of N-(1-((1S,2R,3R)-2,3-bis((benzyloxy)methyl)-4-methylenecyclobutyl)-2-oxo-1,2-dihydropyrimidin-4-yl)benzamide (1.8 g, 3.45 mmol) in dichloromethane (20 mL) was added boron trichloride (1 M, 34.51 mL) dropwise at −78° C. The mixture was stirred at −78° C. for 1 h. The reaction mixture was quenched with methanol at −78° C. TEA was added to adjust the pH to pH=6. The solvent was removed under reduced pressure. Purification (MPLC, C18 Flash Column, Agela Technologies, 20 g, 20 mL/min, ACN:H₂O=25:75) afforded N-(1-((1S,2R,3R)-2,3-bis(hydroxymethyl)-4-methylenecyclobutyl)-2-oxo-1,2-dihydropyrimidin-4-yl)benzamide (1.0 g, 2.90 mmol, 84.0% yield, 99% purity) as a white solid. ESI-LCMS m/z=342.1 [M+H]⁺. ¹H-NMR (400 MHz, DMSO-d₆): δ 11.25 (s, 1H), 8.19 (d, J=7.4 Hz, 1H), 8.02-8.00 (m, 2H), 7.65-7.61 (m, 1H), 7.54-7.50 (m, 2H), 7.37 (d, J=7.4 Hz, 1H), 5.41-5.39 (m, 1H), 5.14 (t, J=2.4 Hz, 1H), 4.87 (t, J=2.4 Hz, 1H), 4.75 (s, 2H), 3.65 (t, J=5.2 Hz, 2H), 3.57 (t, J=4.8 Hz, 2H), 3.11-3.04 (m, 1H), 2.77-2.72 (m, 1H).

Step F. 4-Amino-1-((1S,2R,3R)-2,3-bis(hydroxymethyl)-4-methylenecyclobutyl)pyrimidin-2(1H)-one. A solution of N-(1-((1S,2R,3R)-2,3-bis(hydroxymethyl)-4-methylenecyclobutyl)-2-oxo-1,2-dihydropyrimidin-4-yl)benzamide (50 mg, 146.47 μmol) in methylamine/ethanol (1.5 mL) was stirred at r.t. for 30 min. The reaction mixture was concentrated under reduced pressure. Purification (MPLC, C18 Flash Column, Agela Technologies, 4 g, 4 mL/min, ACN:H₂O=10:90) afforded 4-amino-1-((1S,2R,3R)-2,3-bis(hydroxymethyl)-4-methylenecyclobutyl)pyrimidin-2(1H)-one (10 mg, 41.31 μmol, 28.2% yield, 98% purity) as a white solid. ESI-LCMS m/z=238.1 [M+H]⁺. NMR (400 MHz, DMSO-d₆): δ 7.61 (d, J=7.4 Hz, 1H), 7.12 (d, J=21.8 Hz, 2H), 5.73 (d, J=7.4 Hz, 1H), 5.28-5.25 (m, 1H), 5.08 (t, J=2.5 Hz, 1H), 4.77 (t, J=2.5 Hz, 1H), 4.70-4.67 (m, 2H), 3.59 (t, J=5.5 Hz, 2H), 2.65-2.61 (m, 1H), 2.37-2.33 (m, 1H).

### Example 5: 1-((1S,2R,3R)-2,3-Bis(hydroxymethyl)-4-methylenecyclobutyl)-5-methylpyrimidine-2,4(1H,3H)-dione

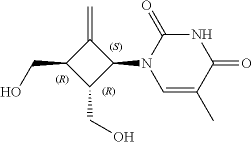

Step A. 3-Benzoyl-1-((1S,2R,3R)-2,3-bis((benzyloxy)methyl)-4-methylenecyclobutyl)-5-methylpyrimidine-2,4(1H,3H)-dione. A solution of (1R,2S,3R)-2,3-bis((benzyloxy)methyl)-4-methylenecyclobutanol (Intermediate 5, 350 mg, 1.08 mmol) and 3-benzoyl-5-methylpyrimidine-2,4(1H,3H)-dione (496 mg, 2.16 mmol) in dry THF (5 mL) was added PPh₃ (566 mg, 2.16 mmol) at r.t. then diisopropyl azodicarboxylate (DIAD) (436 mg, 2.16 mmol) was added dropwise at 0° C. under N₂. The resulted suspension was stirred at 55° C. for 2 h. The reaction mixture was concentrated under reduced pressure. Purification (MPLC, C18 Flash Column, Agela Technologies, 12 g, 12 mL/min, ACN:H₂O=40:60) and further purification (FCC, SiO₂, PE:EA=3:1) afforded 3-benzoyl-1-((1S,2R,3R)-2,3-bis((benzyloxy)methyl)-4-methylenecyclobutyl)-5-methylpyrimidine-2,4(1H,3H)-dione (240 mg, 447 μmol) as a white solid. ¹H-NMR (400 MHz, CDCl₃): δ ppm 7.98-7.92 (m, 2H), 7.68-7.62 (m, 1H), 7.52-7.42 (m, 3H), 7.42-7.24 (m, 10H), 5.55-5.50 (m, 1H), 5.27-5.22 (m, 1H), 5.11-5.07 (m, 1H), 4.63-4.53 (m, 4H), 3.83-3.78 (m, 1H), 3.75-3.69 (m, 1H), 3.68-3.64 (m, 2H), 2.99-2.85 (m, 2H), 1.71 (s, 3H). ESI-LCMS: m/z 537 [M+H]⁺.

Step B. 1-((1S,2R,3R)-2,3-Bis(hydroxymethyl)-4-methylenecyclobutyl)-5-methylpyrimidine-2,4(1H,3H)-dione. To a solution of 3-benzoyl-1-((1S,2R,3R)-2,3-bis((benzyloxy)methyl)-4-methylenecyclobutyl)-5-methylpyrimidine-2,4(1H,3H)-dione (400 mg, 745 μmol) in dry DCM (10 mL) was added dropwise BCl₃ (7.5 mmol, 7.5 mL) at −78° C. under N₂. The mixture was stirred at −78° C. for 1 h. The mixture was quenched by adding ice water at −78° C. The mixture was extracted with DCM and washed with water. The organic layer was concentrated in vacuo. Purification (MPLC, C18 Flash Column, Agela Technologies, 4 g, 4 mL/min, ACN:H₂O=10:90) afforded 1-((1S,2R,3R)-2,3-bis(hydroxymethyl)-4-methylenecyclobutyl)-5-methylpyrimidine-2,4(1H,3H)-dione (100 mg, 390 μmol) as a white solid. ¹H-NMR (400 MHz, DMSO-d₆): δ ppm 7.41 (s, 1H), 5.25-5.19 (m, 1H), 5.09-5.05 (m, 1H), 4.82-4.80 (m, 1H), 3.62 (d, J=5.8, 2H), 3.50 (d, J=5.8, 2H), 2.66-2.60 (m, 1H), 2.48-2.41 (m, 1H), 1.75 (s, 3H). ESI-LCMS: m/z 253 [M+H]⁺.

### Example 6: ((1R,2R,3S)-3-(4-Amino-7H-pyrrolo[2,3-d]pyrimidin-7-yl)-4-methylenecyclobutane-1,2-diyl)dimethanol

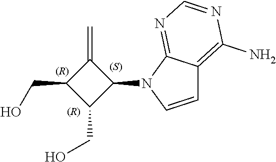

Step A. 7-((1S,2R,3R)-2,3-Bis((benzyloxy)methyl)-4-methylenecyclobutyl)-4-chloro-7H-pyrrolo[2,3-d]pyrimidine. To a solution of (1R,2S,3R)-2,3-bis((benzyloxy)methyl)-4-methylenecyclobutanol (Intermediate 5, 1.0 g, 3.1 mmol) in THF (20 mL) at r.t. was added 4-chloro-7H-pyrrolo[2,3-d]pyrimidine (1.0 g, 6.2 mmol) and PPh₃ (1.6 g, 6.2 mmol), then at 0° C. was added DIAD (1.3 g, 6.2 mmol) under N₂. The reaction mixture was warmed to 55° C. and stirred for 2 h. The mixture was concentrated under reduced pressure. Purification (FCC, SiO₂, PE:EA=4:1) afforded 7-((1S,2R,3R)-2,3-bis((benzyloxy)methyl)-4-methylenecyclobutyl)-4-chloro-7H-pyrrolo[2,3-d]pyrimidine (1.3 g, 2.8 mmol, 91.7% yield) as a white solid. ESI LC-MS m/z=460.2 [M+H]⁺. ¹H-NMR (400 MHz, DMSO-d₆): δ8.66 (s, 1H), 7.82 (d, J=3.7 Hz, 1H), 7.41-7.12 (m, 9H), 6.70 (d, J=3.7 Hz, 1H), 5.76-5.71 (m, 1H), 5.10 (d, J=2.8 Hz, 1H), 4.73 (d, J=2.7 Hz, 1H), 4.55 (s, 2H), 4.43 (s, 2H), 3.83-3.72 (m, 2H), 3.64 (d, J=5.4 Hz, 2H), 3.11-3.02 (m, 1H), 3.01-2.91 (m, 1H), 1.42 (d, J=6.2 Hz, 1H).

Step B. ((1R,2R,3S)-3-(4-Chloro-7H-pyrrolo[2,3-d]pyrimidin-7-yl)-4-methylenecyclobutane-1,2-diyl)dimethanol. To a solution of 7-((1S,2R,3R)-2,3-bis((benzyloxy)methyl)-4-methylenecyclobutyl)-4-chloro-7H-pyrrolo[2,3-d]pyrimidine (1.3 g, 2.8 mmol) in DCM (20 mL) at −70° C. was added 1N BCl₃ (16.8 mL, 16.8 mmol) under N₂. The mixture was stirred at −70° C. for 1 h and then was quenched with MeOH. The mixture was concentrated under reduced pressure. Purification (MPLC, C18 Flash Column, Agela Technologies, 12 g, 12 mL/min, ACN:H₂O=20:80) afforded ((1R,2R,3S)-3-(4-chloro-7H-pyrrolo[2,3-d]pyrimidin-7-yl)-4-methylenecyclobutane-1,2-diyl)dimethanol (500 mg, 1.8 mmol, 63.3% yield) as a white solid. ESI LC-MS m/z=280.0 [M+H]⁺. ¹H-NMR (400 MHz, DMSO-d₆): δ 8.64 (s, 1H), 7.82 (d, J=3.8 Hz, 1H), 6.72 (d, J=3.7 Hz, 1H), 5.63 (dt, J=7.8, 2.6 Hz, 1H), 5.07 (t, J=2.6 Hz, 1H), 4.82-4.73 (m, 2H), 4.66 (t, J=2.6 Hz, 1H), 3.70 (td, J=5.5, 1.5 Hz, 2H), 3.57 (t, J=4.9 Hz, 2H), 2.94-2.71 (m, 2H).

Step C. ((1R,2R,3S)-3-(4-Chloro-7H-pyrrolo[2,3-d]pyrimidin-7-yl)-2-(((4-methoxyphenyl)diphenylmethoxy)methyl)-4-methylenecyclobutyl)methanol. To a solution of ((1R,2R,3S)-3-(4-chloro-7H-pyrrolo[2,3-d]pyrimidin-7-yl)-4-methylenecyclobutane-1,2-diyl)dimethanol (500 mg, 1.8 mmol) in DCM (20 mL) at r.t. was added pyridine (0.3 mL) and MMTrCl (0.6 g, 2.0 mmol) under N₂. Then the reaction mixture was stirred at r.t. for 2 h. The mixture was quenched with MeOH and then concentrated under reduced pressure. Purification (FCC, SiO₂, DCM:MeOH=10:1) afforded ((1R,2R,3S)-3-(4-chloro-7H-pyrrolo[2,3-d]pyrimidin-7-yl)-2-(((4-methoxyphenyl)diphenylmethoxy)methyl)-4-methylenecyclobutyl)methanol (240 mg, 0.4 mmol, 24.3% yield) as a white solid. ESI LC-MS m/z=552.1 [M+H]⁺. ¹H-NMR (400 MHz, DMSO-d₆): δ 8.64 (s, 1H), 7.90 (d, J=3.7 Hz, 1H), 7.25-7.12 (m, 10H), 7.10-7.01 (m, 2H), 6.85-6.74 (m, 3H), 5.79-5.72 (m, 1H), 5.09 (d, J=2.4 Hz, 1H), 4.78-4.69 (m, 2H), 3.72 (s, 3H), 3.70-3.58 (m, 2H), 3.25-3.13 (m, 2H), 2.96-2.77 (m, 2H).

Step D. ((1R,2R,3S)-3-(4-Amino-7H-pyrrolo[2,3-d]pyrimidin-7-yl)-2-(((4-methoxyphenyl)diphenylmethoxy)methyl)-4-methylenecyclobutyl)methanol. To a solution of ((1R,2R,3S)-3-(4-chloro-7H-pyrrolo[2,3-d]pyrimidin-7-yl)-2-(((4-methoxyphenyl)diphenylmethoxy)methyl)-4-methylenecyclobutyl)methanol (240 mg, 0.4 mmol) in dioxane (10 mL) was added ammonia aq. solution (30 mL). Then the mixture was warmed to 100° C. and stirred for 24 h. The mixture was cooled, and extracted with EtOAc. The organic layer was washed with H₂O and brine, dried over Na₂SO₄, filtered, and concentrated under reduced pressure. Purification (MPLC, C18 Flash Column, Agela Technologies, 4 g, 4 mL/min, ACN:H₂O=45:55) afforded ((1R,2R,3S)-3-(4-amino-7H-pyrrolo[2,3-d]pyrimidin-7-yl)-2-(((4-methoxyphenyl)diphenylmethoxy)methyl)-4-methylenecyclobutyl)methanol (140 mg, 0.3 mmol, 60.5% yield) as a white solid. ESI LC-MS m/z=533.2 [M+H]⁺. ¹H-NMR (400 MHz, DMSO-d₆): δ 8.07 (s, 1H), 7.30-7.15 (m, 11H), 7.15-7.08 (m, 2H), 7.00 (s, 2H), 6.84-6.78 (m, 2H), 6.63 (d, J=3.6 Hz, 1H), 5.67 (dt, J=8.0, 2.7 Hz, 1H), 5.05 (t, J=2.5 Hz, 1H), 4.74-4.62 (m, 2H), 3.72 (s, 3H), 3.69-3.56 (m, 2H), 3.14 (d, J=5.5 Hz, 2H), 2.86-2.65 (m, 2H).

Step E. ((1R,2R,3S)-3-(4-Amino-7H-pyrrolo[2,3-d]pyrimidin-7-yl)-4-methylenecyclobutane-1,2-diyl)dimethanol. To a solution of ((1R,2R,3S)-3-(4-amino-7H-pyrrolo[2,3-d]pyrimidin-7-yl)-2-(((4-methoxyphenyl)diphenylmethoxy)methyl)-4-methylenecyclobutyl)methanol (40 mg, 0.08 mmol) in DCM (1 mL) was added trichloroacetic acid (30 mg). The reaction mixture was stirred at r.t. for 1 h. The reaction mixture was quenched with NaHCO₃ aqueous solution. Purification (MPLC, C18 Flash Column, Agela Technologies, 4 g, 4 mL/min, ACN:H₂O=40:60) afforded ((1R,2R,3S)-3-(4-amino-7H-pyrrolo[2,3-d]pyrimidin-7-yl)-4-methylenecyclobutane-1,2-diyl)dimethanol (15 mg, 0.06 mmol, 76.7% yield) as a white solid. ESI LC-MS m/z=261.1 [M+H]⁺. ¹H-NMR (400 MHz, DMSO-d₆): δ 8.05 (d, J=1.8 Hz, 1H), 7.19 (dd, J=3.6, 1.1 Hz, 1H), 6.99 (s, 2H), 6.58 (dd, J=3.6, 1.4 Hz, 1H), 5.47 (d, J=7.8 Hz, 1H), 5.05 (t, J=2.6 Hz, 1H), 4.77 (s, 2H), 4.67-4.63 (m, 1H), 3.75-3.62 (m, 2H), 3.61-3.49 (m, 2H), 2.81 (d, J=7.2 Hz, 1H), 2.67-2.57 (m, 1H).

### Example 7: ((1R,2R,3S)-3-(4-Amino-5-fluoro-7H-pyrrolo[2,3-d]pyrimidin-7-yl)-4-methylenecyclobutane-1,2-diyl)dimethanol. #60480#

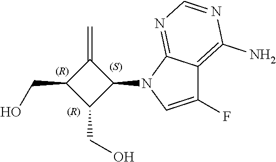

Step A. 7-((1S,2R,3R)-2,3-Bis((benzyloxy)methyl)-4-methylenecyclobutyl)-4-chloro-5-fluoro-7H-pyrrolo[2,3-d]pyrimidine. To a solution of (1R,2S,3R)-2,3-bis((benzyloxy)methyl)-4-methylenecyclobutanol (Intermediate 5, 1.0 g, 3.1 mmol) in THF (20 mL) at r.t. was added 4-chloro-5-fluoro-7H-pyrrolo[2,3-d]pyrimidine (1.1 g, 6.2 mmol) and PPh₃ (1.6 g, 6.2 mmol). At 0° C. DIAD (1.3 g, 6.2 mmol) was added under N₂. The reaction mixture was stirred at r.t. for 2 h. The mixture was concentrated under reduced pressure. Purification (FCC, SiO₂, PE:EA=4:1) afforded 7-((1S,2R,3R)-2,3-bis((benzyloxy)methyl)-4-methylenecyclobutyl)-4-chloro-5-fluoro-7H-pyrrolo[2,3-d]pyrimidine (1.3 g, 2.7 mmol, 88.2% yield) as a white solid. ESI LC-MS m/z=478.1 [M+H]⁺.

Step B. ((1R,2R,3S)-3-(4-Chloro-5-fluoro-7H-pyrrolo[2,3-d]pyrimidin-7-yl)-4-methylenecyclobutane-1,2-diyl)dimethanol. To a solution of 7-((1S,2R,3R)-2,3-bis((benzyloxy)methyl)-4-methylenecyclobutyl)-4-chloro-5-fluoro-7H-pyrrolo[2,3-d]pyrimidine (1.3 g, 2.7 mmol) in DCM (20 mL) at −70° C. was added 1N BCl₃ (16.2 mL, 16.2 mmol) under N₂. The mixture was stirred at −70° C. for 1 h. The reaction mixture was quenched with MeOH. The reaction mixture was concentrated under reduced pressure. Purification (MPLC, C18 Flash Column, Agela Technologies, 12 g, 12 mL/min, H₂O:ACN=5:1) afforded ((1R,2R,3S)-3-(4-chloro-5-fluoro-7H-pyrrolo[2,3-d]pyrimidin-7-yl)-4-methylenecyclobutane-1,2-diyl)dimethanol (400 mg, 1.3 mmol, 64.2% yield) as a white solid. ESI LC-MS m/z=298.0 [M+H]⁺.

Step C. ((1R,2R,3S)-3-(4-Chloro-5-fluoro-7H-pyrrolo[2,3-d]pyrimidin-7-yl)-2-(((4-methoxyphenyl)diphenylmethoxy)methyl)-4-methylenecyclobutyl)methanol. To a solution of ((1R,2R,3S)-3-(4-chloro-5-fluoro-7H-pyrrolo[2,3-d]pyrimidin-7-yl)-4-methylenecyclobutane-1,2-diyl)dimethanol (400 mg, 1.3 mmol) in DCM (20 mL) at r.t. was added pyridine (0.3 mL) and MMTrCl (440 mg, 1.4 mmol) under N₂. The reaction mixture was stirred at r.t. for 2 h. The mixture was quenched with MeOH and concentrated under reduced pressure. Purification (FCC, SiO₂, PE:EA=3:1) afforded ((1R,2R,3S)-3-(4-chloro-5-fluoro-7H-pyrrolo[2,3-d]pyrimidin-7-yl)-2-(((4-methoxyphenyl)diphenylmethoxy)methyl)-4-methylenecyclobutyl)methanol (200 mg, 0.4 mmol, 26.1% yield) as a white solid. ESI LC-MS m/z=570.1 [M+H]⁺. ¹H-NMR (400 MHz, DMSO-d₆): δ 8.67 (s, 1H), 7.95 (d, J=1.9 Hz, 1H), 7.29-7.12 (m, 10H), 7.10-7.02 (m, 2H), 6.87-6.76 (m, 2H), 5.78 (s, 1H), 5.09 (s, 1H), 4.81 (s, 1H), 4.70 (t, J=5.3 Hz, 1H), 4.11-3.95 (m, 1H), 3.72 (s, 3H), 3.70-3.56 (m, 1H), 3.24-3.11 (m, 2H), 2.81 (s, 2H).

Step D. ((1R,2R,3S)-3-(4-Amino-5-fluoro-7H-pyrrolo[2,3-d]pyrimidin-7-yl)-2-(((4-methoxyphenyl)diphenylmethoxy)methyl)-4-methylenecyclobutyl)methanol. To a solution of ((1R,2R,3S)-3-(4-chloro-5-fluoro-7H-pyrrolo[2,3-d]pyrimidin-7-yl)-2-(((4-methoxyphenyl)diphenylmethoxy)methyl)-4-methylenecyclobutyl)methanol (200 mg, 0.4 mmol) in dioxane (10 mL) was added ammonia aq. solution (30 mL). The reaction mixture was warmed to 100° C. and stirred for 24 h. The resulting mixture was extracted with EtOAc and the organic layer was washed with H₂O and brine, dried over Na₂SO₄, filtered and concentrated under reduced pressure. Purification (MPLC, C18 Flash Column, Agela Technologies, 4 g, 4 mL/min, H₂O:ACN=2:1) afforded ((1R,2R,3S)-3-(4-amino-5-fluoro-7H-pyrrolo[2,3-d]pyrimidin-7-yl)-2-(((4-methoxyphenyl)diphenylmethoxy)methyl)-4-methylenecyclobutyl)methanol (120 mg, 0.2 mmol, 62.2% yield) as a white solid. ESI LC-MS m/z=551.2 [M+H]⁺. ¹H-NMR (400 MHz, DMSO-d₆): δ 8.09 (s, 1H), 7.30-7.16 (m, 11H), 7.16-7.09 (m, 2H), 7.01 (s, 2H), 6.89-6.76 (m, 2H), 5.71 (dd, J=7.9, 2.3 Hz, 1H), 5.06 (t, J=2.7 Hz, 1H), 4.74 (t, J=2.6 Hz, 1H), 4.67 (t, J=5.3 Hz, 1H), 3.73 (s, 3H), 3.69-3.53 (m, 2H), 3.14 (d, J=5.6 Hz, 2H), 2.83-2.66 (m, 2H). ¹⁹FNMR (400 MHz, DMSO-d₆): δ −167.525 (s).

Step E. ((1R,2R,3S)-3-(4-Amino-5-fluoro-7H-pyrrolo[2,3-d]pyrimidin-7-yl)-4-methylenecyclobutane-1,2-diyl)dimethanol. To a solution of ((1R,2R,3S)-3-(4-amino-5-fluoro-7H-pyrrolo[2,3-d]pyrimidin-7-yl)-2-(((4-methoxyphenyl)diphenylmethoxy)methyl)-4-methylenecyclobutyl)methanol (40 mg, 0.07 mmol) in DCM (1 mL) was added trichloroacetic acid (30 mg). The reaction mixture was stirred at r.t. for 1 h. The reaction mixture was quenched with NaHCO₃ aqueous solution. Purification (MPLC, C18 Flash Column, Agela Technologies, 4 g, 4 mL/min, H₂O:ACN=5:1) afforded ((1R,2R,3S)-3-(4-amino-5-fluoro-7H-pyrrolo[2,3-d]pyrimidin-7-yl)-4-methylenecyclobutane-1,2-diyl)dimethanol (16 mg, 0.06 mmol, 79.2% yield) as a white solid. ESI LC-MS m/z=279.1 [M+H]⁺. ¹H-NMR (400 MHz, DMSO-d₆): δ 8.06 (d, J=1.3 Hz, 1H), 7.16 (d, J=2.0 Hz, 1H), 6.97 (s, 2H), 5.52 (d, J=7.9 Hz, 1H), 5.04 (t, J=2.6 Hz, 1H), 4.75 (s, 2H), 4.68 (s, 1H), 3.76-3.61 (m, 2H), 3.55 (d, J=5.0 Hz, 2H), 2.85-2.73 (m, 1H), 2.65-2.55 (m, 1H). ¹⁹FNMR (400 MHz, DMSO-d₆): δ −167.764 (s).

### Example 8: ((1R,2R,3S)-3-(6-Amino-9H-purin-9-yl)-4-methylenecyclobutane-1,2-diyl)dimethanol

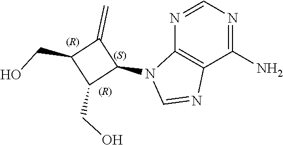

Step A. (1R,2R,3S,4R)-2-(6-Amino-9H-purin-9-yl)-3,4-bis((benzyloxy)methyl)cyclobutanol. To a suspension of adenine (36.1 g, 266.8 mmol) in DMF (1.0 L) was added NaH (10.7 g, 266.8 mmol, 60% with mineral oil) at 0° C. The reaction mixture was stirred at 100° C. for 2 h. A solution of a mixture of (1R,2S,3S,4S)-2,3-bis((benzyloxy)methyl)-5-oxabicyclo[2.1.0]pentane (Intermediate 3) and its isomers (20.7 g, 66.7 mmol) in DMF (200 mL) were added to the reaction mixture at 80° C. The resulting mixture was stirred at 110° C. for 48 h. The reaction mixture was cooled to r.t., quenched by sat. NH₄Cl aq., diluted with water (3 L), and extracted with EA (1 L×3). The combined organic extracts were washed with brine, dried over anhydrous sodium sulfate, filtered and concentrated under reduced pressure. Purification (FCC, SiO₂, DCM:MeOH=20:1) afforded (1R,2R,3S,4R)-2-(6-amino-9H-purin-9-yl)-3,4-bis((benzyloxy)methyl)cyclobutanol and its isomer as a mixture (17.6 g, 39.5 mmol, 59% yield) as a colorless oil. ESI-LCMS: m/z 446 [M+H]⁺.

Step B. (2R,3S,4R)-2-(6-Amino-9H-purin-9-yl)-3,4-bis((benzyloxy)methyl)cyclobutanone and (2S,3R,4S)-2-(6-Amino-9H-purin-9-yl)-3,4-bis((benzyloxy)methyl)cyclobutanone. To a solution of a mixture of (1R,2R,3S,4R)-2-(6-amino-9H-purin-9-yl)-3,4-bis((benzyloxy)methyl)cyclobutanol and its isomers (17.6 g, 39.5 mmol) in DCM (300 mL) was added DMP (33.5 g, 79.0 mmol) at rt. The reaction mixture was stirred at r.t. for 1.5 h. Purification (FCC, SiO₂, DCM:MeOH=50:1) afforded (2R,3S,4R)-2-(6-amino-9H-purin-9-yl)-3,4-bis((benzyloxy)methyl)cyclobutanone (15.6 g, 28.1 mmol, 71% yield, 80% purity) and its isomer (2S,3R,4S)-2-(6-amino-9H-purin-9-yl)-3,4-bis((benzyloxy)methyl)cyclobutanone) as a yellow solid mixture. ESI-LCMS: m/z 444 [M+H]⁺.

Step C. 9-((1R,2S,3S)-2,3-Bis((benzyloxy)methyl)-4-methylenecyclobutyl)-9H-purin-6-amine. To a suspension of PPh₃CH₃Br (50.3 g, 140.7 mmol) in THF (1.0 L) was added t-BuOK (15.8 g, 140.7 mmol) at 0° C. The reaction mixture was stirred at 0° C. for 1.5 h, then a solution of a mixture of (2R,3S,4R)-2-(6-amino-9H-purin-9-yl)-3,4-bis((benzyloxy)methyl)cyclobutanone and (2S,3R,4S)-2-(6-amino-9H-purin-9-yl)-3,4-bis((benzyloxy)methyl)cyclobutanone (15.6 g, 35.2 mmol) in THF (100 mL) was added to the mixture. The resulting mixture was stirred at 40° C. for 1.5 h. The reaction mixture was quenched with sat. NH₄Cl aq., extracted with EA (1 L×2). The combined extracts were washed with brine, dried over anhydrous sodium sulfate, filtered and concentrated under reduced pressure. Purification (FCC, SiO₂, DCM:MeOH=100:1 to 30:1) afforded 3.5 g, then purification (MPLC, ACN:0.5% NH₄HCO₃ in water=70:30) afforded 9-((1R,2S,3S)-2,3-bis((benzyloxy)methyl)-4-methylenecyclobutyl)-9H-purin-6-amine and its isomer as a mixture (2.0 g, 4.5 mmol, 13% yield) as a yellow oil. ESI-LCMS: m/z 442 [M+H]⁺

Step D. N-(9-((1R,2S,3S)-2,3-Bis((benzyloxy)methyl)-4-methylenecyclobutyl)-9H-purin-6-yl)benzamide. To a solution of a mixture of 9-((1R,2S,3S)-2,3-bis((benzyloxy)methyl)-4-methylenecyclobutyl)-9H-purin-6-amine and its isomer (2.0 g, 4.5 mmol) in pyridine (40 mL) was added BzCl (9.06 mmol, 1.05 mL) at r.t. The reaction mixture was stirred at r.t. The reaction mixture was quenched with MeOH, and concentrated under reduced pressure. The crude reaction product was dissolved in THF (40 mL) and MeOH (10 ml), 30% NH₄OH (10 mL) was added to the mixture at 0° C. The resulting mixture was stirred at 0° C. for 1.5 h. The mixture was acidified using citric acid aq. to pH=5, and extracted with EA (100 mL×2). The combined organic layers were washed with brine, dried over anhydrous sodium sulfate, filtered and concentrated under reduced pressure. Purification (FCC, SiO₂, EA:PE=1:1) afforded 2.3 g (85% purity on HPLC) as a yellow oil. Purification (MPLC, ACN:5% HCOOH in Water=95:5) afforded N-(9-((1R,2S,3S)-2,3-bis((benzyloxy)methyl)-4-methylenecyclobutyl)-9H-purin-6-yl)benzamide and its isomer as a mixture (2.05 g, 3.76 mmol, 83% yield) as a yellowish oil. EST-LCMS: m/z 546 [M+H]⁺.

Step E and F. N-(9-((1S,2R,3R)-2,3-Bis(hydroxymethyl)-4-methylenecyclobutyl)-9H-purin-6-yl)benzamide. To a solution of a mixture of N-(9-((1R,2S,3S)-2,3-bis((benzyloxy)methyl)-4-methylenecyclobutyl)-9H-purin-6-yl)benzamide and its isomer (1.5 g, 2.8 mmol) in DCM (30 mL) was added BCl₃ (1 M, 14.1 mL) at −75° C. under N₂. The resulting mixture was stirred at −78˜−40° C. for 3.5 h. The reaction mixture was quenched with MeOH (30 mL) at 75° C., then warm to r.t. The reaction mixture was basified with sat. NaHCO₃ aq. to pH=6, and concentrated under reduced pressure. Purification (MPLC, C18 Flash Column, Agela Technologies, 12 g, 12 mL/min, ACN:0.5% NH₄HCO₃ in Water=50:50) afforded N-(9-((1R,2S,3S)-2,3-bis(hydroxymethyl)-4-methylenecyclobutyl)-9H-purin-6-yl)benzamide and its isomer as a mixture (703 mg) as a white solid. The mixture product of N-(9-((1R,2S,3S)-2,3-bis(hydroxymethyl)-4-methylenecyclobutyl)-9H-purin-6-yl)benzamide and its isomer (1.25 g) was separated by Supercritical fluid chromatography (SFC) (OZ-H, 2 mL/min, (MeOH70ACN30)/CO₂=35/65) to give N-(9-((1S,2R,3R)-2,3-bis(hydroxymethyl)-4-methylenecyclobutyl)-9H-purin-6-yl)benzamide (509 mg, 1.39 mmol, retention time 3.2 min) as a white solid, ESI-LCMS: 366 [M+H]⁺.

Step G. ((1R,2R,3S)-3-(6-Amino-9H-purin-9-yl)-4-methylenecyclobutane-1,2-diyl)dimethanol. To a solution of N-(9-((1S,2R,3R)-2,3-bis(hydroxymethyl)-4-methylenecyclobutyl)-9H-purin-6-yl)benzamide (35.0 mg, 95.8 μmol) in CH₃NH₂/EtOH (3 mL) was stirred at r.t. for 1.5 h. The reaction mixture was concentrated in vacuo. Purification (MPLC, C18 Flash Column, Agela Technologies, 4 g, 4 mL/min, 0.05% NH₄HCO₃ in H₂O:ACN=20:80) afforded ((1R,2R,3S)-3-(6-amino-9H-purin-9-yl)-4-methylenecyclobutane-1,2-diyl)dimethanol (15.0 mg, 57.4 μmol, 59.9% yield) as a white solid. ¹H-NMR (400 MHz, D₂O): δ 8.19 (s, 1H), 8.10 (s, 1H), 5.23 (d, J=8.4 Hz, 1H), 5.11-5.13 (m, 1H), 4.88-4.90 (m, 1H), 3.73-3.82 (m, 2H), 3.71 (d, J=6 Hz, 2H), 2.85-2.90 (m, 1H). 2.73-3, (m, 1H). ESI-LCMS: m/z 262 [M+H]⁺.

### Example 9: ((1S,2S,3R)-3-(6-Amino-9H-purin-9-yl)-4-methylenecyclobutane-1,2-diyl)dimethanol

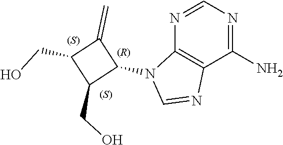

Step A and B. N-(9-((1R,2S,3S)-2,3-Bis(hydroxymethyl)-4-methylenecyclobutyl)-9H-purin-6-yl)benzamide and its isomer. To a solution of a mixture of N-(9-((1R,2S,3S)-2,3-bis((benzyloxy)methyl)-4-methylenecyclobutyl)-9H-purin-6-yl)benzamide and its isomer (Example 8, product from Step D, 1.5 g, 2.8 mmol) in DCM (30 mL) was added BCl₃ (1 M, 14.1 mL) at −75° C. under N₂. The resulting mixture was stirred at −78˜−40° C. for 3.5 h. The reaction mixture was quenched MeOH (30 mL) at −75° C., then warmed to r.t. The reaction mixture was basified with sat. NaHCO₃ aq. to pH=6, and concentrated under reduced pressure. Purification (MPLC, ACN:0.5% NH₄HCO₃ in Water=50:50) afforded N-(9-((1R,2S,3S)-2,3-bis(hydroxymethyl)-4-methylenecyclobutyl)-9H-purin-6-yl)benzamide and its isomer (703 mg) as a white solid. The mixture product (1.25 g) was separated by SFC (OZ-H, 2 mL/min, MeOH(70)ACN(30))/CO₂=35/65) to give N-(9-((1R,2S,3S)-2,3-bis(hydroxymethyl)-4-methylenecyclobutyl)-9H-purin-6-yl)benzamide (493 mg, 1.35 mmol, retention time 3.8 min) as a white solid, ESI-LCMS: m/z 366 [M+H]⁺.

Step C. 41S,2S,3R)-3-(6-Amino-9H-purin-9-yl)-4-methylenecyclobutane-1,2-diyl)dimethanol. A solution of N-(9-((1R,2S,3S)-2,3-bis(hydroxymethyl)-4-methylenecyclobutyl)-9H-purin-6-yl)benzamide (30 mg, 82.1 μmol) in CH₃NH₂/EtOH (3 mL) was stirred at r.t. for 1.5 h. The reaction mixture was concentrated in vacuo. Purification (MPLC, 18 Flash Column, Agela Technologies, 4 g, 4 mL/min, 0.5% HCOOH/H₂O:ACN=1:9) afforded ((1S,2S,3R)-3-(6-amino-9H-purin-9-yl)-4-methylenecyclobutane-1,2-diyl)dimethanol (10 mg, 38.3 μmol, 47% yield) as a white solid. ¹H-NMR (400 MHz, D₂O): δ 8.19 (s, 1H), 8.10 (s, 1H), 5.23 (d, J=8.4 Hz, 1H), 5.11-5.13 (m, 1H), 4.88-4.90 (m, 1H), 3.73-3.82 (m, 2H), 3.71 (d, J=6 Hz, 2H), 2.85-290 (m, 1H), 2.73-3.77 (m, 1H). ESI-LCMS: m/z 262 [M+H]⁺.

### Example 10: ((1R,2R,3S)-3-(2-Amino-6-hydroxy-9H-purin-9-yl)-4-methylenecyclobutane-1,2-diyl)dimethanol

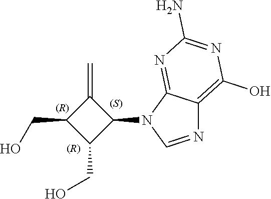

Step A. tert-Butyl (9-((1S,2R,3R,4S)-2,3-bis((benzyloxy)methyl)-4-(diethoxymethyl)cyclobutyl)-6-chloro-9H-purin-2-yl)carbamate. To a solution of (1R,2S,3R,4S)-2,3-bis((benzyloxy)methyl)-4-(diethoxymethyl)cyclobutanol (Intermediate 4, 8.0 g, 19.3 mmol) in 10 mL) was added tert-butyl (6-chloro-9H-purin-2-yl)carbamate (10.4 g, 38.6 mmol), and PPh₃ (10.1 g, 38.6 mmol). The reaction mixture was stirred at 0° C. under N₂ atmosphere, and DIAD (7.8 g, 38.6 mmol) was added slowly. The reaction mixture was stirred at r.t. overnight. The reaction mixture was concentrated under reduced pressure. Purification (FCC, SiO₂, PE:EA=4:1) afforded tert-butyl (9-((1S,2R,3R,4S)-2,3-bis((benzyloxy)methyl)-4-(diethoxymethyl)cyclobutyl)-6-chloro-9H-purin-2-yl)carbamate (9.5 g) as a yellow oil. LC-MS m/z=666.3 [M+H]⁺.

Step B. tert-Butyl (9-((1S,2R,3R,4S)-2,3-bis((benzyloxy)methyl)-4-formylcyclobutyl)-6-chloro-9H-purin-2-yl)carbamate. To a solution of tert-butyl (9-((1S,2R,3R,4S)-2,3-bis((benzyloxy)methyl)-4-(diethoxymethyl)cyclobutyl)-6-chloro-9H-purin-2-yl)carbamate (8.5 g, 12.7 mmol) in CH₃CN (30 mL) was added 0.5 N H₂SO₄ (20 mL). The reaction mixture was stirred at r.t. for 2 h. The reaction mixture was diluted with EtOAc (15 mL) washed with water (2×10 mL), saturated sodium bicarbonate (10 mL), H₂O (2×10 mL), and brine (10 mL). The organic phase was dried (Na₂SO₄) and concentrated in vacuo to give the product tert-butyl (9-((1S,2R,3R,4S)-2,3-bis((benzyloxy)methyl)-4-formylcyclobutyl)-6-chloro-9H-purin-2-yl)carbamate. The crude product was used directly in the next step without further purification. LC-MS m/z=592.2 [M+H]⁺.

Step C. tert-Butyl (9-((1R,2R,3S,4S)-2,3-bis((benzyloxy)methyl)-4-(hydroxymethyl)cyclobutyl)-6-chloro-9H-purin-2-yl)carbamate. To a solution of tert-butyl (9-((1S,2R,3R,4S)-2,3-bis((benzyloxy)methyl)-4-formylcyclobutyl)-6-chloro-9H-purin-2-yl)carbamate in MeOH was added NaBH₄ (723.9 mg, 19.1 mmol) at 0° C. The reaction mixture was stirred at r.t. for 30 min and quenched with H₂O. The resulting mixture was concentrated under reduced pressure. EA (15 mL) was added, the organic layer was washed with water, and brine, dried over anhydrous sodium sulfate, filtered, and concentrated under reduced pressure. Purification (FCC, SiO₂, PE:EA=1:1) afforded tert-butyl (9-((1R,2R,3S,4S)-2,3-bis((benzyloxy)methyl)-4-(hydroxymethyl)cyclobutyl)-6-chloro-9H-purin-2-yl)carbamate (4.5 g, 7.6 mmol, 38.7% yield) as a colorless oil. ¹H NMR (400 MHz, DMSO-d₆) δ 7.40-7.24 (m, 10H), 4.72 (d, J=4.9 Hz, 1H), 4.44 (d, J=10.5 Hz, 4H), 4.30 (q, J=5.9 Hz, 1H), 4.12 (t, J=4.9 Hz, 1H), 3.74-3.56 (m, 2H), 3.44-3.33 (m, 2H), 2.31 (ddd, J=14.5, 8.2, 6.3 Hz, 1H), 2.16 (tt, J=8.0, 6.0 Hz, 1H), 2.05 (tt, J=8.7, 5.8 Hz, 1H). LCMS m/z=594.2 [M+H]⁺.

Step D. tert-Butyl (9-((1S,2R,3R,4S)-2,3-bis((benzyloxy)methyl)-4-(((2-nitrophenyl)selanyl)methyl)cyclobutyl)-6-chloro-9H-purin-2-yl)carbamate. To a solution of tert-butyl (9-((1R,2R,3S,4S)-2,3-bis((benzyloxy)methyl)-4-(hydroxymethyl)cyclobutyl)-6-chloro-9H-purin-2-yl)carbamate (1.0 g, 1.7 mmol) in THF (20 mL) was added 1-nitro-2-selenocyanatobenzene (839.9 mg, 3.4 mmol), followed by PBu₃ (686.8 mg, 3.4 mmol). The reaction mixture was stirred at 55° C. overnight. The reaction mixture was concentrated under reduced pressure. Purification (FCC, SiO₂, PE:EA=2:1) afforded tert-butyl (9-((1S,2R,3R,4S)-2,3-bis((benzyloxy)methyl)-4-(((2-nitrophenyl)selanyl)methyl)cyclobutyl)-6-chloro-9H-purin-2-yl)carbamate (1.1 g, 1.4 mmol, 83.1% yield) as a yellow solid. ¹H NMR (400 MHz, DMSO-d₆) δ 10.30 (s, 1H), 8.41 (s, 1H), 8.09 (dd, J=8.2, 1.5 Hz, 1H), 7.70 (dd, J=8.2, 1.3 Hz, 1H), 7.47-7.37 (m, 1H), 7.37-7.19 (m, 10H), 4.58-4.39 (m, 5H), 3.81-3.53 (m, 4H), 3.18 (p, J=8.0 Hz, 1H), 3.03-2.89 (m, 1H), 2.18 (ddd, J=15.0, 8.7, 6.2 Hz, 1H), 1.44 (s, 9H). LCMS m/z=779.2 [M+H]⁺.

Step E. tert-Butyl (9-((1S,2R,3R)-2,3-bis((benzyloxy)methyl)-4-methylenecyclobutyl)-6-chloro-9H-purin-2-yl)carbamate. To a solution of tert-butyl (9-((1S,2R,3R,4S)-2,3-bis((benzyloxy)methyl)-4-(((2-nitrophenyl)selanyl)methyl)cyclobutyl)-6-chloro-9H-purin-2-yl)carbamate (1.1 g, 1.4 mmol) in THF (30 mL) was added H₂O₂ (5 mL). The reaction mixture was stirred at 55° C. overnight. H₂O (40 mL) was added to the reaction mixture and the reaction mixture was extracted with EA (40 mL×2). The combined organic layers were washed with brine, dried over anhydrous sodium sulfate, filtered, and concentrated under reduced pressure. Purification (FCC, SiO₂, PE:EA=2:1) afforded tert-butyl (9-((1S,2R,3R)-2,3-bis((benzyloxy)methyl)-4-methylenecyclobutyl)-6-chloro-9H-purin-2-yl)carbamate (734.0 mg, 1.3 mmol, 92.9% yield) as a yellow solid. ¹H NMR (400 MHz, DMSO-d₆) δ 10.35 (s, 1H), 8.58 (s, 1H), 7.47-7.14 (m, 10H), 5.45 (dt, J=7.9, 2.6 Hz, 1H), 5.12 (d, J=2.8 Hz, 1H), 4.87 (d, J=2.7 Hz, 1H), 4.56 (s, 2H), 4.48 (s, 2H), 3.91-3.74 (m, 2H), 3.66 (h, J=5.4 Hz, 2H), 3.25 (td, J=7.9, 3.9 Hz, 1H), 3.10 (t, J=7.2 Hz, 1H), 1.48 (s, 9H). LCMS m/z=576.2 [M+H]⁺.

Step F. 2-Amino-9-((1S,2R,3R)-2,3-bis((benzyloxy)methyl)-4-methylenecyclobutyl)-9H-purin-6-ol. tert-Butyl (9-((1S,2R,3R)-2,3-bis((benzyloxy)methyl)-4-methylenecyclobutyl)-6-chloro-9H-purin-2-yl)carbamate (734.0 mg, 1.3 mmol) was dissolved in TFA (15 mL) and H₂O (3 mL), after stirring at RT for 15 min, the mixture was warmed to 50° C. and stirred until the starting material was consumed completely monitored by LC-MS. NaHCO₃ was added to the mixture carefully at r.t. to pH=8. The reaction mixture was extracted with EA. The combined organic layers were washed with sat. NaHCO₃, brine, dried over anhydrous sodium sulfate, filtered, and concentrated under reduced pressure. Purification (FCC, SiO₂, DCM:MeOH=10:1) afforded 2-amino-9-((1S,2R,3R)-2,3-bis((benzyloxy)methyl)-4-methylenecyclobutyl)-9H-purin-6-ol (469.4 mg, 1.03 mmol, 79.0% yield) as a white solid. ¹H NMR (400 MHz, DMSO-d₆) δ 10.60 (s, 1H), 7.79 (s, 1H), 7.47-7.05 (m, 10H), 6.46 (s, 2H), 5.17 (dt, J=7.3, 2.5 Hz, 1H), 5.06 (d, J=2.7 Hz, 1H), 4.77 (d, J=2.7 Hz, 1H), 4.54 (s, 2H), 4.47 (s, 2H), 3.82-3.67 (m, 2H), 3.62 (qd, J=9.9, 4.9 Hz, 2H), 3.01 (tdd, J=10.1, 6.8, 3.7 Hz, 2H). LC-MS m/z=458.2 [M+H]⁺.

Step G. ((1R,2R,3S)-3-(2-Amino-6-hydroxy-9H-purin-9-yl)-4-methylenecyclobutane-1,2-diyl)dimethanol. A solution of 2-amino-9-((1S,2R,3R)-2,3-bis((benzyloxy)methyl)-4-methylenecyclobutyl)-9H-purin-6-ol (469.4 mg, 1.03 mmol) dissolved in DCM (20 mL), was stirred at −75° C. BCl₃ (1 M, 10.3 mL) was added slowly to the reaction mixture. The reaction mixture was stirred at −75° C. for 1 h. To the reaction mixture was added saturated aq Na₂CO₃ (4 mL), H₂O (20 mL), and extracted with DCM (20 mL×2). The combined organic layers were washed with brine, and concentrated under reduced pressure. Purification (MPLC, C18 Flash Column, Agela Technologies, 12 g, 12 mL/min, ACN:H₂O=30:70) afforded ((1R,2R,3S)-3-(2-amino-6-hydroxy-9H-purin-9-yl)-4-methylenecyclobutane-1,2-diyl)dimethanol (174.0 mg, 0.63 mmol, 61.2% yield) as a white solid. ¹H NMR (400 MHz, DMSO-d₆) δ 10.56 (s, 1H), 7.77 (s, 1H), 6.43 (s, 2H), 5.21-4.94 (m, 2H), 4.85-4.61 (m, 3H), 3.67 (h, J=5.3 Hz, 2H), 3.56 (t, J=4.8 Hz, 2H), 2.85-2.70 (m, 1H). LC-MS m/z=278.1 [M+H]⁺.

### Example 11: 41R,2R,3S)-3-(6-Amino-2-fluoro-9H-purin-9-yl)-4-methylenecyclobutane-1,2-diyl)dimethanol

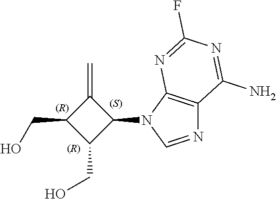

Step A. 9-((1S,2R,3R)-2,3-Bis((benzyloxy)methyl)-4-methylenecyclobutyl)-N,N-Di-Boc-2-fluoro-9H-purin-6-amine. To a solution of (1R,2S,3R)-2,3-bis((benzyloxy)methyl)-4-methylenecyclobutanol (500 mg, 1.54 mmol) in THF (20.0 mL) was added N,N-Di-Boc-2-fluoro-9H-purin-6-amine (1.09 g, 3.08 mmol) and PPh₃ (810.4 mg, 3.08 mmol). The reaction mixture was stirred at 0° C. under N₂ atmosphere, and DIAD (622.2 mg, 3.08 mmol) was added slowly. The reaction mixture was stirred at r.t. for 2 h. The mixture was concentrated in vacuo. Purification (FCC, SiO₂, PE:EA=4:1) afforded 9-((1S,2R,3R)-2,3-Bis((benzyloxy)methyl)-4-methylenecyclobutyl)-N,N-Di-Boc-2-fluoro-9H-purin-6-amine (1.1 g) as a white solid. LC-MS m/z=660.8 [M+H]⁺.

Step B. 9-((1S,2R,3R)-2,3-Bis((benzyloxy)methyl)-4-methylenecyclobutyl)-2-fluoro-9H-purin-6-amine. 9-((1S,2R,3R)-2,3-Bis((benzyloxy)methyl)-4-methylenecyclobutyl)-N,N-Di-Boc-2-fluoro-9H-purin-6-amine (1.1 g crude) was dissolved in TFA (15 mL) and H₂O (3 mL). The reaction mixture was stirred at r.t. for 4 h. NaHCO₃ was added to the mixture carefully at r.t. to pH=8. The reaction mixture was extracted with EA. The combined organic layers were washed with NaHCO₃, and brine, dried over anhydrous sodium sulfate, filtered, and concentrated under reduced pressure. Purification (FCC, SiO₂, PE:EA=1:1) afforded 9-((1S,2R,3R)-2,3-bis((benzyloxy)methyl)-4-methylenecyclobutyl)-2-fluoro-9H-purin-6-amine (450 mg, 0.98 mmol, 63.6% yield in two steps) as a white solid. ¹H NMR (400 MHz, DMSO-d₆) δ 8.21 (s, 1H), 7.84 (s, 2H), 7.40-7.20 (m, 10H), 5.31 (dt, J=7.0, 2.7 Hz, 1H), 5.10 (t, J=2.2 Hz, 1H), 4.83 (d, J=2.2 Hz, 1H), 4.54 (s, 2H), 4.47 (s, 2H), 3.84-3.71 (m, 2H), 3.71-3.60 (m, 2H), 3.05 (tq, J=5.5, 2.7 Hz, 2H). LCMS m/z=460.3 [M+H]⁺.

Step C. ((1R,2R,3S)-3-(6-Amino-2-fluoro-9H-purin-9-yl)-4-methylenecyclobutane-1,2-diyl)dimethanol. A solution of 9-((1S,2R,3R)-2,3-bis((benzyloxy)methyl)-4-methylenecyclobutyl)-2-fluoro-9H-purin-6-amine (450 mg, 0.98 mmol) in DCM (200 mL) was stirred at −75° C. BCl₃ (1 M, 10.3 mL) was added slowly to the reaction mixture. The reaction mixture was stirred at −75° C. for 1 h. To the reaction was added saturated aq. Na₂CO₃ (4 mL), H₂O (20 mL), then extracted with DCM (20 mL×2), washed with brine, and concentrated under reduced pressure. Purification (MPLC, C18 Flash Column, Agela Technologies, 12 g, 12 mL/min, ACN:H₂O=30:70) afforded ((1R,2R,3S)-3-(6-amino-2-fluoro-9H-purin-9-yl)-4-methylenecyclobutane-1,2-diyl)dimethanol (200 mg, 0.72 mmol, 73.5% yield) as a white solid. ¹H NMR (400 MHz, DMSO-d₆) δ 8.20 (s, 1H), 7.81 (s, 2H), 5.20 (dt, J=7.5, 2.6 Hz, 1H), 5.07 (s, 1H), 4.93-4.68 (m, 3H), 3.70 (h, J=5.2 Hz, 2H), 3.58 (h, J=6.0 Hz, 2H), 2.84 (dddd, J=13.9, 8.2, 6.9, 4.1 Hz, 1H). LCMS m/z=280.1 [M+H]⁺.

### Example 12: ((1R,2S,4R)-2-(6-Amino-9H-purin-9-yl)-4-(hydroxymethyl)-3-methylenecyclobutyl)ethan-1-ol

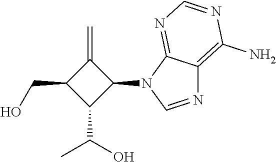 [SMILES: C=C1[C@@H](n2cnc3c(N)ncnc32)[C@H](C(C)O)[C@H]1CO]

Step A. N-(9-((1S,2R,3R)-3-(((tert-Butyldiphenylsilyl)oxy)methyl)-2-(hydroxymethyl)-4-methylenecyclobutyl)-9H-purin-6-yl)benzamide. To a solution of N-(9-((1S,2R,3R)-2,3-bis(hydroxymethyl)-4-methylenecyclobutyl)-9H-purin-6-yl)benzamide (Example 8, Product from Step F, 720 mg, 1.97 mmol) in dry DMF (15 mL) was added imidazole (402.46 mg, 5.91 mmol) followed by TBDPSCl (343.9 mg, 2.96 mmol). The reaction mixture was stirred at r.t. for 2 h. The mixture was quenched with water (20 mL), extracted with EA (20 mL×2), washed with brine, dried over anhydrous Na₂SO₄, filtered and concentrated under reduced pressure. Purification (FCC, SiO₂, DCM:MeOH=80:1) afforded N-(9-((1S,2R,3R)-3-(((tert-butyldiphenylsilyl)oxy)methyl)-2-(hydroxymethyl)-4-methylenecyclobutyl)-9H-purin-6-yl)benzamide (250 mg, 414.1 μmol, 21.0% yield) as a white solid. LCMS m/z=604.2 [M+H]⁺. ¹H NMR (400 MHz, DMSO-d₆) δ (ppm): 11.19 (s, 1H), 8.71 (s, 1H), 8.52 (s, 1H), 8.10-8.02 (m, 2H), 7.72-7.63 (m, 5H), 7.61-7.53 (m, 2H), 7.53-7.44 (m, 6H), 5.55-5.47 (m, 1H), 5.10 (t, J=2.4 Hz, 1H), 4.86 (t, J=5.1 Hz, 1H), 4.82 (d, J=2.3 Hz, 1H), 4.01 (dd, J=7.3, 5.8 Hz, 2H), 3.72-3.57 (m, 2H), 3.07 (dt, J=5.9, 2.7 Hz, 2H), 1.04 (s, 9H). ¹³C NMR (101 MHz, DMSO-d₆) δ (ppm): 165.56, 151.35, 150.18, 148.20, 143.30, 135.09, 133.39, 132.96, 132.37, 129.87, 128.44, 128.42, 127.91, 125.52, 106.11, 64.78, 61.10, 53.76, 45.16, 42.24, 26.63, 18.80.

Step B. N-(9-((1S,2R,3R)-3-(((tert-Butyldiphenylsilyl)oxy)methyl)-2-formyl-4-methylenecyclobutyl)-9H-purin-6-yl)benzamide. To a solution of N-(9-((1S,2R,3R)-3-(((tert-butyldiphenylsilyl)oxy)methyl)-2-(hydroxymethyl)-4-methylenecyclobutyl)-9H-purin-6-yl)benzamide (250 mg, 414.1 μmol) in DCM (10 mL) was added Dess-Martin periodinane (DMP) (379.27 mg, 828.11 μmol) at 0° C. The reaction mixture was stirred at r.t. for 1 h. Purification (FCC, SiO₂, DCM:MeOH=70:1) afforded N-(9-((1S,2R,3R)-3-(((tert-butyldiphenylsilyl)oxy)methyl)-2-formyl-4-methylenecyclobutyl)-9H-purin-6-yl)benzamide (241 mg, 400.5 μmol, 96.7% yield) as a yellow oil. LCMS m/z=620.3 [M+H₂O]⁺.

Step C. N-(9-((1S,2R,3R)-3-(((tert-Butyldiphenylsilyl)oxy)methyl)-2-(1-hydroxyethyl)-4-methylenecyclobutyl)-9H-purin-6-yl)benzamide. To a solution of N-(9-((1S,2R,3R)-3-(((tert-butyldiphenylsilyl)oxy)methyl)-2-formyl-4-methylenecyclobutyl)-9H-purin-6-yl)benzamide (240 mg, 398.8 μmol) in THF (10 mL) was added MeMgBr (1 M, 1.40 mL) by syringe at 0° C. under N₂ atmosphere. The reaction mixture was stirred at 0° C. for 1 h. The mixture was quenched with aq. saturated NH₄Cl (20 mL), extracted with EA (20 mL×2), washed with brine, dried over Na₂SO₄, filtered and concentrated under reduced pressure. Purification (MPLC, C18 Flash Column, Agela Technologies, 12 g, 12 mL/min, MeCN 97% no buffer) afforded N-(9-((1S,2R,3R)-3-(((tert-butyldiphenylsilyl)oxy)methyl)-2-(1-hydroxyethyl)-4-methylenecyclobutyl)-9H-purin-6-yl)benzamide (210 mg, 339.9 μmol, 85.2% yield) as a white solid. LC-MS m/z=618.3 [M+H]⁺. ¹H NMR (400 MHz, DMSO-d₆) δ (ppm): 11.18 (d, J=5.9 Hz, 1H), 8.71 (d, J=4.1 Hz, 1H), 8.52 (d, J=6.7 Hz, 1H), 8.11-7.98 (m, 2H), 7.73-7.62 (m, 5H), 7.56 (t, J=7.6 Hz, 2H), 7.52-7.42 (m, 6H), 5.62-5.49 (m, 1H), 5.15 (m, 1H), 4.91-4.75 (m, 2H), 3.98 (q, J=5.6, 4.8 Hz, 2H), 3.84 (m, 1H), 3.33 (s, 2H), 3.04 (d, J=7.7 Hz, 1H), 2.92-2.82 (m, 1H), 1.04 (s, 9H), 0.94 (m, 3H).

Step D. N-(9-((1S,2R,3R)-3-(((tert-Butyldiphenylsilyl)oxy)methyl)-2-(1-((4-methoxyphenyl)diphenylmethoxy)ethyl)-4-methylenecyclobutyl)-9H-purin-6-yl)benzamide. To a solution of N-(9-((1S,2R,3R)-3-(((tert-butyldiphenylsilyl)oxy)methyl)-2-(1-hydroxyethyl)-4-methylenecyclobutyl)-9H-purin-6-yl)benzamide (200 mg, 323.7 μmol) in DCM (10 mL) was added 2,4,6-collidine (78.46 mg, 647.45 μmol), MMTrCl (149.95 mg, 485.59 μmol) followed by AgNO₃ (54.99 mg, 323.72 μmol). The reaction mixture was stirred at rt. for 2 h. Purification (FCC, SiO₂, PE:EA=1:1) afforded N-(9-((1S,2R,3R)-3-(((tert-butyldiphenylsilyl)oxy)methyl)-2-(1-((4-methoxyphenyl)diphenylmethoxy)ethyl)-4-methylenecyclobutyl)-9H-purin-6-yl)benzamide (200 mg, 224.7 μmol, 69.4% yield) as a yellow oil. LC-MS m/z=890.4 [M+H]⁺.

Step E. N-(9-((1S,2R,3R)-3-(Hydroxymethyl)-2-(1-((4-methoxyphenyl)diphenylmethoxy)ethyl)-4-methylenecyclobutyl)-9H-purin-6-yl)benzamide. To a solution of N-(9-((1S,2R,3R)-3-(((tert-butyldiphenylsilyl)oxy)methyl)-2-(1-((4-methoxyphenyl)diphenylmethoxy)ethyl)-4-methylenecyclobutyl)-9H-purin-6-yl)benzamide (200 mg, 224.68 μmol) in THF (10 mL) was added TBAF (58.75 mg, 224.7 μmol). The reaction mixture was stirred at 35° C. for 2 h. The mixture was concentrated under reduced pressure. Purification (MPLC, C18 Flash Column, Agela Technologies, 4 g, 4 mL/min, MeCN 92% no buffer) afforded N-(9-((1S,2R,3R)-3-(hydroxymethyl)-2-(1-((4-methoxyphenyl)diphenylmethoxy)ethyl)-4-methylenecyclobutyl)-9H-purin-6-yl)benzamide (120 mg, 184.1 μmol, 82.0% yield) as a white solid. LC-MS m/z=652.3 (M+H)⁺.

Step F. ((1R,2R,3S)-3-(6-Amino-9H-purin-9-yl)-2-(1-((4-methoxyphenyl)diphenylmethoxy)ethyl)-4-methylenecyclobutyl)methanol. A solution of N-(9-((1S,2R,3R)-3-(hydroxymethyl)-2-(1-((4-methoxyphenyl)diphenylmethoxy)ethyl)-4-methylenecyclobutyl)-9H-purin-6-yl)benzamide (120 mg, 184.1 μmol) dissolved in 30% MeNH₂/EtOH solution (10 mL), was stirred at r.t. for 1 h. The reaction mixture was concentrated under reduced pressure. Purification (MPLC, C18 Flash Column, Agela Technologies, 4 g, 4 mL/min, MeCN 65% no buffer) afforded ((1R,2R,3S)-3-(6-amino-9H-purin-9-yl)-2-(1-((4-methoxyphenyl)diphenylmethoxy)ethyl)-4-methylenecyclobutyl)methanol (75 mg, 137.0 μmol, 74.4% yield) as a white solid. LC-MS m/z=548.2 (M+H)⁺. ¹H NMR (400 MHz, DMSO-d₆) δ (ppm): 8.23 (d, J=14.5 Hz, 1H), 8.11 (d, J=17.3 Hz, 1H), 7.43-7.39 (m, 1H), 7.34-7.11 (m, 12H), 6.85-6.74 (m, 2H), 5.39 (dd, J=12.8, 8.5 Hz, 1H), 5.13 (dt, J=5.5, 2.6 Hz, 1H), 4.78 (dt, J=5.6, 2.6 Hz, 1H), 4.70 (dt, J=17.8, 5.3 Hz, 1H), 3.73 (d, J=8.5 Hz, 3H), 3.67-3.46 (m, 3H), 3.25 (t, J=6.0 Hz, 1H), 2.92-2.78 (m, 1H), 2.64 (dd, J=52.3, 5.7 Hz, 1H), 0.82 (dd, J=39.2, 6.2 Hz, 3H).

Step G. 1-((1R,2S,4R)-2-(6-Amino-9H-purin-9-yl)-4-(hydroxymethyl)-3-methylenecyclobutyl)ethanol. To a solution of ((1R,2R,3S)-3-(6-amino-9H-purin-9-yl)-2-(1-((4-methoxyphenyl)diphenylmethoxy)ethyl)-4-methylenecyclobutyl)methanol (25 mg, 45.65 μmol) in DCM (5 mL) was added CCl₃COOH (200 mg, 1.22 mmol). The reaction mixture was stirred at r.t. for 1 h. The reaction mixture was concentrated under reduced pressure. Purification (MPLC, C18 Flash Column, Agela Technologies, 4 g, 4 mL/min, MeCN 20% no buffer) afforded 1-((1R,2S,4R)-2-(6-amino-9H-purin-9-yl)-4-(hydroxymethyl)-3-methylenecyclobutyl)ethanol (8.0 mg, 29.1 μmol, 63.7% yield) as a white solid. LC-MS m/z=276.1 (M+H)⁺. ¹H NMR (400 MHz, DMSO-d₆) δ (ppm): 8.22 (d, J=6.5 Hz, 1H), 8.14 (s, 1H), 7.26 (d, J=7.3 Hz, 2H), 5.42-5.27 (m, 1H), 5.09 (dt, J=13.5, 2.6 Hz, 1H), 4.82 (s, 1H), 4.73 (dt, J=5.3, 2.6 Hz, 1H), 3.80 (dt, J=9.4, 5.9 Hz, 1H), 3.71 (dd, J=5.9, 4.3 Hz, 2H), 2.86 (dd, J=5.5, 2.6 Hz, 1H), 2.71 (m, 1H), 0.95 (dd, J=6.3, 1.3 Hz, 3H).

### Example 13: 41R,2R,3S)-3-(6-Amino-9H-purin-9-yl)-2-(fluoromethyl)-4-methylenecyclobutyl)methanol

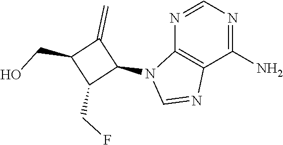 [SMILES: C=C1[C@@H](n2cnc3c(N)ncnc32)[C@H](CF)[C@H]1CO]

Step A. N-(9-((1S,2R,3R)-2-(Hydroxymethyl)-3-(((4-methoxyphenyl)diphenylmethoxy)methyl)-4-methylenecyclobutyl)-9H-purin-6-yl)benzamide. To a solution of N-(9-((1S,2R,3R)-2,3-bis(hydroxymethyl)-4-methylenecyclobutyl)-9H-purin-6-yl)benzamide (Example 8, Product from Step F, 225 mg, 615.8 μmol) in DCM (5 mL) was added pyridine (3.08 mmol, 250 μL) followed by MMTrCl (190 mg, 615.8 μmol) at r.t. The resulting mixture was stirred at r.t. for 18 h. The reaction mixture was diluted with DCM, washed with citric acid aq. solution, brine, dried over anhydrous sodium sulfate, filtered and concentrated in vacu. Purification (FCC, SiO₂, DCM:MeOH=50:1) afforded N-(9-((1S,2R,3R)-2-(hydroxymethyl)-3-(((4-methoxyphenyl)diphenylmethoxy)methyl)-4-methylenecyclobutyl)-9H-purin-6-yl)benzamide (85 mg, 133.3 μmol, 22% yield) as a white solid, ESI-LCMS: m/z 638 [M+H]⁺. ¹H-NMR (400 MHz, DMSO-d₆): δ 11.15 (s, 1H), 8.63 (s, 1H), 8.46 (s, 1H), 8.03 (d, J=8.1 Hz, 2H), 7.66-7.62 (m, 1H), 7.58-7.53 (m, 2H), 7.44-7.24 (m, 12H), 6.93 (d, J=8.8 Hz, 1H), 5.48 (s, 1H), 4.95 (s, 1H), 4.88-4.85 (m, 1H), 4.75 (s, 1H), 3.75 (s, 3H), 3.66-3.62 (m, 2H), 3.40-3.34 (m, 2H), 3.04 (s, 2H).

Step B. ((1R,2S,4R)-2-(6-Benzamido-9H-purin-9-yl)-4-(((4-methoxyphenyl)diphenylmethoxy)methyl)-3-methylenecyclobutyl)methyl 4-methylbenzenesulfonate. To a solution of N-(9-((1S,2R,3R)-2-(hydroxymethyl)-3-(((4-methoxyphenyl)diphenylmethoxy)methyl)-4-methylenecyclobutyl)-9H-purin-6-yl)benzamide (420 mg, 658.59 μmol), DMAP (8.05 mg, 65.86 μmol) and TEA (166.61 mg, 1.65 mmol, 229.64 μL) in dichloromethane (8 mL) was added paratoluensulfonyl chloride (188.34 mg, 987.89 μmol) at 0° C. The reaction mixture was warmed to r.t. and stirred for 16 h. The reaction was quenched with water, and the mixture was extracted with EA. The organic layer was concentrated under reduced pressure. Purification (MPLC, C18 Flash Column, Agela Technologies, 12 g, 12 mL/min, ACN:Water=76:24) afforded ((1R,2S,4R)-2-(6-benzamido-9H-purin-9-yl)-4-(((4-methoxyphenyl)diphenylmethoxy)methyl)-3-methylenecyclobutyl)methyl 4-methylbenzenesulfonate (367 mg, 463.4 μmol, 70.4% yield) as yellow solid. ESI-LCMS m/z=792.3[M+H]⁺

Step C. N-(9-((1S,2R,3R)-2-(Fluoromethyl)-3-(((4-methoxyphenyl)diphenylmethoxy)methyl)-4-methylenecyclobutyl)-9H-purin-6-yl)benzamide. To a solution of ((1R,2S,4R)-2-(6-benzamido-9H-purin-9-yl)-4-(((4-methoxyphenyl)diphenylmethoxy)methyl)-3-methylenecyclobutyl)methyl 4-methylbenzenesulfonate (367 mg, 463.44 μmol) in tetrahydrofuran (8 mL) was added TBAF (484.68 mg, 1.85 mmol) at r.t. The reaction mixture was stirred at 50° C. for 16 h. The reaction mixture was concentrated under reduced pressure. Purification (MPLC, C18 Flash Column, Agela Technologies, 4 g, 4 mL/min, ACN:Water=80:20) afforded N-(9-((1S,2R,3R)-2-(fluoromethyl)-3-(((4-methoxyphenyl)diphenylmethoxy)methyl)-4-methylenecyclobutyl)-9H-purin-6-yl)benzamide (120 mg, 187.6 μmol, 40.5% yield) as a yellow solid. ESI-LCMS m/z=640.2 [M+H]⁺.

Step D. N-(9-((1S,2R,3R)-2-(Fluoromethyl)-3-(hydroxymethyl)-4-methylenecyclobutyl)-9H-purin-6-yl)benzamide. A solution of N-(9-((1S,2R,3R)-2-(fluoromethyl)-3-(((4-methoxyphenyl)diphenylmethoxy)methyl)-4-methylenecyclobutyl)-9H-purin-6-yl)benzamide (120 mg, 187.58 μmol) in 3% trichloroacetic acid/DCM (8 mL) was stirred at r.t. for 10 min. The reaction mixture was quenched with saturated solution of sodium bicarbonate and the reaction mixture was concentrated under reduced pressure. Purification (MPLC, C18 Flash Column, Agela Technologies, 4 g, 4 mL/min, ACN:Water=30:70) afforded N-(9-((1S,2R,3R)-2-(fluoromethyl)-3-(hydroxymethyl)-4-methylenecyclobutyl)-9H-purin-6-yl)benzamide (65 mg, 175.2 μmol, 93.4% yield, 99% purity) as a yellow solid. ESI-LCMS m/z=368.1 [M+H]⁺

Step E. ((1R,2R,3S)-3-(6-Amino-9H-purin-9-yl)-2-(fluoromethyl)-4-methylenecyclobutyl)methanol. A solution of N-(9-((1S,2R,3R)-2-(fluoromethyl)-3-(hydroxymethyl)-4-methylenecyclobutyl)-9H-purin-6-yl)benzamide (68 mg, 185.10 μmol) in methylamine/ethanol (5 mL) was stirred for 30 min. The reaction mixture was concentrated under reduced pressure. Purification (MPLC, C18 Flash Column, Agela Technologies, 4 g, 4 mL/min, ACN:Water=10:90) afforded ((1R,2R,3S)-3-(6-amino-9H-purin-9-yl)-2-(fluoromethyl)-4-methylenecyclobutyl)methanol (42 mg, 159.5 μmol, 86.2% yield) as white solid. ESI-LCMS m/z=261.1 [M+H]⁺. ¹H-NMR (400 MHz, DMSO-d₆): δ 8.22 (s, 1H), 8.13 (s, 1H), 7.24 (s, 2H), 5.39-5.37 (m, 1H), 5.12 (t, J=2.2 Hz, 1H), 4.81 (s, 2H), 4.68 (d, J=4.8 Hz, 1H), 4.56 (d, J=4.8 Hz, 1H), 3.73 (d, J=5.6 Hz, 1H), 3.19-3.09 (m, 1H), 2.89-2.87 (m, 1H). ¹⁹FNMR (400 MHz, DMSO-d₆): δ −223.95 (s).

### Example 14: (1R,2S,4R)-2-(6-Amino-9H-purin-9-yl)-4-(hydroxymethyl)-3-methylenecyclobutanecarbonitrile

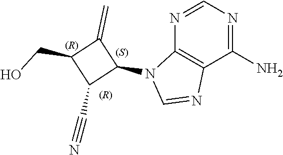

Step A. N-(9-((1S,2R,3R)-3-(Hydroxymethyl)-2-(((4-methoxyphenyl)diphenylmethoxy)methyl)-4-methylenecyclobutyl)-9H-purin-6-yl)benzamide. To a solution of N-(9-((1S,2R,3R)-2,3-bis(hydroxymethyl)-4-methylenecyclobutyl)-9H-purin-6-yl)benzamide (Example 8, Product from Step F, 240 mg, 656 μmol) in dry DCM (10 mL) was added pyridine (259 mg, 3.3 mmol, 264 μL). A solution of MMTrCl (202 mg, 656.84 μmol) in DCM at 0° C. was added dropwise to the reaction mixture. The reaction mixture was stirred at 0° C. for 1 h. The mixture was quenched with methanol. The reaction mixture was concentrated under reduced pressure. Purification (MPLC, C18 Flash Column, Agela Technologies, 12 g, 12 mL/min, ACN:H₂O=40:60) afforded N-(9-((1S,2R,3R)-3-(hydroxymethyl)-2-(((4-methoxyphenyl)diphenylmethoxy)methyl)-4-methylenecyclobutyl)-9H-purin-6-yl)benzamide (160 mg, 250 μmol) ESI LC-MS: m/z 638 [M+H]⁺.

Step B. ((1R,2R,3S)-3-(6-Benzamido-9H-purin-9-yl)-2-(((4-methoxyphenyl)diphenylmethoxy)methyl)-4-methylenecyclobutyl)methyl benzoate. To a solution of N-(9-((1S,2R,3R)-3-(hydroxymethyl)-2-(((4-methoxyphenyl)diphenylmethoxy)methyl)-4-methylenecyclobutyl)-9H-purin-6-yl)benzamide (360 mg, 564 μmol) in dry pyridine (8 mL) was added a solution of benzoyl chloride (119 mg, 846 μmol) in DCM dropwise at 0° C. under N₂. The reaction mixture was stirred for 1 h at 0° C., then cold water was added to quench the reaction. The reaction mixture was concentrated under reduced pressure. Purification (MPLC, C18 Flash Column, Agela Technologies, 12 g, 12 mL/min, ACN:H₂O=50:50) afforded ((1R,2R,3S)-3-(6-benzamido-9H-purin-9-yl)-2-(((4-methoxyphenyl)diphenylmethoxy)methyl)-4-methylenecyclobutyl)methyl benzoate (140 mg, 165 μmol) as a white solid. ESI-LCMS: m/z 742 [M+H]⁺.

Step C. ((1R,2R,3S)-3-(6-Benzamido-9H-purin-9-yl)-2-hydroxymethyl)-4-methylenecyclobutyl)methyl benzoate. To a solution of ((I R,2R,3S)-3-(6-benzamido-9H-purin-9-yl)-2-(((4-methoxyphenyl)diphenylmethoxy)methyl)-4-methylenecyclobutyl)methyl benzoate (240 mg, 323 μmol) in DCM (10 mL) was added trichloroacetic acid (TCA) (0.3 g) at r.t. The reaction mixture was stirred for 1 h at r.t. To the reaction mixture was added sat. NaHCO₃. The reaction mixture was concentrated in vacuo. Purification (MPLC, C18 Flash Column, Agela Technologies, 12 g, 12 mL/min, ACN:H₂O=50:50) afforded ((1R,2R,3S)-3-(6-benzamido-9H-purin-9-yl)-2-(hydroxymethyl)-4-methylenecyclobutyl)methyl benzoate (150 mg, 319 μmol) as a white solid. ESI-LCMS: m/z 470 [M+H]⁺.

Step D. ((1R,2R,3S)-3-(6-Benzamido-9H-purin-9-yl)-2-formyl-4-methylenecyclobutyl)methyl benzoate. To a solution of ((1R,2R,3S)-3-(6-benzamido-9H-purin-9-yl)-2-(hydroxymethyl)-4-methylenecyclobutyl)methyl benzoate (210 mg, 447 μmol) in DCM (5 mL) was added Dess-Martin periodinane (284 mg, 670 μmol) at r.t. The reaction mixture was stirred for 1 h at r.t. The reaction mixture was washed sat. NaHCO₃ and sat. Na₂SO₃, and extracted with DCM. The organic layer was washed with brine, dried over Na₂SO₄, filtered, and concentrated in vacuo. The title compound, ((1R,2R,3S)-3-(6-benzamido-9H-purin-9-yl)-2-formyl-4-methylenecyclobutyl)methyl benzoate (230 mg, 393 μmol, 80% purity) as a whiter solid was used directly in the next step without further purification. ESI-LCMS: m/z 468 [M+H]⁺, 486[M+H+H₂O]⁺.

Step E. ((1R,2R,3S)-3-(6-Benzamido-9H-purin-9-yl)-2-((E)-(hydroxyimino)methyl)-4-methylenecyclobutyl)methyl benzoate. To s solution of ((1R,2R,3S)-3-(6-benzamido-9H-purin-9-yl)-2-formyl-4-methylenecyclobutyl)methyl benzoate (230 mg, 393 μmol) in pyridine (5 mL) was added hydroxylamine hydrochloride (82 mg, 1.18 mmol) at r.t. The reaction mixture was quenched with ice water, and extracted with DCM (20 ml×4). The combined organic extracts were dried over Na₂SO₄, filtered, and concentrated in vacuo to give crude product ((1R,2R,3S)-3-(6-benzamido-9H-purin-9-yl)-2-((E)-(hydroxyimino)methyl)-4-methylenecyclobutyl)methyl benzoate (240 mg, 348 μmol, 70% purity) as a white solid, used directly in the next step. ESI-LCMS: m/z 483 [M+H]⁺.

Step F. ((1R,2R,3S)-3-(6-Benzamido-9H-purin-9-yl)-2-cyano-4-methylenecyclobutyl)methyl benzoate. To a solution of ((1R,2R,3S)-3-(6-benzamido-9H-purin-9-yl)-2-((E)-(hydroxyimino)methyl)-4-methylenecyclobutyl)methyl benzoate (240 mg, 348 μmol) in dry pyridine (5 mL) was added dropwise a solution of methanesulfonyl chloride (200 mg, 1.74 mmol) in pyridine at 0° C. under N₂. The mixture was stirred for 1 h at 0° C. To the reaction mixture was added dropwise cold 4N HCl to quench the reaction. The reaction mixture was extracted with DCM (20 mL×4) and washed with sat. NaHCO₃ and brine. The combined organic layers were concentrated under reduced pressure. Purification (MPLC, C18 Flash Column, Agela Technologies, 4 g, 4 mL/min, ACN:H₂O=40:60) afforded ((1R,2R,3S)-3-(6-benzamido-9H-purin-9-yl)-2-cyano-4-methylenecyclobutyl)methyl benzoate (100 mg, 215. μmol) as a white solid. ¹H-NMR (400 MHz, DMSO): δ ppm 11.26 (br s, 1H), 8.70 (s, 1H), 8.60 (s, 1H), 8.1-8.1 (m, 4H), 7.75-7.62 (m, 2H), 7.62-7.53 (m, 4H), 6.23-6.17 (m, 1H), 5.40-5.34 (m, 1H), 5.10-5.16 (m, 1H), 4.78-4.68 (m, 2H), 4.42-4.32 (m, 1H), 3.95-3.85 (m, 1H). ESI-LCMS: m/z 465 [M+H]⁺.

Step G. (1R,2S,4R)-2-(6-Amino-9H-purin-9-yl)-4-(hydroxymethyl)-3-methylenecyclobutanecarbonitrile. ((1R,2R,3S)-3-(6-benzamido-9H-purin-9-yl)-2-cyano-4-methylenecyclobutyl)methyl benzoate (100 mg, 215.30 μmol) was dissolved in methylamine/methanol (5 mL) at r.t. The reaction mixture was stirred at r.t. for 4 h. The reaction mixture was concentrated in vacuo. Purification (MPLC, C18 Flash Column, Agela Technologies, 4 g, 4 mL/min, ACN:H₂O=20:80) afforded (1R,2S,4R)-2-(6-amino-9H-purin-9-yl)-4-(hydroxymethyl)-3-methylenecyclobutanecarbonitrile (25 mg, 97.6 μmol) as a whiter solid. ¹H-NMR (400 MHz, DMSO): δ ppm 8.22 (s, 1H), 8.17 (s, 1H), 7.36 (br s, 2H), 5.93-5.87 (m, 1H), 5.24-5.19 (m, 1H), 5.07 (t, J=5.5, 1H), 4.97-4.93 (m, 1H), 3.95 (t, J=8.16, 1H), 3.85-3.72 (m, 2H), 3.95-3.85 (m, 1H). ESI-LCMS: m/z 257 [M+H]⁺.

### Example 15: ((1S,2S,3R,Z)-3-(6-Amino-9H-purin-9-yl)-4-(fluoromethylene)cyclobutane-1,2-diyl)dimethanol

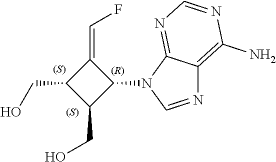

Step A. 9-((1S,2R,3R,Z)-2,3-bis((benzyloxy)methyl)-4-ffluoro(phenylsulfonyl)methylene)cyclobutyl)-9H-purin-6-amine and its isomers. To a solution of fluoromethyl phenyl sulfone (1.65 g, 9.47 mmol) and diethyl chlorophosphite (1.48 g, 9.47 mmol) in tetrahydrofuran (55 mL) was added lithium bis(trimethylsilyl)amide (1.58 g, 9.47 mmol) dropwise via a syringe at −78° C. The reaction mixture was stirred at −78° C. for 50 min, then a solution of (2S,3R,4S)-2-(6-amino-9H-purin-9-yl)-3,4-bis((benzyloxy)methyl)cyclobutanone and (2R,3S,4R)-2-(6-amino-9H-purin-9-yl)-3,4-bis((benzyloxy)methyl)cyclobutanone (Example 8, Product from Step B, 2.8 g, 6.31 mmol) in tetrahydrofuran (15 mL) was added to the mixture dropwise via a syringe. The reaction mixture was warmed to r.t. and stirred for 16 h. The reaction mixture was quenched with saturated solution of ammonium chloride at 0° C. The reaction mixture was extracted with EA, and the organic layer was washed with brine and concentrated under reduced pressure. Purification (FCC, SiO₂, DCM:MeOH=100:1) afforded 9-((1S,2R,3R,Z)-2,3-bis((benzyloxy)methyl)-4-(fluoro(phenylsulfonyl)methylene)cyclobutyl)-9H-purin-6-amine and its isomers as a mixture (800 mg, 1.13 mmol, 18.0% yield, 85% purity) as a white solid.

Step B. 9-((1S,2R,3R,Z)-2,3-Bis((benzyloxy)methyl)-4-ffluoro(tributylstannyl)methylene)cyclobutyl)-9H-purin-6-amine and its enantiomer, 9-(1S,2R,3R,E)-2,3-bi s ((benzyloxy)methyl)-4-(fluoro(tributyl stannyl)methylene)cyclobutyl)-9H-purin-6-amine and its enantiomer. To a solution of a mixture of 9-((1S,2R,3R,Z)-2,3-bis((benzyloxy)methyl)-4-(fluoro(phenylsulfonyl)methylene)cyclobutyl)-9H-purin-6-amine and its isomers (2.0 g, 3.34 mmol) and azobisisobutyronitrile (AIBN) (219.07 mg, 1.33 mmol) in toluene (20 mL) was added tri-n-butyltin hydride (2.90 g, 10.01 mmol, 2.69 mL) at r.t. under N₂. The reaction mixture was refluxed for 3 h. The reaction mixture was concentrated under reduced pressure. Purification (FCC, SiO₂, DCM:MeOH=100:1) afforded 9-((1S,2R,3R,Z)-2,3-bis((benzyloxy)methyl)-4-(fluoro(tributylstannyl)methylene)cyclobutyl)-9H-purin-6-amine and its enantiomer (0.88 g, 1.12 mmol, 33.5% yield, 95% purity) and 9-((1S,2R,3R,E)-2,3-bis((benzyloxy)methyl)-4-(fluoro(tributylstannyl)methylene)cyclobutyl)-9H-purin-6-amine and its enantiomer as a mixture (0.91 g, 1.15 mmol, 34.6% yield, 95% purity) as a colorless oil. ESI-LCMS m/z=750.2 [M+H]⁺.

9-((1S,2R,3R,Z)-2,3-bis((benzyloxy)methyl)-4-(fluoro(tributylstannyl)methylene)cyclobutyl)-9H-purin-6-amine and its enantiomer. ¹H-NMR (400 MHz, DMSO-d₆): δ 8.20 (s, 1H), 8.15 (s, 1H) 7.36-7.27 (m, 12H), 5.53-5.51 (m, 1H), 4.58-4.57 (m, 2H), 4.46 (s, 1H), 4.01-3.97 (m, 1H), 3.83-3.80 (m, 1H), 3.61-3.60 (m, 2H), 4.01-3.97 (m, 1H), 3.18 (s, 1H), 2.90-2.87 (m, 1H), 1.25-1.21 (m, 6H), 1.13-1.08 (m, 6H), 0.77 (s, 9H), 0.61-0.55 (m, 6H). ¹⁹FNMR (400 MHz, DMSO-d₆): δ −107.29 (s).

9-((1S,2R,3R,Z)-2,3-bis((benzyloxy)methyl)-4-(fluoro(tributylstannyl)methylene)cyclobutyl)-9H-purin-6-amine and its enantiomer. ¹H-NMR (400 MHz, DMSO-d₆): δ 8.12 (s, 1H), 8.11 (s, 1H) 7.38-7.21 (m, 12H), 5.53-5.51 (m, 1H), 4.58-4.46 (m, 4H), 3.85-3.81 (m, 1H), 3.68-3.62 (m, 3H), 3.09-2.99 (m, 2H), 1.54-1.36 (m, 6H), 1.28-1.18 (m, 6H), 0.96-0.92 (m, 6H), 0.95-0.81 (m, 9H). ¹⁹FNMR (400 MHz, DMSO-d₆): δ −106.70 (s).

Step C. 9-((1S,2R,3R,Z)-2,3-bis((benzyloxy)methyl)-4-(fluoromethylene)cyclobutyl)-9H-purin-6-amine and its enantiomer. To a solution of a mixture of 9-((1S,2R,3R,Z)-2,3-bis((benzyloxy)methyl)-4-(fluoro(tributylstannyl)methylene)cyclobutyl)-9H-purin-6-amine and its enantiomer (910 mg, 1.22 mmol) in methanol (30 mL) was added sodium methoxide (328.4 mg, 6.1 mmol) at r.t The reaction mixture was stirred at r.t. for 16 h. The reaction mixture was quenched by HCl (aq. 6N) to pH=7, and concentrated under reduced pressure. Purification (MPLC, C18 Flash Column, Agela Technologies, 12 g, 12 mL/min, ACN:Water=75:25) afforded 9-((1S,2R,3R,Z)-2,3-bis((benzyloxy)methyl)-4-(fluoromethylene)cyclobutyl)-9H-purin-6-amine and its enantiomer as a mixture (550 mg, 1.1 mmol, 88.6% yield, 90% purity) as a white solid. ESI LC-MS m/z=460.2 [M+H]⁺.

Step D. N-(9-((1S,2R,3R,Z)-2,3-bis((benzyloxy)methyl)-4-(fluoromethylene)cyclobutyl)-9H-purin-6-yl)benzamide and its enantiomer. To a solution of 9-((1S,2R,3R,Z)-2,3-bis((benzyloxy)methyl)-4-(fluoromethylene)cyclobutyl)-9H-purin-6-amine and its enantiomer (550 mg, 1.2 mmol) in pyridine (8 mL) was added benzoyl chloride (252.4 mg, 1.8 mmol, 208.6 μL) dropwise at 0° C. The reaction mixture was stirred at r.t. for 6 h. The reaction mixture was quenched by NH₄OH, then the mixture was concentrated under reduced pressure. Purification (MPLC, C18 Flash Column, Agela Technologies, 12 g, 12 mL/min, ACN:Water=85:15) afforded N-(9-((1S,2R,3R,Z)-2,3-bis((benzyloxy)methyl)-4-(fluoromethylene)cyclobutyl)-9H-purin-6-yl)benzamide and its enantiomer as a mixture (480 mg, 766.5 μmol, 64.0% yield, 90% purity) as a white solid. ESI-LCMS m/z=564.2 [M+H]⁺

Step E. N-(9-((1S,3R,4R,Z)-2-(Fluoromethylene)-3,4-bis(hydroxymethyl)cyclobutyl)-9H-purin-6-yl)benzamide and its enantiomer. To a solution of N-(9-((1S,2R,3R,Z)-2,3-bis((benzyloxy)methyl)-4-(fluoromethylene)cyclobutyl)-9H-purin-6-yl)benzamide and its enantiomer (920 mg, 1.6 mmol) in dichloromethane (30 mL) was added boron trichloride (1 M, 19.6 mL) dropwise at −78° C. The reaction mixture was stirred at −78° C. for 1 h. The reaction mixture was quenched by methanol at −78° C., and added TEA to pH=6. The reaction mixture was concentrated under reduced pressure. Purification (MPLC, C18 Flash Column, Agela Technologies, 12 g, 12 mL/min, ACN:Water=34:66) afforded N-(9-((1S,3R,4R,Z)-2-(fluoromethylene)-3,4-bis(hydroxymethyl)cyclobutyl)-9H-purin-6-yl)benzamide and its enantiomer as a mixture (480 mg, 1.2 mmol, 72.9% yield, 95% purity) as a white solid. ESI LC-MS m/z=384.1 [M+H]⁺. ¹H-NMR (400 MHz, DMSO-d₆): δ 8.76 (s, 1H), 8.61 (s, 1H), 8.10-7.54 (m, 5H), 7.03-6.82 (m, 1H), 5.63-5.61 (m, 1H), 4.92-4.86 (m, 2H), 3.78-3.73 (m, 2H), 3.62-3.59 (m, 2H), 2.99-2.90 (m, 2H). The material was further separated by SFC (AS-H, 2 mL/min, CO₂:MeOH=85:15), to give N-(9-((1S,3R,4R,Z)-2-(fluoromethylene)-3,4-bis(hydroxymethyl)cyclobutyl)-9H-purin-6-yl)benzamide (270 mg) and N-(9-((1R,3S,4S,Z)-2-(fluoromethylene)-3,4-bis(hydroxymethyl)cyclobutyl)-9H-purin-6-yl)benzamide (270 mg).

Step F. N-(9-((1R,3S,4S,Z)-2-(Fluoromethylene)-3-(hydroxymethyl)-4-(((4-methoxyphenyl)diphenylmethoxy)methyl)cyclobutyl)-9H-purin-6-yl)benzamide and its isomer. To a solution of N-(9-((1R,3S,4S,Z)-2-(fluoromethylene)-3,4-bis(hydroxymethyl)cyclobutyl)-9H-purin-6-yl)benzamide (270 mg, 704.3 μmol) and pyridine (278.5 mg, 3.5 mmol, 283.7 μL) in dichloromethane (20 mL) was added 4-methoxytriphenylmethyl chloride (239.2 mg, 774.7 μmol) at 0° C. The reaction mixture was allowed warm to r.t. and stirred for 3 h. The reaction was quenched with methanol. The reaction mixture was concentrated under reduced pressure. Purification (MPLC, C18 Flash Column, Agela Technologies, 4 g, 4 mL/min, ACN:Water=70:30) and further purification (FCC, SiO₂, DCM:MeOH=100:1) afforded N-(9-((1R,3S,4S,Z)-2-(fluoromethylene)-3-(hydroxymethyl)-4-(((4-methoxyphenyl)diphenylmethoxy)methyl)cyclobutyl)-9H-purin-6-yl)benzamide (120 mg, 168.4 μmol, 23.9% yield, 92% purity) and N-(9-((1R,3S,4S,Z)-2-(fluoromethylene)-4-(hydroxymethyl)-3-(((4-methoxyphenyl)diphenylmethoxy)methyl)cyclobutyl)-9H-purin-6-yl)benzamide (80 mg, 115.90 μmol, 16.5% yield, 95% purity) as white solid ESI LC-MS m/z=656.3 [M+H]⁺.

Step G. ((1S,2R,4S,Z)-2-(6-Amino-9H-purin-9-yl)-3-(fluoromethylene)-4-(((4-methoxyphenyl)diphenylmethoxy)methyl)cyclobutyl)methanol. A solution of N-(9-((1R,3S,4S,Z)-2-(fluoromethylene)-4-(hydroxymethyl)-3-(((4-methoxyphenyl)diphenylmethoxy)methyl)cyclobutyl)-9H-purin-6-yl)benzamide (80 mg, 122.0 μmol) in methylamine/ethanol (2 mL) was stirred at r.t. for 30 min. The reaction mixture was concentrated under reduced pressure. Purification (MPLC, C18 Flash Column, Agela Technologies, 4 g, 4 mL/min, ACN:Water=50:50) afforded 41S,2R,4S,Z)-2-(6-amino-9H-purin-9-yl)-3-(fluoromethylene)-4-(((4-methoxyphenyl)diphenylmethoxy)methyl)cyclobutyl)methanol (40 mg, 68.9 μmol, 56.5% yield, 95% purity) as a white solid. ESI LC-MS m/z=552.2 [M+H]⁺.

Step H. ((1S,2S,3R,Z)-3-(6-Amino-9H-purin-9-yl)-4-fluoromethylene)cyclobutane-1,2-diyl)dimethanol. A solution of ((1S,2R,4S,Z)-2-(6-amino-9H-purin-9-yl)-3-(fluoromethylene)-4-(((4-methoxyphenyl)diphenylmethoxy)methyl)cyclobutyl)methanol (50 mg, 90.6 μmol) in 3% trichloroacetic acid/DCM (3 mL) was stirred at r.t. for 30 min. The reaction mixture was concentrated under reduced pressure. Purification (MPLC, C18 Flash Column, Agela Technologies, 4 g, 4 mL/min, ACN:Water=15:85) afforded 41S,2S,3R,Z)-3-(6-amino-9H-purin-9-yl)-4-(fluoromethylene)cyclobutane-1,2-diyl)dimethanol (20 mg, 71.6 μmol, 79.0% yield) as a white solid. ESI LC-MS m/z=280.1 [M+H]⁺. ¹H-NMR (400 MHz, DMSO-d₆): δ 8.23 (s, 1H), 8.14 (s, 1H), 7.24 (s, 2H), 6.98 (t, J=2.1 Hz, 0.5H), 6.77 (t, J=2.1 Hz, 0.5H), 5.45-5.43 (m, 1H), 4.89 (t, J=5.0 Hz, 1H), 4.84 (t, J=5.0 Hz, 1H), 3.73-3.68 (m, 2H), 3.56 (t, J=4.3 Hz, 2H), 2.88-2.87 (m, 2H). ¹⁹FNMR (400 MHz, DMSO-d₆): δ −138.71 (s).

### Example 16: ((1R,2R,3S,Z)-3-(6-Amino-9H-purin-9-yl)-4-(fluoromethylene)cyclobutane-1,2-diyl)dimethanol

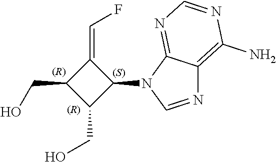

Step A. N-(9-((1S,3R,4R,Z)-2-(Fluoromethylene)-3-(hydroxymethyl)-4-(((4-methoxyphenyl)diphenylmethoxy)methyl)cyclobutyl)-9H-purin-6-yl)benzamide and its isomer. To a solution of N-(9-((1S,3R,4R,Z)-2-(fluoromethylene)-3,4-bis(hydroxymethyl)cyclobutyl)-9H-purin-6-yl)benzamide (Example 15, Step E) (270 mg, 704.3 μmol) and pyridine (278.5 mg, 3.5 mmol, 283.7 μL) in dichloromethane (20 mL) was added 4-methoxytriphenylmethyl chloride (239.2 mg, 774.7 μmol) at 0° C. The reaction mixture was allowed warm to r.t. and stirred for 3 h. The reaction mixture was quenched by methanol. The reaction mixture was concentrated under reduced pressure. Purification (MPLC, C18 Flash Column, Agela Technologies, 4 g, 4 mL/min, ACN:Water=70:30) and further purification (FCC, SiO₂, DCM:MeOH=100:1) afforded N-(9-((1S,3R,4R,Z)-2-(fluoromethylene)-3-(hydroxymethyl)-4-(((4-methoxyphenyl)diphenylmethoxy)methyl)cyclobutyl)-9H-purin-6-yl)benzamide (120 mg, 168.4 μmol, 23.9% yield, 92% purity) and N-(9-((1S,3R,4R,Z)-2-(fluoromethylene)-4-(hydroxymethyl)-3-(((4-methoxyphenyl)diphenylmethoxy)methyl)cyclobutyl)-9H-purin-6-yl)benzamide (80 mg, 115.9 μmol, 16.5% yield, 95% purity) as white solid ESI LC-MS m/z=656.3 [M+H]⁺.

Step B. ((1R,2S,4R,Z)-2-(6-Amino-9H-purin-9-yl)-3-(fluoromethylene)-4-(((4-methoxyphenyl)diphenylmethoxy)methyl)cyclobutyl)methanol. A solution of N-(9-41S,3R,4R,Z)-2-(fluoromethylene)-4-(hydroxymethyl)-3-(((4-methoxyphenyl)diphenylmethoxy)methyl)cyclobutyl)-9H-purin-6-yl)benzamide (80 mg, 122.0 μmol) in methylamine/ethanol (4 mL) was stirred at r.t. for 30 min. The reaction mixture was concentrated under reduced pressure. Purification (MPLC, C18 Flash Column, Agela Technologies, 4 g, 4 mL/min, ACN:Water=50:50) afforded ((1R,2S,4R,Z)-2-(6-amino-9H-purin-9-yl)-3-(fluoromethylene)-4-(((4-methoxyphenyl)diphenylmethoxy)methyl)cyclobutyl)methanol (40 mg, 69.6 μmol, 57.1% yield, 96% purity) as a white solid. ESI LC-MS m/z=552.2[M+H]⁺.

Step C. ((1R,2R,3S,Z)-3-(6-Amino-9H-purin-9-yl)-4-(fluoromethylene)cyclobutane-1,2-diyl)dimethanol. A solution of ((1R,2S,4R,Z)-2-(6-amino-9H-purin-9-yl)-3-(fluoromethylene)-4-(((4-methoxyphenyl)diphenylmethoxy)methyl)cyclobutyl)methanol (71.9 mg, 130.4 μmol) in 3% trichloroacetic acid/DCM (3 mL) was stirred at r.t. for 30 min. The reaction mixture was concentrated under reduced pressure. Purification (MPLC, C18 Flash Column, Agela Technologies, 4 g, 4 mL/min, ACN:Water=15:85) afforded ((1R,2R,3S,Z)-3-(6-amino-9H-purin-9-yl)-4-(fluoromethylene)cyclobutane-1,2-diyl)dimethanol (10 mg, 35.5 μmol, 27.2% yield, 99% purity) as a white solid. ESI-LCMS m/z=280.1 [M+H]⁺. ¹H-NMR (400 MHz, DMSO-d₆): δ 8.23 (s, 1H), 8.14 (s, 1H), 7.24 (s, 2H), 6.98 (t, J=2.1 Hz, 0.5H), 6.77 (t, J=2.1 Hz, 0.5H), 5.45-5.43 (m, 1H), 4.89 (t, J=5.0 Hz, 1H), 4.84 (t, J=5.0 Hz, 1H), 3.73-3.68 (m, 2H), 3.56 (t, J=4.3 Hz, 2H), 2.88-2.87 (m, 2H). ¹⁹FNMR (400 MHz, DMSO-d₆): δ −138.71 (s).

### Example 17: ((1R,2R,3S,E)-3-(6-Amino-9H-purin-9-yl)-4-(fluoromethylene)cyclobutane-1,2-diyl)dimethanol

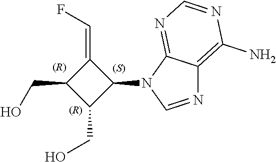

Step A. 9-((1S,2R,3R,E)-2,3-Bis((benzyloxy)methyl)-4-(fluoromethylene)cyclobutyl)-9H-purin-6-amine and its enantiomer. To a solution of 9-((1S,2R,3R,Z)-2,3-bi s ((benzyloxy)methyl)-4-(fluoro(tributyl stannyl)methylene)cyclobutyl)-9H-purin-6-amine and its enantiomer (880 mg, 1.18 mmol) in methanol (30 mL) was added sodium methoxide (317.6 mg, 5.9 mmol) at r.t. The reaction mixture was stirred at r.t. for 16 h. The reaction mixture was quenched by HCl (aq. 6N) to pH=7, and the reaction mixture was concentrated under reduced pressure. Purification (MPLC, C18 Flash Column, Agela Technologies, 12 g, 12 mL/min, ACN:Water=75:25) afforded 9-((1S,2R,3R,E)-2,3-bis((benzyloxy)methyl)-4-(fluoromethylene)cyclobutyl)-9H-purin-6-amine and its enantiomer as a mixture (400 mg, 800.9 μmol, 68.1% yield, 92% purity) as white solid. ESI-LCMS m/z=460.2 [M+H]⁺

Step B. N-(9-((1S,2R,3R,E)-2,3-Bis((benzyloxy)methyl)-4-(fluoromethylene)cyclobutyl)-9H-purin-6-yl)benzamide and its enantiomer. To a solution of a mixture of 9-((1S,2R,3R,E)-2,3-bis((benzyloxy)methyl)-4-(fluoromethylene)cyclobutyl)-9H-purin-6-amine and its enantiomer (400 mg, 870.48 μmol) in pyridine (8 mL) was added benzoyl chloride (183.5 mg, 1.31 mmol, 151.7 μL) dropwise at 0° C. The reaction mixture was stirred at r.t. for 6 h. The reaction mixture was quenched with NH₄OH, then the mixture was concentrated under reduced pressure. Purification (MPLC, C18 Flash Column, Agela Technologies, 12 g, 12 mL/min, ACN:Water=85:15) afforded N-(9-((1S,2R,3R,E)-2,3-bis((benzyloxy)methyl)-4-(fluoromethylene)cyclobutyl)-9H-purin-6-yl)benzamide and its enantiomer as a mixture (470 mg, 792.2 μmol, 91.0% yield, 95% purity) as white solid. ESI-LCMS m/z=564.2 [M+H]⁺.

Step C. N-(9-((1S,3R,4R,E)-2-(Fluoromethylene)-3,4-bis(hydroxymethyl)cyclobutyl)-9H-purin-6-yl)benzamide and its enantiomer. To a solution of a mixture of N-(9-((1S,2R,3R,E)-2,3-bis((benzyloxy)methyl)-4-(fluoromethylene)cyclobutyl)-9H-purin-6-yl)benzamide and its enantiomer (880 mg, 1.6 mmol) in dichloromethane (30 mL) was added boron trichloride (1 M, 1.87 mL) dropwise at −78° C. The reaction mixture was stirred at −78° C. for 1 h. The reaction was quenched by methanol at −78° C., and added TEA to pH=6. The reaction mixture was concentrated under reduced pressure. Purification (MPLC, C18 Flash Column, Agela Technologies, 12 g, 12 mL/min, ACN:Water=34:66) afforded N-(9-((1S,3R,4R,E)-2-(fluoromethylene)-3,4-bis(hydroxymethyl)cyclobutyl)-9H-purin-6-yl)benzamide and its enantiomer as a mixture (450 mg, 1.2 mmol, 72.2% yield, 96% purity) as white solid. ESI-LCMS m/z=384.1[M+H]⁺. ¹H-NMR (400 MHz, DMSO-d₆): δ 8.75 (s, 1H), 8.63 (s, 1H), 8.06-7.54 (m, 5H), 6.99-6.78 (m, 1H), 5.52-5.50 (m, 1H), 4.91-4.83 (m, 2H), 3.86-3.80 (m, 2H), 3.68-3.57 (m, 2H), 3.08-2.97 (m, 2H). The material was further separated by SFC (OD-H, 2 mL/min, CO₂:0.1% DEA in MeOH=65:35), to give N-(9-((1S,3R,4R,E)-2-(fluoromethylene)-3,4-bis(hydroxymethyl)cyclobutyl)-9H-purin-6-yl)benzamide (242 mg) and N-(9-((1R,3S,4S,E)-2-(fluoromethylene)-3,4-bis(hydroxymethyl)cyclobutyl)-9H-purin-6-yl)benzamide (242 mg).

Step D. N-(9-((1S,3R,4R,E)-2-(Fluoromethylene)-3-(hydroxymethyl)-4-(((4-methoxyphenyl)diphenylmethoxy)methyl)cyclobutyl)-9H-purin-6-yl)benzamide and its isomer. To a solution of N-(9-((1S,3R,4R,E)-2-(fluoromethylene)-3,4-bis(hydroxymethyl)cyclobutyl)-9H-purin-6-yl)benzamide (242.0 mg, 631.2 μmol) and pyridine (249.7 mg, 3.2 mmol, 254.3 μL) in dichloromethane (10 mL) was added 4-methoxytriphenylmethyl chloride (214.4 mg, 694.4 μmol) at 0° C. The reaction mixture was warmed to r.t. and stirred for 3 h. The reaction mixture was quenched by methanol, and the mixture was concentrated under reduced pressure. Purification (MPLC, C18 Flash Column, Agela Technologies, 4 g, 4 mL/min, ACN:Water=70:30) and (FCC, SiO₂, DCM:MeOH=100:1) afforded N-(9-((1S,3R,4R,E)-2-(fluoromethylene)-3-(hydroxymethyl)-4-(((4-methoxyphenyl)diphenylmethoxy)methyl)cyclobutyl)-9H-purin-6-yl)benzamide (130 mg, 188.3 μmol, 29.8% yield, 95% purity) and N-(9-((1S,3R,4R,E)-2-(fluoromethylene)-4-(hydroxymethyl)-3-(((4-methoxyphenyl)diphenylmethoxy)methyl)cyclobutyl)-9H-purin-6-yl)benzamide (80 mg, 115.9 μmol, 18.4% yield, 95% purity) as a white solid. ESI-LCMS m/z=656.3 [M+H]⁺.

Step E. ((1R,2S,4R,E)-2-(6-Amino-9H-purin-9-yl)-3-(fluoromethylene)-4-(((4-methoxyphenyl)diphenylmethoxy)methyl)cyclobutyl)methanol. A solution of N-(9-((1S,3R,4R,E)-2-(fluoromethylene)-4-(hydroxymethyl)-3-(((4-methoxyphenyl)diphenylmethoxy)methyl)cyclobutyl)-9H-purin-6-yl)benzamide (50 mg, 76.3 μmol) in methylamine/ethanol (2 mL) was stirred at r.t. for 30 min. The reaction mixture was concentrated under reduced pressure. Purification (MPLC, C18 Flash Column, Agela Technologies, 4 g, 4 mL/min, ACN:Water=50:50) afforded ((1R,2S,4R,E)-2-(6-amino-9H-purin-9-yl)-3-(fluoromethylene)-4-(((4-methoxyphenyl)diphenylmethoxy)methyl)cyclobutyl)methanol (30 mg, 51.7 μmol, 67.8% yield, 95% purity) as a white solid. ESI LC-MS m/z=552.2 [M+H]⁺.

Step F. ((1R,2R,3S,E)-3-(6-Amino-9H-purin-9-yl)-4-(fluoromethylene)cyclobutane-1,2-diyl)dimethanol. A solution of ((1R,2S,4R,E)-2-(6-amino-9H-purin-9-yl)-3-(fluoromethylene)-4-(((4-methoxyphenyl)diphenylmethoxy)methyl)cyclobutyl)methanol (30 mg, 54.4 μmol) in 3% trichloroacetic acid/DCM was stirred at r.t. for 30 min. The reaction mixture was concentrated under reduced pressure. Purification (MPLC, C18 Flash Column, Agela Technologies, 4 g, 4 mL/min, ACN:Water=15:85) afforded ((1R,2R,3S,E)-3-(6-amino-9H-purin-9-yl)-4-(fluoromethylene)cyclobutane-1,2-diyl)dimethanol (10 mg, 35.1 μmol, 64.5% yield, 98% purity) as a white solid. ESI LC-MS m/z=280.1 [M+H]⁺. ¹H-NMR (400 MHz, DMSO-d₆): δ 8.28 (s, 1H), 8.14 (s, 1H), 7.27 (s, 2H), 6.93 (t, J=2.4 Hz, 0.5H), 6.73 (t, J=2.4 Hz, 0.5H), 4.91 (t, J=5.3 Hz, 1H), 4.85 (t, J=5.0 Hz, 1H), 3.81-3.77 (m, 2H), 3.61-3.56 (m, 2H), 2.99-2.96 (m, 2H). ¹⁹FNMR (400 MHz, DMSO-d₆): δ −138.27 (s).

### Example 18: ((1S,2S,3R,E)-3-(6-Amino-9H-purin-9-yl)-4-(fluoromethylene)cyclobutane-1,2-diyl)dimethanol

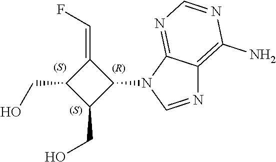

Step A. N-(9-((1R,3S,4S,E)-2-(Fluoromethylene)-3-(hydroxymethyl)-4-(((4-methoxyphenyl)diphenylmethoxy)methyl)cyclobutyl)-9H-purin-6-yl)benzamide and its isomer. To a solution of N-(9-((1R,3S,4S,E)-2-(fluoromethylene)-3,4-bis(hydroxymethyl)cyclobutyl)-9H-purin-6-yl)benzamide (Example 17, Step E.) (242.0 mg, 631.2 μmol) and pyridine (249.7 mg, 3.2 mmol, 254.3 μL) in dichloromethane (10 mL) was added 4-methoxytriphenylmethyl chloride (214.4 mg, 694.4 μmol) at 0° C. The reaction mixture was warmed to r.t. and stirred for 3 h. The reaction mixture was quenched by methanol, and concentrated under reduced pressure. Purification (MPLC, C18 Flash Column, Agela Technologies, 12 g, 12 mL/min, ACN:Water=70:30) and further purification (FCC, SiO₂, DCM:MeOH=100:1) afforded N-(9-((1R,3S,4S,E)-2-(fluoromethylene)-3-(hydroxymethyl)-4-(((4-methoxyphenyl)diphenylmethoxy)methyl)cyclobutyl)-9H-purin-6-yl)benzamide (130 mg, 188.3 μmol, 29.8% yield, 95% purity) and N-(9-((1R,3S,4S,E)-2-(fluoromethylene)-4-(hydroxymethyl)-3-(((4-methoxyphenyl)diphenylmethoxy)methyl)cyclobutyl)-9H-purin-6-yl)benzamide (80 mg, 115.9 μmol, 18.4% yield, 95% purity) as a white solid. ESI-LCMS m/z=656.3[M+H]⁺.

Step B. ((1S,2R,4S,E)-2-(6-Amino-9H-purin-9-yl)-3-(fluoromethylene)-4-(((4-methoxyphenyl)diphenylmethoxy)methyl)cyclobutyl)methanol. A solution of N-(9-((1R,3S,4S,E)-2-(fluoromethylene)-4-(hydroxymethyl)-3-(((4-methoxyphenyl)diphenylmethoxy)methyl)cyclobutyl)-9H-purin-6-yl)benzamide (80 mg, 122.0 μmol) in methylamine/ethanol (2 mL) was stirred at r.t. for 30 min. The reaction mixture was concentrated under reduced pressure. Purification (MPLC, C18 Flash Column, Agela Technologies, 4 g, 4 mL/min, ACN:Water=50:50) afforded 41S,2R,4S,E)-2-(6-amino-9H-purin-9-yl)-3-(fluoromethylene)-4-(((4-methoxyphenyl)diphenylmethoxy)methyl)cyclobutyl)methanol. (50 mg, 85.2 μmol, 69.8% yield, 94% purity) as a white solid. ESI LC-MS m/z=552.2 [M+H]⁺

Step C. ((1S,2S,3R,E)-3-(6-Amino-9H-purin-9-yl)-4-(fluoromethylene)cyclobutane-1,2-diyl)dimethanol. A solution of ((1S,2R,4S,E)-2-(6-amino-9H-purin-9-yl)-3-(fluoromethylene)-4-(((4-methoxyphenyl)diphenylmethoxy)methyl)cyclobutyl)methanol (50 mg, 90.6 μmol) in 3% trichloroacetic acid/DCM (2 mL) was stirred at r.t. for 30 min. The reaction mixture was concentrated under reduced pressure. Purification (MPLC, C18 Flash Column, Agela Technologies, 4 g, 4 mL/min, ACN:Water=15:85) afforded 41S,2S,3R,E)-3-(6-amino-9H-purin-9-yl)-4-(fluoromethylene)cyclobutane-1,2-diyl)dimethanol (10 mg, 35.1 μmol, 38.7% yield, 98% purity) as a white solid. ESI LC-MS m/z=280.1 [M+H]⁺. ¹H-NMR (400 MHz, DMSO-d₆): δ 8.38 (s, 1H), 8.18 (s, 1H), 7.33 (s, 1H), 7.26-7.19 (m, 10H), 7.16-7.13 (m, 2H), 6.95 (t, J=2.4 Hz, 0.5H), 6.84-6.82 (m, 2H), 6.75 (t, J=2.4 Hz, 0.5H), 5.50-5.49 (m, 1H), 4.87 (t, J=5.4 Hz, 1H), 3.78-3.75 (m, 2H), 3.72 (s, 3H), 3.22-3.20 (m, 2H), 3.14-3.12 (m, 1H), 2.95 (s, 1H). ¹⁹FNMR (400 MHz, DMSO-d₆): δ −138.28 (s). For Examples 15-18, configurations were assigned arbitrarily.

### Example 19: ((1S,2R,3S)-3-(6-Amino-9H-purin-9-yl)-1-ethynyl-4-methylenecyclobutane-1,2-diyl)dimethanol

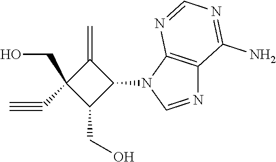 [SMILES: C#C[C@]1(CO)C(=C)[C@H](n2cnc3c(N)ncnc32)[C@@H]1CO]

Step A. 9-((1S,2R,3R,4S)-2,3-Bis((benzyloxy)methyl)-4-diethoxymethyl)cyclobutyl)-N,N-Di-Boc-9H-purin-6-amine. To a suspension of (1R,2S,3R,4S)-2,3-bis((benzyloxy)methyl)-4-(diethoxymethyl)cyclobutanol (Intermediate 4, 20 g, 48.2 mmol), N,N-diboc-9H-purin-6-amine (32.4 g, 96.5 mmol) and PPh₃ (25.3 g, 96.5 mmol) in THF (300 mL) was added DIAD (19.5 g, 96.5 mmol) at 0° C. under N₂. The resulting mixture was stirred at 50° C. for 18 h. The reaction mixture was concentrated in vacuo. Purification (FCC, SiO₂, EA:PE=1:10 to 1:3) afforded 56 g. Purification (MPLC, C18 Flash Column, Agela Technologies, 800 g, 200 mL/min, ACN:H₂O=80:20) afforded 9-((1S,2R,3R,4S)-2,3-Bis((benzyloxy)methyl)-4-(diethoxymethyl)cyclobutyl)-N,N-Di-Boc-9H-purin-6-amine (23.9 g, 32.3 mmol, 67% yield, 99% purity) as yellow oil. ESI-LCMS m/z=732.3 [M+H]⁺

Step B. (1S,2S,3R,4R)-2-(6-Amino-9H-purin-9-yl)-3,4-bis((benzyloxy)methyl)cyclobutanecarbaldehyde. To a solution of 9-((1S,2R,3R,4S)-2,3-Bis((benzyloxy)methyl)-4-(diethoxymethyl)cyclobutyl)-N,N-Di-Boc-9H-purin-6-amine (23.9 g, 32.7 mmol) in DCM (150 mL) was added TFA (50 mL) at r.t. The resulting mixture stirred at r.t. for 1.5 h. The reaction mixture was diluted with DCM (500 mL), and washed with water (250 mL). The organic phase was washed with sat. NaHCO₃ and brine, and concentrated in vacuo to give crude (1S,2S,3R,4R)-2-(6-amino-9H-purin-9-yl)-3,4-bis((benzyloxy)methyl)cyclobutanecarbaldehyde (14.9 g, 32.6 mmol, 99.7% yield) as white solid. ESI-LCMS m/z=458.3 [M+H]⁺.

Step C. ((1S,2R,3R,4S)-2-(6-Amino-9H-purin-9-yl)-3,4-bis((benzyloxy)methyl)cyclobutyl)methanol. To a solution of (1S,2S,3R,4R)-2-(6-amino-9H-purin-9-yl)-3,4-bis((benzyloxy)methyl)cyclobutanecarbaldehyde (14.9 g, 32.6 mmol) in a mixture of THF (50 mL) and MeOH (100 mL) was added NaBH₄ (1.85 g, 48.9 mmol) in portions at 0° C. After stirred at 0° C. for 0.5 h. The reaction mixture was quenched with 1 N HCl, diluted with water (300 mL), extracted with DCM (300 ml×2). The combined organic extracts were washed with brine, and concentrated in vacuo. Purification (MPLC, C18 Flash Column, Agela Technologies, 330 g, 100 mL/min, ACN:H₂O=30:70) afforded ((1S,2R,3R,4S)-2-(6-amino-9H-purin-9-yl)-3,4-bis((benzyloxy)methyl)cyclobutyl)methanol (11.3 g, 24.6 mmol, 76% yield) as colorless oil.

Step D. 9-((1S,2R,3R,4S)-2,3-Bis((benzyloxy)methyl)-4-(((tert-butyldiphenylsilyl)oxy)methyl)cyclobutyl)-9H-purin-6-amine. To a solution of ((1S,2R,3R,4S)-2-(6-amino-9H-purin-9-yl)-3,4-bis((benzyloxy)methyl)cyclobutyl)methanol (23 g, 50.1 mmol) in DCM (250 mL) was added tert-butylchlorodiphenylsilane (41.27 g, 150.15 mmol) at 0° C. The reaction mixture was allowed warm to r.t. and stirred for 3 h. The reaction was quenched with methanol, diluted with water and extracted with EA. The organic layer was washed with brine and dried over sodium sulfate, and concentrated under reduced pressure. Purification (FCC, SiO₂, DCM:MeOH=100:1) afforded 9-((1S,2R,3R,4S)-2,3-bis((benzyloxy)methyl)-4-(((tert-butyldiphenylsilyl)oxy)methyl)cyclobutyl)-9H-purin-6-amine (33 g, 42.55 mmol, 85.0% yield, 90% purity) as white solid. ESI-LCMS m/z=698.3 [M+H]⁺⁹

Step E. N-(9-((1S,2R,3R,4S)-2,3-Bis((benzyloxy)methyl)-4-(((tert-butyldiphenylsilyl)oxy)methyl)cyclobutyl)-9H-purin-6-yl)benzamide. To a solution of 9-((1S,2R,3R,4S)-2,3-bis((benzyloxy)methyl)-4-(((tert-butyldiphenylsilyl)oxy)methyl)cyclobutyl)-9H-purin-6-amine (33 g, 47.28 mmol) in pyridine (250 mL) was added benzoyl chloride (7.98 g, 56.74 mmol) dropwise via a syringe at 0° C. After stirred at r.t. for 2 h. The reaction mixture was quenched with methanol, followed by addition of ammonium hydroxide (4 mL). The mixture was stirred at r.t. for 30 min, then diluted with water and extracted with EA. The organic layer was washed with brine and dried over sodium sulfate, and concentrated under reduced pressure. Purification (FCC, SiO₂, DCM:MeOH=200:1) afforded N-(9-((1S,2R,3R,4S)-2,3-bis((benzyloxy)methyl)-4-(((tert-butyldiphenylsilyl)oxy)methyl)cyclobutyl)-9H-purin-6-yl)benzamide (36 g, 41.29 mmol, 87.3% yield, 92% purity) as white solid. ESI-LCMS m/z=802.4 [M+H]⁺

Step F. N-(9-((1S,2S,3R,4R)-2-(((tert-Butyldiphenylsilyl)oxy)methyl)-3,4-bis(hydroxymethyl)cyclobutyl)-9H-purin-6-yl)benzamide. To a solution of N-(9-((1S,2R,3R,4S)-2,3-bis((benzyloxy)methyl)-4-(((tert-butyldiphenylsilyl)oxy)methyl)cyclobutyl)-9H-purin-6-yl)benzamide (31 g, 38.65 mmol) in dichloromethane (200 mL) was added boron trichloride (1 M, 309.21 mL) dropwise at −78° C. The reaction mixture was stirred at −78° C. for 1 h. The reaction was quenched with methanol at −78° C., then TEA was added to the reaction mixture to adjust the pH of the reaction mixture to pH=6. The solvent was removed in vacuo. Purification (MPLC, C18 Flash Column, Agela Technologies, 330 g, 100 mL/min, ACN:H₂O=50:50) afforded N-(9-((1S,2S,3R,4R)-2-(((tert-butyldiphenylsilyl)oxy)methyl)-3,4-bis(hydroxymethyl)cyclobutyl)-9H-purin-6-yl)benzamide (20 g, 30.56 mmol, 79.1% yield, 95% purity) as white solid. ESI-LCMS m/z=622.2[M+H]⁺, ¹H-NMR (400 MHz, DMSO-d₆): δ 11.12 (s, 1H), 8.72 (s, 1H), 8.62 (s, 1H), 8.05-8.03 (m, 2H), 7.66-7.62 (m, 1H), 7.58-7.53 (m, 6H), 7.43-7.32 (m, 6H), 4.81 (t, J=8.8 Hz, 1H), 4.70 (t, J=4.9 Hz, 1H), 4.64 (t, J=5.2 Hz, 1H), 3.84-3.73 (m, 2H), 3.62-3.54 (m, 4H), 2.90-2.86 (m, 1H), 2.78-2.74 (m, 1H), 2.11-2.07 (m, 1H), 0.90 (s, 9H).

Step G. N-(9-((1S,2S,3R,4R)-2-(((tert-Butyldiphenylsilyl)oxy)methyl)-3-(hydroxymethyl)-4-(((4-methoxyphenyl)diphenylmethoxy)methyl)cyclobutyl)-9H-purin-6-yl)benzamide. To a solution of N-(9-((1S,2S,3R,4R)-2-(((tert-butyldiphenylsilyl)oxy)methyl)-3,4-bis(hydroxymethyl)cyclobutyl)-9H-purin-6-yl)benzamide (20 g, 32.16 mmol) and pyridine (12.72 g, 160.82 mmol, 12.96 mL) in dichloromethane (200 mL) was added 4-methoxytriphenylmethyl chloride (9.93 g, 32.16 mmol) at 0° C. The reaction mixture was warmed to r.t. and stirred for 3 h. The reaction mixture was quenched with methanol, and concentrated under reduced pressure. Purification (MPLC, C18 Flash Column, Agela Technologies, 50 g, 50 mL/min, ACN:H₂O=86:14) afforded N-(9-((1S,2S,3R,4R)-2-(((tert-butyldiphenylsilyl)oxy)methyl)-3-(hydroxymethyl)-4-(((4-methoxyphenyl)diphenylmethoxy)methyl)cyclobutyl)-9H-purin-6-yl)benzamide (4.5 g, 4.63 mmol, 14.4% yield, 92% purity) as white solid. ESI-LCMS m/z=894.3 [M+H]⁺, NMR (400 MHz, DMSO-d₆): δ 11.23 (s, 1H), 8.80 (s, 1H), 8.75 (s, 1H), 8.10-8.08 (m, 2H), 7.68-7.64 (m, 1H), 7.59-7.57 (m, 6H), 7.22-7.21 (m, 10H), 7.10-7.08 (m, 2H), 6.82-6.80 (m, 2H), 5.00 (t, J=8.8 Hz, 1H), 4.66 (t, J=5.2 Hz, 1H), 3.80-3.71 (m, 5H), 3.59-3.56 (m, 2H), 3.23-3.11 (m, 2H), 3.00-2.92 (m, 1H), 2.90-2.84 (m, 1H), 2.10-2.06 (m, 1H), 0.88 (s, 9H).

Step H. N-(9-((1S,2S,3R,4R)-2-(((tert-Butyldiphenylsilyl)oxy)methyl)-3-formyl-4-(((4-methoxyphenyl)diphenylmethoxy)methyl)cyclobutyl)-9H-purin-6-yl)benzamide. To a solution of N-(9-((1S,2S,3R,4R)-2-(((tert-butyldiphenylsilyl)oxy)methyl)-3-(hydroxymethyl)-4-(((4-methoxyphenyl)diphenylmethoxy)methyl)cyclobutyl)-9H-purin-6-yl)benzamide (7.0 g, 7.83 mmol) in dichloromethane (100 mL) was added Dess-Martin periodinane (4.98 g, 11.74 mmol) in portions at 0° C. The reaction mixture was allowed warmed to r.t. and stirred for 1 h. The reaction mixture was quenched by saturated solution of sodium bicarbonate, and extracted with DCM. The organic layer was separated and washed with brine, dried over sodium sulfate, filtered and concentrated under reduced pressure. Purification (MPLC, C18 Flash Column, Agela Technologies, 50 g, 50 mL/min, ACN=100%) afforded N-(9-((1S,2S,3R,4R)-2-(((tert-butyldiphenylsilyl)oxy)methyl)-3-formyl-4-(((4-methoxyphenyl)diphenylmethoxy)methyl)cyclobutyl)-9H-purin-6-yl)benzamide (5.44 g, 4.70 mmol, 60.0% yield, 77% purity) as white solid. ESI-LCMS m/z=892.3[M+H]⁺.

Step I. N-(9-((1S,2S,4R)-2-(((tert-Butyldiphenylsilyl)oxy)methyl)-3-formyl-3-(hydroxymethyl)-4-(((4-methoxyphenyl)diphenylmethoxy)methyl)cyclobutyl)-9H-purin-6-yl)benzamide. To a solution of N-(9-((1S,2S,3R,4R)-2-(((tert-butyldiphenylsilyl)oxy)methyl)-3-formyl-4-(((4-methoxyphenyl)diphenylmethoxy)methyl)cyclobutyl)-9H-purin-6-yl)benzamide (5.44 g, 6.10 mmol) in dioxane (81 mL) was added formaldehyde (12 M, 40.67 mL) and NaOH (2 M, 48.80 mL) at r.t. The reaction mixture was stirred at 40° C. for 16 h. The reaction mixture was quenched with saturated solution of ammonium chloride, and extracted with EA. The organic layers were combined and concentrated under reduced pressure. afforded N-(9-((1S,2S,4R)-2-(((tert-butyldiphenylsilyl)oxy)methyl)-3-formyl-3-(hydroxymethyl)-4-(((4-methoxyphenyl)diphenylmethoxy)methyl)cyclobutyl)-9H-purin-6-yl)benzamide as white solid (8.0 g crude) ESI-LCMS m/z=922.2[M+H]⁺.

Step J. N-(9-((1S,2S,4R)-2-(((tert-Butyldiphenylsilyl)oxy)methyl)-3,3-bis(hydroxyl methyl)-4-(((4-methoxyphenyl)diphenylmethoxy)methyl)cyclobutyl)-9H-purin-6-yl)benzamide. The resulting N-(9-((1S,2S,4R)-2-(((tert-butyldiphenylsilyl)oxy)methyl)-3-formyl-3-(hydroxymethyl)-4-(((4-methoxyphenyl)diphenylmethoxy)methyl)cyclobutyl)-9H-purin-6-yl)benzamide was redissolved in dioxane (81 mL). NaBH₄ (1.85 g, 48.80 mmol) was added at 0° C. and stirred for 30 min. The reaction mixture was then diluted with saturated solution of ammonium chloride, and extracted with EA. The organic phase was washed with brine and concentrated in vacuo. afforded N-(9-((1S,2S,4R)-2-(((tert-butyldiphenylsilyl)oxy)methyl)-3,3-bis(hydroxymethyl)-4-(((4-methoxyphenyl)diphenylmethoxy)methyl)cyclobutyl)-9H-purin-6-yl)benzamide (crude 8.2 g) as white solid, ESI-LCMS m/z=924.2[M+H]⁺, including formylated byproduct.

Step K. ((2S,3S,4R)-3-(6-Amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol, (including formylated byproduct). The resulting N-(9-((1S,2S,4R)-2-(((tert-butyldiphenylsilyl)oxy)methyl)-3,3-bis(hydroxymethyl)-4-(((4-methoxyphenyl)diphenylmethoxy)methyl)cyclobutyl)-9H-purin-6-yl)benzamide, including formylated byproduct, was dissolved in CH₃NH₂/C₂H₅OH and stirred for 30 min to remove the Bz protecting group. The reaction mixture was concentrated under reduced and purified (MPLC, C18 Flash Column, Agela Technologies, 50 g, 25 mL/min, ACN:H₂O=54:46). The collected fraction was dried in vacuo to afford ((2S,3S,4R)-3-(6-amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol (3.1 g) as white solid, ESI-LCMS m/z=820.2[M+H]⁺, including formylated byproduct.

Step L. ((2S,3S,4R)-3-(6-Amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol. The resulting ((2S,3S,4R)-3-(6-amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol (including formylated byproduct) was redissolved in MeCN/water, the mixture was stirred at 80° C. for 1 h to remove the formaldehyde moiety. Then the solvent was removed under reduce pressure to give ((2S,3S,4R)-3-(6-amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol (2.2 g, 2.31 mmol, 37.8% yield, 86% purity) as white solid. ESI-LCMS m/z=820.4 [M+H]⁺, ¹H-NMR (400 MHz, DMSO-d₆): δ 8.43 (s, 1H), 8.11 (s, 1H), 7.50-7.27 (m, 11H), 7.20-7.05 (m, 12H), 6.99-6.97 (m, 2H), 6.77-6.75 (m, 2H), 5.01 (t, J=9.4 Hz, 1H), 4.66 (t, J=5.3 Hz, 1H), 4.49 (t, J=3.8 Hz, 1H), 3.89-380 (m, 2H), 3.72 (S, 3H), 3.56-3.55 (m, 2H), 3.48-2.46 (m, 2H), 3.36-3.33 (m, 1H), 3.12-3.08 (m, 1H), 3.06-2.96 (m, 2H), 0.75 (s, 9H).

Step M. N-(9-((1S,2S,4R)-2-(((tert-Butyldiphenylsilyl)oxy)methyl)-3,3-bis(hydroxyl methyl)-4-(((4-methoxyphenyl)diphenylmethoxy)methyl)cyclobutyl)-9H-purin-6-yl)benzamide. To a solution of ((2S,3S,4R)-3-(6-amino-9H-purin-9-yl)-2-(((tert-butyldiphenylsilyl)oxy)methyl)-4-(((4-methoxyphenyl)diphenylmethoxy)methyl)cyclobutane-1,1-diyl)dimethanol (2.2 g, 2.68 mmol) in pyridine (30 mL) was added trimethylchlorosilane (1.17 g, 10.73 mmol, 1.38 mL) dropwise via a syringe at 0° C. The reaction mixture was stirred at r.t. for 1 h, TLC showed the starting material was consumed completely and the 9-((1S,2S,4R)-2-(((tert-butyldiphenylsilyl)oxy)methyl)-4-(((4-methoxyphenyl)diphenylmethoxy)methyl)-3,3-bis(((trimethylsilyl)oxy)methyl)cyclobutyl)-9H-purin-6-amine was formed. Then benzoyl chloride (1.51 g, 10.73 mmol, 1.25 mL) was added dropwise via syringe at 0° C., and stirred at r.t. for another 3 h. TLC showed the intermediate A was consumed completely. The reaction was quenched by methanol, followed by 0.5 mL of ammonium hydroxide. The mixture was stirred at r.t. for 30 min. The reaction was then diluted with water, extracted with EA. The combined organic layer was washed with brine, dried over sodium sulfate and concentrated in vacuo. afforded N-(9-((1S,2S,3S,4R)-2-(((tert-butyldiphenylsilyl)oxy)methyl)-3-(hydroxymethyl)-4-(((4-methoxyphenyl)diphenylmethoxy)methyl)-3-(((trimethylsilyl)oxy)methyl)cyclobutyl)-9H-purin-6-yl)benzamide (1.8 g crude) as yellow solid. ESI-LCMS m/z=996.4[M+H]⁺

Step N. N-(9-((1S,2S,4R)-2-(((tert-butyldiphenylsilyl)oxy)methyl)-3,3-bis(hydroxymethyl)-4-(((4-methoxyphenyl)diphenylmethoxy)methyl)cyclobutyl)-9H-purin-6-yl)benzamide. N-(9-((1S,2S,3S,4R)-2-(((tert-butyldiphenylsilyl)oxy)methyl)-3-(hydroxymethyl)-4-(((4-methoxyphenyl)diphenylmethoxy)methyl)-3-(((trimethylsilyl)oxy)methyl)cyclobutyl)-9H-purin-6-yl)benzamide (1.8 g) was dissolved in 1N NaOH at 0° C., and stirred for 20 min to remove all the TMS protecting group. The reaction was then quenched by AcOH, diluted with water, extracted with EA. The combined organic layer was washed with saturated solution of sodium bicarbonate and brine, dried over sodium sulfate and concentrated, the residue was purified (A/PLC, C18 Flash Column, Agela Technologies, 20 g, 20 mL/min, ACN:H₂O=65:35) to give N-(9-((1S,2S,4R)-2-(((tert-butyldiphenylsilyl)oxy)methyl)-3,3-bis(hydroxymethyl)-4-(((4-methoxyphenyl)diphenylmethoxy)methyl)cyclobutyl)-9H-purin-6-yl)benzamide (1.37 g, 1.33 mmol, 49.7% yield, 90% purity) as white solid. ESI-LCMS m/z=924.2 [M+H]⁺

Step O. N-(9-((1S,2S,3R,4R)-3-((Bis(4-methoxyphenyl)(phenyl)methoxy)methyl)-2-(((tert-butyldiphenylsilyl)oxy)methyl)-3-(hydroxymethyl)-4-(((4-methoxyphenyl)diphenylmethoxy)methyl)cyclobutyl)-9H-purin-6-yl)benzamide. To a solution of N-(9-((1S,2S,4R)-2-(((tert-butyldiphenylsilyl)oxy)methyl)-3,3-bis(hydroxymethyl)-4-(((4-methoxyphenyl)diphenylmethoxy)methyl)cyclobutyl)-9H-purin-6-yl)benzamide (1.37 g, 1.48 mmol) in pyridine (15 mL) was added 4,4′-dimethoxytrityl chloride (1 g, 2.96 mmol) at 0° C. The reaction mixture was warmed to r.t. and stirred for an additional 3 h. The reaction was quenched with methanol and concentrated under reduced pressure. Purification (MPLC, C18 Flash Column, Agela Technologies, 20 g, mL/min, ACN:H₂O=100:0) afforded N-(9-((1S,2S,3R,4R)-3-((bis(4-methoxyphenyl)(phenyl)methoxy)methyl)-2-(((tert-butyldiphenylsilyl)oxy)methyl)-3-(hydroxymethyl)-4-(((4-methoxyphenyl)diphenylmethoxy)methyl)cyclobutyl)-9H-purin-6-yl)benzamide (1.4 g, 1.03 mmol, 69.3% yield, 90% purity) as white solid. ESI-LCMS m/z=1226.3 [M+H]⁺, ¹H-NMR (400 MHz, DMSO-d₆): δ 11.23 (s, 1H) 8.64 (s, 1H), 8.62 (s, 1H), 8.01-8.08 (m, 2H), 7.67-7.64 (m, 1H), 7.58-7.55 (m, 2H), 7.44-7.04 (m, 33H), 6.96-6.73 (m, 9H), 5.14 (t, J=9.3 Hz, 1H), 4.66 (t, J=5.3 Hz, 1H), 3.89-3.87 (m, 2H), 3.71-3.50 (m, 12H), 3.49-3.45 (m, 1H), 3.26-3.07 (m, 6H), 0.69 (s, 9H).

Step P. N-(9-((1S,2S,3S,4R)-3-((Bis(4-methoxyphenyl)(phenyl)methoxy)methyl)-2-(((tert-butyldiphenylsilyl)oxy)methyl)-3-formyl-4-(((4-methoxyphenyl)diphenylmethoxy)methyl)cyclobutyl)-9H-purin-6-yl)benzamide. To a solution of N-(9-((1S,2S,3R,4R)-3-((bis(4-methoxyphenyl)(phenyl)methoxy)methyl)-2-(((tert-butyldiphenylsilyl)oxy)methyl)-3-(hydroxymethyl)-4-(((4-methoxyphenyl)diphenylmethoxy)methyl)cyclobutyl)-9H-purin-6-yl)benzamide (500 mg, 407.65 μmol) and EDCI (469.62 mg, 2.45 mmol) in DMSO (8 mL) was added TFA (46.48 mg, 407.65 μmol, 27.34 L) and pyridine (64.49 mg, 815.31 μmol, 65.68 μL). The reaction mixture was stirred at r.t. for 16 h. The reaction was quenched with water, and extracted with EA. The organic phase was concentrated in vacuo. Purification (MPLC, C18 Flash Column, Agela Technologies, 12 g, 12 mL/min, ACN:H₂O=100:0) afforded N-(9-((1S,2S,3S,4R)-3-((bis(4-methoxyphenyl)(phenyl)methoxy)methyl)-2-(((tert-butyldiphenylsilyl)oxy)methyl)-3-formyl-4-(((4-methoxyphenyl)diphenylmethoxy)methyl)cyclobutyl)-9H-purin-6-yl)benzamide (492 mg, 373.7 μmol, 91.7% yield, 93% purity) as white solid. ESI-LCMS m/z=1224.3[M+H]⁺

Step Q. 9-((1S,2S,3R,4R)-3-((Bis(4-methoxyphenyl)(phenyl)methoxy)methyl)-2-(((tert-butyldiphenylsilyl)oxy)methyl)-3-ethynyl-4-(((4-methoxyphenyl)diphenylmethoxy)methyl)cyclobutyl)-9H-purin-6-amine. To a solution of N-(9-((1S,2S,3S,4R)-3-((bi s (4-methoxyphenyl)(phenyl)methoxy)methyl)-2-(((tert-butyldiphenylsilyl)oxy)methyl)-3-formyl-4-(((4-methoxyphenyl)diphenylmethoxy)methyl)cyclobutyl)-9H-purin-6-yl)benzamide (492 mg, 401.79 pimp and potassium carbonate (166.59 mg, 1.21 mmol) in methanol (8 mL) was added dimethyl (1-diazo-2-oxopropyl)phosphonate (192.97 mg, 1 mmol) at 0° C. The reaction mixture was stirred at r.t. for 3 h. The reaction was quenched with saturated solution of sodium bicarbonate, and extracted with EA. The combined organic phase was concentrated in vacuo. Purification (MPLC, C18 Flash Column, Agela Technologies, 12 g, 12 mL/min, ACN:H₂O=100:0) afforded 9-((1S,2S,3R,4R)-3-((bi s (4-methoxyphenyl)(phenyl)methoxy)methyl)-2-(((tert-butyldiphenylsilyl)oxy)methyl)-3-ethynyl-4-(((4-methoxyphenyl)diphenylmethoxy)methyl)cyclobutyl)-9H-purin-6-amine (380 mg, 306.3 μmol, 76.2% yield, 90% purity) as a white solid. ESI-LCMS m/z=1116.2 [M+H]⁺.

Step R. N-(9-((1S,2S,3R,4R)-3-((Bis(4-methoxyphenyl)(phenyl)methoxy)methyl)-2-(((tert-butyldiphenylsilyl)oxy)methyl)-3-ethynyl-4-(((4-methoxyphenyl)diphenyl methoxy)methyl)cyclobutyl)-9H-purin-6-yl)benzamide. To a solution of 9-((1S,2S,3R,4R)-3-((bis(4-methoxyphenyl)(phenyl)methoxy)methyl)-2-(((tert-butyldiphenylsilyl)oxy)methyl)-3-ethynyl-4-(((4-methoxyphenyl)diphenylmethoxy)methyl)cyclobutyl)-9H-purin-6-amine (696 mg, 623.42 μmol) in pyridine was added benzoyl chloride (175.26 mg, 1.25 mmol, 144.73 μL) dropwise via a syringe at 0° C. The reaction mixture was stirred at r.t. for 1 h. Ammonium hydroxide (3 mL) was added to the reaction mixture, and the reaction mixture was stirred at r.t. for an additional 30 min. The reaction was diluted with water and extracted with EA. The organic layer was washed with brine, dried over sodium sulfate, filtered, and concentrated under reduced pressure. Purification (MPLC, C18 Flash Column, Agela Technologies, 12 g, 12 mL/min, ACN:H₂O=100:0) afforded N-(9-((1S,2S,3R,4R)-3-((bis(4-methoxyphenyl)(phenyl)methoxy)methyl)-2-(((tert-butyldiphenylsilyl)oxy)methyl)-3-ethynyl-4-(((4-methoxyphenyl)diphenyl methoxy)methyl)cyclobutyl)-9H-purin-6-yl)benzamide (730 mg, 598.1 μmol, 95.9% yield) as white solid. ESI-LCMS m/z=1221.5 [M+H]⁺.

Step S. N-(9-((1R,2S,3S,4R)-3-((Bis(4-methoxyphenyl)(phenyl)methoxy)methyl)-3-ethynyl-2-(hydroxymethyl)-4-(((4-methoxyphenyl)diphenylmethoxy)methyl)cyclobutyl)-9H-purin-6-yl)benzamide. To a solution of N-(9-((1S,2S,3R,4R)-3-((bis(4-methoxyphenyl)(phenyl)methoxy)methyl)-2-(((tert-butyldiphenylsilyl)oxy)methyl)-3-ethynyl-4-(((4-methoxyphenyl)diphenylmethoxy)methyl)cyclobutyl)-9H-purin-6-yl)benzamide (730 mg, 598.1 μmol) in tetrahydrofuran (15 mL) was added TBAF (1 M, 2.39 mL) at 0° C. The reaction mixture was stirred at r.t. for 16 h. The reaction mixture was quenched with water and extracted with EA. The organic phase was washed with brine, dried over sodium sulfate, filtere, and concentrated in vacu. Purification (MPLC, C18 Flash Column, Agela Technologies, 12 g, 12 mL/min, ACN:H₂O 75:25) afforded N-(9-((1R,2S,3S,4R)-3-((bis(4-methoxyphenyl)(phenyl)methoxy)methyl)-3-ethynyl-2-(hydroxymethyl)-4-(((4-methoxyphenyl)diphenylmethoxy)methyl)cyclobutyl)-9H-purin-6-yl)benzamide (562 mg, 572.2 μmol, 95.7% yield) as white solid. ESI-LCMS m/z=982.4 [M+H]⁺.

Step T. N-(9-((1S,2R,3R,4S)-3-((Bis(4-methoxyphenyl)(phenyl)methoxy)methyl)-3-ethynyl-2-(((4-methoxyphenyl)diphenylmethoxy)methyl)-4-(((2-nitrophenyl)selanyl)methyl)cyclobutyl)-9H-purin-6-yl)benzamide. To a solution of N-(9-((1R,2S,3S,4R)-3-((bis(4-methoxyphenyl)(phenyl)methoxy)methyl)-3-ethynyl-2-(hydroxymethyl)-4-(((4-methoxyphenyl)diphenylmethoxy)methyl)cyclobutyl)-9H-purin-6-yl)benzamide (562 mg, 572.23 μmol) and 1-nitro-2-selenocyanatobenzene (285.87 mg, 1.26 mmol) in tetrahydrofuran (10 mL) was added tributylphosphine (254.70 mg, 1.26 mmol) dropwise via a syringe at 0° C. The reaction mixture was stirred at r.t. for 1 h. The reaction mixture was diluted with water, and extracted with EA. The organic layer was washed with brine, dried over sodium sulfate, filtered then concentrated under reduced pressure. Purification (MPLC, C18 Flash Column, Agela Technologies, 12 g, 12 mL/min, ACN:H₂O=95:5) afforded N-(9-((1S,2R,3R,4S)-3-((bis(4-methoxyphenyl)(phenyl)methoxy)methyl)-3-ethynyl-2-(((4-methoxyphenyl)diphenylmethoxy)methyl)-4-(((2-nitrophenyl)selanyl)methyl)cyclobutyl)-9H-purin-6-yl)benzamide (856 mg, 513.8 μmol, 89.8% yield, 70% purity) as yellow solid. ESI-LCMS m/z=1167.3 [M+H]⁺.

Step U. N-(9-((1S,2R,3S)-3-((Bis(4-methoxyphenyl)(phenyl)methoxy)methyl)-3-ethynyl-2-(((4-methoxyphenyl)diphenylmethoxy)methyl)-4-methylenecyclobutyl)-9H-purin-6-yl)benzamide. To a solution of N-(9-((1S,2R,3R,4S)-3-((bis(4-methoxy phenyl)(phenyl)methoxy)methyl)-3-ethynyl-2-(((4-methoxyphenyl)diphenylmethoxy)methyl)-4-(((2-nitrophenyl)selanyl)methyl)cyclobutyl)-9H-purin-6-yl)benzamide (856 mg, 513.81 μmol) in tetrahydrofuran (10 mL) was added H₂O₂ (17.48 mg, 513.8 μmol, 1.3 mL). The reaction mixture was stirred at 50° C. for 2 h. The reaction mixture was quenched with saturated solution of sodium sulfite, and extracted with EA. The organic layer was washed with brine, dried over sodium sulfate, filtered and concentrated under reduced pressure. Purification (MPLC, C18 Flash Column, Agela Technologies, 12 g, 12 mL/min, ACN:H₂O=90:10) afforded N-(9-((1S,2R,3S)-3-((bis(4-methoxyphenyl)(phenyl)methoxy)methyl)-3-ethynyl-2-(((4-methoxyphenyl)diphenylmethoxy)methyl)-4-methylenecyclobutyl)-9H-purin-6-yl)benzamide (390 mg, 384.3 μmol, 74.8% yield, 95% purity) as yellow solid. ESI-LCMS m/z=964.3 [M+H]⁺, ¹H-NMR (400 MHz, DMSO-d₆): δ 11.23 (s, 1H), 8.50 (s, 1H), 8.44 (s, 1H), 8.07-8.05 (m, 2H), 7.66-7.63 (m, 1H), 7.57-7.54 (m, 2H), 7.40-7.38 (m, 2H), 7.27-7.15 (m, 20H), 6.87-6.84 (m, 4H), 6.75-6.73 (m, 2H), 5.59-5.56 (m, 1H), 5.16 (s, 1H), 4.92 (t, J=1.8 Hz, 1H), 3.74 (s, 6H), 3.68 (s, 3H), 3.57-3.53 (m, 2H), 3.44-3.41 (m, 2H), 3.31 (s, 1H), 3.25-3.19 (m, 1H).

Step V. N-(9-((1S,2R,3S)-3-Ethynyl-2,3-bis(hydroxymethyl)-4-methylenecyclobutyl)-9H-purin-6-yl)benzamide. A solution of N-(9-((1S,2R,3S)-3-((bis(4-methoxyphenyl)(phenyl)methoxy)methyl)-3-ethynyl-2-(((4-methoxyphenyl)diphenylmethoxy)methyl)-4-methylenecyclobutyl)-9H-purin-6-yl)benzamide (390 mg, 404.52 μmol) in 10% TCA dichloromethane (10 mL) was stirred at r.t. for 30 min. The reaction mixture was quenched with saturated solution of sodium bicarbonate, and extracted with DCM. The organic layer was washed with brine, dried over sodium sulfate, filtered, and concentrated under reduced pressure. Purification (MPLC, C18 Flash Column, Agela Technologies, 12 g, 12 mL/min, ACN:H₂O=62:38) afforded N-(9-((1S,2R,3S)-3-ethynyl-2,3-bis(hydroxymethyl)-4-methylenecyclobutyl)-9H-purin-6-yl)benzamide (140 mg, 359.5 μmol, 88.9% yield) as white solid. ESI-LCMS m/z=390.1 [M+H]⁺.

Step W. N-(9-((1S,2R,3S)-3-Ethynyl-3-(hydroxymethyl)-2-(((4-methoxyphenyl)diphenylmethoxy)methyl)-4-methylenecyclobutyl)-9H-purin-6-yl)benzamide. To a solution of N-(9-((1S,2R,3S)-3-ethynyl-2,3-bis(hydroxymethyl)-4-methylene cyclobutyl)-9H-purin-6-yl)benzamide (140 mg, 359.52 μmol) and pyridine (142.19 mg, 1.80 mmol, 144.81 μL) in dichloromethane (8 mL) was added 4-methoxy triphenylmethyl chloride (111.0 mg, 359.5 μmol) at 0° C. The reaction mixture was warmed to r.t. and stirred for an additional 2 h. The reaction mixture was quenched with methanol, and the reaction mixture was concentrated in vacuo. Purification (MPLC, C18 Flash Column, Agela Technologies, 12 g, 12 mL/min, ACN:H₂O=86:14) afforded N-(9-((1S,2R,3S)-3-ethynyl-3-(hydroxyl methyl)-2-(((4-methoxyphenyl)diphenylmethoxy)methyl)-4-methylenecyclobutyl)-9H-purin-6-yl)benzamide (230 mg, 312.8 μmol, 87.0% yield, 90% purity) as white solid. ESI-LCMS m/z=662.2 [M+H]⁺, ¹H-NMR (400 MHz, DMSO-d₆): δ 11.27 (s, 1H), 8.70 (s, 1H), 8.65 (s, 1H), 8.09-8.08 (m, 2H), 7.67-7.64 (m, 1H), 7.59-7.55 (m, 2H), 7.24-7.16 (m, 11H), 7.07-7.05 (m, 2H), 6.82-6.80 (m, 2H), 5.58-5.56 (m, 1H), 5.32-5.30 (m, 2H), 5.03 (s, 1H), 3.82-3.76 (m, 1H), 3.73-3.68 (m, 4H), 3.45-3.33 (m, 3H), 3.16 (s, 1H).

Step X. ((1S,2R,3S)-3-(6-Amino-9H-purin-9-yl)-1-ethynyl-2-(((4-methoxy phenyl)diphenylmethoxy)methyl)-4-methylenecyclobutyl)methanol. N-(9-((1S,2R,3S)-3-ethynyl-3-(hydroxymethyl)-2-(((4-methoxyphenyl)diphenylmethoxy)methyl)-4-methylenecyclobutyl)-9H-purin-6-yl)benzamide (230 mg, 347.57 μmol) was dissolved in methylamine/ethanol (3 mL), and stirred at r.t. for 30 min. The reaction mixture was concentrated under reduced pressure. Purification (MPLC, C18 Flash Column, Agela Technologies, 12 g, 12 mL/min, ACN:H₂O=60:40) afforded ((1S,2R,3S)-3-(6-amino-9H-purin-9-yl)-1-ethynyl-2-(((4-methoxyphenyl)diphenylmethoxy)methyl)-4-methylenecyclobutyl)methanol (120 mg, 206.6 μmol, 59.4% yield, 96% purity) as white solid. ESI-LCMS m/z=558.2 [M+H]⁺, ¹H-NMR (400 MHz, DMSO-d₆): δ 8.31 (s, 1H), 8.13 (s, 1H), 7.35 (s, 2H), 7.22-7.16 (m, 10H), 7.08-7.06 (m, 2H), 6.82-6.79 (m, 2H), 5.45-5.43 (m, 1H), 5.29-5.25 (m, 2H), 4.95 (s, 1H), 3.80-3.76 (m, 1H), 3.73 (s, 3H), 3.69-3.65 (m, 1H), 3.42-3.35 (m, 1H), 3.34-3.27 (m, 2H), 3.15 (s, 1H).

Step Y. ((1S,2R,3S)-3-(6-Amino-9H-purin-9-yl)-1-ethynyl-4-methylenecyclobutane-1,2-diyl)dimethanol. A solution of ((1S,2R,3S)-3-(6-Amino-9H-purin-9-yl)-1-ethynyl-2-(((4-methoxyphenyl)diphenylmethoxy)methyl)-4-methylenecyclobutyl)methanol (30 mg, 53.8 μmol) in 10% TCA dichloromethane (1 mL) was stirred at r.t. for 30 min. The reaction mixture was quenched with a saturated solution of sodium bicarbonate, and concentrated in vacuo. Purification (MPLC, C18 Flash Column, Agela Technologies, 4 g, 4 mL/min, ACN:H₂O=19:81) afforded ((1S,2R,3S)-3-(6-amino-9H-purin-9-yl)-1-ethynyl-4-methylenecyclobutane-1,2-diyl)dimethanol (10 mg, 34.4 μmol, 63.9% yield, 98% purity) as white solid. ESI-LCMS m/z=286.1 [M+H]⁺. ¹H-NMR (400 MHz, DMSO-d₆): δ 8.19 (s, 1H), 8.14 (s, 1H), 7.28 (s, 2H), 5.32-5.26 (m, 3H), 4.95 (t, J=2.2 Hz, 1H), 4.64 (s, 1H), 3.78 (t, J=2.76 Hz, 2H), 3.70 (t, J=10.6 Hz, 2H), 3.27 (s, 1H), 3.22-3.16 (m, 1H).

### Example 20: (2S)-Isopropyl 2-(((((1R,3S)-3-(4-amino-2-oxopyrimidin-1(2H)-yl)-2-methylenecyclobutyl)methoxy)(phenoxy)phosphoryl)amino)propanoate

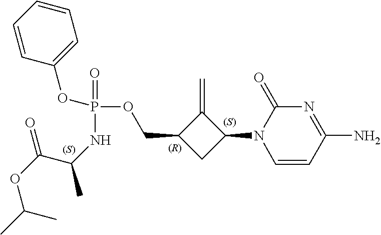

To a solution of 4-amino-1-((1S,3R)-3-(hydroxymethyl)-2-methylenecyclobutyl)pyrimidin-2(1H)-one (Example 1, 100 mg, 0.48 mmol) in THF (2.5 mL) was added t-BuMgCl (1.93 mL, 1.0 M, 1.93 mmol). The reaction mixture was stirred at r.t. under N₂ for 1.0 h. (2S)-Isopropyl 2-(((perfluorophenyl)(phenoxy)phosphoryl)amino)propanoate (263 mg, 0.58 mmol) in THF (1.0 mL) was added dropwise to the reaction mixture. The reaction mixture was stirred at r.t. overnight. The reaction mixture was concentrated under reduced pressure. Purification (MPLC, C18 Flash Column, Agela Technologies, 4 g, 4 mL/min, ACN:0.5‰ HCOOH buffer=46:54) afforded (2S)-isopropyl 2-(((((1R,3S)-3-(4-amino-2-oxopyrimidin-1(2H)-yl)-2-methylenecyclobutyl)methoxy)(phenoxy)phosphoryl)amino)propanoate (20 mg, 9.3% yield) as a mixture of isomers at the phosphorous center (Rₚ and Sₚ). LCMS: m/z=447.2 [M+H]⁺. ¹H NMR (400 MHz, CD₃OD) δ ppm 7.91-8.02 (m, 1H), 7.35-7.39 (m, 2H), 7.18-7.26 (m, 3H), 6.03-6.10 (m, 1H), 5.57-5.60 (m, 1H), 5.23-5.29 (m, 1H), 5.05-5.07 (m, 1H), 4.94-5.01 (m, 1H), 4.32-4.34 (m, 1H), 3.88-3.92 (m, 1H), 2.57-2.65 (m, 1H), 2.13-2.25 (m, 1H), 1.30-1.35 (m, 1H), 1.22-1.24 (m, 1H); ³¹P NMR (162 MHz, CD₃OD) δ ppm 4.05, 3.60, 3.55.

### Example 21: Isopropyl 2-(((((1R,3S)-3-(2-amino-6-oxo-1H-purin-9(6H)-yl)-2-methylenecyclobutyl)methoxy)(phenoxy)phosphoryl)amino)propanoate

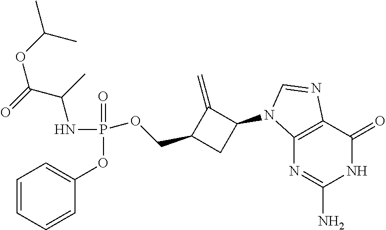 [SMILES: C=C1[C@H](COP(=O)(NC(C)C(=O)OC(C)C)Oc2ccccc2)C[C@@H]1n1cnc2c(=O)[nH]c(N)nc21]

To the solution of 2-amino-9-((1S,3R)-3-(hydroxymethyl)-2-methylenecyclobutyl)-1H-purin-6(9H)-one (Example 2, 80 mg, 323.56 μmol) in THF (2 mL) was added t-BuMgCl (1 M, 1.29 mL) within 10 min at r.t., the mixture was stirred at r.t. for min, isopropyl 2-(((S)-(perfluorophenoxy)(phenoxy)phosphoryl)amino)propanoate (146.67 mg, 323.56 μmol) dissolved in THF (1 mL) was added slowly within 10 min. The mixture was stirred at r.t. overnight. The reaction mixture was quenched with MeOH, and concentrated under reduced pressure. Purification (MPLC, C18 Flash Column, Agela Technologies, 4 g, 4 mL/min, ACN:0.5‰ HCOOH buffer=42:58) afforded isopropyl 2-(((((1R,3S)-3-(2-amino-6-oxo-1H-purin-9(6H)-yl)-2-methylenecyclobutyl)methoxy)(phenoxy)phosphoryl)amino)propanoate (60 mg, 116.17 μmol, 35.90% yield) as a white powder. ¹H NMR (400 MHz, DMSO-d₆) δ 8.34 (s, 1H), 7.36 (t, J=7.7 Hz, 2H), 7.23-7.11 (m, 3H), 5.35 (s, 1H), 5.09 (d, J=10.0 Hz, 1H), 4.92 (s, 1H), 4.89-4.77 (m, 1H), 4.25 (m, 2H), 3.83-3.73 (m, 1H), 3.23 (s, 1H), 2.62 (m, 1H), 2.38 (d, J=10.7 Hz, 1H), 1.22 (t, J=7.0 Hz, 3H), 1.19-1.04 (m, 6H). ESI-LCMS: m/z 517.2 [M+H]⁺.

### Example 22: Isopropyl (2S)-2-(((((1R,3S)-3-(6-amino-9H-purin-9-yl)-2-methylenecyclobutyl)methoxy)(phenoxy)phosphoryl)amino)propanoate

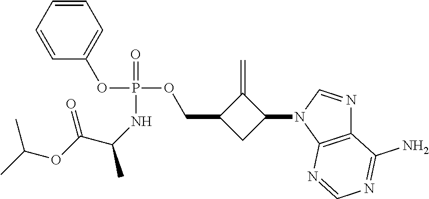 [SMILES: C=C1[C@H](COP(=O)(N[C@@H](C)C(=O)OC(C)C)Oc2ccccc2)C[C@@H]1n1cnc2c(N)ncnc21]

To a solution of ((1R,3S)-3-(6-amino-9H-purin-9-yl)-2-methylenecyclobutyl)methanol (Example 3, 50 mg, 216.22 μmol) in THF (1 mL) was added t-BuMgCl (1 M, 648.66 μL) within 10 min at r.t. The mixture was stirred for 30 min, (2S)-isopropyl 2-(((perfluorophenoxy)(phenoxy)phosphoryl)amino)propanoate (117.62 mg, 259.46 μmol) dissolved in THF (1 mL) as added slowly within 10 min. The mixture was stirred at r.t. overnight. Purification (MPLC, C18 Flash Column, Agela Technologies, 4 g, 4 mL/min, ACN:0.5‰ HCOOH buffer=46:54) afforded isopropyl (2S)-2-(((((1R,3S)-3-(6-amino-9H-purin-9-yl)-2-methylenecyclobutyl) methoxy)(phenoxy)phosphoryl)amino)propanoate as a mixture of isomers at the phosphorous center (Rₚ and Sₚ) (50 mg, 99.9 μmol, 46.2% yield) as a white solid. 41 NMR (400 MHz, CD₃OD) δ 8.29-8.16 (m, 2H), 7.37 (m, 2H), 7.29-7.19 (m, 3H), 5.63 (m, 1H), 5.26-5.16 (m, 1H), 5.03-4.93 (m, 2H), 4.51-4.30 (m, 2H), 4.00-3.86 (m, 1H), 3.33 (s, 1H), 2.79 (m, 1H), 2.50 (s, 1H), 1.40-1.32 (m, 3H), 1.25-1.19 (m, 6H). ESI-LCMS: m/z 501.3 [M+H]⁺.

### Example 23: Isopropyl ((((1S,3R,4S,E)-3-(6-amino-9H-purin-9-yl)-2-(fluoromethylene)-4-(hydroxymethyl)cyclobutyl)methoxy)(phenoxy)phosphoryl)-L-alaninate

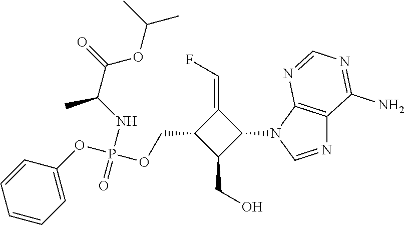 [SMILES: CC(C)OC(=O)[C@H](C)NP(=O)(OC[C@@H]1/C(=C\F)[C@H](n2cnc3c(N)ncnc32)[C@H]1CO)Oc1ccccc1]

Step A. ((1S,3R,4S,E)-3-(6-Amino-9H-purin-9-yl)-2-(fluoromethylene)-4-(((4-methoxyphenyl)diphenylmethoxy)methyl)cyclobutyl)methanol. A solution of N-(9-((1R,3S,4S,E)-2-(fluoromethylene)-3-(hydroxymethyl)-4-(((4-methoxyphenyl)diphenylmethoxy)methyl)cyclobutyl)-9H-purin-6-yl)benzamide (Example 18, Step A, 130 mg, 198.3 μmol) in methylamine/ethanol (3 mL) was stirred at r.t. for 30 min. The reaction mixture was concentrated under reduced pressure. Purification (MPLC, C18 Flash Column, Agela Technologies, 4 g, 4 mL/min, ACN:Water=50:50) afforded 41S,3R,4S,E)-3-(6-amino-9H-purin-9-yl)-2-(fluoromethylene)-4-(((4-methoxyphenyl)diphenylmethoxy)methyl)cyclo butyl)methanol (85 mg, 149.5 μmol, 75.4% yield, 97% purity) as a white solid. ESI LC-MS m/z=552.2 [M+H]⁺. ¹H-NMR (400 MHz, DMSO-d₆): δ 8.38 (s, 1H), 8.18 (s, 1H), 7.33 (s, 1H), 7.26-7.19 (m, 10H), 7.16-7.13 (m, 2H), 6.95 (t, J=2.4 Hz, 0.5H), 6.84-6.82 (m, 2H), 6.75 (t, J=2.4 Hz, 0.5H), 5.50-5.49 (m, 1H), 4.87 (t, J=5.4 Hz, 1H), 3.78-3.75 (m, 2H), 3.72 (s, 3H), 3.22-3.20 (m, 2H), 3.14-3.12 (m, 1H), 2.95 (s, 1H). ¹⁹FNMR (400 MHz, DMSO-d₆): δ −138.87 (s).

Step B: (Z)-N′-(9-((1R,3S,4S,E)-2-(Fluoromethylene)-3-hydroxymethyl)-4-(((4-methoxyphenyl)diphenylmethoxy)methyl)cyclobutyl)-9H-purin-6-yl)-N,N-dimethylformimidamide. To a solution of ((1S,3R,4S,E)-3-(6-amino-9H-purin-9-yl)-2-(fluoromethylene)-4-(((4-methoxyphenyl)diphenylmethoxy)methyl)cyclobutyl)methanol (16 mg, 0.03 mmol) in MeOH (1.0 mL) at r.t. was added dimethylformamide dimethylacetal (0.1 mL, 89.4 mg, 0.75 mmol) under Ar. The reaction mixture was stirred at rt for 16 h. Concentration under reduced pressure afforded (Z)-N′-(9-((1R,3S,4S,E)-2-(fluoromethylene)-3-(hydroxymethyl)-4-(((4-methoxyphenyl)diphenylmethoxy)methyl)cyclobutyl)-9H-purin-6-yl)-N,N-dimethylformimidamide, which was further dried under high vacuum overnight and used crude in the next step without further purification. MS [M+1]=607.15.

Step C: Isopropyl ((((1S,3R,4S,E)-3-(6-(((Z)-(dimethylamino)methylene)amino)-9H-purin-9-yl)-2-(fluoromethylene)-4-(((4-methoxyphenyl)diphenylmethoxy)methyl)cyclobutyl)methoxy)(phenoxy)phosphoryl)-L-alaninate. (Z)-N′-(9-((1R,3S,4S,E)-2-(Fluoromethylene)-3-(hydroxymethyl)-4-(((4-methoxyphenyl)diphenylmethoxy)methyl)cyclobutyl)-9H-purin-6-yl)-N,N-dimethylformimidamide was dissolved in anhydrous THF (0.5 mL), and 1-methylimidazole (NMI) (30 mg, 29 μL, 0.36 mmol) was added at rt. The resulting mixture in the small vial (4 mL) was stirred, and (2S)-isopropyl 2-((chloro(phenoxy)phosphoryl)amino)propanoate (68 mg, 0.22 mmol) was then added. The reaction mixture was stirred at rt for 16 h, and concentrated at 35° C. under vacuum, and then dried under high vacuum. The title compound was used crude in the next step without further purification. MS [M+1]⁺=876.25.

Step D: Isopropyl ((((1S,3R,4S,E)-3-(6-amino-9H-purin-9-yl)-2-(fluoromethylene)-4-(hydroxymethyl)cyclobutyl)methoxy)(phenoxy)phosphoryl)-L-alaninate. To isopropyl ((((1S,3R,4S,E)-3-(6-(((Z)-(dimethylamino)methylene)amino)-9H-purin-9-yl)-2-(fluoromethylene)-4-(((4-methoxyphenyl)diphenylmethoxy)methyl)cyclobutyl)methoxy)(phenoxy)phosphoryl)-L-alaninate was added 0.377 M TFA solution in MeOH—H₂O (2.0 mL). The reaction was stirred at rt for 16 h, and then concentrated under reduced pressure. Purification (FCC, SiO₂, MeOH/DCM, 0 to 20%) and further by preparative HPLC (CH₃CN—H₂O, 5 to 95%, including 0.1% formic acid) afforded the title compound. The correct fractions were combined, and dried by lyophilization to give Isopropyl ((((1S,3R,4S,E)-3-(6-amino-9H-purin-9-yl)-2-(fluoromethylene)-4-(hydroxymethyl)cyclobutyl)methoxy)(phenoxy)phosphoryl)-L-alaninate as a white fluffy solid (6.4 mg) as a mixture of isomers at the phosphorous center (Rₚ and Sₚ). P³¹-NMR (CD₃OD) δ ppm: 3.92, 3.55. MS [M+1]⁺=549.1.

### Example 24: Isopropyl ((((1R,2R,3S)-2-(hydroxymethyl)-3-(5-methyl-2,4-dioxo-3,4-dihydropyrimidin-1(2H)-yl)-4-methylenecyclobutyl)methoxy)(phenoxy)phosphoryl)-L-alaninate

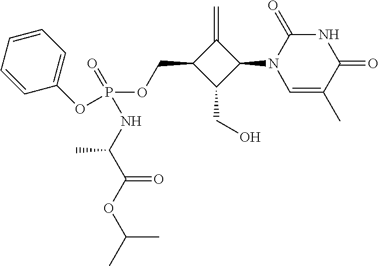 [SMILES: C=C1[C@@H](n2cc(C)c(=O)[nH]c2=O)[C@H](CO)[C@H]1COP(=O)(N[C@@H](C)C(=O)OC(C)C)Oc1ccccc1]

Step A. 1-((1S,2R,3R)-3-(Hydroxymethyl)-2-(((4-methoxyphenyl)diphenylmethoxy)methyl)-4-methylenecyclobutyl)-5-methylpyrimidine-2,4(1H,3H)-dione. To a solution of 1-((1S,2R,3R)-2,3-bis(hydroxymethyl)-4-methylenecyclobutyl)-5-methylpyrimidine-2,4(1H,3H)-dione (Example 5) (100 mg, 390 μmol) in dry DCM (2 mL) was added pyridine (157 mg, 1.4 mmol, 113.0 μL) at r.t., followed by MMTrCl (122 mg, 1.98 mmol) at 0° C. under N₂. The mixture was stirred at r.t. for 1 h. The mixture was extracted by DCM and water. The organic phase was concentrated in vacuo. Purification (FCC, SiO₂, DCM:MeOH=10:1 and MPLC, C18 Flash Column, Agela Technologies, 4 g, 4 mL/min, ACN:H₂O=20:80) afforded 1-((1S,2R,3R)-3-(hydroxymethyl)-2-(((4-methoxyphenyl)diphenylmethoxy)methyl)-4-methylenecyclobutyl)-5-methylpyrimidine-2,4(1H,3H)-dione (45 mg, 82 μmol) as a white solid. ¹H-NMR (400 MHz, DMSO-d₆): δ ppm 11.37 (s, 1H), 7.55-7.51 (m, 1H), 7.37-7.17 (m, 12H), 6.91-6.86 (m, 2H), 5.42-5.35 (m, 1H), 5.09-5.04 (m, 1H), 4.87-4.83 (m, 1H), 4.67 (t, J=5.3, 1H), 3.74 (s, 3H), 3.59 (t, J=5.2, 2H), 3.21-3.09 (m, 2H), 2.71-2.60 (m, 2H), 1.82 (s, 3H). ESI-LCMS: m/z 547 [M+H]⁺.

Step B. Isopropyl ((((1R,2R,3S)-2-(hydroxymethyl)-3-(5-methyl-2,4-dioxo-3,4-dihydropyrimidin-1 (2H)-yl)-4-methylenecyclobutyl)methoxy)(phenoxy)phosphoryl)-L-alaninate. The title compound as a mixture of isomers at the phosphorous center, (Rₚ and Sₚ), was prepared in a manner analogous to Example 23, Steps B-C using 1-((1S,2R,3R)-3-(Hydroxymethyl)-2-(((4-methoxyphenyl)diphenylmethoxy)methyl)-4-methylenecyclobutyl)-5-methylpyrimidine-2,4(1H,3H)-dione. P³¹-NMR (CD₃OD) δ ppm: 4.07, 3.72. MS [M+H]⁺ 522.1.

### Example 25: Isopropyl ((((1R,2R,3S)-3-(4-amino-2-oxopyrimidin-1(2H)-yl)-2-(hydroxymethyl)-4-methylenecyclobutyl)methoxy)(phenoxy)phosphoryl)-L-alaninate

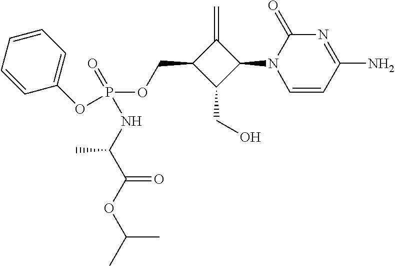 [SMILES: C=C1[C@@H](n2ccc(N)nc2=O)[C@H](CO)[C@H]1COP(=O)(N[C@@H](C)C(=O)OC(C)C)Oc1ccccc1]

Step A. N-(1-((1S,2R,3R)-3-(Hydroxymethyl)-2-(((4-methoxyphenyl)diphenylmethoxy)methyl)-4-methylenecyclobutyl)-2-oxo-1,2-dihydropyrimidin-4-yl)benzamide. To a solution of N-(1-((1S,2R,3R)-2,3-bis(hydroxymethyl)-4-methylenecyclobutyl)-2-oxo-1,2-dihydropyrimidin-4-yl)benzamide (Example 4, Step E.) (900 mg, 2.64 mmol) in pyridine (15 mL) was added 4-methoxytriphenylmethyl chloride (976.99 mg, 3.16 mmol) at 0° C., the mixture was allowed warm to r.t. and stirred for 3 h. The reaction was quenched with methanol. The reaction mixture was concentrated under reduced pressure. Purification (MPLC, C18 Flash Column, Agela Technologies, 12 g, 12 mL/min, ACN:H₂O=70:30) afforded N-(1-((1S,2R,3R)-3-(hydroxymethyl)-2-(((4-methoxyphenyl)diphenylmethoxy)methyl)-4-methylenecyclobutyl)-2-oxo-1,2-dihydropyrimidin-4-yl)benzamide (630 mg, 944.4 μmol, 35.8% yield, 92% purity) as a white solid. ESI-LCMS m/z=614.2 [M+H]⁺

Step B. 4-Amino-1-((1S,2R,3R)-3-(hydroxymethyl)-2-(((4-methoxyphenyl)diphenylmethoxy)methyl)-4-methylenecyclobutyl)pyrimidin-2(1H)-one. A solution of N-(1-((1S,2R,3R)-3-(hydroxymethyl)-2-(((4-methoxyphenyl)diphenylmethoxy)methyl)-4-methylenecyclobutyl)-2-oxo-1,2-dihydropyrimidin-4-yl)benzamide (630 mg, 1.03 mmol) in methylamine/ethanol (8 mL) was stirred at r.t. for 30 min. The reaction mixture was concentrated under reduced pressure. Purification (MPLC, C18 Flash Column, Agela Technologies, 12 g, 12 mL/min, ACN:H₂O=50:50) afforded 4-amino-1-((1S,2R,3R)-3-(hydroxymethyl)-2-(((4-methoxyphenyl)diphenylmethoxy)methyl)-4-methylenecyclobutyl)pyrimidin-2(1H)-one (400 mg, 769.2 μmol, 74.9% yield, 98% purity) as a white solid. ESI LC-MS m/z=610.2 [M+H]⁺. ¹H-NMR (400 MHz, DMSO-d₆): δ 7.62 (d, J=7.4 Hz, 1H), 7.37-7.16 (m, 12H), 7.14 (d, J=16.4 Hz, 2H), 6.88 (d, J=8.9 Hz, 2H), 5.77 (d, J=7.4 Hz, 1H), 5.51-5.49 (m, 1H), 5.04 (t, J=2.2 Hz, 1H), 4.73 (t, J=2.4 Hz, 1H), 4.68 (t, J=5.2 Hz, 1H), 3.75 (s, 3H), 3.62-3.56 (m, 2H), 3.14-3.11 (m, 2H), 2.68-2.66 (m, 1H), 2.61-2.55 (m, 1H).

Step C. Isopropyl ((((1R,2R,3S)-3-(4-amino-2-oxopyrimidin-1(2H)-yl)-2-(hydroxymethyl)-4-methylenecyclobutyl)methoxy)(phenoxy)phosphoryl)-L-alaninate. The title compound as a mixture of isomers at the phosphorous center, (Rₚ and Sₚ), was prepared in a manner analogous to Example 23 using 4-amino-1-((1S,2R,3R)-3-(hydroxymethyl)-2-(((4-methoxyphenyl)diphenylmethoxy)methyl)-4-methylenecyclobutyl)pyrimidin-2(1H)-one. P³¹-NMR (CD₃OD) δ ppm: 3.95, 3.56. MS [M+1]⁺ 507.1.

### Example 26: Isopropyl ((((1R,2R,3S)-3-(4-amino-7H-pyrrolo[2,3-d]pyrimidin-7-yl)-2-(hydroxymethyl)-4-methylenecyclobutyl)methoxy)(phenoxy)phosphoryl)-L-alaninate

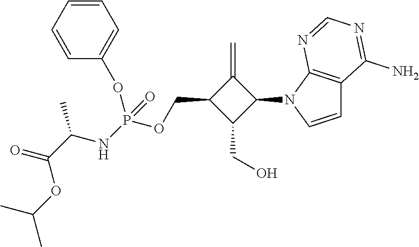 [SMILES: C=C1[C@@H](n2ccc3c(N)ncnc32)[C@H](CO)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 using ((1R,2R,3S)-3-(4-amino-7H-pyrrolo[2,3-d]pyrimidin-7-yl)-2-(((4-methoxyphenyl)diphenylmethoxy)methyl)-4-methylenecyclobutyl)methanol (Example 6, Step D). P³¹-NMR (CD₃OD) δ ppm: 3.93, 3.59. MS [M+1]⁺530.1.

### Example 27: Isopropyl ((((1R,2R,3S)-3-(4-amino-5-fluoro-7H-pyrrolo[2,3-d]pyrimidin-7-yl)-2-(hydroxymethyl)-4-methylenecyclobutyl)methoxy)(phenoxy)phosphoryl)-L-alaninate

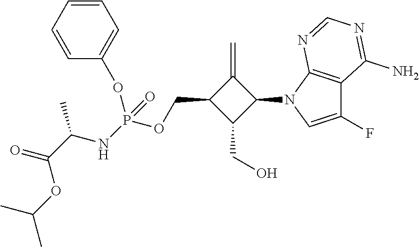 [SMILES: C=C1[C@@H](n2cc(F)c3c(N)ncnc32)[C@H](CO)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 ((1R,2R,3S)-3-(4-amino-5-fluoro-7H-pyrrolo[2,3-d]pyrimidin-7-yl)-2-(((4-methoxyphenyl)diphenylmethoxy)methyl)-4-methylenecyclobutyl)methanol (Example 7, Step D). P³¹-NMR (CD₃OD) δ ppm: 3.99, 3.62. MS [M+1]⁺548.2.

### Example 28: Isopropyl ((((1R,2R,3S)-3-(6-amino-9H-purin-9-yl)-2-(hydroxymethyl)-4-methylenecyclobutyl)methoxy)(phenoxy)phosphoryl)-L-alaninate

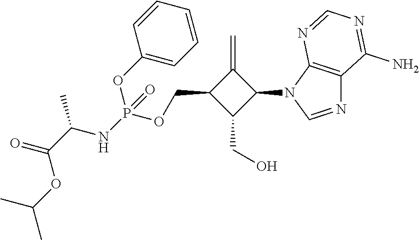 [SMILES: C=C1[C@@H](n2cnc3c(N)ncnc32)[C@H](CO)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 using ((1R,2R,3S)-3-(6-amino-9H-purin-9-yl)-2-(((4-methoxyphenyl)diphenylmethoxy)methyl)-4-methylenecyclobutyl)methanol (Intermediate 6). P³¹-NMR (CD₃OD) δ ppm: 2.90, 2.54. MS [M+1]⁺531.1.

### Example 29: Isopropyl ((((1R,2R,3S)-3-(2-amino-6-oxo-1,6-dihydro-9H-purin-9-yl)-2-(hydroxymethyl)-4-methylenecyclobutyl)methoxy)(phenoxy)phosphoryl)-L-alaninate

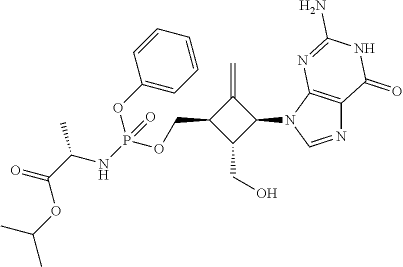 [SMILES: C=C1[C@@H](n2cnc3c(=O)[nH]c(N)nc32)[C@H](CO)[C@H]1COP(=O)(N[C@@H](C)C(=O)OC(C)C)Oc1ccccc1]

Step A. (E)-N′-(9-((1S,2R,3R)-2,3-Bis(hydroxymethyl)-4-methylenecyclobutyl)-6-hydroxy-9H-purin-2-yl)-N,N-dimethylformimidamide. ((1R,2R,3S)-3-(2-amino-6-hydroxy-9H-purin-9-yl)-4-methylenecyclobutane-1,2-diyl)dimethanol (Example 10) (160 mg, 0.58 mmol) was dissolved in MeOH (10 mL), then DMF-DMA (690.2 mg, 5.8 mmol) was added slowly. After stirring at r.t. for 4 h, the mixture was evaporated under reduced pressure. Purification (MPLC, C18 Flash Column, Agela Technologies, 12 g, 12 mL/min, ACN:H₂O=40:60) afforded (E)-N′-(9-((1S,2R,3R)-2,3-bis(hydroxymethyl)-4-methylenecyclobutyl)-6-hydroxy-9H-purin-2-yl)-N,N-dimethylformimidamide (190.2 mg, 0.57 mmol, 98%) as a white solid. ¹H NMR (400 MHz, DMSO-d₆) δ 11.27 (s, 1H), 8.55 (s, 1H), 7.86 (s, 1H), 5.18 (dd, J=7.9, 2.7 Hz, 1H), 5.04 (t, J=2.6 Hz, 1H), 4.77 (t, J=5.1 Hz, 1H), 4.72 (s, OH), 3.74-3.61 (m, 2H), 3.58 (td, J=5.0, 2.8 Hz, 2H), 3.15 (s, 3H), 3.03 (s, 3H), 2.91-2.83 (m, 1H), 2.83-2.76 (m, 1H). LCMS m/z=385.2 [M+H]⁺. LCMS m/z=333.2 [M+H]⁺.

Step B. (E)-N′-(6-Hydroxy-9-((1S,2R,3R)-3-(hydroxymethyl)-2-(((4-methoxyphenyl)diphenylmethoxy)methyl)-4-methylenecyclobutyl)-9H-purin-2-yl)-N,N-dimethylformimidamide. To a solution of (E)-N′-(9-((1S,2R,3R)-2,3-bis(hydroxymethyl)-4-methylenecyclobutyl)-6-hydroxy-9H-purin-2-yl)-N,N-dimethylformimidamide (190.2 mg, 0.57 mmol) in pyridine (10 mL) was added MMTrCl (193.1 mg, 0.63 mmol). The reaction mixture was stirred at r.t. for 2 h, then MeOH (1 mL) was added to quench the reaction. The reaction mixture was concentrated under reduced pressure. Purification (MPLC, C18 Flash Column, Agela Technologies, 4 g, 4 mL/min) and further purification (FCC, SiO₂, DCM:MeOH=10:1) afforded (E)-N′-(6-hydroxy-9-((1S,2R,3R)-3-(hydroxymethyl)-2-(((4-methoxyphenyl)diphenylmethoxy)methyl)-4-methylenecyclobutyl)-9H-purin-2-yl)-N,N-dimethylformimidamide (110.2 mg, 0.18 mmol, 28.9%) as a white solid. ¹H NMR (400 MHz, DMSO-d₆) δ 11.33 (s, 1H), 8.38 (s, 1H), 7.95 (s, 1H), 7.37-7.11 (m, 9H), 6.86-6.78 (m, 2H), 5.45 (d, J=7.9 Hz, 1H), 5.11 (d, J=2.6 Hz, 1H), 4.84 (d, J=2.5 Hz, 1H), 4.69 (t, J=5.4 Hz, 1H), 3.73 (s, 2H), 3.62 (dp, J=11.0, 5.6 Hz, 2H), 3.18 (d, J=5.8 Hz, 2H), 2.97 (s, 2H), 2.91-2.84 (m, 1H), 2.84 (s, 3H), 2.76 (d, J=15.3 Hz, 1H). LC-MS m/z=605.3 [M+H]⁺.

Step C. 2-Amino-9-((1S,2R,3R)-3-(hydroxymethyl)-2-(((4-methoxyphenyl)diphenylmethoxy)methyl)-4-methylenecyclobutyl)-9H-purin-6-ol. (E)-N′-(6-hydroxy-9-((1S,2R,3R)-3-(hydroxymethyl)-2-(((4-methoxyphenyl)diphenylmethoxy)methyl)-4-methylenecyclobutyl)-9H-purin-2-yl)-N,N-dimethylformimidamide (80 mg, 0.13 mmol) was dissolved in ammonia solution (5 mL, 7 N). After stirring at r.t. for 4 h, the excess reactant was removed to get the crude product. Purification (MPLC, C18 Flash Column, Agela Technologies, 4 g, 4 mL/min, ACN:H₂O=45:55) afforded 2-amino-9-((1S,2R,3R)-3-(hydroxymethyl)-2-(((4-methoxyphenyl)diphenylmethoxy)methyl)-4-methylenecyclobutyl)-9H-purin-6-ol (42 mg, 0.077 mmol, 58.8%) as a white solid. ¹H NMR (400 MHz, DMSO-d₆) δ 10.59 (s, 1H), 7.85 (s, 1H), 7.41-7.16 (m, 9H), 7.19-7.09 (m, 2H), 6.94-6.80 (m, 2H), 6.42 (s, 2H), 5.16 (d, J=8.4 Hz, 1H), 5.04 (d, J=2.8 Hz, 1H), 4.83-4.73 (m, 1H), 4.68 (t, J=5.3 Hz, 1H), 3.74 (s, 3H), 3.62 (dh, J=22.4, 5.7 Hz, 2H), 3.21-3.11 (m, 2H), 2.87 (p, J=7.3 Hz, 1H), 2.77 (d, J=5.9 Hz, 1H). LC-MS m/z=550.2 [M+H]⁺.

Step D. Isopropyl ((((1R,2R,3S)-3-(2-amino-6-oxo-1,6-dihydro-9H-purin-9-yl)-2-(hydroxymethyl)-4-methylenecyclobutyl)methoxy)(phenoxy)phosphoryl)-L-alaninate. The title compound as a mixture of isomers at the phosphorous center, (Rₚ and Sₚ), was prepared in a manner analogous to Example 23 using 2-amino-9-((1S,2R,3R)-3-(hydroxymethyl)-2-(((4-methoxyphenyl)diphenylmethoxy)methyl)-4-methylenecyclobutyl)-9H-purin-6-ol). P³¹-NMR (CD₃OD) δ ppm: 4.15, 3.75. MS [M+1]⁺547.1.

### Example 30: Isopropyl ((((1R,2R,3S)-3-(6-amino-2-fluoro-9H-purin-9-yl)-2-(hydroxymethyl)-4-methylenecyclobutyl)methoxy)(phenoxy)phosphoryl)-L-alaninate

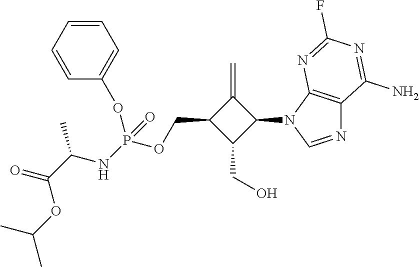 [SMILES: C=C1[C@@H](n2cnc3c(N)nc(F)nc32)[C@H](CO)[C@H]1COP(=O)(N[C@@H](C)C(=O)OC(C)C)Oc1ccccc1]

Step A. (E)-N′-(9-((1S,2R,3R)-2,3-Bis(hydroxymethyl)-4-methylenecyclobutyl)-2-fluoro-9H-purin-6-yl)-N,N-dimethylformimidamide. ((1R,2R,3S)-3-(6-amino-2-fluoro-9H-purin-9-yl)-4-methylenecyclobutane-1,2-diyl)dimethanol (Example 11) (190 mg, 0.71 mmol) was dissolved in MeOH (10 mL), then DMF-DMA (844.9 mg, 7.1 mmol) was added slowly. After stirring at r.t. for 4 h, the mixture was evaporated under reduced pressure to get the crude product. Purification (MPLC, C18 Flash Column, Agela Technologies, 12 g, 12 mL/min, ACN:H₂O=40:60) afforded (E)-N′-(9-((1S,2R,3R)-2,3-bis(hydroxymethyl)-4-methylenecyclobutyl)-2-fluoro-9H-purin-6-yl)-N,N-dimethylformimidamide (210 mg, 0.63 mmol, 88.5%) as a white solid. ¹H NMR (400 MHz, DMSO-d₆) δ 8.91 (s, 1H), 8.30 (s, 1H), 5.26 (dt, J=7.7, 2.6 Hz, 1H), 5.09-5.04 (m, 1H), 4.79 (t, J=5.0 Hz, 1H), 4.75-4.70 (m, 2H), 3.59 (h, J=6.0 Hz, 2H), 3.23 (s, 3H), 3.15 (d, J=0.7 Hz, 3H), 2.94-2.85 (m, 1H), 2.82 (dq, J=8.3, 3.0 Hz, 1H). LCMS m/z=335.1 [M+H]⁺

Step B. (E)-N′-(2-Fluoro-9-((1S,2R,3R)-3-(hydroxymethyl)-2-(((4-methoxyphenyl)diphenylmethoxy)methyl)-4-methylenecyclobutyl)-9H-purin-6-yl)-N,N-dimethylformimidamide. To a solution of (E)-N′-(9-((1S,2R,3R)-2,3-bis(hydroxymethyl)-4-methylenecyclobutyl)-2-fluoro-9H-purin-6-yl)-N,N-dimethylformimidamide (210 mg, 0.63 mmol) in DCM (10 mL) was added pyridine (5 eq.) and MMTrCl (225.0 mg, 0.69 mmol). The reaction mixture was stirred at r.t. for 2 h, then MeOH (1 mL) was added to quench the reaction. The reaction mixture was concentrated under reduced pressure. Purification (MPLC, C18 Flash Column, Agela Technologies, 4 g, 4 mL/min, ACN:H₂O=45:55) and purification (FCC, SiO₂, DCM:MeOH=10:1) afforded (E)-N′-(2-fluoro-9-((1S,2R,3R)-3-(hydroxymethyl)-2-(((4-methoxyphenyl)diphenylmethoxy)methyl)-4-methylenecyclobutyl)-9H-purin-6-yl)-N,N-dimethylformimidamide (110 mg, 0.18 mmol, 28.5%) as a white solid. ¹H NMR (400 MHz, DMSO-d₆) δ 8.95 (s, 1H), 8.39 (s, 1H), 7.35-7.17 (m, 10H), 7.12 (d, J=8.9 Hz, 1H), 6.82 (d, J=9.0 Hz, 2H), 5.36 (d, J=8.1 Hz, 1H), 5.11 (d, J=2.8 Hz, 1H), 4.82 (d, J=2.8 Hz, 1H), 4.71 (t, J=5.3 Hz, 1H), 3.73 (s, 3H), 3.65 (dq, J=10.7, 5.3 Hz, 2H), 3.26 (s, 3H), 3.21 (t, J=6.1 Hz, 1H), 3.18 (s, 2H), 2.95 (dd, J=13.3, 7.1 Hz, 1H), 2.83 (d, J=5.8 Hz, 1H). LCMS m/z=607.2 [M+H]⁺.

Step C. ((1R,2R,3S)-3-(6-Amino-2-fluoro-9H-purin-9-yl)-2-(((4-methoxyphenyl)diphenylmethoxy)methyl)-4-methylenecyclobutyl)methanol. (E)-N′-(2-fluoro-9-((1S,2R,3R)-3-(hydroxymethyl)-2-(((4-methoxyphenyl)diphenylmethoxy)methyl)-4-methylenecyclobutyl)-9H-purin-6-yl)-N,N-dimethylformimidamide (80 mg, 0.13 mmol) was dissolved in ammonia methanol solution (5 mL, 7 N). After stirring at r.t. for 4 h. Purification (MPLC, C18 Flash Column, Agela Technologies, 4 g, 4 mL/min, ACN:H₂O=40:60) afforded ((1R,2R,3S)-3-(6-amino-2-fluoro-9H-purin-9-yl)-2-(((4-methoxyphenyl)diphenylmethoxy)methyl)-4-methylenecyclobutyl)methanol (42 mg, 0.076 mmol, 58.5%) as white solid. ¹H NMR (400 MHz, DMSO-d₆) δ 8.27 (s, 1H), 7.85 (s, 2H), 7.33-7.16 (m, 11H), 7.11 (d, J=8.9 Hz, 2H), 6.82 (d, J=8.9 Hz, 2H), 5.29 (d, J=8.2 Hz, 1H), 5.09 (d, J=2.7 Hz, 1H), 4.82 (t, J=2.6 Hz, 1H), 4.67 (t, J=5.3 Hz, 1H), 3.72 (s, 3H), 3.64 (tt, J=10.7, 5.2 Hz, 2H), 3.26-3.10 (m, 2H), 3.03-2.86 (m, 1H), 2.80 (d, J=7.3 Hz, 1H). LCMS m/z=552.2 [M+H]⁺.

Step D. Isopropyl ((((1R,2R,3S)-3-(6-amino-2-fluoro-9H-purin-9-yl)-2-(hydroxymethyl)-4-methylenecyclobutyl)methoxy)(phenoxy)phosphoryl)-L-alaninate. The title compound as a mixture of isomers at the phosphorous center, (Rₚ and Sₚ), was prepared in a manner analogous to Example 23 using ((1R,2R,3S)-3-(6-Amino-2-fluoro-9H-purin-9-yl)-2-(((4-methoxyphenyl)diphenylmethoxy)methyl)-4-methylenecyclobutyl)methanol. P³¹-NMR (CD₃OD) δ ppm: 3.99, 3.62. MS [M+1]⁺549.1.

### Example 31: Isopropyl ((((1R,2R,3S)-3-(6-amino-9H-purin-9-yl)-2-(1-hydroxyethyl)-4-methylenecyclobutyl)methoxy)(phenoxy)phosphoryl)-L-alaninate

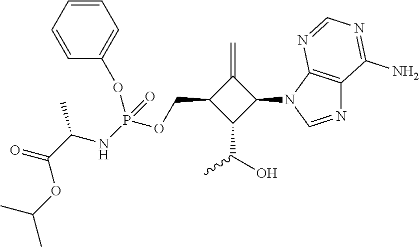 [SMILES: C=C1[C@@H](n2cnc3c(N)ncnc32)[C@H](C(C)O)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 using ((1R,2R,3S)-3-(6-amino-9H-purin-9-yl)-2-(1-((4-methoxyphenyl)diphenylmethoxy)ethyl)-4-methylenecyclobutyl)methanol (Example 12, Step F). P³¹-NMR (CD₃OD) δ ppm: 3.97, 3.65. MS [M+1]⁺545.4.

### Example 32: Isopropyl ((((1R,2R,3S)-3-(6-amino-9H-purin-9-yl)-2-(fluoromethyl)-4-methylenecyclobutyl)methoxy)(phenoxy)phosphoryl)-L-alaninate

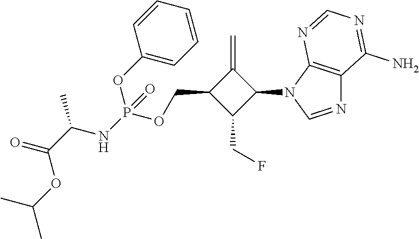 [SMILES: C=C1[C@@H](n2cnc3c(N)ncnc32)[C@H](CF)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 using ((1R,2R,3S)-3-(6-amino-9H-purin-9-yl)-2-(fluoromethyl)-4-methylenecyclobutyl)methanol (Example 13). P³¹-NMR (CD₃OD) δ ppm: 4.03, 3.62. MS [M+1]⁺533.1.

### Example 33: Isopropyl ((((1R,2R,3S)-3-(6-amino-9H-purin-9-yl)-2-cyano-4-methylenecyclobutyl)methoxy)(phenoxy)phosphoryl)-L-alaninate

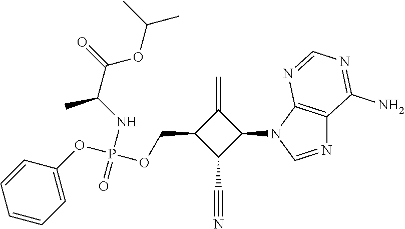 [SMILES: C=C1[C@@H](n2cnc3c(N)ncnc32)[C@H](C#N)[C@H]1COP(=O)(N[C@@H](C)C(=O)OC(C)C)Oc1ccccc1]

The title compound as a mixture of isomers at the phosphorous center, (Rₚ and Sₚ), was prepared in a manner analogous to Example 23 using (1R,2S,4R)-2-(6-amino-9H-purin-9-yl)-4-(hydroxymethyl)-3-methylenecyclobutanecarbonitrile (Example 14). P³¹-NMR (CD₃OD) δ ppm: 4.03, 3.69. MS [M+1]⁺526.0.

### Example 34: Isopropyl ((((1R,3S,4R,E)-3-(6-amino-9H-purin-9-yl)-2-(fluoromethylene)-4-(hydroxymethyl)cyclobutyl)methoxy)(phenoxy)phosphoryl)-L-alaninate

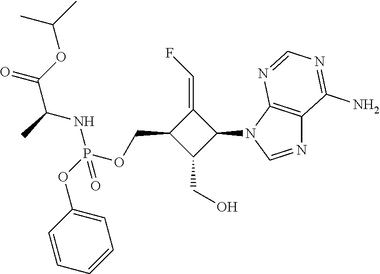 [SMILES: CC(C)OC(=O)[C@H](C)NP(=O)(OC[C@H]1/C(=C\F)[C@@H](n2cnc3c(N)ncnc32)[C@@H]1CO)Oc1ccccc1]

Step A. ((1R,3S,4R,E)-3-(6-Amino-9H-purin-9-yl)-2-(fluoromethylene)-4-(((4-methoxyphenyl)diphenylmethoxy)methyl)cyclobutyl)methanol. A solution of N-(9-((1S,3R,4R,E)-2-(fluoromethylene)-3-(hydroxymethyl)-4-(((4-methoxyphenyl)diphenylmethoxy)methyl)cyclobutyl)-9H-purin-6-yl)benzamide (Example 17, Step D.) (180 mg, 274.5 μmol) in methylamine/ethanol (3 mL) was stirred at r.t. for 30 min. The reaction mixture was concentrated under reduced pressure. Purification (MPLC, C18 Flash Column, Agela Technologies, 12 g, 12 mL/min, ACN:Water=50:50) afforded ((1R,3S,4R,E)-3-(6-amino-9H-purin-9-yl)-2-(fluoromethylene)-4-(((4-methoxyphenyl)diphenylmethoxy)methyl)cyclobutyl)methanol (120 mg, 213.2 μmol, 77.7% yield, 98% purity) as a white solid. ESI-LCMS m/z=552.2 [M+H]⁺. ¹H-NMR (400 MHz, DMSO-d₆): δ 8.38 (s, 1H), 8.18 (s, 1H), 7.33 (s, 1H), 7.26-7.19 (m, 10H), 7.16-7.13 (m, 2H), 6.95 (t, J=2.4 Hz, 0.5H), 6.84-6.82 (m, 2H), 6.75 (t, J=2.4 Hz, 0.5H), 5.50-5.49 (m, 1H), 4.87 (t, J=5.4 Hz, 1H), 3.78-3.75 (m, 2H), 3.72 (s, 3H), 3.22-3.20 (m, 2H), 3.14-3.12 (m, 1H), 2.95 (s, 1H). ¹⁹FNMR (400 MHz, DMSO-d₆): δ −138.15 (s).

Step B. Isopropyl ((((I R,3S,4R,E)-3-(6-amino-9H-purin-9-yl)-2-(fluoromethylene)-4-(hydroxymethyl)cyclobutyl)methoxy)(phenoxy)phosphoryl)-L-alaninate. The title compound as a mixture of isomers at the phosphorous center, (Rₚ and Sₚ), was prepared in a manner analogous to Example 23 using ((1R,3S,4R,E)-3-(6-amino-9H-purin-9-yl)-2-(fluoromethylene)-4-(((4-methoxyphenyl)diphenylmethoxy)methyl)cyclobutyl)methanol. P³¹-NMR (CD₃OD) δ ppm: 3.91, 3.67. MS [M+1]⁺549.1.

### Example 35: Isopropyl ((((1R,3S,4R,Z)-3-(6-amino-9H-purin-9-yl)-2-(fluoromethylene)-4-(hydroxymethyl)cyclobutyl)methoxy)(phenoxy)phosphoryl)-L-alaninate

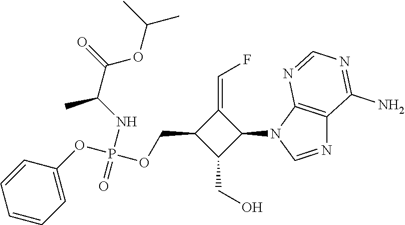 [SMILES: CC(C)OC(=O)[C@H](C)NP(=O)(OC[C@H]1/C(=C/F)[C@@H](n2cnc3c(N)ncnc32)[C@@H]1CO)Oc1ccccc1]

Step A. ((1R,3S,4R,Z)-3-(6-Amino-9H-purin-9-yl)-2-(fluoromethylene)-4-(((4-methoxyphenyl)diphenylmethoxy)methyl)cyclobutyl)methanol. A solution of N-(9-41S,3R,4R,Z)-2-(fluoromethylene)-3-(hydroxymethyl)-4-(((4-methoxyphenyl)diphenylmethoxy)methyl)cyclobutyl)-9H-purin-6-yl)benzamide (Example 16, Step A) (202 mg, 308.1 μmol) in methylamine/ethanol (4 mL) was stirred at r.t. for 30 min The reaction mixture was concentrated under reduced pressure. Purification (MPLC, C18 Flash Column, Agela Technologies, 4 g, 4 mL/min, ACN:Water=50:50) afforded ((1R,3S,4R,Z)-3-(6-amino-9H-purin-9-yl)-2-(fluoromethylene)-4-(((4-methoxyphenyl)diphenylmethoxy)methyl)cyclobutyl)methanol (80 mg, 145.0 μmol, 47.1% yield, 97% purity) as a white solid. ESI LC-MS m/z=552.2 [M+H]⁺. ¹H-NMR (400 MHz, DMSO-d₆): δ 8.31 (s, 1H), 8.16 (s, 1H), 7.28-7.21 (m, 12H), 7.16-7.14 (m, 2H), 7.01 (t, J=2.5 Hz, 0.5H), 6.85-6.83 (m, 2H), 6.80 (t, J=2.5 Hz, 0.5H), 5.59-5.57 (m, 1H), 4.86 (t, J=5.3 Hz, 1H), 3.73 (s, 3H), 3.67-3.66 (m, 2H), 3.19-3.17 (m, 2H), 3.01-2.97 (m, 1H), 2.89-2.87 (m, 1H). ¹⁹FNMR (400 MHz, DMSO-d₆): δ −139.33 (s).

Step B. Isopropyl ((((1R,3S,4R,Z)-3-(6-amino-9H-purin-9-yl)-2-(fluoromethylene)-4-(hydroxymethyl)cyclobutyl)methoxy)(phenoxy)phosphoryl)-L-alaninate. The title compound as a mixture of isomers at the phosphorous center, (Rₚ and Sₚ), was prepared in a manner analogous to Example 23 using ((1R,3S,4R,Z)-3-(6-amino-9H-purin-9-yl)-2-(fluoromethylene)-4-(((4-methoxyphenyl)diphenylmethoxy)methyl)cyclobutyl)methanol. P³¹-NMR (CD₃OD) δ ppm: 4.00, 3.62; MS [M+1]⁺549.1.

### Example 36: Isopropyl ((((1S,3R,4S,Z)-3-(6-amino-9H-purin-9-yl)-2-(fluoromethylene)-4-(hydroxymethyl)cyclobutyl)methoxy)(phenoxy)phosphoryl)-L-alaninate

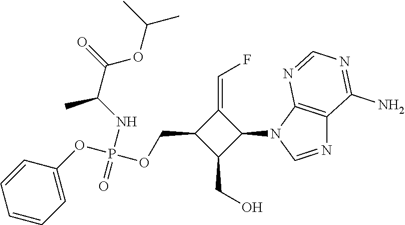 [SMILES: CC(C)OC(=O)[C@H](C)NP(=O)(OC[C@H]1/C(=C/F)[C@@H](n2cnc3c(N)ncnc32)[C@H]1CO)Oc1ccccc1]

Step A. ((1S,3R,4S,Z)-3-(6-Amino-9H-purin-9-yl)-2-(fluoromethylene)-4-(((4-methoxyphenyl)diphenylmethoxy)methyl)cyclobutyl)methanol. A solution of N-(9-((1R,3S,4S,Z)-2-(fluoromethylene)-3-(hydroxymethyl)-4-(((4-methoxyphenyl)diphenylmethoxy)methyl)cyclobutyl)-9H-purin-6-yl)benzamide (Example 16, Step F) (196 mg, 298.9 μmol) in methylamine/ethanol (4 mL) was stirred at r.t. for 30 min. The reaction mixture was concentrated under reduced pressure. Purification (MPLC, C18 Flash Column, Agela Technologies, 12 g, 12 mL/min, ACN:Water=50:50) afforded 41S,3R,4S,Z)-3-(6-amino-9H-purin-9-yl)-2-(fluoromethylene)-4-(((4-methoxyphenyl)diphenylmethoxy)methyl)cyclobutyl)methanol (120 mg, 213.2 μmol, 71.3% yield, 98% purity) as a white solid. ESI LC-MS m/z=552.2 [M+H]⁺. ¹H-NMR (400 MHz, DMSO-d₆): δ 8.31 (s, 1H), 8.16 (s, 1H), 7.28-7.21 (m, 12H), 7.16-7.14 (m, 2H), 7.01 (t, J=2.5 Hz, 0.5H), 6.85-6.83 (m, 2H), 6.80 (t, J=2.5 Hz, 0.5H), 5.59-5.57 (m, 1H), 4.86 (t, J=5.3 Hz, 1H), 3.73 (s, 3H), 3.67-3.66 (m, 2H), 3.19-3.17 (m, 2H), 3.01-2.97 (m, 1H), 2.89-2.87 (m, 1H).

Step B. Isopropyl ((((1S,3R,4S,Z)-3-(6-amino-9H-purin-9-yl)-2-(fluoromethylene)-4-(hydroxymethyl)cyclobutyl)methoxy)(phenoxy)phosphoryl)-L-alaninate. The title compound as a mixture of isomers at the phosphorous center, (Rₚ and Sₚ), was prepared in a manner analogous to Example 23 using ((1S,3R,4S,Z)-3-(6-amino-9H-purin-9-yl)-2-(fluoromethylene)-4-(((4-methoxyphenyl)diphenylmethoxy)methyl)cyclobutyl)methanol. P³¹-NMR (CD₃OD) δ ppm: 4.02, 3.58; MS [M+1]⁺549.1.

### Example 37: (2S)-Isopropyl 2-(((((1S,2R,3S)-3-(6-amino-9H-purin-9-yl)-1-ethynyl-2-(hydroxymethyl)-4-methylenecyclobutyl)methoxy)(phenoxy)phosphoryl)amino)propanoate

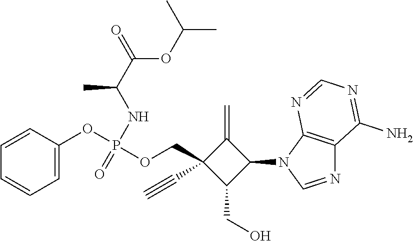 [SMILES: C#C[C@]1(COP(=O)(N[C@@H](C)C(=O)OC(C)C)Oc2ccccc2)C(=C)[C@@H](n2cnc3c(N)ncnc32)[C@@H]1CO]

The title compound was prepared in a manner analogous Example 23, using ((1S,2R,3S)-3-(6-amino-9H-purin-9-yl)-1-ethynyl-2-(((4-methoxyphenyl)diphenylmethoxy)methyl)-4-methylenecyclobutyl)methanol. (Example 19, Step T) as the nucleoside starting material. P³¹-NMR (CD₃OD) δ ppm: 3.57, 3.13; MS [M+1]⁺556.

### Example 38: Synthesis of Nucleoside 5′-triphosphates

Dry nucleoside (0.05 mmol) was dissolved in dry PO(OMe)₃ (1 mL). N-Methylimidazole (0.009 mL, 0.11 mmol) was added followed by POCl₃ (0.009 mL, 0.11 mmol). The reaction mixture was stirred at rt for 20-40 minutes. The reaction was monitored by LCMS (by the appearance of corresponding nucleoside 5′-monophosphate). Upon completion of the reaction tetrabutylammonium salt of pyrophosphate (150 mg) was added, followed by DMF (0.5 mL) to get a homogeneous solution. The reaction mixture was stirred at rt for 1.5 h, then diluted with water (10 mL). Purification (column HiLoad 16/10 with Q Sepharose High Performance: Separation was done in a linear gradient of NaCl from 0 (buffer A) to 1N in 50 mM TRIS-buffer (pH7.5) (buffer B). Triphosphate was eluted at 75-80% of buffer B. Corresponding fractions were concentrated. Desalting was achieved by RP HPLC on Synergy 4 micron Hydro-RP column (Phenominex). A linear gradient of acetonitrile from 0 to 30% in 10 mM triethylammonium acetate buffer (pH 7.5) was used for elution. The corresponding fractions were combined, concentrated and lyophilized 3 times to remove excess of buffer to afford the desired nucleoside 5′-triphosphate.

### Example 39: ((1R,3S)-3-(4-Amino-2-oxopyrimidin-1(2H)-yl)-2-methylenecyclobutyl)methyl tetrahydrogen triphosphate

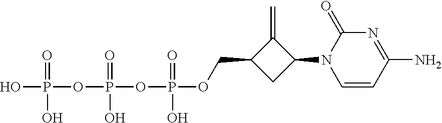 [SMILES: C=C1[C@H](COP(=O)(O)OP(=O)(O)OP(=O)(O)O)C[C@@H]1n1ccc(N)nc1=O]

The title compound was prepared in a manner analogous to Example 38, using 4-amino-1-((1S,3R)-3-(hydroxymethyl)-2-methylenecyclobutyl)pyrimidin-2(1H)-one (Example 1) as the nucleoside starting material.

### Example 40: ((1R,3S)-3-(2-Amino-6-oxo-1,6-dihydro-9H-purin-9-yl)-2-methylenecyclobutyl)methyl tetrahydrogen triphosphate

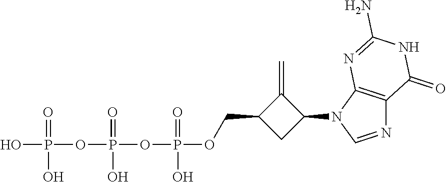 [SMILES: C=C1[C@H](COP(=O)(O)OP(=O)(O)OP(=O)(O)O)C[C@@H]1n1cnc2c(=O)[nH]c(N)nc21]

The title compound was prepared in a manner analogous to Example 38, using ((1R,3S)-3-(6-Amino-9H-purin-9-yl)-2-methylenecyclobutyl)methanol (Example 2) as the nucleoside starting material.

### Example 41: ((1R,3S)-3-(6-Amino-9H-purin-9-yl)-2-methylenecyclobutyl)methyl tetrahydrogen triphosphate

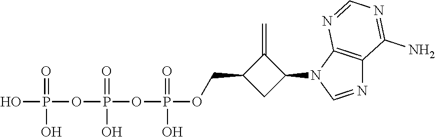 [SMILES: C=C1[C@H](COP(=O)(O)OP(=O)(O)OP(=O)(O)O)C[C@@H]1n1cnc2c(N)ncnc21]

The title compound was prepared in a manner analogous to Example 38, using ((1R,3S)-3-(6-amino-9H-purin-9-yl)-2-methylenecyclobutyl)methanol (Example 3) as the nucleoside starting material.

### Example 42: ((1R,2R,3S)-3-(6-Amino-9H-purin-9-yl)-2-(fluoromethyl)-4-methylenecyclobutyl)methyl tetrahydrogen triphosphate

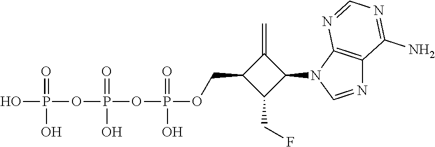 [SMILES: C=C1[C@@H](n2cnc3c(N)ncnc32)[C@H](CF)[C@H]1COP(=O)(O)OP(=O)(O)OP(=O)(O)O]

The title compound was prepared in a manner analogous to Example 38, using ((1R,2R,3S)-3-(6-amino-9H-purin-9-yl)-2-(fluoromethyl)-4-methylenecyclobutyl)methanol (Example 13) as the nucleoside starting material.

### Example 43: ((1R,2R,3S)-3-(6-Amino-9H-purin-9-yl)-2-cyano-4-methylenecyclobutyl)methyl tetrahydrogen triphosphate

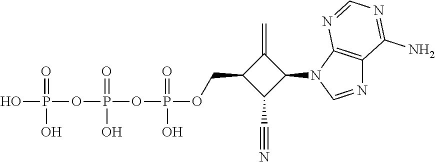 [SMILES: C=C1[C@@H](n2cnc3c(N)ncnc32)[C@H](C#N)[C@H]1COP(=O)(O)OP(=O)(O)OP(=O)(O)O]

The title compound was prepared in a manner analogous to Example 38, using (1R,2S,4R)-2-(6-amino-9H-purin-9-yl)-4-(hydroxymethyl)-3-methylenecyclobutanecarbonitrile (Example 14) as the nucleoside starting material.

| Ex. | | MS | P(α) | |
|---|---|---|---|---|
| # | Structure | (M − H) | and P(γ) | P(β) |
| 39 | 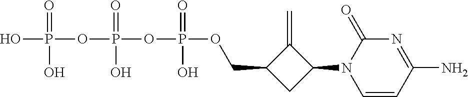 [SMILES: C=C1[C@H](COP(=O)(O)OP(=O)(O)OP(=O)(O)O)C[C@@H]1n1ccc(N)nc1=O] | 446.2 | −11.04 (d, 2P) | −23.51(t) |
| 40 | 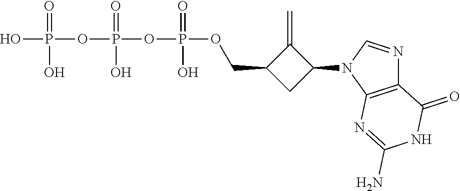 [SMILES: C=C1[C@H](COP(=O)(O)OP(=O)(O)OP(=O)(O)O)C[C@@H]1n1cnc2c(=O)[nH]c(N)nc21] | 486.4 | −11.01(d); −11.15 (d) | −23.48(t) |
| 41 | 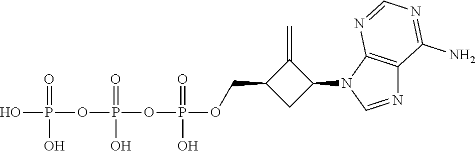 [SMILES: C=C1[C@H](COP(=O)(O)OP(=O)(O)OP(=O)(O)O)C[C@@H]1n1cnc2c(N)ncnc21] | 469.6 | −10.77(d); −11.01 (d) | −23.16(t) |
| 42 | 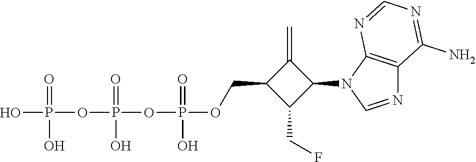 [SMILES: C=C1[C@@H](n2cnc3c(N)ncnc32)[C@H](CF)[C@H]1COP(=O)(O)OP(=O)(O)OP(=O)(O)O] | 502.6 | −11.00(d); −11.12 (d) | −23.41(t) |
| 43 | 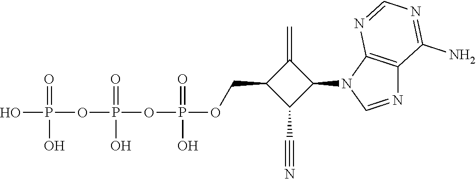 [SMILES: C=C1[C@@H](n2cnc3c(N)ncnc32)[C@H](C#N)[C@H]1COP(=O)(O)OP(=O)(O)OP(=O)(O)O] | 495.3 | −10.95(d); −11.40 (d) | −23.41(t) |

### Example 44: Nucleoside 5′-triphosphates

1,2,4-Triazole (21 mg, 0.3 mmol) was suspended in dry CH₃CN (0.7 mL). Triethylamine was added (0.046 mL, 0.33 mmol) and the mixture was vortexed to obtain a clear solution. After addition of POCl₃ (0.01 ml, 0.1 mmol) the mixture was vortexed and left for 20 min, then centrifugated. Supernatant was added to dry 2′-O-4,4′-dimethoxytrityl (DMTr) protected nucleoside (0.05 mmol) and the mixture was kept at ambient temperature for 0.5 h. Tetrabutylammonium salt of pyrophosphate (150 mg) was added, followed by DMF (0.5 mL) to get a homogeneous solution. The reaction mixture was kept for 1.5 h at ambient temperature. The reaction mixture was quenched with water. The phosphate was isolated by IE chromatography on AKTA Explorer using column HiLoad 16/10 with Q Sepharose High Performance. Separation was done in linear gradient of NaCl from 0 to 1N in 50 mM TRIS-buffer (pH7.5). Fractions eluted at 60-70% NaCl were combined, concentrated and desalted by Reverse phase HPLC on Synergy 4 micron Hydro-RP column (Phenominex). Linear gradient of acetonitrile from 0 to 90% in 50 mM triethylammonium buffer was used for elution over 20 min, flow 10 mL/min. Corresponding fractions were concentrated and treated with 80% HCOOH for 15 min at RT. Solvent was evaporated, the residue was suspended in water. Suspension was spinned and supernatant was purified by Reverse Phase HPLC as described above with gradient of acetonitrile from 0 to 30%. The corresponding fractions were combined, concentrated and lyophilized 3 times to remove excess of buffer.

### Example 45: ((1R,2R,3S)-3-(4-Amino-2-oxopyrimidin-1(2H)-yl)-2-(hydroxymethyl)-4-methylenecyclobutyl)methyl tetrahydrogen triphosphate

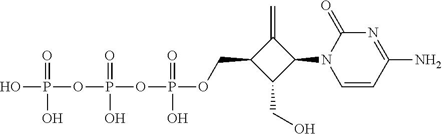 [SMILES: C=C1[C@@H](n2ccc(N)nc2=O)[C@H](CO)[C@H]1COP(=O)(O)OP(=O)(O)OP(=O)(O)O]

The title compound was prepared in a manner analogous to Example 44, using 4-amino-1-((1S,2R,3R)-3-(hydroxymethyl)-2-(((4-methoxyphenyl)diphenylmethoxy)methyl)-4-methylenecyclobutyl)pyrimidin-2(1H)-one (Example 1, Step B) as the nucleoside starting material.

### Example 46: ((1R,2R,3S)-2-(Hydroxymethyl)-3-(5-methyl-2,4-dioxo-3,4-dihydropyrimidin-1(2H)-yl)-4-methylenecyclobutyl)methyl tetrahydrogen triphosphate

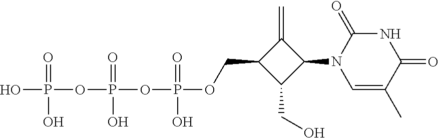 [SMILES: C=C1[C@@H](n2cc(C)c(=O)[nH]c2=O)[C@H](CO)[C@H]1COP(=O)(O)OP(=O)(O)OP(=O)(O)O]

The title compound was prepared in a manner analogous to Example 44, using 1-((1S,2R,3R)-3-(hydroxymethyl)-2-(((4-methoxyphenyl)diphenylmethoxy)methyl)-4-methylenecyclobutyl)-5-methylpyrimidine-2,4(1H,3H)-dione (Example 24, Step A) as the nucleoside starting material.

### Example 47: ((1R,2R,3S)-3-(4-Amino-7H-pyrrolo[2,3-d]pyrimidin-7-yl)-2-(hydroxymethyl)-4-methylenecyclobutyl)methyl tetrahydrogen triphosphate

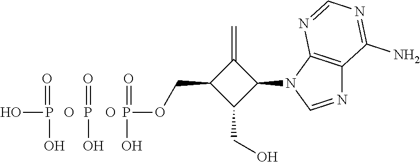

The title compound was prepared in a manner analogous to Example 44, using ((1R,2R,3S)-3-(4-amino-7H-pyrrolo[2,3-d]pyrimidin-7-yl)-2-(((4-methoxyphenyl)diphenylmethoxy)methyl)-4-methylenecyclobutyl)methanol (Example 6, Step D) as the nucleoside starting material.

### Example 48: ((1R,2R,3S)-3-(4-Amino-5-fluoro-7H-pyrrolo[2,3-d]pyrimidin-7-yl)-2-(hydroxymethyl)-4-methylenecyclobutyl)methyl tetrahydrogen triphosphate

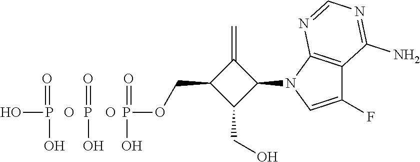

The title compound was prepared in a manner analogous to Example 44, using ((1R,2R,3S)-3-(4-amino-5-fluoro-7H-pyrrolo[2,3-d]pyrimidin-7-yl)-2-(((4-methoxyphenyl)diphenylmethoxy)methyl)-4-methylenecyclobutyl)methanol (Example 7, Step D) as the nucleoside starting material.

### Example 49: ((1R,2R,3S)-3-(6-Amino-9H-purin-9-yl)-2-(hydroxymethyl)-4-methylenecyclobutyl)methyl tetrahydrogen triphosphate

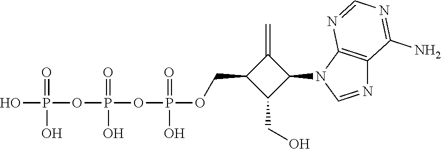 [SMILES: C=C1[C@@H](n2cnc3c(N)ncnc32)[C@H](CO)[C@H]1COP(=O)(O)OP(=O)(O)OP(=O)(O)O]

The title compound was prepared in a manner analogous to Example 44, using ((1R,2R,3S)-3-(6-amino-9H-purin-9-yl)-2-(((4-methoxyphenyl)diphenylmethoxy)methyl)-4-methylenecyclobutyl)methanol (Intermediate 6) as the nucleoside starting material.

### Example 50: ((1S,2S,3R)-3-(6-Amino-9H-purin-9-yl)-2-(hydroxymethyl)-4-methylenecyclobutyl)methyl tetrahydrogen triphosphate

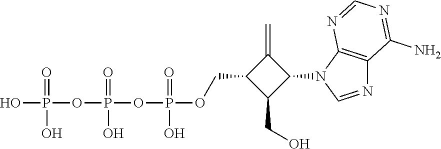 [SMILES: C=C1[C@@H](COP(=O)(O)OP(=O)(O)OP(=O)(O)O)[C@H](CO)[C@H]1n1cnc2c(N)ncnc21]

The title compound was prepared in a manner analogous to Example 44, using ((1S,2S,3R)-3-(6-amino-9H-purin-9-yl)-2-(((4-methoxyphenyl)diphenylmethoxy)methyl)-4-methylenecyclobutyl)methanol (Intermediate 7) as the nucleoside starting material.

### Example 51: ((1R,2R,3S)-3-(2-Amino-6-oxo-1,6-dihydro-9H-purin-9-yl)-2-(hydroxymethyl)-4-methylenecyclobutyl)methyl tetrahydrogen triphosphate

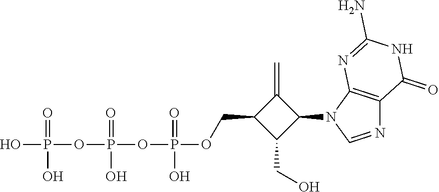 [SMILES: C=C1[C@@H](n2cnc3c(=O)[nH]c(N)nc32)[C@H](CO)[C@H]1COP(=O)(O)OP(=O)(O)OP(=O)(O)O]

The title compound was prepared in a manner analogous to Example 44, using 2-amino-9-((1S,2R,3R)-3-(hydroxymethyl)-2-(((4-methoxyphenyl)diphenylmethoxy)methyl)-4-methylenecyclobutyl)-9H-purin-6-ol (Example 29, Step C) as the nucleoside starting material.

### Example 52: ((1R,2R,3S)-3-(6-Amino-2-fluoro-9H-purin-9-yl)-2-(hydroxymethyl)-4-methylenecyclobutyl)methyl tetrahydrogen triphosphate

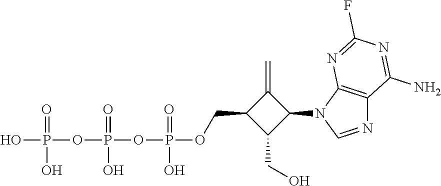 [SMILES: C=C1[C@@H](n2cnc3c(N)nc(F)nc32)[C@H](CO)[C@H]1COP(=O)(O)OP(=O)(O)OP(=O)(O)O]

The title compound was prepared in a manner analogous to Example 44, using ((1R,2R,3S)-3-(6-amino-2-fluoro-9H-purin-9-yl)-2-(((4-methoxyphenyl)diphenylmethoxy)methyl)-4-methylenecyclobutyl)methanol (Example 30, Step C) as the nucleoside starting material.

### Example 53: ((1R,2R,3S)-3-(6-Amino-9H-purin-9-yl)-2-((S)-1-hydroxyethyl)-4-methylenecyclobutyl)methyl tetrahydrogen triphosphate

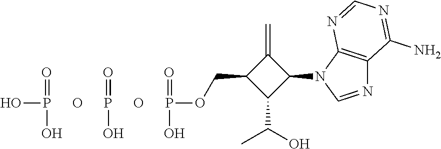

The title compound was prepared in a manner analogous to Example 44, using ((1R,2R,3S)-3-(6-amino-9H-purin-9-yl)-2-(1-((4-methoxyphenyl)diphenylmethoxy)ethyl)-4-methylenecyclobutyl)methanol (Example 12, Step F) as the nucleoside starting material.

### Example 54: ((1R,3S,4R,E)-3-(6-Amino-9H-purin-9-yl)-2-(fluoromethylene)-4-(hydroxymethyl)cyclobutyl)methyl tetrahydrogen triphosphate

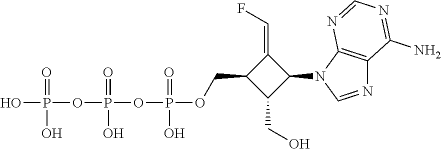 [SMILES: Nc1ncnc2c1ncn2[C@@H]1/C(=C/F)[C@H](COP(=O)(O)OP(=O)(O)OP(=O)(O)O)[C@H]1CO]

The title compound was prepared in a manner analogous to Example 44, using ((1R,3S,4R,E)-3-(6-amino-9H-purin-9-yl)-2-(fluoromethylene)-4-(((4-methoxyphenyl)diphenylmethoxy)methyl)cyclobutyl)methanol (Example 34, Step A) as the nucleoside starting material.

### Example 55: ((1S,3R,4S,E)-3-(6-Amino-9H-purin-9-yl)-2-(fluoromethylene)-4-(hydroxymethyl)cyclobutyl)methyl tetrahydrogen triphosphate

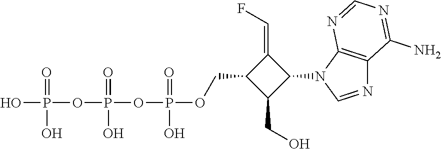 [SMILES: Nc1ncnc2c1ncn2[C@H]1/C(=C/F)[C@@H](COP(=O)(O)OP(=O)(O)OP(=O)(O)O)[C@@H]1CO]

The title compound was prepared in a manner analogous to Example 44, ((1S,3R,4S,E)-3-(6-amino-9H-purin-9-yl)-2-(fluoromethylene)-4-(((4-methoxyphenyl)diphenylmethoxy)methyl)cyclobutyl)methanol (Example 23, Step A) as the nucleoside starting material.

### Example 56: ((1R,3S,4R,Z)-3-(6-Amino-9H-purin-9-yl)-2-(fluoromethylene)-4-(hydroxymethyl)cyclobutyl)methyl tetrahydrogen triphosphate

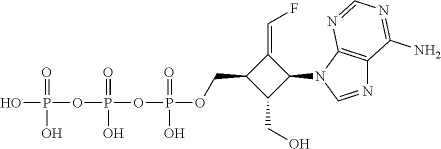 [SMILES: Nc1ncnc2c1ncn2[C@@H]1/C(=C\F)[C@H](COP(=O)(O)OP(=O)(O)OP(=O)(O)O)[C@H]1CO]

The title compound was prepared in a manner analogous to Example 44, using ((1R,3S,4R,Z)-3-(6-amino-9H-purin-9-yl)-2-(fluoromethylene)-4-(((4-methoxyphenyl)diphenylmethoxy)methyl)cyclobutyl)methanol (Example 35, Step A) as the nucleoside starting material.

### Example 57: ((1S,3R,4S,Z)-3-(6-Amino-9H-purin-9-yl)-2-(fluoromethylene)-4-(hydroxymethyl)cyclobutyl)methyl tetrahydrogen triphosphate

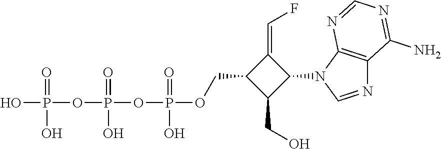 [SMILES: Nc1ncnc2c1ncn2[C@H]1/C(=C\F)[C@@H](COP(=O)(O)OP(=O)(O)OP(=O)(O)O)[C@@H]1CO]

The title compound was prepared in a manner analogous to Example 44, using ((1S,3R,4S,Z)-3-(6-amino-9H-purin-9-yl)-2-(fluoromethylene)-4-(((4-methoxyphenyl)diphenylmethoxy)methyl)cyclobutyl)methanol (Example 36, Step A) as the nucleoside starting material.

### Example 58: ((1S,2R,3S)-3-(6-Amino-9H-purin-9-yl)-1-ethynyl-2-(hydroxymethyl)-4-methylenecyclobutyl)methyl tetrahydrogen triphosphate

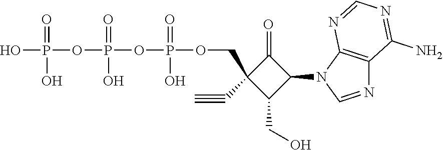 [SMILES: C#C[C@]1(COP(=O)(O)OP(=O)(O)OP(=O)(O)O)C(=O)[C@@H](n2cnc3c(N)ncnc32)[C@@H]1CO]

The title compound was prepared in a manner analogous to Example 44, using ((1S,2R,3S)-3-(6-amino-9H-purin-9-yl)-1-ethynyl-2-(((4-methoxyphenyl)diphenylmethoxy)methyl)-4-methylenecyclobutyl)methanol. (Example 19, Step X) as the nucleoside starting material.

| Ex. | | MS | P(α) | |
|---|---|---|---|---|
| # | Structure | (M − H) | and P(γ) | P(β) |
| 45 | 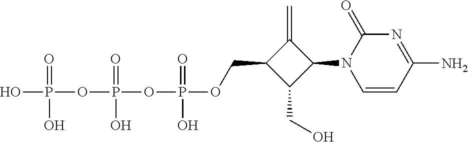 [SMILES: C=C1[C@@H](n2ccc(N)nc2=O)[C@H](CO)[C@H]1COP(=O)(O)OP(=O)(O)OP(=O)(O)O] | 476.2 | −6.43(d); −11.01(d) | −22.68(t) |
| 46 | 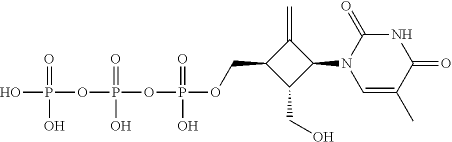 [SMILES: C=C1[C@@H](n2cc(C)c(=O)[nH]c2=O)[C@H](CO)[C@H]1COP(=O)(O)OP(=O)(O)OP(=O)(O)O] | 491.5 | −6.38(d); −11.05 (d) | −22.67(t) |
| 47 | 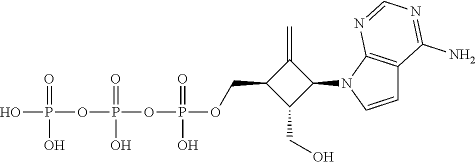 [SMILES: C=C1[C@@H](n2ccc3c(N)ncnc32)[C@H](CO)[C@H]1COP(=O)(O)OP(=O)(O)OP(=O)(O)O] | 499.2 | −10.88 (br.s. 2P) | −23.09 (br.s) |
| 48 | 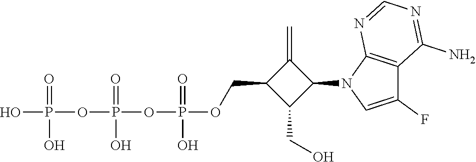 [SMILES: C=C1[C@@H](n2cc(F)c3c(N)ncnc32)[C@H](CO)[C@H]1COP(=O)(O)OP(=O)(O)OP(=O)(O)O] | 516.9 | −6.51(d); −11.01(d) | −22.65(d) |
| 49 | 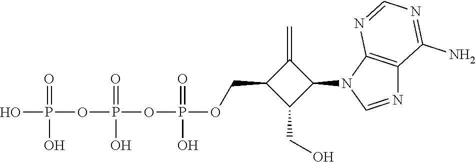 [SMILES: C=C1[C@@H](n2cnc3c(N)ncnc32)[C@H](CO)[C@H]1COP(=O)(O)OP(=O)(O)OP(=O)(O)O] | 500.0 | −10.82(d); −11.01 (d) | −23.39(t) |
| 50 | 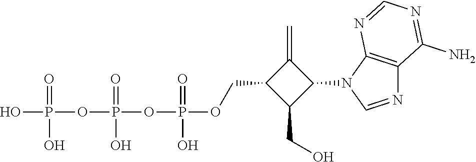 [SMILES: C=C1[C@@H](COP(=O)(O)OP(=O)(O)OP(=O)(O)O)[C@H](CO)[C@H]1n1cnc2c(N)ncnc21] | 500.1 | −6.58(d); −11.02 (d) | −22.75(t) |
| 51 | 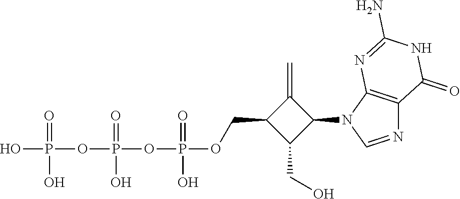 [SMILES: C=C1[C@@H](n2cnc3c(=O)[nH]c(N)nc32)[C@H](CO)[C@H]1COP(=O)(O)OP(=O)(O)OP(=O)(O)O] | 515.9 | −10.94(d); −11.13(d) | −23.42(t) |
| 52 | 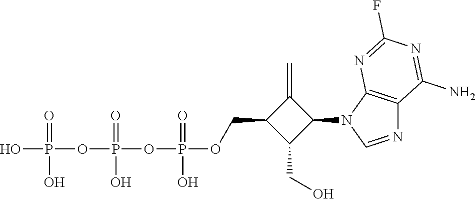 [SMILES: C=C1[C@@H](n2cnc3c(N)nc(F)nc32)[C@H](CO)[C@H]1COP(=O)(O)OP(=O)(O)OP(=O)(O)O] | 518.5 | −8.50 (br.s); −11.00(d) | −22.87(t) |
| 53 | 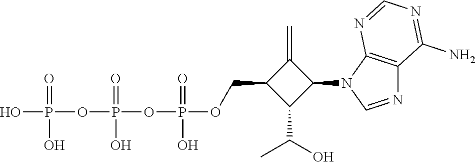 [SMILES: C=C1[C@@H](n2cnc3c(N)ncnc32)[C@H](C(C)O)[C@H]1COP(=O)(O)OP(=O)(O)OP(=O)(O)O] | 514.3 | −6.48(d); −11.10(d) | −22.72(t) |
| 54 | 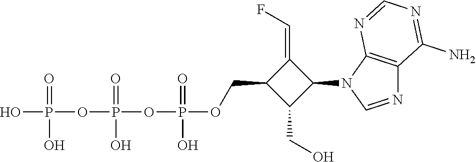 [SMILES: Nc1ncnc2c1ncn2[C@@H]1/C(=C/F)[C@H](COP(=O)(O)OP(=O)(O)OP(=O)(O)O)[C@H]1CO] | 518.5 | −6.46 (br.s, 2P) | −22.44(t) |
| 55 | 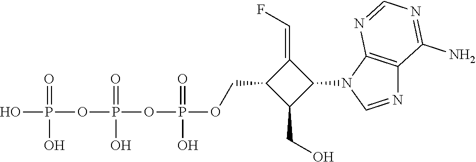 [SMILES: Nc1ncnc2c1ncn2[C@H]1/C(=C/F)[C@@H](COP(=O)(O)OP(=O)(O)OP(=O)(O)O)[C@@H]1CO] | 518.4 | −9.59 (br.s.); −11.05 (d) | −23.13(t) |
| 56 | 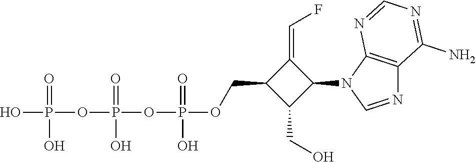 [SMILES: Nc1ncnc2c1ncn2[C@@H]1/C(=C\F)[C@H](COP(=O)(O)OP(=O)(O)OP(=O)(O)O)[C@H]1CO] | 518.6 | −6.54(d); −11.10(d) | −22.64(t) |
| 57 | 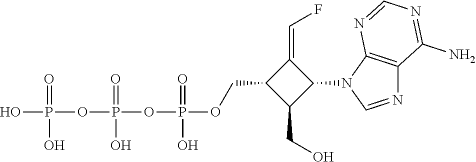 [SMILES: Nc1ncnc2c1ncn2[C@H]1/C(=C\F)[C@@H](COP(=O)(O)OP(=O)(O)OP(=O)(O)O)[C@@H]1CO] | 518.7 | −11.02(d); −11.25(d) | −23.44(t) |
| 58 | 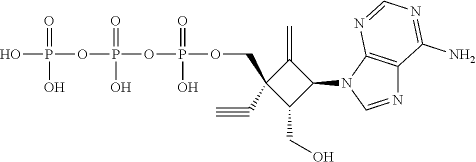 [SMILES: C#C[C@]1(COP(=O)(O)OP(=O)(O)OP(=O)(O)O)C(=C)[C@@H](n2cnc3c(N)ncnc32)[C@@H]1CO] | 524.8 | −10.69(d), −11.84(d) | −23.39(t) |

### Biology Assays

### Example A. HIV Single-Cycle Assay

24 h prior to infection, CEM human T lymphoblast cells (ATCC, Manassas, Va.) were plated in assay media (MEM supplemented with 10% FBS, 1% penicillin/streptomycin (all Mediatech, Manassas, Va.) and 1% DMSO (Sigma-Aldrich, St Louis, Mo.)) were plated at a density of 5×10⁵ cells/mL (5×10⁴ cells/well) in white 96-well plates. Serially diluted compounds were added to cells and incubated overnight at 37° C., 5% CO₂. The following day, cells were infected with VSV-G pseudotyped HIV NL4-3, in which parts of the env and nef were genes replaced with Renilla-luciferase, and infected cells were incubated for 72 h at 37° C., 5% CO₂. Viral inoculum was titrated to achieve a Renilla-luciferase signal of approximately 100× fold over background. Antiviral activity was measured by addition of 100 ul of Renilla-Glo® reagent (Promega, Madison, Wis.) to infected cells. After a 10-min incubation at RT, luminescence was measured on a Victor X3 multi-label plate reader (Perkin Elmer, Waltham, Mass.). Cytotoxicity of uninfected parallel cultures was determined by addition of 100 μL CellTiter-Glo® reagent (Promega, Madison, Wis.), and incubation for 10 mins at RT. Luminescence was measured on a Victor X3 multi-label plate reader.

### Example B. Inhibition of HIV Reverse Transcriptase

Recombinant full-length HIV-1 Reverse Transcriptase (HIVrt) was purchased from Abcam, catalog #ab63979. The last 385 nucleotide region of the HCV anti-genome complementary to the 5′ untranslated region (c5′UTR) was synthesized using T7 RNA polymerase Megascript kit from Ambion (Cat #AM1333). A DNA oligo served as an internal initiation primer and was purchased from IDT. Unless otherwise specified, reaction samples consisted of 20 nM c5′UTR RNA, 100 nM DNA primer, and 1 nM HIVrt, mixed together in a buffer containing 50 mM Tris pH 7.5, 100 mM KCl, 4 mM dithiothreitol (DTT), and 12.5 mM MgCl₂. Reactions were initiated at 30° C. by adding 0.1 μM adenosine triphosphate (dATP), 0.1 μM cytosine triphosphate (dCTP), 1 μM guanosine triphosphate (dGTP), and 0.32 μM ³H-thymidine triphosphate (³H-TTP), in a final volume of 50 μL. After 40-mins incubation, the reaction was terminated by adding 60 μL of chilled 20% (w/v) trichloroacetic acid with 500 μM ATP to precipitate nucleic acids. After incubation at 4° C. for 1 h, the sample underwent filtration on a multiscreen BV 1.2-μm 96-well plate (Millipore). 40 μL Microscint-20 (Perkin Elmer) was added to the well and the counts in the sample were determined by a Trilux Microbeta microplate scintillation reader (Wallac).

All data were analyzed with GraphPad Prism. The compound concentration at which the enzyme-catalyzed rate was reduced by 50% (IC₅₀) was calculated by fitting the data to the equation Y=% Min+(% Max−% Min)/(1+X/IC₅₀), where Y corresponds to the percent relative enzyme activity, % Min is the residual relative activity at saturating compound concentration, % Max is the relative maximum enzyme activity, and X corresponds to the compound concentration. The Kᵢ was calculated using the Cheng-Prusoff equation assuming competitive inhibition relative to natural dNTP incorporation: K=IC₅₀/(1+[dNTP]/Kₘ), where [dNTP] is the concentration of natural dNTP and Kₘ is the apparent Kₘ for dNTP. The standard HIVrt RNA-dependent DNA polymerization (RdDp) assay was used to determine the IC₅₀ values.

### Example C. Inhibition of HBV Polymerase

Recombinant full-length HBV polymerase (HBVpol) was expressed in SF9 cells and purified according to Lanford et al (Nucleotide priming and reverse transcriptase activity of hepatitis B virus polymerase expressed in insect cells. (Lanford et al., J Virol. 1995; 69(7):4431-4439). The last 385 nucleotide region of the HCV anti-genome complementary to the 5′ untranslated region (c5′UTR) was synthesized using T7 RNA polymerase Megascript kit from Ambion (Cat #AM1333). A DNA oligo served as an internal initiation primer and was purchased from IDT. Unless otherwise specified, reaction samples consisted of 50 nM c5′UTR RNA, 500 nM DNA primer, and 1 uL HIVrt, mixed together in a buffer containing 50 mM Tris pH 7.5, 100 mM KCl, 4 mM dithiothreitol (DTT), 10% DMSO and 12.5 mM MgCl₂. Reactions were initiated at 30° C. by adding 46 nM adenosine triphosphate (dATP), 17 nM cytosine triphosphate (dCTP), 57 nM guanosine triphosphate (dGTP), and 0.32 μM ³H-thymidine triphosphate (³H-TTP), in a final volume of 50 μL. After 120-mins incubation, the reaction was terminated by adding 60 μL of chilled 20% (w/v) trichloroacetic acid with 500 μM ATP to precipitate nucleic acids. After incubation at 4° C. for 1 h, the sample underwent filtration on a multiscreen BV 1.2-μm 96-well plate (Millipore). 40 μL Microscint-20 (Perkin Elmer) was added to the well and the counts in the sample were determined by a Trilux Microbeta microplate scintillation reader (Wallac).

All data were analyzed with GraphPad Prism. The compound concentration at which the enzyme-catalyzed rate was reduced by 50% (IC₅₀) was calculated by fitting the data to the equation Y=% Min+(% Max−% Min)/(1+X/IC₅₀), where Y corresponds to the percent relative enzyme activity, % Min is the residual relative activity at saturating compound concentration, % Max is the relative maximum enzyme activity, and X corresponds to the compound concentration. The Kᵢ was calculated using the Cheng-Prusoff equation assuming competitive inhibition relative to natural dNTP incorporation: Kᵢ=IC₅₀/(1+[dNTP]/Kₘ), where [dNTP] is the concentration of natural dNTP and Kₘ is the apparent Kₘ for dNTP. The standard HBVpol RNA-dependent DNA polymerization (RdDp) assay was used to determine the IC₅₀ values.

### Example D. Inhibition of HBV in Hepg2.117 Cells

HepG2.117 cells (use passage less than 25 passages) were cultured in DMEM/F12 50/50 medium (Corning, REF 10-092-CM) with 10% FBS (Corning REF 35-011-CV), 250 ug/mL G418 Sulfate (Corning, REF 30-234-CI), 2 ug/mL Tetracycline (TEKNOVA, cat #T3325) and 1× Penicillin/Streptomycin (Corning, 30-002-CI), (Corning, 30-002-CI). For each assay, cells were plated in assay medium: DMEM/F12 50/50 (Corning, REF 10-092-CM), 2% Tet-system approved FBS (Clontech, Cat #631106) and 1× Penicillin/Streptomycin (Corning, 30-002-CI).

### Determination of Anti-HBV Activity

Determination of 50% inhibitory concentration (EC₅₀) of compounds in HepG2.117 cells were performed by the following procedure. On the first day, cells were washed with PBS two times after trypsinizing the cells. Then cells were washed once with the assay medium. Cells were seeded at 30,000-35,000 cells per 100 μL per well in Biocoat collage coated flat bottom 96 well plates. Test compounds were solubilized in 100% DMSO to 100× the desired final testing concentration. Each compound was then serially diluted (1:3) up to 9 different concentrations. Compounds in 100% DMSO are reduced to 10% DMSO by diluting 1:10 in assay media. After incubation of the cells in a 37 C, 5% CO₂ incubator for 4 h, 10 uL test compounds diluted in assay media were added into cell plate. The final DMSO concentration was 1%. The cells were incubated at 37° C. for 96 h.

The antiviral activity was measured using a Real Time quantitative polymerase chain reaction (RT qPCR) assay directly measuring the HBV viral DNA copy numbers from the supernatant of HepG2.117 cells. The HBV Core primers and probes used in qPCR: core forward primer was 5′-CTGTGCCTTGGGTGGCTTT-3′ (SEQ. ID. NO. 1); the core reverse primer was 5′-AAGGAAAGAAGTCAGAAGGCAAAA-3′ (SEQ. ID. NO. 2); the core probe was 5′/FAM/AGCTCCAAA/ZEN/TCCTTTATAAGGGTCGATGTCCATG/31ABKFQ/-3′ (SEQ. ID. NO. 3). The core forward and core reverse probes were used at a final concentration of 1 μM and the core probe was used at a final concentration of 0.5 μM. The RT qPCR assay was set up with 10 μL 2× Quanta Perfecta qPCR ToughMix ROX, 0.1 μL 200× primer/probe mix, 4.0 μL HepG2.117 cell supernatant (or standard for control wells) and 5.9 μL dH₂O, for a total reagent volume of 20 μL per well. The standard was prepared by diluting HBV DNA Plasmid, Psi Check, in 10 mM TE buffer in a 1:5 ratio in 6 concentrations: 1E6, 0.2E6, 0.04E6, 0.008E6, 0.0016E6, 0.00032E6 of viral DNA copy numbers. The RT qPCR (Applied Biosystems and “Quant Studio 6 Flex” from Life Technology) was run for 5 mins at 95° C., then 15 mins at 95° C. and 20 mins at 60° C. for each cycle, 40 cycles in total.

HBV viral DNA copy numbers are normalized to the level observed in the absence of inhibitor, which was defined as 100%. EC₅₀ was defined as the concentration of compound at which the HBV viral DNA copy numbers from the HepG2.117 cells was reduced 50% relative to its level in the absence of compound.

### Determination of Cytotoxicity in HepG2 Cells

Cell cytotoxicity (CC₅₀) against HepG2 cells was measured using a luminescent cell viability assay to determine the number of viable cells in the culture based on quantitation of the adenosine triphosphate (ATP) present after a 4-day incubation period. On the first day, HepG2 cells were seeded at 15,000/100 uL/well with assay media containing DEME (Corning, REF 10-013-CV), 3% FBS (Coning REF 35-011-CV), 1× Penicillin/Streptomycin (Corning, 30-002-O), and 1× Non-Essential Amino Acid in Biocoat college 96-well flat bottom plates. Cells were incubated in a 37° C., 5% CO₂ incubator for 4 h before compound dosing. The compound dilution and dosing procedure were identical to that outlined with respect to determine anti-HBV activity. After 96 h incubation, cell viability is normalized to the level observed in the absence of inhibitor, which was defined as 100%. No cytotoxic effect on the HepG2 cells was defined as a 50% cytotoxic concentration (CC₅₀)>100 μM.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound of Formula (I), or a pharmaceutically acceptable salt thereof, having the structure: 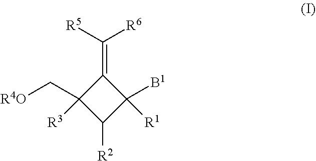
wherein:
B¹ is an optionally substituted N-linked heterocyclic base or an optionally substituted C-linked heterocyclic base;
R¹ is selected from the group consisting of hydrogen, halogen, cyano, an optionally substituted C₁₋₆ alkyl, an unsubstituted C₂₋₆ alkenyl and an unsubstituted C₂₋₆ alkynyl, wherein when the C₁₋₆ alkyl is substituted, the C₁₋₆ alkyl is substituted with at least one halogen;
R² is selected from the group consisting of hydrogen, halogen, hydroxy, cyano and an optionally substituted C₁₋₄ alkyl, wherein when the C₁₋₄ alkyl is substituted, the C₁₋₄ alkyl is substituted with a hydroxy or at least one halogen;
R³ is selected from the group consisting of hydrogen, halogen, cyano, an optionally substituted C₁₋₄ alkyl, an optionally substituted C₂₋₄ alkenyl and an unsubstituted C₂₋₄ alkynyl, wherein when the C₁₋₄ alkyl or C₂₋₄ alkenyl are substituted, the C₁₋₄ alkyl and C₂₋₄ alkenyl are independently substituted with at least one halogen;
R⁴ is selected from the group consisting of hydrogen, an optionally substituted acyl, an optionally substituted O-linked α-amino acid, 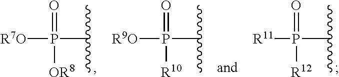
R⁵ and R⁶ are independently hydrogen or halogen;
R⁷ and R⁸ are independently selected from the group consisting of absent, hydrogen, 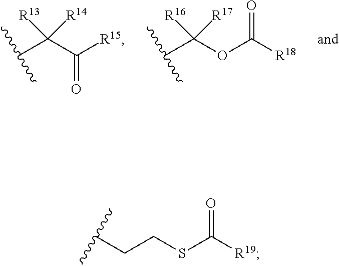
or
R⁷ is 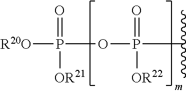
and R⁸ is absent or hydrogen;
R⁹ is absent, hydrogen, an optionally substituted aryl or an optionally substituted heteroaryl;
R¹⁰ is an optionally substituted N-linked α-amino acid or an optionally substituted N-linked α-amino acid ester derivative;
R¹¹ and R¹² are independently an optionally substituted N-linked α-amino acid or an optionally substituted N-linked α-amino acid ester derivative;
R¹³, R¹⁴, R¹⁶ and R¹⁷ are independently selected from the group consisting of hydrogen, an optionally substituted C₁₋₂₄ alkyl and an optionally substituted aryl;
R¹⁵ and R¹⁸ are independently selected from the group consisting of hydrogen, an optionally substituted C₁₋₂₄ alkyl, an optionally substituted aryl, an optionally substituted —O—C₁₋₂₄ alkyl and an optionally substituted —O-aryl;
R¹⁹ is selected from the group consisting of hydrogen, an optionally substituted C₁₋₂₄ alkyl and an optionally substituted aryl;
R²⁰, R²¹ and R²² are independently absent or hydrogen;
R⁵ and R⁶ are independently hydrogen or halogen; and
m is 0 or 1; and
provided that when R¹ is hydrogen; R² is hydroxy; R⁵ and R⁶ are each hydrogen; and
B¹ is adenine; then R³ is not hydrogen.

2. The compound of claim 1, wherein the compound of Formula (I) is selected from the group consisting of: 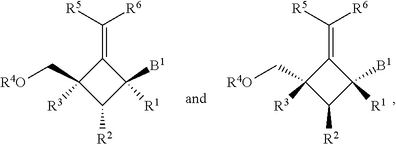
or a pharmaceutically acceptable salt of any of the foregoing.

3. The compound of claim 1 or 2, wherein R³ is halogen.

4. The compound of claim 3, wherein the halogen is fluoro.

5. The compound of claim 1 or 2, wherein R³ is cyano.

6. The compound of claim 1 or 2, wherein R³ is an optionally substituted C₁₋₄ alkyl, wherein when the C₁₋₄ alkyl is substituted, the C₁₋₄ alkyl is substituted with at least one halogen.

7. The compound of claim 6, wherein R³ is an unsubstituted C₁₋₄ alkyl.

8. The compound of claim 6, wherein R³ is a fluoro-substituted C₁₋₄ alkyl.

9. The compound of claim 6, wherein R³ is a chloro-substituted C₁₋₄ alkyl.

10. The compound of claim 8 or 9, wherein R³ is —CH₂F or —CH₂Cl.

11. The compound of claim 1 or 2, wherein R³ is an optionally substituted C₂₋₄ alkenyl, wherein when the C₂₋₄ alkenyl is substituted, C₂₋₄ alkenyl is substituted with at least one halogen.

12. The compound of claim 11, wherein R³ is an unsubstituted C₂₋₄ alkenyl.

13. The compound of claim 11, wherein R³ is a fluoro-substituted C₂₋₄ alkenyl.

14. The compound of claim 11, wherein R³ is a chloro-substituted C₂₋₄ alkenyl.

15. The compound of claim 1 or 2, wherein R³ is hydrogen.

16. The compound of any one of claims 1-15, wherein R² is halogen.

17. The compound of any one of claims 1-14, wherein R² is hydroxy.

18. The compound of any one of claims 1-15, wherein R² is cyano.

19. The compound of any one of claims 1-15, wherein R² is an optionally substituted C₁₋₄ alkyl, wherein when the C₁₋₄ alkyl is substituted, the C₁₋₄ alkyl is substituted with a hydroxy or at least one halogen.

20. The compound of claim 19, wherein R² is an unsubstituted C₁₋₄ alkyl.

21. The compound of claim 19, wherein R² is a fluoro-substituted C₁₋₄ alkyl.

22. The compound of claim 21, wherein R² is —CH₂F.

23. The compound of claim 19, wherein R² is a chloro-substituted C₁₋₄ alkyl.

24. The compound of claim 23, wherein R² is —CH₂Cl.

25. The compound of claim 19, wherein R² is a hydroxy-substituted C₁₋₄ alkyl.

26. The compound of claim 25, wherein R² is —CH₂OH.

27. The compound of any one of claims 1-14, wherein R² is hydrogen.

28. The compound of any one of claims 1-27, wherein R¹ is hydrogen.

29. The compound of any one of claims 1-27, wherein R¹ is halogen.

30. The compound of any one of claims 1-27, wherein R¹ is cyano.

31. The compound of any one of claims 1-27, wherein R¹ is an optionally substituted C₁₋₆ alkyl, wherein when the C₁₋₆ alkyl is substituted, the C₁₋₆ alkyl is substituted with at least one halogen.

32. The compound of any one of claims 1-27, wherein R¹ is an unsubstituted C₂₋₆ alkenyl.

33. The compound of any one of claims 1-27, wherein R¹ is an unsubstituted C₂₋₆ alkynyl.

34. The compound of any one of claims 1-33, wherein R⁵ and R⁶ are each hydrogen.

35. The compound of any one of claims 1-33, wherein R⁵ and R⁶ are each halogen.

36. The compound of any one of claims 1-33, wherein one of R⁵ and R⁶ is hydrogen, and the other of R⁵ and R⁶ is halogen.

37. The compound of claim 35 or 36, wherein the halogen is fluoro.

38. The compound of any one of claims 1-37, wherein R⁴ is hydrogen.

39. The compound of any one of claims 1-37, wherein R⁴ is an optionally substituted acyl.

40. The compound of claim 39, wherein R⁴ is an unsubstituted acyl.

41. The compound of any one of claims 1-37, wherein R⁴ is an optionally substituted O-linked α-amino acid.

42. The compound of any one of claims 1-37, wherein R⁴ is an unsubstituted O-linked α-amino acid.

43. The compound of claim 42, wherein R⁴ is selected from unsubstituted O-linked alanine, unsubstituted O-linked valine, unsubstituted O-linked leucine and unsubstituted O-linked glycine.

44. The compound of any one of claims 1-37, wherein R⁴ is

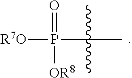

45. The compound of claim 44, wherein R⁷ and R⁸ are each absent or hydrogen.

46. The compound of claim 44, wherein R⁷ is 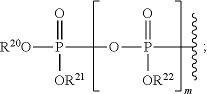
and R⁸ is absent or hydrogen.

47. The compound of claim 44, wherein m is 0; R⁸, R²⁰ and R²¹ are independently absent or hydrogen.

48. The compound of claim 44, wherein m is 1; R⁸, R²⁰, R²¹ and R²² are independently absent or hydrogen.

49. The compound of claim 44, wherein one of R⁷ and R⁸ is absent, hydrogen or 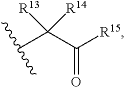
and the other of R⁷ and R⁸ is

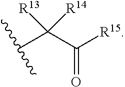

50. The compound of claim 44, wherein R⁷ and R⁸ are each

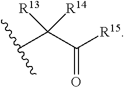

51. The compound of claim 44, wherein one of R⁷ and R⁸ is absent, hydrogen or 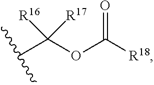
and the other of R⁷ and R⁸ is

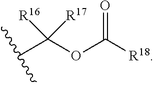

52. The compound of claim 44, wherein R⁷ and R⁸ are each

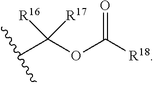

53. The compound of claim 44, wherein one of R⁷ and R⁸ is absent, hydrogen or 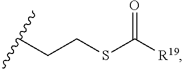
and the other of R⁷ and R⁸ is

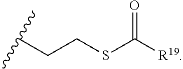

54. The compound of claim 44, wherein R⁷ and R⁸ are each

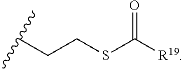

55. The compound of any one of claims 1-37, wherein R⁴ is

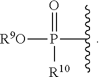

56. The compound of claim 55, wherein R⁹ is an optionally substituted aryl.

57. The compound of claim 55, wherein the optionally substituted aryl is an optionally substituted phenyl or any optionally substituted naphthyl.

58. The compound of claim 57, wherein the optionally substituted phenyl is an unsubstituted phenyl.

59. The compound of claim 55, wherein R⁹ is an optionally substituted heteroaryl.

60. The compound of claim 59, wherein R⁹ is an optionally substituted monocyclic heteroaryl.

61. The compound of any one of claim 55-60, wherein R¹⁰ is an optionally substituted N-linked α-amino acid.

62. The compound of any one of claim 55-60, wherein R¹⁰ is an optionally substituted N-linked α-amino acid ester derivative.

63. The compound of claim 61 or 62, wherein R¹⁰ is N-linked alanine, N-linked alanine isopropyl ester, N-linked alanine cyclohexyl ester and N-linked alanine neopentyl ester.

64. The compound of any one of claims 1-37, wherein R⁴ is

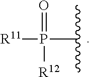

65. The compound of claim 64, wherein R¹¹ and R¹² are independently an optionally substituted N-linked α-amino acid ester derivative.

66. The compound of claim 64 or 65, wherein R¹¹ and R¹² are independently selected from the group consisting of N-linked alanine, N-linked alanine isopropyl ester, N-linked alanine cyclohexyl ester and N-linked alanine neopentyl ester.

67. The compound of any one of claim 1-66, wherein B¹ is an optionally substituted purine.

68. The compound of any one of claim 1-66, wherein B¹ is an optionally substituted pyrimidine.

69. The compound of any one of claim 1-66, wherein B¹ is selected from the group consisting of: 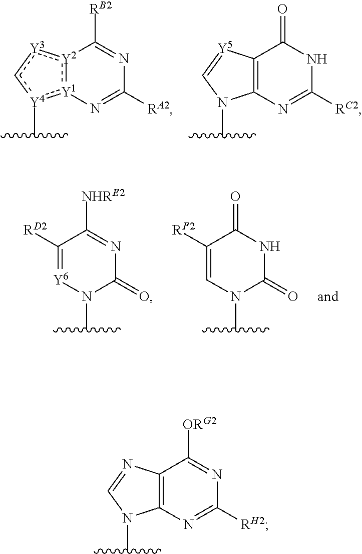
wherein:
R^{A2} is selected from the group consisting of hydrogen, halogen and NHR^{J2}, wherein R^{J2} is selected from the group consisting of hydrogen, —C(═O)R^{K2} and —C(═O)OR^{L2};
R^{H2} is halogen or NHR^{W2}, wherein R^{W2} is selected from the group consisting of hydrogen, an optionally substituted C₁₋₆ alkyl, an optionally substituted C₂₋₆ alkenyl, an optionally substituted C₃₋₈ cycloalkyl, —C(═O)R^{M2} and —C(═O)OR^{N2};
R^{C2} is hydrogen or NHR^{O2}, wherein R^{O2} is selected from the group consisting of hydrogen, —C(═O)R^{P2} and —C(═O)OR^{Q2};
R^{D2} is selected from the group consisting of hydrogen, deuterium, halogen, an optionally substituted C₁₋₆ alkyl, an optionally substituted C₂₋₆ alkenyl and an optionally substituted C₂₋₆ alkynyl;
R^{E2} is selected from the group consisting of hydrogen, hydroxy, an optionally substituted C₁₋₆ alkyl, an optionally substituted C₃₋₈ cycloalkyl, —C(═O)R^{R2} and —C(═O)OR^{S2};
R^{F2} is selected from the group consisting of hydrogen, halogen, an optionally substituted C₁₋₆ alkyl, an optionally substituted C₂₋₆ alkenyl and an optionally substituted C₂₋₆ alkynyl;
Y¹, Y² and Y⁴ are independently N or C, provided that at least one of Y¹, Y² and Y⁴ is N;
Y³ is N or CR^{I2}, wherein R^{I2} is selected from the group consisting of hydrogen, halogen, an unsubstituted C₁₋₆-alkyl, an unsubstituted C₂₋₆-alkenyl and an unsubstituted C₂₋₆-alkynyl;
Y⁵ and Y⁶ are independently N or CH;
each [symbol] is independently a single or double bond, provided that the single bonds and the double bonds are situated in the ring so that each ring is aromatic;
R^{G2} is an optionally substituted C₁₋₆ alkyl;
R^{H2} is hydrogen or NHR^{T2}, wherein R^{T2} is independently selected from the group consisting of hydrogen, —C(═O)R^{U2} and —C(═O)OR^{V2}; and
R^{K2}, R^{L2}, R^{M2}, R^{N2}, R^{P2}, R^{Q2}, R^{R2}, R^{S2}, R^{U2} and R^{V2} are independently selected from the group consisting of an unsubstituted C₁₋₆ alkyl, an unsubstituted C₂₋₆ alkenyl, an unsubstituted C₂₋₆ alkynyl, an optionally substituted C₃₋₆ cycloalkyl, an optionally substituted C₃₋₆ cycloalkenyl, an optionally substituted C₆₋₁₀ aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl).

70. The compound of claim 1 or 2, wherein B¹ is an optionally substituted N-linked heterocyclic base.

71. The compound of claim 70, wherein B¹ is an optionally substituted purine.

72. The compound of claim 70, wherein B¹ is an optionally substituted pyrimidine.

73. The compound of claim 69, wherein B¹ is selected from the group consisting of:

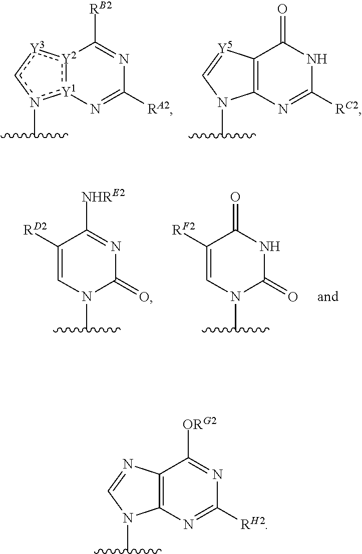

74. The compound of claim 73, wherein B¹ is

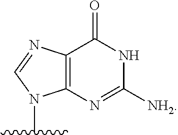 [SMILES: *n1cnc2c(=O)[nH]c(N)nc21]

75. The compound of claim 73, wherein B¹ is

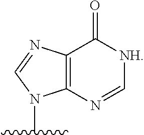 [SMILES: *n1cnc2c(=O)[nH]cnc21]

76. The compound of claim 73, wherein B¹ is

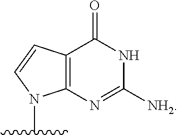 [SMILES: *n1ccc2c(=O)[nH]c(N)nc21]

77. The compound of claim 73, wherein B¹ is

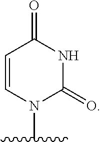 [SMILES: *n1ccc(=O)[nH]c1=O]

78. The compound of claim 73, wherein B¹ is

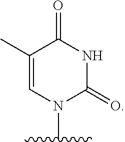 [SMILES: *n1cc(C)c(=O)[nH]c1=O]

79. The compound of claim 73, wherein B¹ is

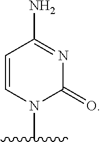 [SMILES: *n1ccc(N)nc1=O]

80. The compound of claim 73, wherein B¹ is

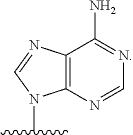 [SMILES: *n1cnc2c(N)ncnc21]

81. The compound of claim 73, wherein B¹ is

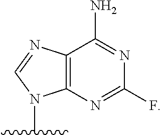 [SMILES: *n1cnc2c(N)nc(F)nc21]

82. The compound of claim 73, wherein B¹ is

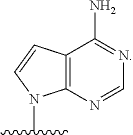 [SMILES: *n1ccc2c(N)ncnc21]

83. The compound of claim 73, wherein B^{1A} is

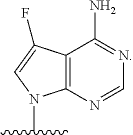 [SMILES: *n1cc(F)c2c(N)ncnc21]

84. The compound of claim 73, wherein B¹ is

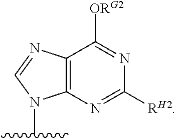

85. The compound of claim 1 or 2, wherein B¹ is an optionally substituted C-linked heterocyclic base.

86. The compound of claim 69, wherein B¹ is

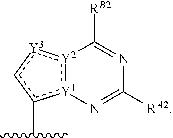

87. The compound of claim 86, wherein B¹ is selected from the group consisting of:

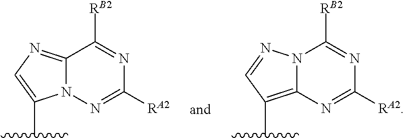

88. The compound of claim 86, wherein B¹ is selected from the group consisting of:

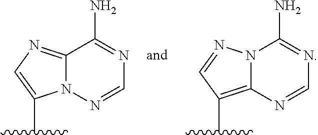 [SMILES: *c1cnc2c(N)ncnn12 ; *c1cnn2c(N)ncnc12]

89. The compound of claim 1, selected from the group consisting of: 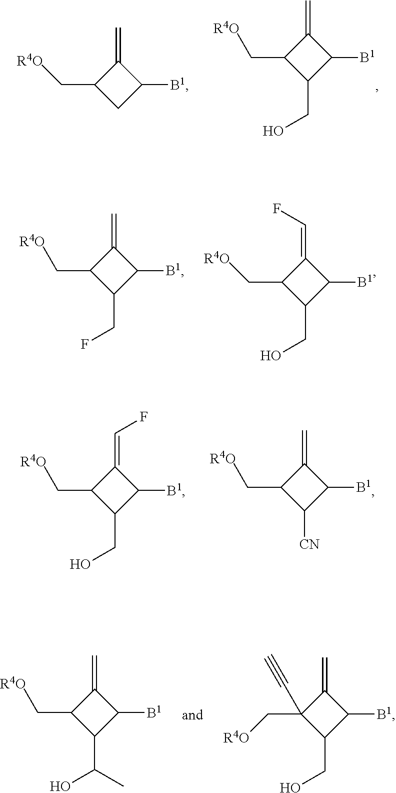
or a pharmaceutically acceptable salt of any of the foregoing.

90. The compound of claim 1, selected from the group consisting of: 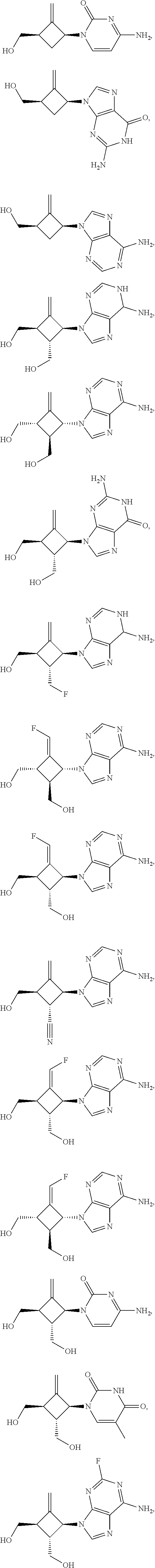 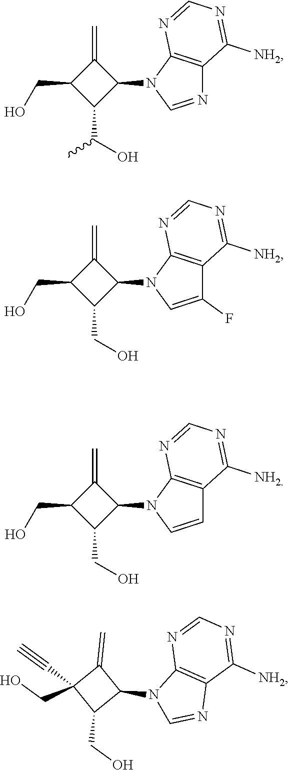 [SMILES: C=C1[C@@H](n2cnc3c(N)ncnc32)[C@H](C(C)O)[C@H]1CO ; C=C1[C@@H](n2cc(F)c3c(N)ncnc32)[C@H](CO)[C@H]1CO ; C=C1[C@@H](n2ccc3c(N)ncnc32)[C@H](CO)[C@H]1CO ; C#C[C@]1(CO)C(=C)[C@@H](n2cnc3c(N)ncnc32)[C@@H]1CO] 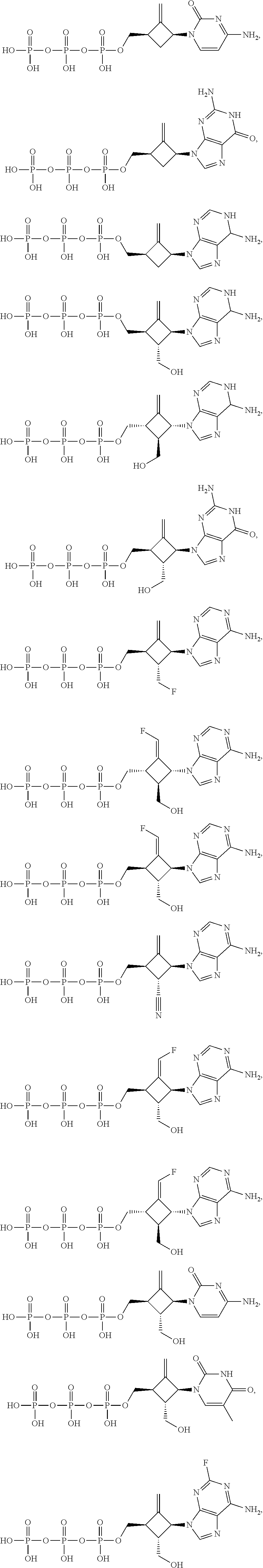 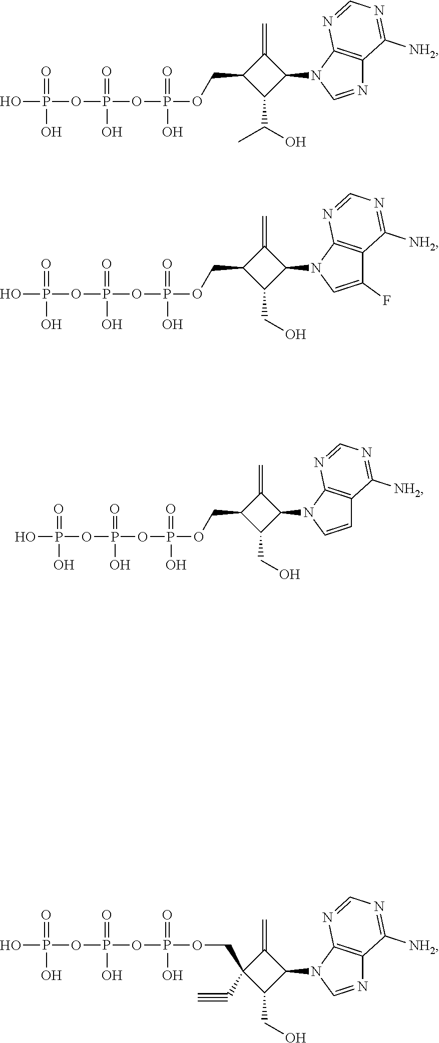 [SMILES: C=C1[C@@H](n2cnc3c(N)ncnc32)[C@H](C(C)O)[C@H]1COP(=O)(O)OP(=O)(O)OP(=O)(O)O ; C=C1[C@@H](n2cc(F)c3c(N)ncnc32)[C@H](CO)[C@H]1COP(=O)(O)OP(=O)(O)OP(=O)(O)O ; C=C1[C@@H](n2ccc3c(N)ncnc32)[C@H](CO)[C@H]1COP(=O)(O)OP(=O)(O)OP(=O)(O)O ; C#C[C@]1(COP(=O)(O)OP(=O)(O)OP(=O)(O)O)C(=C)[C@@H](n2cnc3c(N)ncnc32)[C@@H]1CO] 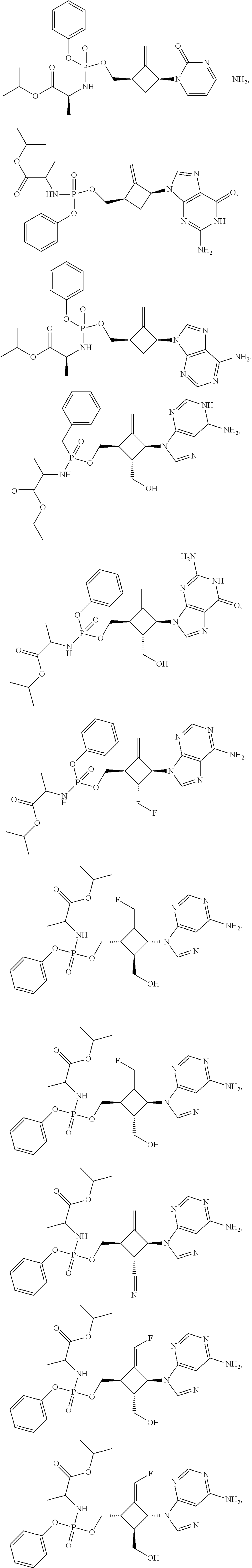 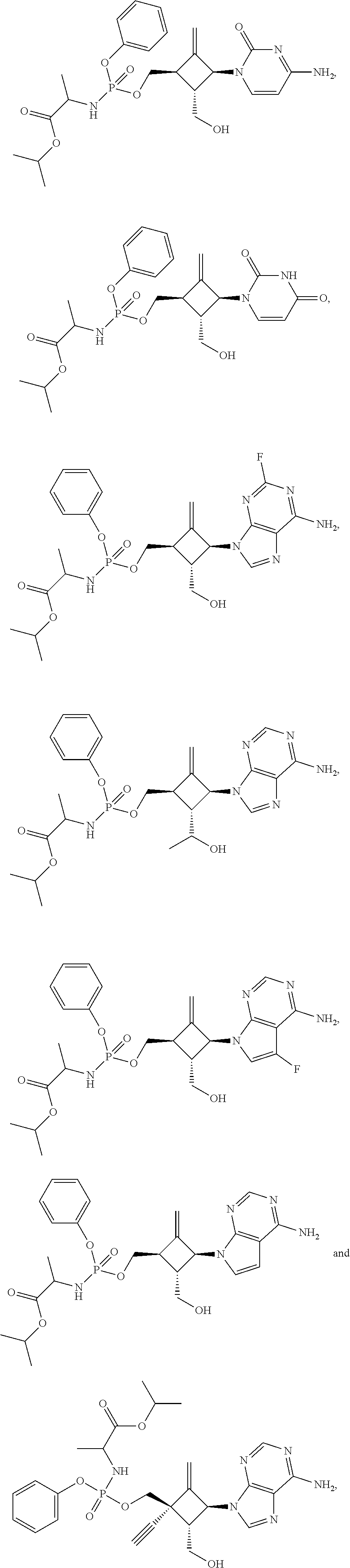
or a pharmaceutically acceptable salt of any of the foregoing.

91. A pharmaceutical composition comprising an effective amount of a compound of any one of claims 1-90, or a pharmaceutically acceptable salt thereof, and a pharmaceutically acceptable carrier, diluent, excipient or combination thereof.

92. Use of a compound of any one of claims 1-90, or a pharmaceutically acceptable salt thereof, or the pharmaceutical composition of claim 91, for preparing a medicament for treating a HBV and/or HDV infection.

93. Use of a compound of any one of claims 1-90, or a pharmaceutically acceptable salt thereof, or the pharmaceutical composition of claim 91, for preparing a medicament for reducing the reoccurrence of a HBV and/or HDV infection.

94. Use of a compound of any one of claims 1-90, or a pharmaceutically acceptable salt thereof, or the pharmaceutical composition of claim 91, for preparing a medicament for inhibiting replication of a HBV and/or HDV virus.

95. The use of any one of claims 92-94, further comprising the use of one or more agents selected from the group consisting of a HBV and/or HDV polymerase inhibitor, an immunomodulatory agent, an interferon, a pegylated interferon, a viral fusion/entry inhibitor, a viral maturation inhibitor, a capsid assembly modulator, a reverse transcriptase inhibitor, a cyclophilin/TNF inhibitor, a FXR agonist, a TLR-agonist, an siRNA or ASO cccDNA inhibitor, a gene silencing agent, an HBx inhibitor, an sAg secretion inhibitor, and an HBV vaccine, or a pharmaceutically acceptable salt of any of the aforementioned.

96. A method of ameliorating or treating a HBV and/or HDV infection, comprising administering to a subject suffering from the HBV and/or HDV infection an effective amount of a compound of any one of claims 1-90, or a pharmaceutically acceptable salt thereof, or the pharmaceutical composition of claim 91.

97. A method of ameliorating or treating a HBV and/or HDV infection, comprising contacting a cell infected with HBV and/or HDV with a compound of any one of claims 1-90, or a pharmaceutically acceptable salt thereof, or the pharmaceutical composition of claim 91.

98. A method of reducing the reoccurrence of a HBV and/or HDV infection, comprising contacting a cell infected with HBV and/or HDV with a compound of any one of claims 1-90, or a pharmaceutically acceptable salt thereof, or the pharmaceutical composition of claim 91.

99. A method of inhibiting replication of a HBV and/or HDV virus, comprising contacting a cell infected with HBV and/or HDV with a compound of any one of claims 1-90, or a pharmaceutically acceptable salt thereof, or the pharmaceutical composition of claim 91.

100. The method of any one of claims 96-99, further comprising the use of one or more agents selected from the group consisting of a HBV and/or HDV polymerase inhibitor, an immunomodulatory agent, an interferon, a pegylated interferon, a viral fusion/entry inhibitor, a viral maturation inhibitor, a capsid assembly modulator, a reverse transcriptase inhibitor, a cyclophilin/TNF inhibitor, a FXR agonist, a TLR-agonist, an siRNA or ASO cccDNA inhibitor, a gene silencing agent, an HBx inhibitor, an sAg secretion inhibitor, and an HBV vaccine, or a pharmaceutically acceptable salt of any of the aforementioned.

101. Use of a compound of any one of claims 1-90, or a pharmaceutically acceptable salt thereof, or the pharmaceutical composition of claim 91, for preparing a medicament for ameliorating or treating a HIV infection.

102. Use of a compound of any one of claims 1-90, or a pharmaceutically acceptable salt thereof, or the pharmaceutical composition of claim 91, for preparing a medicament for inhibiting replication of a HIV virus.

103. The use of any one of claims 101-102, further comprising the use of one or more antiretroviral therapy (ART) agents selected from the group consisting of a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy, or a pharmaceutically acceptable salt of any of the aforementioned.

104. A method of ameliorating or treating a HIV infection comprising administering to a subject suffering from the HIV infection an effective amount of a compound of any one of claims 1-90, or a pharmaceutically acceptable salt thereof, or the pharmaceutical composition of claim 91.

105. A method for inhibiting replication of a HIV virus comprising contacting a cell infected with the HIV virus with a compound of any one of claims 1-90, or a pharmaceutically acceptable salt thereof, or the pharmaceutical composition of claim 91.

106. A method for ameliorating or treating a HIV infection comprising contacting a cell infected with the HIV with a compound of any one of claims 1-90, or a pharmaceutically acceptable salt thereof, or the pharmaceutical composition of claim 91.

107. The method of any one of claims 104-106, further comprising one or more antiretroviral therapy (ART) agents selected from the group consisting of a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy, or a pharmaceutically acceptable salt of any of the aforementioned.
